(19) United States
(12) Patent Application Publication
(10) Pub. No.: US 2020/0057061 A1
(43) Pub. Date: Feb. 20, 2020

(54) BIOMARKER SIGNATURES OF SYSTEMIC LUPUS ERYTHEMATOSUS AND USES THEREOF

(71) Applicant: IMMUNOVIA AB, Lund (SE)

(72) Inventors: Carl BORREBAECK, Lund (SE); Payam DELFANI, Malmö (SE); Linda Dexlin MELLBY, Dalby (SE); Christer WINGREN, Södra Sandby (SE)

(21) Appl. No.: 16/308,258

(22) PCT Filed: Jun. 7, 2017
(86) PCT No.: PCT/EP2017/063852

§ 371 Date: Dec. 7, 2018

(30) Foreign Application Priority Data
Jun. 7, 2016 (GB) 1609950.9

(51) Int. Cl. G01N 33/564

(52) U.S. Cl. CPC G01N 33/564; G01N 2560/00; G01N 2800/60; G01N 2800/104

(57) ABSTRACT

The invention provides a method for determining a systemic lupus erythematosus-associated disease state in a subject comprising the steps of (a) providing a sample to be tested; and (b) measuring the presence and/or amount in the test sample of one or more biomarker(s) selected from the group defined in Table A, wherein the presence and/or amount in the test sample of the one or more biomarker(s) selected from the group defined in Table A is indicative of a systemic lupus. The invention also provides an array and a kit suitable for use in the methods of the invention.

### FIELD OF INVENTION

The present invention relates to biomarkers for the diagnosis, characterisation and prognosis of systemic lupus erythematosus (SLE), as well as signatures and arrays thereof and methods for use of the same.

### BACKGROUND

Systemic lupus erythematosus (SLE) is a chronic, multisystem, autoimmune connective tissue disease with a broad range of clinical manifestations. The disease aetiology is linked to multiple factors, including genetic, environmental, and hormonal factors, but the underlying mechanism is still largely unknown. Up to 30-50% of the SLE patients might suffer from glomerulonephritis, a condition of renal involvement and considered one of the most severe manifestation of SLE. Renal involvement in SLE carries significant morbidity and mortality.

Clinical manifestations vary widely among patients, and the signs and symptoms evolve over time, and overlap with those of other autoimmune diseases, why SLE is often misdiagnosed and/or overlooked. In fact, patients may spend up to four years and see three or more physicians before the disease is correctly diagnosed. On the other hand, SLE is also often over-diagnosed. The diagnosis of SLE in clinical practice is usually made according to the principles outlined by Fries and Holman; presence of typical manifestations from at least two organ systems in combination with immunological abnormality consistent with SLE in the absence of a better diagnostic alternative. However, during last years it has been concluded that a biopsy verified lupus glomerulonephritis in combination with immunological abnormality should be accepted for SLE diagnosis. Hence, novel means for improved diagnosis of SLE are needed.

Further, SLE classification criteria have been defined by the American College for Rheumatology (ACR) and more recently from systemic lupus International Collaborating Clinics (SLICC). According to ACR, SLE is classified when at least 4 of 11 clinical and/or immunological criteria, shared by many diseases, are fulfilled. In the case of SLICC, SLE is classified if (i) at least 4 of 17 clinical and immunological criteria, or (ii) biopsy verified lupus nephritis in the presence of antinuclear antibodies (ANA) or anti-dsDNA antibodies are met. In practice, this means that patients can display a very diverse set of symptoms, but all still be classified as similar.

Although major efforts have been made to decipher SLE-associated biomarkers, the output of validated and clinically useful biomarkers is still limited. In fact, there is no single laboratory blood- or urine-based test yet at hand that specifically and accurately can confirm or rule out the diagnosis of SLE. This lack of adequate biomarkers for SLE has hampered proper clinical management of patients with SLE. Considering the complexity and heterogeneity of SLE, a multiplex biomarker panel, rather than a single biomarker will be required to resolve this clinical need, placing high demands on the technologies used for biomarker discovery.

### SUMMARY OF THE INVENTION

The present invention provides an optimized recombinant antibody microarray platform. An optimized procedure for handling and analysing the microarray data was adopted. Further, the method allows SLE to be classified irrespective of the phenotype.

Accordingly, the first aspect the invention provides a method for determining a systemic lupus erythematosus-associated disease state in a subject comprising measuring the presence and/or amount in a test sample of one or more biomarker selected from the group defined in Table A, wherein the presence and/or amount in the one more test sample of the one or more biomarker(s) selected from the group defined in Table A is indicative of a systemic lupus erythematosus-associated disease state.

Alternatively or additionally, the first aspect the invention provides a method for determining a systemic lupus erythematosus-associated disease state in a subject comprising the steps of:
a) providing one or more sample to be tested; and
b) measuring the presence and/or amount in the test sample of one or more biomarker(s) selected from the group defined in Table A;

wherein the presence and/or amount in the one more test sample of the one or more biomarker(s) selected from the group defined in Table A is indicative of a systemic lupus erythematosus-associated disease state.

Thus, the invention provides biomarkers and biomarker signatures for determining a systemic lupus erythematosus-associated disease state in a subject.

By “systemic lupus erythematosus-associated disease state” we include the diagnosis, prognosis and/or characterisation of phenotypic subtype of SLE in the subject.

Thus, in one embodiment, the method is for diagnosing SLE in a subject.

Preferably, the individual is a human, but may be any mammal such as a domesticated mammal (preferably of agricultural or commercial significance including a horse, pig, cow, sheep, dog and cat).

For the avoidance of doubt, test samples from more than one disease state may be provided in step (a), for example, ≥2, ≥3, ≥4, ≥5, ≥6 or ≥7 different disease states. Step (a) may provide at least two test samples, for example, ≥3, ≥4, ≥5, ≥6, ≥7, ≥9, ≥10, ≥15, ≥20, ≥25, ≥50 or ≥100 test samples. Where multiple test samples are provided, they may be of the same type (e.g., all serum or urine samples) or of different types (e.g., serum and urine samples).

In one embodiment, the method further comprises the steps of:
c) providing one or more control sample from one or more individual with a different systemic lupus erythematosus-associated disease state to the test subject (i.e., a negative control); and
d) measuring the presence and/or amount in the control sample of the one or more biomarkers measured in step (b);

wherein the systemic lupus erythematosus-associated disease state is identified in the event that the presence and/or amount in the one or more test sample of the one or more biomarkers measured in step (b) is different from the presence and/or amount in the control sample.

For the avoidance of doubt, control samples from more than one disease state may be provided in step (c), for example, ≥2, ≥3, ≥4, ≥5, ≥6 or ≥7 different disease states. Step (c) may provide at least two control samples, for example, ≥3, ≥4, ≥5, ≥6, ≥7, ≥8, ≥9, ≥10, ≥15, ≥20, ≥25, ≥50 or ≥100 control samples Where multiple control samples are provided, they may be of the same type (e.g., all serum or urine samples) or of different types (e.g., serum and urine samples). Preferably the test samples types and control samples types are matched/corresponding.

The healthy individual may be free from SLE, autoimmune disease and/or renal disease. The healthy individual may be free from any form of disease.

The control sample of step (c) may be provided from an individual with:
(i) active (i.e. flaring) systemic lupus erythematosus (i.e. a SLEDAI score of greater than 4); and/or
(ii) passive/remissive systemic lupus erythematosus (i.e. a SLEDAI of 4 or below).

The control sample of step (c) may be provided from an individual with systemic lupus erythematosus subtype 1 (SLE-1), systemic lupus erythematosus subtype 2 (SLE-2) or systemic lupus erythematosus subtype 3 (SLE-3).

Alternatively or additionally the test sample of step (a) and/or the control sample of step (c) or step (e) is/are individually provided from:
a) an individual with SLE subtype 1 (SLE1);
b) an individual with SLE subtype 2 (SLE2); or
c) an individual with SLE subtype 3 (SLE3).

SLE1 comprises skin and musculoskeletal involvement but lacks serositis, systemic vasculitis and kidney involvement. SLE2 comprises skin and musculoskeletal involvement, serositis and systemic vasculitis but lacks kidney involvement. SLE3 comprises skin and musculoskeletal involvement, serositis, systemic vasculitis and SLE glomerulonephritis. SLE1, SLE2 and SLE3 represent mild/absent, moderate and severe SLE disease states, respectively (e.g., see Sturfelt G, Sjöholm AG.

Complement components, complement activation, and acute phase response in systemic lupus erythematosus. Int Arch Allergy Appi Immunol 1984; 75:75-83 which is incorporated herein by reference).

By “is different to the presence and/or amount in a control sample” we mean or include the presence and/or amount of the one or more biomarker in the test sample differs from that of the one or more control sample (or to predefined reference values representing the same). Preferably the presence and/or amount in the test sample differs from the presence or amount in the one or more control sample (or mean of the control samples) by at least ±5%, for example, at least ±6%, ±7%, ±8%, ±9%, ±10%, ±11%, ±12%, ±13%, ±14%, ±15%, ±16%, ±17%, ±18%, ±19%, ±20%, ±21%, ±22%, ±23%, ±24%, ±25%, ±26%, ±27%, ±28%, ±29%, ±30%, ±31%, ±32%, ±33%, ±34%, ±35%, 36%, ±37%, ±38%, ±39%, ±40%, ±41%, ±42%, ±43%, ±44%, ±45%, ±41%, ±42%, ±43%, ±44%, ±55%, ±60%, ±65%, ±66%, ±67%, ±68%, ±69%, ±70%, ±71%, ±72%, ±73%, ±74%, ±75%, ±76%, ±77%, ±78%, ±79%, ±80%, ±81%, ±82%, ±83%, ±84%, ±85%, ±86%, ±87%, ±88%, ±89%, ±90%, ±91%, ±92%, ±93%, ±94%, ±95%, ±96%, ±97%, ±98%, ±99%, ±100%, ±125%, ±150%, ±175%, ±200%, ±225%, ±250%, ±275%, ±300%, ±350%, ±400%, ±500% or at least ±1000% of the one or more control sample (e.g., the negative control sample).

Alternatively or additionally, the presence or amount in the test sample differs from the mean presence or amount in the control samples by at least >1 standard deviation from the mean presence or amount in the control samples, for example, ≥1.5, ≥2, ≥3, ≥4, ≥5, ≥6, ≥7, ≥8, ≥9, ≥10, ≥11, ≥12, ≥13, ≥14 or ≥15 standard deviations from the from the mean presence or amount in the control samples. Any suitable means may be used for determining standard deviation (e.g., direct, sum of square, Welford's), however, in one embodiment, standard deviation is determined using the direct method (i.e., the square root of [the sum the squares of the samples minus the mean, divided by the number of samples]).

Alternatively or additionally, by “is different to the presence and/or amount in a control sample” we mean or include that the presence or amount in the test sample does not correlate with the amount in the control sample in a statistically significant manner. By “does not correlate with the amount in the control sample in a statistically significant manner” we mean or include that the presence or amount in the test sample correlates with that of the control sample with a p-value of >0.001, for example, >0.002, >0.003, >0.004, >0.005, >0.01, >0.02, >0.03, >0.04 >0.05, >0.06, >0.07, >0.08, >0.09 or >0.1. Any suitable means for determining p-value known to the skilled person can be used, including z-test, t-test, Student's t-test, f-test, Mann-Whitney U test, Wilcoxon signed-rank test and Pearson's chi-squared test.

In an alternative or additional embodiment the method comprises the steps comprising or consisting of:
e) providing one or more control sample from an individual afflicted with the same systemic lupus erythematosus-associated disease state to the test subject (i.e., a positive control); and
f) measuring the presence and/or amount in the control sample of the one or more biomarkers measured in step (b);

wherein the systemic lupus erythematosus-associated disease state is identified in the event that the presence and/or amount in the test sample of the one or more biomarkers measured in step (b) corresponds to the presence and/or amount in the control sample of the one or more biomarkers measured in step (f).

By “corresponds to the presence and/or amount in a control sample” we mean or include the presence and/or amount is identical to that of a positive control sample; or closer to that of one or more positive control sample than to one or more negative control sample (or to predefined reference values representing the same). Preferably the presence and/or amount is within ±40% of that of the one or more control sample (or mean of the control samples), for example, within ±39%, ±38%, +37%, ±36%, ±35%, ±34%, ±33%, ±32%, ±31%, ±30%, +29%, ±28%, ±27%, ±26%, ±25%, ±24%, ±23%, ±22%, ±21%, ±20%, ±19%, ±18%, ±17%, ±16%, ±15%, ±14%, ±13%, ±12%, +11%, ±10%, ±9%, ±8%, ±7%, ±6%, ±5%, ±4%, ±3%, ±2%, ±1%, ±0.05% or within 0% of the one or more control sample (e.g., the positive control sample).

Alternatively or additionally, the difference in the presence or amount in the test sample is ≤5 standard deviation from the mean presence or amount in the control samples, for example, ≤4.5, ≤4, ≤3.5, 53, ≤2.5, ≤2, ≤1.5, ≤1.4, ≤1.3, ≤1.2, ≤1.1, 51, ≤0.9, ≤0.8, ≤0.7, ≤0.6, ≤0.5, ≤0.4, ≤0.3, ≤0.2, ≤0.1 or 0 standard deviations from the from the mean presence or amount in the control samples, provided that the standard deviation ranges for differing and corresponding biomarker expressions do not overlap (e.g., abut, but no not overlap).

Alternatively or additionally, by “corresponds to the presence and/or amount in a control sample” we mean or include that the presence or amount in the test sample correlates with the amount in the control sample in a statistically significant manner.

By “correlates with the amount in the control sample in a statistically significant manner” we mean or include that the presence or amount in the test sample correlates with the that of the control sample with a p-value of ≤0.05, for example, ≤0.04, ≤0.03, ≤0.02, ≤0.01, ≤0.005, ≤0.004, ≤0.003, ≤0.002, ≤0.001, ≤0.0005 or ≤0.0001.

Differential expression (up-regulation or down regulation) of biomarkers, or lack thereof, can be determined by any suitable means known to a skilled person. Differential expression is determined to a p value of a least less than 0.05 (p=<0.05), for example, at least <0.04, <0.03, <0.02, <0.01, <0.009, <0.005, <0.001, <0.0001, <0.00001 or at least <0.000001. Alternatively or additionally, differential expression is determined using a support vector machine (SVM). Alternatively or additionally, the SVM is an SVM as described below.

It will be appreciated by persons skilled in the art that differential expression may relate to a single biomarker or to multiple biomarkers considered in combination (i.e. as a biomarker signature). Thus, a p value may be associated with a single biomarker or with a group of biomarkers. Indeed, proteins having a differential expression p value of greater than 0.05 when considered individually may nevertheless still be useful as biomarkers in accordance with the invention when their expression levels are considered in combination with one or more other biomarkers.

As exemplified in the accompanying examples, the expression of certain biomarkers in a tissue, blood, serum or plasma test sample may be indicative of an SLE-associated disease state in an individual. For example, the relative expression of certain serum proteins in a single test sample may be indicative of the activity of SLE in an individual.

In an alternative or additional embodiment the presence and/or amount in the test sample of the one or more biomarkers measured in step (b) are compared against predetermined reference values representative of the measurements in steps (d) and/or (f).

In one embodiment, step (b) comprises or consists of measuring the presence and/or amount in the test sample of one or more of the biomarkers defined in Table A, for example, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62 or 63 of the biomarkers defined in Table A.

In one embodiment, step (b) comprises or consists of measuring the presence and/or amount in the test sample of one or more of the biomarkers defined in Table A(i), for example, two of the biomarkers defined in Table A(i).

In one embodiment, step (b) comprises or consists of measuring the presence and/or amount in the test sample of one or more of the biomarkers defined in Table A(ii), for example, 2, 3, 4, 5, 6, 7, 8, 9, 10, 11, 12, 13, 14, 15, 16, 17, 18 or 19 of the biomarkers defined in Table A(ii).

In one embodiment, step (b) comprises or consists of measuring the presence and/or amount in the test sample of one or more of the biomarkers defined in Table A(iii), for example, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41 or 42 of the biomarkers defined in Table A(iii).

Alternatively or additionally, step (b) comprises, consists of or excludes measuring the presence and/or amount in the test sample of MYOM2. Alternatively or additionally, step (b) comprises, consists of or excludes measuring the presence and/or amount in the test sample of ORP-3. Alternatively or additionally, step (b) comprises, consists of or excludes measuring the presence and/or amount in the test sample of APOA1. Alternatively or additionally, step (b) comprises, consists of or excludes measuring the presence and/or amount in the test sample of APOA4. Alternatively or additionally, step (b) comprises, consists of or excludes measuring the presence and/or amount in the test sample of ATP5B. Alternatively or additionally, step (b) comprises, consists of or excludes measuring the presence and/or amount in the test sample of CHX10. Alternatively or additionally, step (b) comprises, consists of or excludes measuring the presence and/or amount in the test sample of TBC1 D9. Alternatively or additionally, step (b) comprises, consists of or excludes measuring the presence and/or amount in the test sample of UPF3B. Alternatively or additionally, step (b) comprises, consists of or excludes measuring the presence and/or amount in the test sample of LUM. Alternatively or additionally, step (b) comprises, consists of or excludes measuring the presence and/or amount in the test sample of Digoxin. Alternatively or additionally, step (b) comprises, consists of or excludes measuring the presence and/or amount in the test sample of Surface Ag X. Alternatively or additionally, step (b) comprises, consists of or excludes measuring the presence and/or amount in the test sample of Motif (10). Alternatively or additionally, step (b) comprises, consists of or excludes measuring the presence and/or amount in the test sample of Motif (13). Alternatively or additionally, step (b) comprises, consists of or excludes measuring the presence and/or amount in the test sample of Motif (14). Alternatively or additionally, step (b) comprises, consists of or excludes measuring the presence and/or amount in the test sample of Motif (15). Alternatively or additionally, step (b) comprises, consists of or excludes measuring the presence and/or amount in the test sample of Motif (2). Alternatively or additionally, step (b) comprises, consists of or excludes measuring the presence and/or amount in the test sample of Motif (4). Alternatively or additionally, step (b) comprises, consists of or excludes measuring the presence and/or amount in the test sample of Motif (5). Alternatively or additionally, step (b) comprises, consists of or excludes measuring the presence and/or amount in the test sample of Motif (6). Alternatively or additionally, step (b) comprises, consists of or excludes measuring the presence and/or amount in the test sample of Motif (7). Alternatively or additionally, step (b) comprises, consists of or excludes measuring the presence and/or amount in the test sample of Motif (8). Alternatively or additionally, step (b) comprises, consists of or excludes measuring the presence and/or amount in the test sample of Angiomotin. Alternatively or additionally, step (b) comprises, consists of or excludes measuring the presence and/or amount in the test sample of C1-INH. Alternatively or additionally, step (b) comprises, consists of or excludes measuring the presence and/or amount in the test sample of C1q. Alternatively or additionally, step (b) comprises, consists of or excludes measuring the presence and/or amount in the test sample of C3. Alternatively or additionally, step (b) comprises, consists of or excludes measuring the presence and/or amount in the test sample of C4. Alternatively or additionally, step (b) comprises, consists of or excludes measuring the presence and/or amount in the test sample of CD40. Alternatively or additionally, step (b) comprises, consists of or excludes measuring the presence and/or amount in the test sample of CD40 ligand. Alternatively or additionally, step (b) comprises, consists of or excludes measuring the presence and/or amount in the test sample of Cystatin C. Alternatively or additionally, step (b) comprises, consists of or excludes measuring the presence and/or amount in the test sample of Factor B. Alternatively or additionally, step (b) comprises, consists of or excludes measuring the presence and/or amount in the test sample of GLP-1. Alternatively or additionally, step (b) comprises, consists of or excludes measuring the presence and/or amount in the test sample of GLP-1R. Alternatively or additionally, step (b) comprises, consists of or excludes measuring the presence and/or amount in the test sample of IgM. Alternatively or additionally, step (b) comprises, consists of or excludes measuring the presence and/or amount in the test sample of IL-11. Alternatively or additionally, step (b) comprises, consists of or excludes measuring the presence and/or amount in the test sample of IL-12. Alternatively or additionally, step (b) comprises, consists of or excludes measuring the presence and/or amount in the test sample of IL-13. Alternatively or additionally, step (b) comprises, consists of or excludes measuring the presence and/or amount in the test sample of IL-16. Alternatively or additionally, step (b) comprises, consists of or excludes measuring the presence and/or amount in the test sample of IL-18. Alternatively or additionally, step (b) comprises, consists of or excludes measuring the presence and/or amount in the test sample of IL-1ra. Alternatively or additionally, step (b) comprises, consists of or excludes measuring the presence and/or amount in the test sample of IL-2. Alternatively or additionally, step (b) comprises, consists of or excludes measuring the presence and/or amount in the test sample of IL-3. Alternatively or additionally, step (b) comprises, consists of or excludes measuring the presence and/or amount in the test sample of IL-4. Alternatively or additionally, step (b) comprises, consists of or excludes measuring the presence and/or amount in the test sample of IL-5. Alternatively or additionally, step (b) comprises, consists of or excludes measuring the presence and/or amount in the test sample of IL-6. Alternatively or additionally, step (b) comprises, consists of or excludes measuring the presence and/or amount in the test sample of IL-8. Alternatively or additionally, step (b) comprises, consists of or excludes measuring the presence and/or amount in the test sample of IL-9. Alternatively or additionally, step (b) comprises, consists of or excludes measuring the presence and/or amount in the test sample of Integrin α-10. Alternatively or additionally, step (b) comprises, consists of or excludes measuring the presence and/or amount in the test sample of JAK3. Alternatively or additionally, step (b) comprises, consists of or excludes measuring the presence and/or amount in the test sample of LDL. Alternatively or additionally, step (b) comprises, consists of or excludes measuring the presence and/or amount in the test sample of Lewis X. Alternatively or additionally, step (b) comprises, consists of or excludes measuring the presence and/or amount in the test sample of Lewis Y. Alternatively or additionally, step (b) comprises, consists of or excludes measuring the presence and/or amount in the test sample of MCP-1. Alternatively or additionally, step (b) comprises, consists of or excludes measuring the presence and/or amount in the test sample of MCP-3. Alternatively or additionally, step (b) comprises, consists of or excludes measuring the presence and/or amount in the test sample of MCP-4. Alternatively or additionally, step (b) comprises, consists of or excludes measuring the presence and/or amount in the test sample of Procathepsin W. Alternatively or additionally, step (b) comprises, consists of or excludes measuring the presence and/or amount in the test sample of Properdine. Alternatively or additionally, step (b) comprises, consists of or excludes measuring the presence and/or amount in the test sample of RANTES. Alternatively or additionally, step (b) comprises, consists of or excludes measuring the presence and/or amount in the test sample of Sialle Lewis X. Alternatively or additionally, step (b) comprises, consists of or excludes measuring the presence and/or amount in the test sample of TGF-31. Alternatively or additionally, step (b) comprises, consists of or excludes measuring the presence and/or amount in the test sample of TM peptide. Alternatively or additionally, step (b) comprises, consists of or excludes measuring the presence and/or amount in the test sample of TNF-α. Alternatively or additionally, step (b) comprises, consists of or excludes measuring the presence and/or amount in the test sample of TNF-β. Alternatively or additionally, step (b) comprises, consists of or excludes measuring the presence and/or amount in the test sample of VEGF.

In one embodiment, the biomarker mRNA and/or amino acid sequences correspond to those available on the GenBank database (http://www.ncbi.nlm.nih.gov/genbank/) and natural variants thereof. In a further embodiment, the biomarker mRNA and/or amino acid sequences correspond to those available on the GenBank database on 7 Jun. 2016.

Alternatively or additionally, the method excludes the use of biomarkers that are not listed in Table A and/or the present Examples section.

By ‘TM peptide’ we mean a peptide derived from a 10TM protein, to which the scFv antibody construct of SEQ ID NO:1 below has specificity (wherein the CDR sequences are underlined):

[SEQ ID NO: 1]
MAEVQLLESGGGLVQPGGSLRLSCAASGFTFSSYGFHWVRQAPGKGLEWV
SLISWDGGSTYYADSVKGRFTISRDNSKNTLYLQMNSLRAEDTAVYYCAR
GTWFDPWGQGTLVTVSSGGGGSGGGGSGGGGSQSVLTQPPSASGTPGQRV
TISCSGSSSNIGNNAVNWYQQLPGTAPKLLIYRNNQRPSGVPDRFSGSKS
GTSASLAISGLRSEDEADYYCAAWDDSLSWVFGGGTKLTVLG

Hence, this scFv may be used or any antibody, or antigen binding fragment thereof, that competes with this scFv for binding to the 10TM protein. For example, the antibody, or antigen binding fragment thereof, may comprise the same CDRs as present in SEQ ID NO:1.

It will be appreciated by persons skilled in the art that such an antibody may be produced with an affinity tag (e.g., at the C-terminus) for purification purposes. For example, an affinity tag of SEQ ID NO:2 below may be utilised:

[SEQ ID NO: 2]
DYKDHDGDYKDHDIDYKDDDDKAAAHHHHHH

By ‘Motif #’ (wherein ‘#’ represents a number) we include a protein comprising the selection motif shown in Table B. Alternatively or additionally we include a protein specifically bound by an antibody having the CDRs defined in Table B in respect of the motif in question. Alternatively or additionally the antibody has a framework region as defined in Olsson et al., 2012, ‘Epitope-specificity of recombinant antibodies reveals promiscuous peptide-binding properties.’ Protein Sci., 21(12):1897-910.

By “expression” we include the level or amount of a gene product such as mRNA or protein.

Generally, the systemic lupus erythematosus-associated disease state in a subject is determined with an ROC AUC of at least 0.55, for example with an ROC AUC of at least, 0.60, 0.65, 0.70, 0.75, 0.80, 0.85, 0.90, 0.95, 0.96, 0.97, 0.98 or with an ROC AUC of at least 0.99. Preferably, the systemic lupus erythematosus-associated disease state in an individual is determined with an ROC AUC of at least 0.85.

Typically, the systemic lupus erythematosus-associated disease state in a subject is determined using a support vector machine (SVM), such as those available from http://cran.r-project.org/web/packages/el071/index.html (e.g. e1071 1.5-24). However, any other suitable means may also be used.

Support vector machines (SVMs) are a set of related supervised learning methods used for classification and regression. Given a set of training examples, each marked as belonging to one of two categories, an SVM training algorithm builds a model that predicts whether a new example falls into one category or the other. Intuitively, an SVM model is a representation of the examples as points in space, mapped so that the examples of the separate categories are divided by a clear gap that is as wide as possible. New examples are then mapped into that same space and predicted to belong to a category based on which side of the gap they fall on.

More formally, a support vector machine constructs a hyperplane or set of hyperplanes in a high or infinite dimensional space, which can be used for classification, regression or other tasks. Intuitively, a good separation is achieved by the hyperplane that has the largest distance to the nearest training datapoints of any class (so-called functional margin), since in general the larger the margin the lower the generalization error of the classifier. For more information on SVMs, see for example, Burges, 1998, Data Mining and Knowledge Discovery, 2:121-167.

In one embodiment of the invention, the SVM is ‘trained’ prior to performing the methods of the invention using proteome samples from subjects assigned to known patient groups (namely, those patients in which the systemic lupus erythematosus-associated disease state is present versus those patients in which it is absent). By running such training samples, the SVM is able to learn what biomarker profiles are associated with the systemic lupus erythematosus-associated disease state. Once the training process is complete, the SVM is then able whether or not the proteome sample tested is from a subject a systemic lupus erythematosus-associated disease state.

However, this training procedure can be by-passed by pre-programming the SVM with the necessary training parameters. For example, a systemic lupus erythematosus-associated disease state in a subject can be determined using SVM parameters based on the measurement of some or all the biomarkers listed in Table A.

It will be appreciated by skilled persons that suitable SVM parameters can be determined for any combination of the biomarkers listed Table A by training an SVM machine with the appropriate selection of data (i.e. biomarker measurements in samples from known patient groups.

Alternatively, the data provided in the present figures and tables may be used to determine a particular SLE-associated disease state according to any other suitable statistical method known in the art, such as Principal Component Analysis (PCA) Orthogonal PCA (OPLS) and other multivariate statistical analyses (e.g., backward stepwise logistic regression model). For a review of multivariate statistical analysis see, for example, Schervish, Mark J. (November 1987). “A Review of Multivariate Analysis”. Statistical Science 2 (4): 396-413 which is incorporated herein by reference.

Preferably, the method of the invention has an accuracy of at least 51%, for example 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% accuracy.

Preferably, the method of the invention has a sensitivity of at least 51%, for example 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% sensitivity.

Preferably, the method of the invention has a specificity of at least 51%, for example 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% specificity.

By “accuracy” we mean the proportion of correct outcomes of a method, by “sensitivity” we mean the proportion of all positive chemicals that are correctly classified as positives, and by “specificity” we mean the proportion of all negative chemicals that are correctly classified as negatives.

Alternatively or additionally, the method is for diagnosing systemic lupus erythematosus in an individual; wherein the presence and/or amount in the test sample of the one or more biomarker(s) selected from the group defined in Table A is indicative of whether the individual has systemic lupus erythematosus. For example, step (b) may comprise or consist of measuring the presence and/or amount in the test sample of all of the biomarkers defined in Table A(i), Table A(iii) and/or Table A(iv).

By “diagnosing” we mean determining whether a subject is suffering from SLE. Conventional methods of diagnosing SLE are well known in the art.

The American College of Rheumatology established eleven criteria in 1982 (see Tan et al., 1982, The 1982 revised criteria for the classification of systemic lupus erythematosus, Arthritis. Rheum., 25:1271-7), which were revised in 1997 as a classificatory instrument to operationalise the definition of SLE in clinical trials (see Hochberg, 1997, Updating the American College of Rheumatology revised criteria for the classification of systemic lupus erythematosus, Arthritis. Rheum., 40:1725). For the purpose of identifying patients for clinical studies, a person is taken to have SLE if any 4 out of 11 symptoms are present simultaneously or serially on two separate occasions.

| Criterion | Definition |
|---|---|
| 1. Malar Rash | Fixed erythema, flat or raised, over the malar eminences, |
| | tending to spare the nasolabial folds |
| 2. Discoid rash | Erythematous raised patches with adherent keratotic |
| | scaling and follicular plugging; atrophic scarring may |
| | occur in older lesions |
| 3. Photosensitivity | Skin rash as a result of unusual reaction to sunlight, by |
| | patient history or physician observation |
| 4. Oral ulcers | Oral or nasopharyngeal ulceration, usually painless, |
| | observed by physician |
| 5. Nonerosive Arthritis | Involving 2 or more peripheral joints, characterized by |
| | tenderness, swelling, or effusion |
| 6. Pleuritis or Pericarditis | Pleuritis--convincing history of pleuritic pain or rubbing |
| | heard by a physician or evidence of pleural effusion |
| | OR |
| | Pericarditis--documented by electrocardigram or rub or |
| | evidence of pericardial effusion |
| 7. Renal Disorder | Persistent proteinuria > 0.5 grams per day or > than 3+ |
| | if quantitation not performed |
| | OR |
| | Cellular casts--may be red cell, hemoglobin, granular, |
| | tubular, or mixed |
| 8. Neurologic Disorder | Seizures in the absence of offending drugs or known |
| | metabolic derangements; e.g., uremia, ketoacidosis, or |
| | electrolyte imbalance |
| | OR |
| | Psychosis in the absence of offending drugs or known |
| | metabolic derangements, e.g., uremia, ketoacidosis, or |
| | electrolyte imbalance |
| 9. Hematologic Disorder | Hemolytic anemia--with reticulocytosis |
| | OR |
| | Leukopenia--<4,000/mm3 on ≥2 occasions |
| | OR |
| | Lyphopenia--<1,500/mm3 on ≥2 occasions |
| | OR |
| | Thrombocytopenia--<100,000/mm3 in the absence of |
| | offending drugs |
| 10. Immunologic Disorder | Anti-DNA: antibody to native DNA in abnormal titer |
| | OR |
| | Anti-Sm: presence of antibody to Sm nuclear antigen |
| | OR |
| | Positive finding of antiphospholipid antibodies on: |
| | (a) an abnormal serum level of IgG or IgM |
| | anticardiolipin antibodies, |
| | (b) a positive test result for lupus anticoagulant using |
| | a standard method, or |
| | (c) a false-positive test result for at least 6 months |
| | confirmed by Treponema pallidum immobilization or |
| | fluorescent treponemal antibody absorption test |
| 11. Antinuclear Antibody | An abnormal titer of antinuclear antibody by |
| | immunofluorescence or an equivalent assay at any point |
| | in time and in the absence of drugs |

Some people, especially those with antiphospholipid syndrome, may have SLE without four of the above criteria, and also SLE may present with features other than those listed in the criteria (see Asherson et al., 2003, Catastrophic antiphospholipid syndrome: international consensus statement on classification criteria and treatment guidelines, Lupus, 12(7):530-4; Sangle et al., 2005, Livedo reticularis and pregnancy morbidity in patients negative for antiphospholipid antibodies, Ann. Rheum. Dis., 64(1):147-8; and Hughes and Khamashta, 2003, Seronegative antiphospholipid syndrome, Ann. Rheum. Dis., 62(12):1127).

Recursive partitioning has been used to identify more parsimonious criteria (see Edworthy et al., 1988, Analysis of the 1982 ARA lupus criteria data set by recursive partitioning methodology: new insights into the relative merit of individual criteria, J. Rheumatol., 15(10):1493-8). This analysis presented two diagnostic classification trees:
Simplest classification tree: SLE is diagnosed if a person has an immunologic disorder (anti-DNA antibody, anti-Smith antibody, false positive syphilis test, or LE cells) or malar rash.
Full classification tree: Uses 6 criteria.

Alternatively or additionally, the diagnosis of SLE in is made according to the principles outlined by Fries and Holman, in: Smith L H Jr, ed. In: Smith L H Jr, ed. major Problems in Internal Medicine. Vol VI., 1976, which is incorporated herein by reference.

Other alternative set of criteria has been suggested, the St. Thomas' Hospital “alternative” criteria in 1998 (see Hughes, 1998, Is it lupus? The St. Thomas' Hospital “alternative” criteria, Clin. Exp. Rheumatol., 16(3):250-2).

However, these criteria were not intended to be used to diagnose individuals. They are time-consuming, subjective, require a high degree of experience to use effectively and have a high frequency of excluding actual SLE sufferers (i.e., diagnosing SLE patients as non-SLE patients). The present invention addresses these problems, providing objective SLE diagnosis.

Alternatively or additionally the method is for characterising systemic lupus erythematosus in an individual; wherein the presence and/or amount in the test sample of the one or more biomarker(s) selected from the group defined in Table A is indicative of whether the individual has systemic lupus erythematosus, subtype 1, subtype 2 or subtype 3. For example, step (b) may comprise or consist of measuring the presence and/or amount in the test sample of all of the biomarkers defined in Table A(i), Table A(ii) and/or Table A(iii).

By “characterising” or “classifying” we include determining the phenotypic subtype of SLE in a subject. SLE1 comprises skin and musculoskeletal involvement but lacks serositis, systemic vasculitis and kidney involvement. SLE2 comprises skin and musculoskeletal involvement, serositis and systemic vasculitis but lacks kidney involvement. SLE3 comprises skin and musculoskeletal involvement, serositis, systemic vasculitis and SLE glomerulonephritis. SLE1, SLE2 and SLE3 represent mild/absent, moderate and severe SLE disease states, respectively.

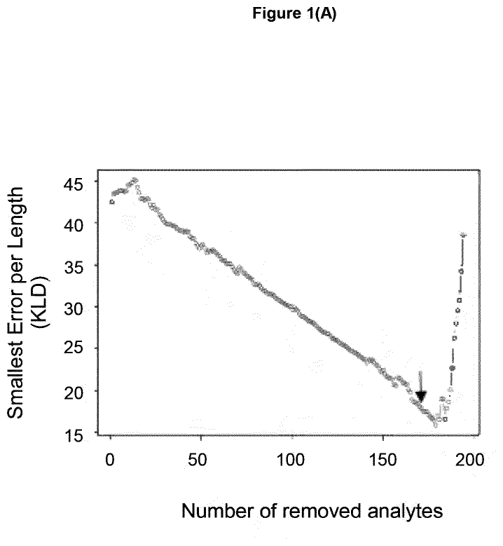

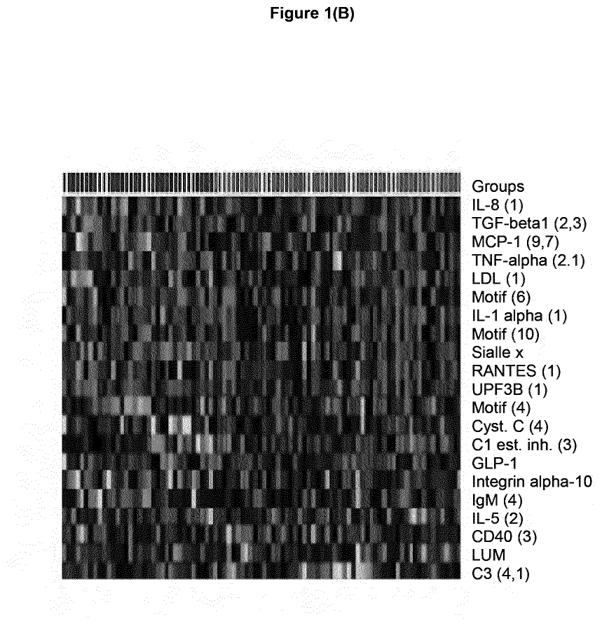

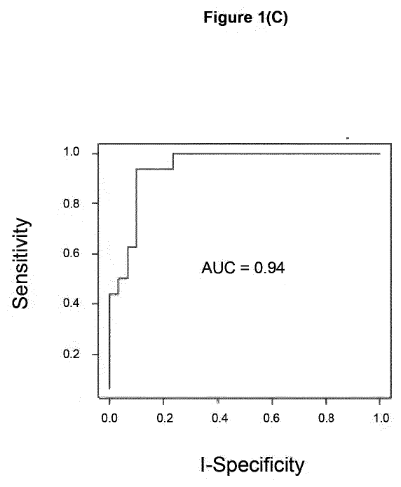

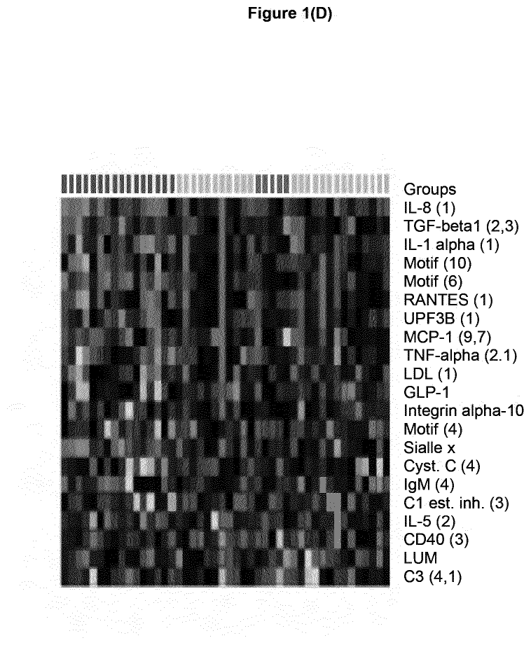

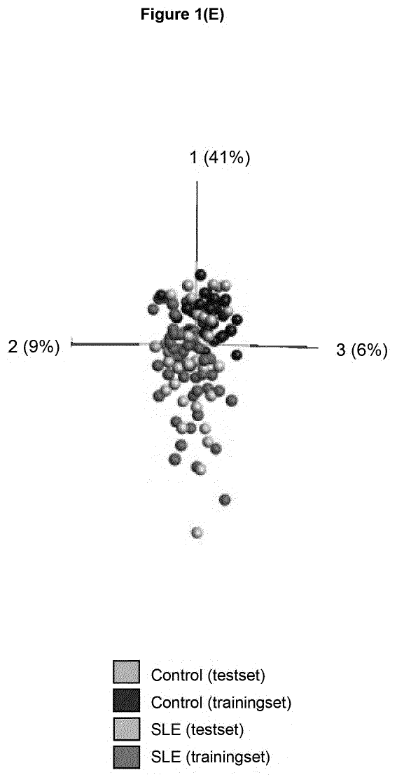

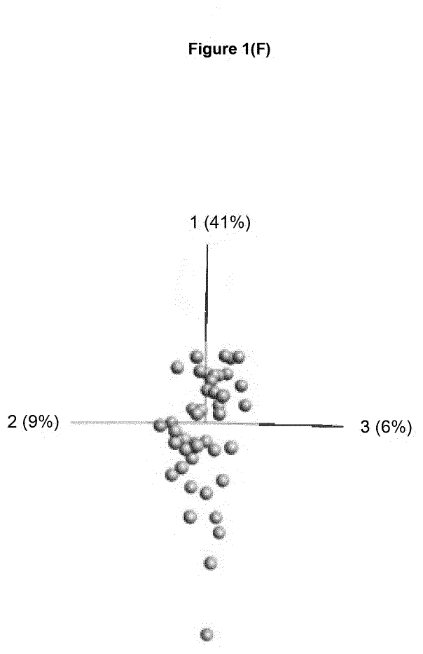

Alternatively or additionally the method is for diagnosing systemic lupus erythematosus in an individual, wherein step (b) comprises or consists of measuring the presence and/or amount in the test sample of one or more of the biomarkers defined in FIG. 1B for example, 2, 3, 4, 5, 6, 7, 8, 9, 10, 11, 12, 13, 14, 15, 16, 17, 18, 19, 20 or 21 of the biomarkers defined in FIG. 1B. The sample provided in step (a) may be an unfractionated blood sample, a plasma sample or a serum sample.

Alternatively or additionally the method is for diagnosing systemic lupus erythematosus in an individual, wherein step (b) comprises or consists of measuring the presence and/or amount in the test sample of one or more of the biomarkers defined in FIG. 1C for example, 2, 3, 4, 5, 6, 7, 8, 9, 10, 11, 12, 13, 14, 15, 16, 17, 18, 19, 20 or 21 of the biomarkers defined in FIG. 1C. The sample provided in step (a) may be an unfractionated blood sample, a plasma sample or a serum sample.

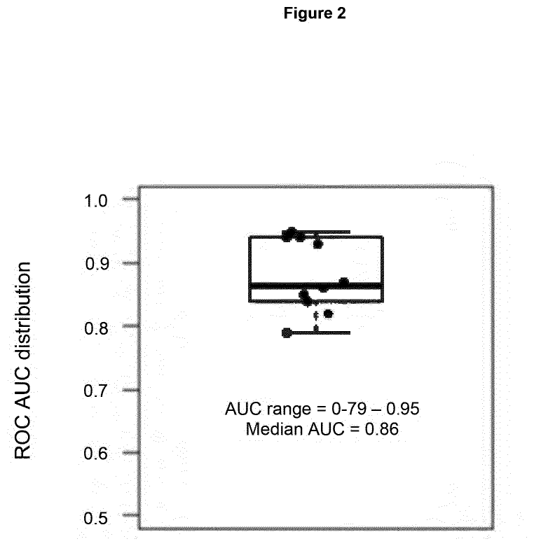

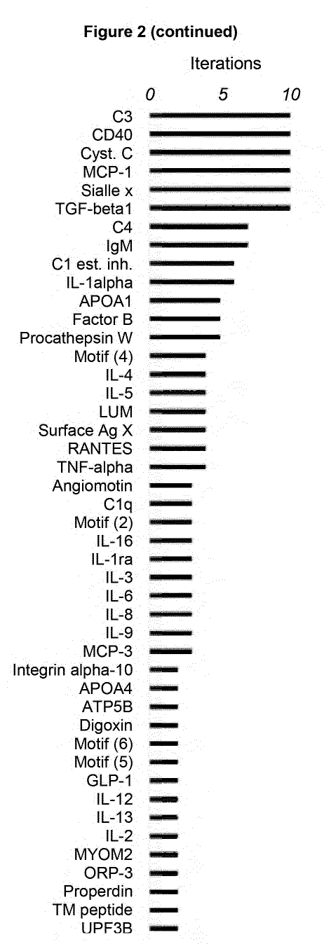

Alternatively or additionally the method is for diagnosing systemic lupus erythematosus in an individual, wherein step (b) comprises or consists of measuring the presence and/or amount in the test sample of one or more of the biomarkers defined in FIG. 2B for example, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44 or 45 of the biomarkers defined in FIG. 2B. The sample provided in step (a) may be an unfractionated blood sample, a plasma sample or a serum sample.

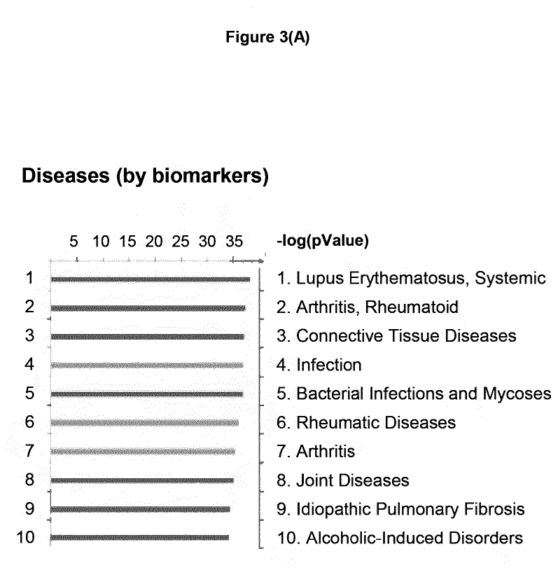

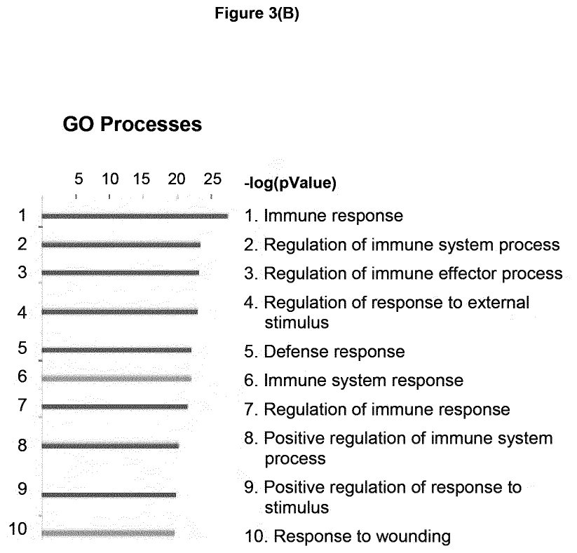

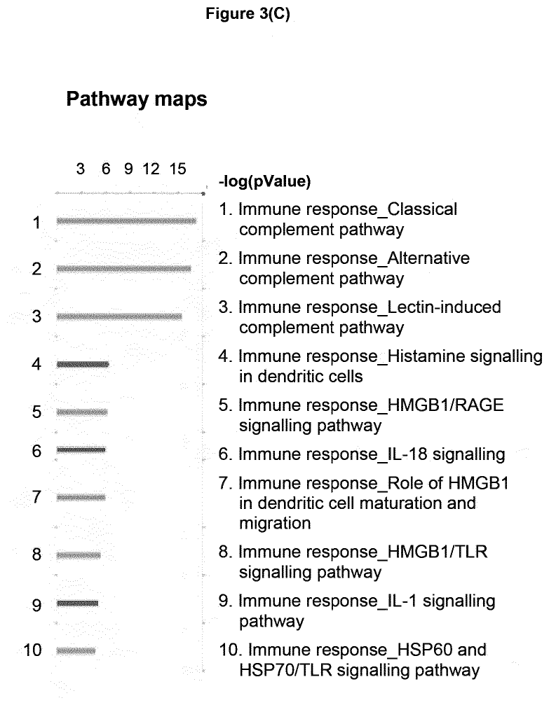

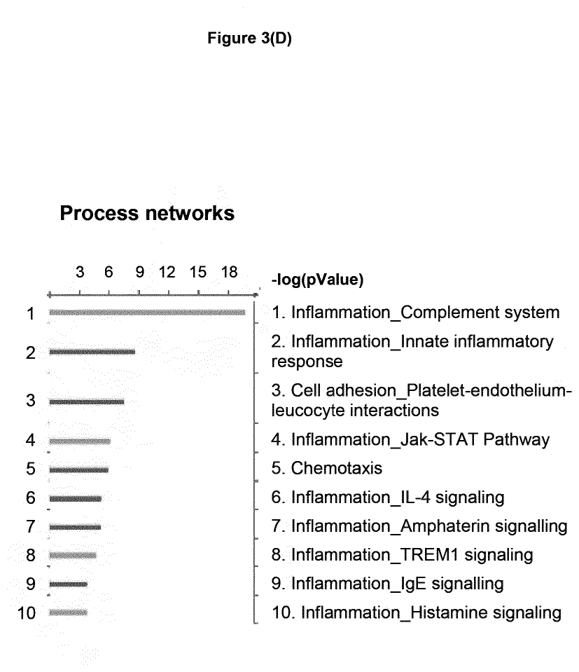

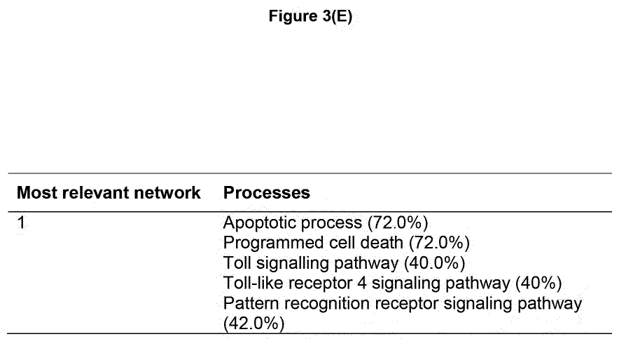

Alternatively or additionally the method is for diagnosing and/or characterising systemic lupus erythematosus type 1 in an individual (SLE1); wherein step (b) comprises or consists of measuring the presence and/or amount in the test sample of one or more of the biomarkers defined in FIG. 3A for example, 2, 3, 4, 5, 6, 7, 8, 9, 10, 11, 12, 13, 14, 15, 16, 17 or 18 of the biomarkers defined in FIG. 3A. The sample provided in step (a) may be an unfractionated blood sample, a plasma sample or a serum sample.

Alternatively or additionally the method is for diagnosing and/or characterising systemic lupus erythematosus type 2 in an individual (SLE2); wherein step (b) comprises or consists of measuring the presence and/or amount in the test sample of one or more of the biomarkers defined in FIG. 3B for example, 2, 3, 4, 5, 6, 7, 8, 9, 10, 11, 12, 13, 14, 15, 16, 17, 18 or 19 of the biomarkers defined in FIG. 3B. The sample provided in step (a) may be an unfractionated blood sample, a plasma sample or a serum sample.

Alternatively or additionally the method is for diagnosing and/or characterising systemic lupus erythematosus type 3 in an individual (SLE3); wherein step (b) comprises or consists of measuring the presence and/or amount in the test sample of one or more of the biomarkers defined in FIG. 3C for example, 2, 3, 4, 5, 6, 7, 8, 9, 10, 11, 12, 13, 14, 15, 16 or 17 of the biomarkers defined in FIG. 3C. The sample provided in step (a) may be an unfractionated blood sample, a plasma sample or a serum sample.

Alternatively or additionally the method is for diagnosing and/or characterising systemic lupus erythematosus type 1 (SLE1), systemic lupus erythematosus type 2 (SLE2) or systemic lupus erythematosus type 3 (SLE3); wherein step (b) comprises or consists of measuring the presence and/or amount in the test sample of one or more of the biomarkers defined in FIG. 3D for example, 2, 3, 4, 5, 6, 7, 8, 9, 10, 11, 12, 13, 14, 15, 16, 17, 18, 19, 20, 21, 22, 23, 24, 25, 26, 27, 28, 29, 30 of the biomarkers defined in FIG. 3D. The sample provided in step (a) may be an unfractionated blood sample, a plasma sample or a serum sample.

SLE disease severity and progression are conventionally determined through a clinical assessment and scoring using the following (SLEDAI-2000) criteria (see Gladman et al., 2002; J. Rheumatol., 29(2):288-91):

| Wt | Descriptor | Definition |
|---|---|---|
| 8 | Seizure | Recent onset. Exclude metabolic, infectious or drug cause. |
| 8 | Psychosis | Altered ability to function in normal activity due to severe |
| | | disturbance in the perception of reality. Include hallucinations, |
| | | incoherence, marked loose associations, impoverished thought |
| | | content, marked illogical thinking, bizarre, disorganized, or |
| | | catatonic behaviour. Excluded uraemia and drug causes. |
| 8 | Organic Brain | Altered mental function with impaired orientation, memory or |
| | Syndrome | other intelligent function, with rapid onset fluctuating clinical |
| | | features. Include clouding of consciousness with reduced |
| | | capacity to focus, and inability to sustain attention to |
| | | environment, plus at least two of the following: perceptual |
| | | disturbance, incoherent speech, insomnia or daytime |
| | | drowsiness, or increased or decreased psychomotor activity. |
| | | Exclude metabolic, infectious or drug causes. |
| 8 | Visual Disturbance | Retinal changes of SLE. Include cytoid bodies, retinal |
| | | hemorrhages, serious exodate or hemorrhages in the choroids, |
| | | or optic neuritis. Exclude hypertension, infection, or drug |
| | | causes. |
| 8 | Cranial Nerve | New onset of sensory or motor neuropathy involving cranial |
| | Disorder | nerves. |
| 8 | Lupus Headache | Severe persistent headache: may be migrainous, but must be |
| | | non-responsive to narcotic analgesia. |
| 8 | CVA | New onset of cerebrovascular accident(s). Exclude |
| | | arteriosclerosis. |
| 8 | Vasculitis | Ulceration, gangrene, tender finger nodules, periungual, |
| | | infarction, splinter hemorrhages, or biopsy or angiogram proof |
| | | of vasculitis. |
| 4 | Arthritis | More than 2 joints with pain and signs of inflammation (i.e. |
| | | tenderness, swelling, or effusion). |
| 4 | Myositis | Proximal muscle aching/weakness, associated with elevated |
| | | creatine phosphokinase/adolase or electromyogram changes |
| | | or a biopsy showing myositis. |
| 4 | Urinary Casts | Heme-granular or red blood cell casts. |
| 4 | Hematuria | >5 red blood cells/high power field. Exclude stone, infection or |
| | | other cause. |
| 4 | Proteinuria | >0.5 gm/24 hours. New onset or recent increase of more than |
| | | 0.5 gm/24 hours. |
| 4 | Pyuria | >5 white blood cells/high power field. Exclude infection. |
| 2 | Rash | Inflammatory type rash. |
| 2 | Alopecia | Abnormal, patchy or diffuse loss of hair. |
| 2 | Mucosal Ulcers | Oral or nasal ulcerations. |
| 2 | Pleurisy | Pleuritic chest pain with pleural rub or effusion, or pleural |
| | | thickening. |
| 2 | Pericarditis | Pericardial pain with at least one of the following: rub, effusion, |
| | | or electrocardiogram confirmation. |
| 2 | Low Complement | Decrease in CH50, C3, or C4 below the lower limit of normal |
| | | for testing laboratory. |
| 2 | Increased DNA | >25% binding by Fan assay or above normal range for testing |
| | binding | laboratory. |
| 1 | Fever | >38° C. Exclude infectious cause |
| 1 | Thrombocytopenia | <100,000 platelets/×10⁹/L. Exclude drug causes. |
| 1 | Leukopenia | <3,000 White blood cell/×10⁹/L. Exclude drug causes. |

The corresponding score/weight is applied if a descriptor is present at the time of visit or in the proceeding 10 to 30 days. The score is then totaled. A skilled person will appreciate that the SLEDAI boundaries of passive (remissive) SLE and active (flaring) SLE may vary according to the patient group being assessed.

Thus, in one embodiment the lower range for passive (remissive) SLE may be any one of 0, 1, 2, 3, 4, 5, 6, 7, 8, 9, 10, 11, 12, 13, 14, 15, 16, 17, 18, 19 or 20; the upper range for passive (remissive) SLE may be any one of 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44 or 45; the lower range for active or high active (flaring) SLE may be any one of 5, 6, 7, 8, 9, 10, 11, 12, 13, 14, 15, 16, 17, 18, 19 or 20; the upper range for mid severity SLE may be any one of 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85; the upper range for active or high active (flaring) SLE may be any one of 15, 16, 17, 18, 19 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 105 or 105; with the provisos that the lower range of a particular severity level must be of a lower score than its higher range and the ranges of each severity level may not overlap.

However, in one embodiment a total SLEDAI score of 0-4 indicates passive (remissive) SLE and a score of 5 or greater indicates active (flaring).

Alternatively or additionally, an increase in SLEDAI score of >3 from the previous assessment indicates mild or moderate flare. An increase in SLEDAI score of >12 from the previous assessment indicates severe flare. A decrease in SLEDAI score of >3 from the previous assessment indicates mild or moderate remission. A decrease in SLEDAI score of >12 from the previous assessment indicates advanced remission. An increase or decrease in SLEDAI score of <3 indicates stable (neither flaring nor non-flaring) SLE.

In one embodiment, the control sample of step (c) is provided from a healthy individual or an individual with systemic lupus erythematosus.

In an alternative or additional embodiment step (b) comprises measuring the expression of the protein or polypeptide of the one or more biomarker(s).

Methods of detecting and/or measuring the concentration of protein and/or nucleic acid are well known to those skilled in the art, see for example Sambrook and Russell, 2001, Cold Spring Harbor Laboratory Press.

Preferred methods for detection and/or measurement of protein include Western blot, North-Western blot, immunosorbent assays (ELISA), antibody microarray, tissue microarray (TMA), immunoprecipitation, in situ hybridisation and other immunohistochemistry techniques, radioimmunoassay (RIA), immunoradiometric assays (IRMA) and immunoenzymatic assays (IEMA), including sandwich assays using monoclonal and/or polyclonal antibodies. Exemplary sandwich assays are described by David et al., in U.S. Pat. Nos. 4,376,110 and 4,486,530, hereby incorporated by reference. Antibody staining of cells on slides may be used in methods well known in cytology laboratory diagnostic tests, as well known to those skilled in the art.

Typically, ELISA involves the use of enzymes which give a coloured reaction product, usually in solid phase assays. Enzymes such as horseradish peroxidase and phosphatase have been widely employed. A way of amplifying the phosphatase reaction is to use NADP as a substrate to generate NAD which now acts as a coenzyme for a second enzyme system. Pyrophosphatase from Escherichia coli provides a good conjugate because the enzyme is not present in tissues, is stable and gives a good reaction colour. Chemi-luminescent systems based on enzymes such as luciferase can also be used.

Conjugation with the vitamin biotin is frequently used since this can readily be detected by its reaction with enzyme-linked avidin or streptavidin to which it binds with great specificity and affinity.

Preferred methods for detection and/or measurement of nucleic acid (e.g. mRNA) include southern blot, northern blot, polymerase chain reaction (PCR), reverse transcriptase PCR (RT-PCR), quantitative real-time PCR (qRT-PCR), nanoarray, microarray, macroarray, autoradiography and in situ hybridisation.

In one embodiment, step (b), (d) and/or step (f) is performed using a first binding agent capable of binding to the one or more biomarker(s).

Binding agents (also referred to as binding molecules) can be selected from a library, based on their ability to bind a given motif, as discussed below.

In one embodiment, the first binding agent is an antibody or a fragment thereof. Thus, a fragment may contain one or more of the variable heavy (V_{H}) or variable light (V_{L}) domains. For example, the term antibody fragment includes Fab-like molecules (Better et al (1988) Science 240, 1041); Fv molecules (Skerra et al (1988) Science 240, 1038); single-chain Fv (ScFv) molecules where the V_{H} and V_{L} partner domains are linked via a flexible oligopeptide (Bird et al (1988) Science 242, 423; Huston et al (1988) Proc. Natl. Acad. Sci. USA 85, 5879) and single domain antibodies (dAbs) comprising isolated V domains (Ward et al (1989) Nature 341, 544).

The term “antibody variant” includes any synthetic antibodies, recombinant antibodies or antibody hybrids, such as but not limited to, a single-chain antibody molecule produced by phage-display of immunoglobulin light and/or heavy chain variable and/or constant regions, or other immunointeractive molecule capable of binding to an antigen in an immunoassay format that is known to those skilled in the art.

A general review of the techniques involved in the synthesis of antibody fragments which retain their specific binding sites is to be found in Winter & Milstein (1991) Nature 349, 293-299.

Additionally or alternatively at least one type, more typically all of the types, of the binding molecules is an aptamer.

Molecular libraries such as antibody libraries (Clackson et al, 1991, Nature 352, 624-628; Marks et al, 1991, J Mol Biol 222(3): 581-97), peptide libraries (Smith, 1985, Science 228(4705): 1315-7), expressed cDNA libraries (Santi et al (2000) J Mol Biol 296(2): 497-508), libraries on other scaffolds than the antibody framework such as affibodies (Gunneriusson et al, 1999, Appl Environ Microbiol 65(9): 4134-40) or libraries based on aptamers (Kenan et al, 1999, Methods Mol Biol 118, 217-31) may be used as a source from which binding molecules that are specific for a given motif are selected for use in the methods of the invention.

The molecular libraries may be expressed in vivo in prokaryotic (Clackson et al, 1991, op. cit.; Marks et al, 1991, op. cit.) or eukaryotic cells (Kieke et al, 1999, Proc Nat Acad Sci USA, 96(10):5651-6) or may be expressed in vitro without involvement of cells (Hanes & Pluckthun, 1997, Proc Natl Acad Sci USA 94(10):4937-42; He & Taussig, 1997, Nucleic Acids Res 25(24):5132-4; Nemoto et al, 1997, FEBS Lett, 414(2):405-8).

In cases when protein based libraries are used often the genes encoding the libraries of potential binding molecules are packaged in viruses and the potential binding molecule is displayed at the surface of the virus (Clackson et al, 1991, op. cit.; Marks et al, 1991, op. cit; Smith, 1985, op. cit.).

The most commonly used such system today is filamentous bacteriophage displaying antibody fragments at their surfaces, the antibody fragments being expressed as a fusion to the minor coat protein of the bacteriophage (Clackson et al, 1991, op. cit.; Marks et al, 1991, op. cit). However, also other systems for display using other viruses (EP 39578), bacteria (Gunneriusson et al, 1999, op. cit.; Daugherty et al, 1998, Protein Eng 11(9):825-32; Daugherty et al, 1999, Protein Eng 12(7):613-21), and yeast (Shusta et al, 1999, J Mol Biol 292(5):949-56) have been used.

In addition, recently, display systems utilising linkage of the polypeptide product to its encoding mRNA in so called ribosome display systems (Hanes & Pluckthun, 1997, op. cit.; He & Taussig, 1997, op. cit.; Nemoto et al, 1997, op. cit.), or alternatively linkage of the polypeptide product to the encoding DNA (see U.S. Pat. No. 5,856,090 and WO 98/37186) have been presented.

When potential binding molecules are selected from libraries one or a few selector peptides having defined motifs are usually employed. Amino acid residues that provide structure, decreasing flexibility in the peptide or charged, polar or hydrophobic side chains allowing interaction with the binding molecule may be used in the design of motifs for selector peptides. For example—
(i) Proline may stabilise a peptide structure as its side chain is bound both to the alpha carbon as well as the nitrogen;
(ii) Phenylalanine, tyrosine and tryptophan have aromatic side chains and are highly hydrophobic, whereas leucine and isoleucine have aliphatic side chains and are also hydrophobic;
(iii) Lysine, arginine and histidine have basic side chains and will be positively charged at neutral pH, whereas aspartate and glutamate have acidic side chains and will be negatively charged at neutral pH;
(iv) Asparagine and glutamine are neutral at neutral pH but contain a amide group which may participate in hydrogen bonds;
(v) Serine, threonine and tyrosine side chains contain hydroxyl groups, which may participate in hydrogen bonds.

Typically selection of binding molecules may involve the use of array technologies and systems to analyse binding to spots corresponding to types of binding molecules.

In one embodiment, the antibody or fragment thereof is a recombinant antibody or fragment thereof (such as an scFv).

By “ScFv molecules” we mean molecules wherein the V_{H} and V_{L} partner domains are linked via a flexible oligopeptide.

The advantages of using antibody fragments, rather than whole antibodies, are several-fold. The smaller size of the fragments may lead to improved pharmacological properties, such as better penetration of solid tissue. Effector functions of whole antibodies, such as complement binding, are removed. Fab, Fv, ScFv and dAb antibody fragments can all be expressed in and secreted from E. coli, thus allowing the facile production of large amounts of the said fragments.

Whole antibodies, and F(ab′)₂ fragments are “bivalent”. By “bivalent” we mean that the said antibodies and F(ab′)₂ fragments have two antigen combining sites. In contrast, Fab, Fv, ScFv and dAb fragments are monovalent, having only one antigen combining sites.

The antibodies may be monoclonal or polyclonal. Suitable monoclonal antibodies may be prepared by known techniques, for example those disclosed in “Monoclonal Antibodies: A manual of techniques”, H Zola (CRC Press, 1988) and in “Monoclonal Hybridoma Antibodies: Techniques and applications”, J G R Hurrell (CRC Press, 1982), both of which are incorporated herein by reference.

In one embodiment, the antibody or fragment thereof is selected from the group consisting of: scFv; Fab; a binding domain of an immunoglobulin molecule.

Alternatively or additionally, antibody or antigen-binding fragment is capable of competing for binding to a biomarker specified in Table A with an antibody for that biomarker defined in Table E.

By “capable of competing” for binding to a biomarker specified in Table A with an antibody molecule as defined herein (or a variant, fusion or derivative of said antibody or antigen-binding fragment, or a fusion of a said variant or derivative thereof, which retains the binding specificity for the required biomarker) we mean or include that the tested antibody or antigen-binding fragment is capable of inhibiting or otherwise interfering, at least in part, with the binding of an antibody molecule as defined herein.

For example, the antibody or antigen-binding fragment may be capable of inhibiting the binding of an antibody molecule defined herein by at least 10%, for example at least 20%, 30%, 40%, 50%, 60%, 70%, 80%, 90%, 35% or even by 100%.

Competitive binding may be determined by methods well known to those skilled in the art, such as ELISA (as described herein) and/or SPR (as described in the accompanying Examples).

Alternatively or additionally, the antibody or antigen-binding fragment is an antibody defined in Table E or an antigen-binding fragment thereof, or a variant thereof.

Alternatively or additionally, the antibody the antibody or antigen-binding fragment comprises a VH and VL domain specified in Table E, or a variant thereof.

By ‘variants’ of the antibody or antigen-binding fragment of the invention we include insertions, deletions and substitutions, either conservative or non-conservative. In particular we include variants of the sequence of the antibody or antigen-binding fragment where such variations do not substantially alter the activity of the antibody or antigen-binding fragment. In particular, we include variants of the antibody or antigen-binding fragment where such changes do not substantially alter the binding specificity for the respective biomarker specified in Table E.

The polypeptide variant may have an amino acid sequence which has at least 70% identity with one or more of the amino acid sequences of the antibody or antigen-binding fragment of the invention as defined herein—for example, at least 75%, at least 80%, at least 90%, at least 95%, at least 96%, at least 97%, at least 98%, at least 99% identity or 100% with one or more of the amino acid sequences of the antibody or antigen-binding fragment of the invention as defined herein.

The percent sequence identity between two polypeptides may be determined using suitable computer programs, for example the GAP program of the University of Wisconsin Genetic Computing Group and it will be appreciated that percent identity is calculated in relation to polypeptides whose sequences have been aligned optimally.

The alignment may alternatively be carried out using the Clustal W program (as described in Thompson et al., 1994, Nucl. Acid Res. 22:4673-4680, which is incorporated herein by reference).

The parameters used may be as follows:
Fast pair-wise alignment parameters: K-tuple(word) size; 1, window size; 5, gap penalty; 3, number of top diagonals; 5. Scoring method: x percent.
Multiple alignment parameters: gap open penalty; 10, gap extension penalty; 0.05.
Scoring matrix: BLOSUM.

Alternatively, the BESTFIT program may be used to determine local sequence alignments.

The antibodies may share CDRs (e.g., 1, 2, 3, 4, 5 or 6) CDRs with one or more of the antibodies defined in Table E.

CDRs can be defined using any suitable method known in the art. Commonly used methods include Paratome (Kunik, Ashkenazi and Ofran, 2012, ‘Paratome: an online tool for systematic identification of antigen-binding regions in antibodies based on sequence or structure’ Nucl. Acids Res., 40:W521-W524; http://www.ofranlab.org/paratome/), Kabat (Wu and Kabat, 1970, ‘An analysis of the sequences of the variable regions of Bence Jones proteins and myeloma light chains and their implications for antibody complementarity.’ J. Exp. Med., 132:211-250), Chothia (Chothia and Lesk, 1987 ‘Canonical structures for the hypervariable regions of immunoglobulins’ J. Mol. Biol., 196:901-917; Chothia et al., 1989 ‘Conformations of immunoglobulin hypervariable regions’ Nature, 342:877-883) and IMGT (Lefranc et al., 2003 ‘IMGT unique numbering for immunoglobulin and T cell receptor variable domains and Ig superfamily V-like domains.’ Dev. Comp. Immunol., 27:55-77; Lefranc et al., 2005 ‘IMGT unique numbering for immunoglobulin and T cell receptor constant domains and Ig superfamily C-like domains’ Dev. Comp. Immunol., 29:185-203; http://www.imgt.org). For example, the method used may be the IMGT method.

Alternatively or additionally, the first binding agent is immobilised on a surface (e.g., on a multiwell plate or array).

In one embodiment, the one or more biomarker(s) in the test sample is labelled with a detectable moiety.

In one embodiment, the one or more biomarker(s) in the control sample is labelled with a detectable moiety (which may be the same or different from the detectable moiety used to label the test sample).

By a “detectable moiety” we include the meaning that the moiety is one which may be detected and the relative amount and/or location of the moiety (for example, the location on an array) determined.

Detectable moieties are well known in the art.

A detectable moiety may be a fluorescent and/or luminescent and/or chemiluminescent moiety which, when exposed to specific conditions, may be detected. For example, a fluorescent moiety may need to be exposed to radiation (i.e. light) at a specific wavelength and intensity to cause excitation of the fluorescent moiety, thereby enabling it to emit detectable fluorescence at a specific wavelength that may be detected.

Alternatively, the detectable moiety may be an enzyme which is capable of converting a (preferably undetectable) substrate into a detectable product that can be visualised and/or detected. Examples of suitable enzymes are discussed in more detail below in relation to, for example, ELISA assays.

Alternatively, the detectable moiety may be a radioactive atom which is useful in imaging. Suitable radioactive atoms include ^{99m}Tc and ¹²³I for scintigraphic studies.

Other readily detectable moieties include, for example, spin labels for magnetic resonance imaging (MRI) such as ¹²³I again, ¹³¹I, ¹¹¹In, ¹⁹F, ¹³C, ¹⁵N, ¹⁷O, gadolinium, manganese or iron. Clearly, the agent to be detected (such as, for example, the one or more proteins in the test sample and/or control sample described herein and/or an antibody molecule for use in detecting a selected protein) must have sufficient of the appropriate atomic isotopes in order for the detectable moiety to be readily detectable.

The radio- or other labels may be incorporated into the agents of the invention (i.e. the proteins present in the samples of the methods of the invention and/or the binding agents of the invention) in known ways. For example, if the binding moiety is a polypeptide it may be biosynthesised or may be synthesised by chemical amino acid synthesis using suitable amino acid precursors involving, for example, fluorine-19 in place of hydrogen. Labels such as ^{99m}Tc, ¹²³I, ¹⁸⁶Rh, ¹⁸⁸Rh and ¹¹¹In can, for example, be attached via cysteine residues in the binding moiety. Yttrium-90 can be attached via a lysine residue. The IODOGEN method (Fraker et al (1978) Biochem. Biophys. Res. Comm. 80, 49-57) can be used to incorporate ¹²³I. Reference (“Monoclonal Antibodies in Immunoscintigraphy”, J-F Chatal, CRC Press, 1989) describes other methods in detail. Methods for conjugating other detectable moieties (such as enzymatic, fluorescent, luminescent, chemiluminescent or radioactive moieties) to proteins are well known in the art.

Preferably, the detectable moiety is selected from the group consisting of: a fluorescent moiety, a luminescent moiety, a chemiluminescent moiety, a radioactive moiety, and an enzymatic moiety.

In an alternative or additional embodiment step (b), (d) and/or (f) comprises measuring the expression of a nucleic acid molecule encoding the one or more biomarkers.

The nucleic acid molecule may be a cDNA molecule or an mRNA molecule. Preferably the nucleic acid molecule is an mRNA molecule. Also preferably the nucleic acid molecule is a cDNA molecule.

Hence, measuring the expression of the one or more biomarker(s) in step (b) may be performed using a method selected from the group consisting of Southern hybridisation, Northern hybridisation, polymerase chain reaction (PCR), reverse transcriptase PCR (RT-PCR), quantitative real-time PCR (qRT-PCR), nanoarray, microarray, macroarray, autoradiography and in situ hybridisation. Preferably measuring the expression of the one or more biomarker(s) in step (b) is determined using a DNA microarray. Hence, the method may comprise or consist of measuring the expression of the one or more biomarker(s) in step (b) using one or more binding moiety, each capable of binding selectively to a nucleic acid molecule encoding one of the biomarkers identified in Table A.

In an alternative or additional embodiment step the one or more binding moieties each comprise or consist of a nucleic acid molecule such as DNA, RNA, PNA, LNA, GNA, TNA or PMO (preferably DNA). Preferably the one or more binding moieties are 5 to 100 nucleotides in length. More preferably, the one or more nucleic acid molecules are 15 to 35 nucleotides in length. The binding moiety may comprise a detectable moiety.

Suitable binding agents (also referred to as binding molecules) may be selected or screened from a library based on their ability to bind a given nucleic acid, protein or amino acid motif.

In an alternative or additional embodiment measuring the expression of the one or more biomarker(s) in step (b), (d) and/or (f) is performed using one or more binding moieties, each individually capable of binding selectively to a nucleic acid molecule encoding one of the biomarkers identified in Table A.

In an alternative or additional embodiment, the nucleic acid binding moiety comprises a detectable moiety as defined above.

In one embodiment, step (b) is performed using an array.

In one embodiment, step (d) is performed using an array.

For example, the array may be a bead-based array or a surface-based array.

In one embodiment, the array is selected from the group consisting of macroarrays, microarrays and nanoarrays.

Arrays per se are well known in the art. Typically they are formed of a linear or two-dimensional structure having spaced apart (i.e. discrete) regions (“spots”), each having a finite area, formed on the surface of a solid support. An array can also be a bead structure where each bead can be identified by a molecular code or colour code or identified in a continuous flow. Analysis can also be performed sequentially where the sample is passed over a series of spots each adsorbing the class of molecules from the solution. The solid support is typically glass or a polymer, the most commonly used polymers being cellulose, polyacrylamide, nylon, polystyrene, polyvinyl chloride or polypropylene. The solid supports may be in the form of tubes, beads, discs, silicon chips, microplates, polyvinylidene difluoride (PVDF) membrane, nitrocellulose membrane, nylon membrane, other porous membrane, non-porous membrane (e.g. plastic, polymer, perspex, silicon, amongst others), a plurality of polymeric pins, or a plurality of microtitre wells, or any other surface suitable for immobilising proteins, polynucleotides and other suitable molecules and/or conducting an immunoassay. The binding processes are well known in the art and generally consist of cross-linking covalently binding or physically adsorbing a protein molecule, polynucleotide or the like to the solid support. By using well-known techniques, such as contact or non-contact printing, masking or photolithography, the location of each spot can be defined. For reviews see Jenkins, R. E., Pennington, S. R. (2001, Proteomics, 2, 13-29) and Lal et al (2002, Drug Discov Today 15; 7(18 Suppl):S143-9).

Typically, the array is a microarray. By “microarray” we include the meaning of an array of regions having a density of discrete regions of at least about 100/cm², and preferably at least about 1000/cm². The regions in a microarray have typical dimensions, e.g., diameters, in the range of between about 10-250 m, and are separated from other regions in the array by about the same distance. The array may also be a macroarray or a nanoarray.

Once suitable binding molecules (discussed above) have been identified and isolated, the skilled person can manufacture an array using methods well known in the art of molecular biology.

In one embodiment, step (b) is performed using an assay comprising a second binding agent capable of binding to the one or more proteins, the second binding agent having a detectable moiety.

In one embodiment, step (d) is performed using an assay comprising a second binding agent capable of binding to the one or more proteins, the second binding agent having a detectable moiety.

In one embodiment, the second binding agent is an antibody or a fragment thereof (for example, as described above in relation to the first binding agent).

Typically, the assay is an ELISA (Enzyme Linked Immunosorbent Assay) which typically involve the use of enzymes which give a coloured reaction product, usually in solid phase assays. Enzymes such as horseradish peroxidase and phosphatase have been widely employed. A way of amplifying the phosphatase reaction is to use NADP as a substrate to generate NAD which now acts as a coenzyme for a second enzyme system. Pyrophosphatase from Escherichia coli provides a good conjugate because the enzyme is not present in tissues, is stable and gives a good reaction colour. Chemi-luminescent systems based on enzymes such as luciferase can also be used.

Conjugation with the vitamin biotin is also employed used since this can readily be detected by its reaction with enzyme-linked avidin or streptavidin to which it binds with great specificity and affinity.

It will be appreciated by persons skilled in the art that there is a degree of fluidity in the biomarker composition of the signatures of the invention. Thus, different combinations of the biomarkers may be equally useful in the diagnosis, prognosis and/or characterisation of SLE. In this way, each biomarker (either alone or in combination with one or more other biomarkers) makes a contribution to the signature.

In an alternative or additional embodiment the sample provided in step (a), (c) and/or (e) is selected from the group consisting of unfractionated blood, plasma, serum, tissue fluid, breast tissue, milk, bile and urine. In an alternative or additional embodiment the sample provided in step (a), (c) and/or (e) is selected from the group consisting of unfractionated blood, plasma and serum. In an alternative or additional embodiment the sample provided in step (a), (c) and/or (e) is serum. In an alternative or additional embodiment the sample provided in step (a), (c) and/or (e) is urine. In an alternative or additional embodiment a serum sample and a urine sample are provided in step (a), (c) and/or (e).

In an alternative or additional embodiment the method comprises recording the diagnosis, prognosis or characterisation on a physical or electronic data carrier (i.e., physical or electronic file).

In an alternative or additional embodiment the method comprises the step of:
(g) determining an/the systemic lupus erythematosus-associated disease state in the subject based on the presence and/or amount in the test sample of the one or more biomarker(s) selected from the group defined in Table A.

In an alternative or additional embodiment in the event that the individual is diagnosed with SLE, the method comprises the step of:
(h) providing the individual with appropriate SLE therapy.

As noted above, in the event that the individual is not diagnosed with SLE, they may be subjected to further monitoring for SLE (for example, using the method of the present invention).

In an alternative or additional embodiment in the event that the individual is characterised or prognosed as having a flare in SLE (mild, moderate or severe), the method comprises the step of:
(h) providing the individual with appropriate SLE flare therapy.

As noted above, in the event that the individual is not diagnosed with SLE flare, they may be subjected to further monitoring for SLE flare (for example, using the method of the present invention).

The repeated monitoring may be repeated at least every 5 days, for example, at least every 10 days, at least every 15 days, at least every 20 days, at least every 25 days, at least every 30 days, at least every 2 months, at least every 3 months, at least every 4 months, at least every 5 months, at least every 6 months, at least every 7 months, at least every 8 months, at least every 9 months, at least every 10 months, at least every 11 months, at least every 12 months, at least every 18 months or at least every 24 months.

Monitoring may also continue in a repeated fashion regardless of whether or not the individual is found to have SLE or SLE flare.

In an alternative or additional embodiment, a more aggressive treatment may be provided for more aggressive SLE types (e.g., SLE3) or during an SLE flare. Suitable therapeutic approaches can be determined by the skilled person according to the prevailing guidance at the time, for example, the American College of Rheumatology Guidelines for Screening, Treatment, and Management of Lupus Nephritis (Hahn et al., 2012, Arthritis Care & Research, 64(6):797-808) which is incorporated herein by reference.

In an alternative or additional embodiment the SLE therapy is selected from the group consisting of systemic inflammation directed treatment (Antimalarials (Hydroxychloroquine), Corticosteroids, Pulse (or mini-pulse) cyclophosphamide (CTX) (with or without corticosteroid co-administration), Mycophenolate mofetil (MMF), Azathioprine (AZA), Methotrexate (MTX)), immune cell targeted therapies (Anti-CD20 antibodies (rituximab, atumumab, ocrelizumab and veltuzumumab), anti-CD22 (Epratuzumab), abetimus (LJP-394), belimumab, atacicept), co-stimulatory signalling pathway targeting (anti-ICOS (inducible costimulator) antibody, anti-ICOS-L (inducible costimulator ligand) antibody, anti-B7RP1 antibody (AMG557)), anti-cytokine therapy (anti-TNF therapy, anti-IL-10, anti-IL-1, anti-IL-18, anti-IL-6, anti-IL-15, memantine, anti-interferon-alpha (IFN-α), plasmapheresis (or plasma exchange), intravenous immunoglobulin (IVIG), DNA vaccination, statins, antioxidants (N-acetylcysteine (NAC), Cysteamine (CYST)), anti-IgE antibodies and anti-FcCRla antibodies, Syk (spleen tyrosine kinase) inhibition, and Jak (Janus kinase) inhibition), kidney excision, kidney transplant.

Accordingly, the present invention comprises an anti-SLE agent for use in treating SLE wherein the dosage regime is determined based on the results of the method of the first aspect of the invention.

The present invention comprises the use of an anti-SLE agent in treating SLE wherein the dosage regime is determined based on the results of the method of the first aspect of the invention.

The present invention comprises the use of an anti-SLE agent in the manufacture of a medicament for treating SLE wherein the dosage regime is determined based on the results of the method of the first aspect of the invention.

The present invention comprises a method of treating SLE comprising providing a sufficient amount of an anti-SLE agent wherein the type and amount of anti-SLE agent sufficient to treat the SLE is determined based on the results of the method of the first aspect of the invention.

A second aspect of the invention provides an array for determining a systemic lupus erythematosus-associated disease state in an individual comprising one or more binding agent as defined above in relation to the first aspect of the invention.

In one embodiment, the array is for use in a method according to the first aspect of the invention.

In another embodiment the array is for determining a disease state defined in the first aspect of the invention comprising or consisting of measuring the presence and/or amount of a corresponding biomarker or group of biomarkers defined in the first aspect of the invention.

In a further embodiment the array is an array defined in the first aspect of the invention.

In one embodiment, the one or more binding agent is capable of binding to all of the proteins defined in Table A.

A third aspect of the invention provides the use of one or more biomarkers selected from the group defined in Table A as a biomarker for determining a systemic lupus erythematosus-associated disease state in an individual. In one embodiment, all of the biomarkers defined in Table A are used as a biomarker for determining a systemic lupus erythematosus-associated disease state in an individual.

A fourth aspect of the invention provides the use of one or more biomarkers selected from the group defined in Table A in the manufacture of a medicament (e.g. a diagnostic agent) for determining a Systemic Lupus Erythematosus-associated disease state in an individual.

A fifth aspect of the invention provides one or more biomarkers selected from the group defined in Table A for determining a Systemic Lupus Erythematosus-associated disease state in an individual.

A sixth aspect of the invention provides use of one or more binding agent as defined in the first aspect of the invention for determining a Systemic Lupus Erythematosus-associated disease state in an individual. Alternatively or additionally all of the biomarkers defined in Table A are used for determining a Systemic Lupus Erythematosus-associated disease state in an individual. In one embodiment, the binding agent(s) is/are antibodies or antigen-binding fragments thereof.

A seventh aspect of the invention provides use of one or more binding agent as defined in the first aspect of the invention for the manufacture of a medicament (e.g. a diagnostic agent) for determining a Systemic Lupus Erythematosus-associated disease state in an individual. In one embodiment, the binding agent(s) is/are antibodies or antigen-binding fragments thereof.

An eighth aspect of the invention provides one or more binding agent as defined in the first aspect of the invention for determining a Systemic Lupus Erythematosus-associated disease state in an individual. In one embodiment, the binding agent(s) is/are antibodies or antigen-binding fragments thereof.

A ninth aspect of the invention provides a kit for determining a systemic lupus erythematosus-associated disease state in an individual comprising:
i) one or more first binding agent as defined above in relation to the first aspect of the invention; and
ii) (optionally) instructions for performing the method of the first aspect of the invention.

A tenth aspect of the invention provides a method of treating systemic lupus erythematosus in an individual comprising the steps of:
(a) determining a systemic lupus erythematosus-associated disease state in an individual according to the method defined in the first aspect of the invention; and
(b) providing the individual with systemic lupus erythematosus therapy.

By “Systemic lupus erythematosus therapy” we include treatment of the symptoms of systemic lupus erythematosus (SLE), most notably fatigue, joint pain/swelling and/or skin rashes.

Other symptoms of SLE can include:
a fever (high temperature)
swollen lymph glands (small glands found throughout your body, including in your neck, armpits and groin)
recurring mouth ulcers
hair loss (alopecia)
high blood pressure (hypertension)
headaches and migraines
stomach (abdominal) pain
chest pain
depression
dry eyes
memory loss
seizures (fits)
problems thinking clearly and difficulty telling the difference between reality and imagination (psychosis)
shortness of breath
Raynaud's phenomenon—a condition that limits the blood supply to your hands and feet when it is cold
ankle swelling and fluid retention (oedema)

Typically, treatment for SLE may include one or more of the following (see also above):
(a) Limiting exposure to the sun;
(b) Vitamin D supplements;
(c) Non-steroidal anti-inflammatory drugs (NSAIDs), such as ibuprofen;
(d) Antimalarial agents, such as hydroxychloroquine;
(e) Corticosteroids;
(f) Immunosuppressants;
(g) Rituximab; and
(h) Belimumab.

An eleventh aspect of the invention provides a computer program for operating the methods the invention, for example, for interpreting the expression data of step (c) (and subsequent expression measurement steps) and thereby diagnosing or determining a pancreatic cancer-associated disease state. The computer program may be a programmed SVM. The computer program may be recorded on a suitable computer-readable carrier known to persons skilled in the art. Suitable computer-readable-carriers may include compact discs (including CD-ROMs, DVDs, Blue Rays and the like), floppy discs, flash memory drives, ROM or hard disc drives. The computer program may be installed on a computer suitable for executing the computer program.

Preferred, non-limiting examples which embody certain aspects of the invention will now be described with reference to the following tables and figures:

FIG. 1.

Serum biomarker panel discriminating SLE vs. healthy control. A. Backward elimination analysis of the training set, resulting in a condensed set of 25 antibodies (marked with an arrow) providing the best classification. B. Heat map for the training set, based on the 25-plex antibody signature (red—up-regulated, green—down-regulated, back—unchanged). C. ROC curve for the test set, based on the frozen SVM model and 25-plex antibody signature. D. Heat map for the test set, based on the frozen SVM model and 25-plex antibody signature. E. Principle component analysis (PCA) plot of the training set onto which the test set was then mapped. F. PCA plot of the test set only, adapted from D.

FIG. 2.

Robustness of the data set on the classification of SLE vs. healthy controls. A. Boxplot of the ROC AUC values for the test, based on the frozen SVM model and 25-plex antibody signature, iterated ten times, i.e. using ten different pairs of training and test sets. B. Frequency at which each biomarker occurred in the ten 25-plex antibody signatures.

FIG. 3.

Biological relevance of the observed serum biomarkers, evaluated using Metacore™ A. Enrichment analysis—diseases by biomarkers. B. Enrichment analysis—gene onthology process. C. Enrichment analysis—pathway maps. D. Enrichment analysis—process networks. E. The most relevant networks.

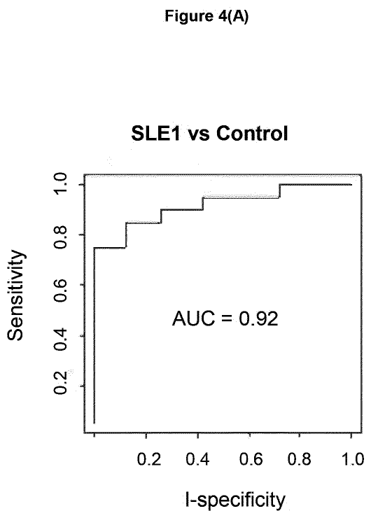

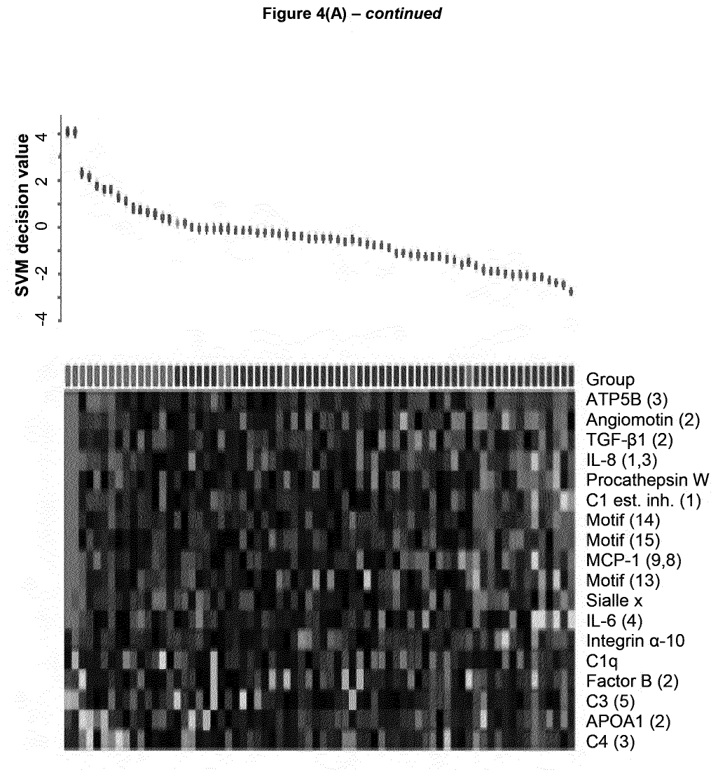

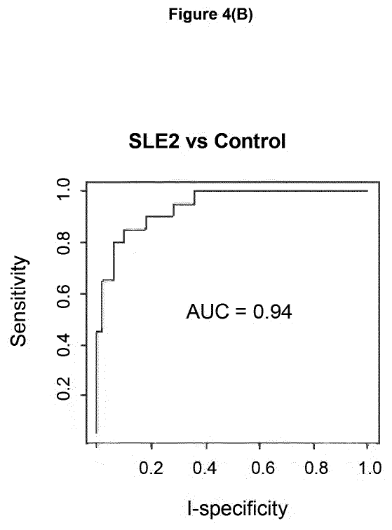

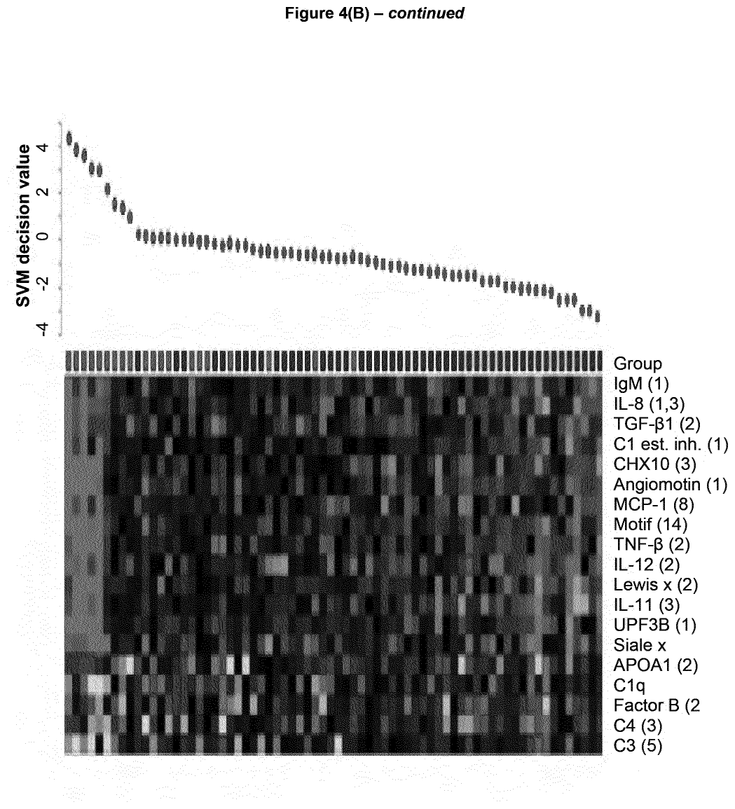

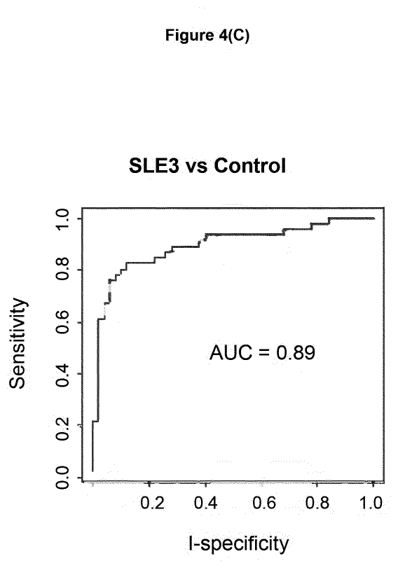

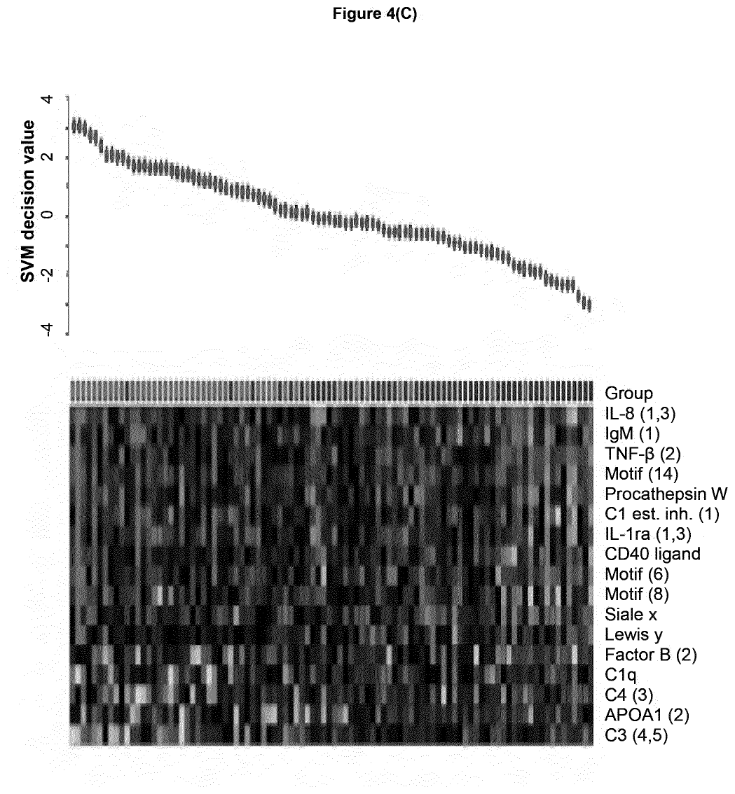

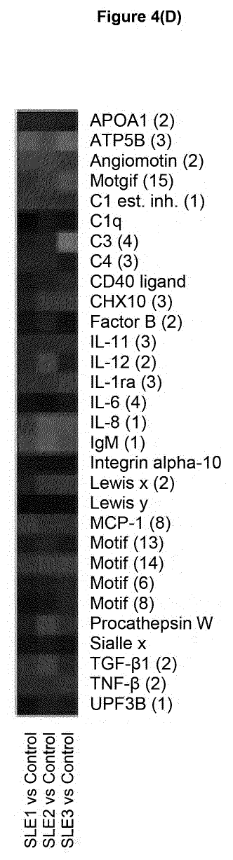

FIG. 4.

Serum biomarker panels discriminating phenotypic subsets of SLE vs. healthy controls. A. SLE1 vs. healthy control, illustrated by ROC AUC curve and heat map (20 top differentially expressed biomarkers; red—up-regulated, green—down-regulated, back—unchanged). B. SLE2 vs. healthy control, illustrated by ROC AUC curve and heat map (20 top differentially expressed biomarkers). C. SLE3 vs. healthy control, illustrated by ROC AUC curve and heat map (20 top differentially expressed biomarkers). D. Comparison of the top 30 differentially expressed biomarkers.

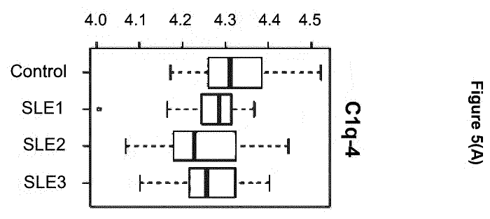

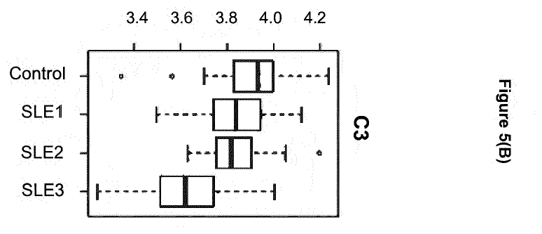

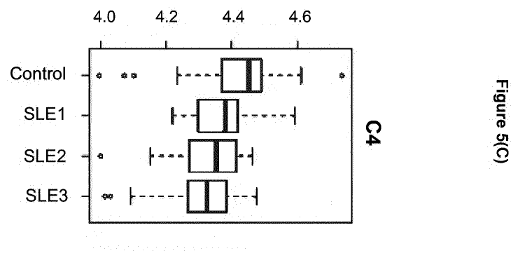

FIG. 5.

Protein expression profiles of five selected key biomarkers. The expression levels are shown for three complement proteins (C1q, C3, and C4) and two cytokines (IL-6 and IL-12).

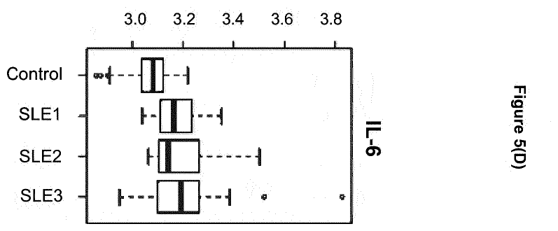

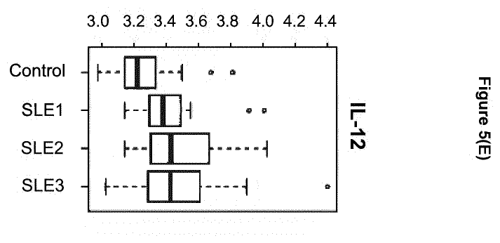

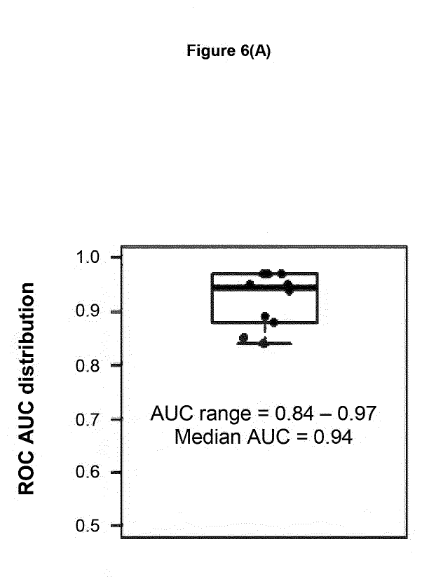

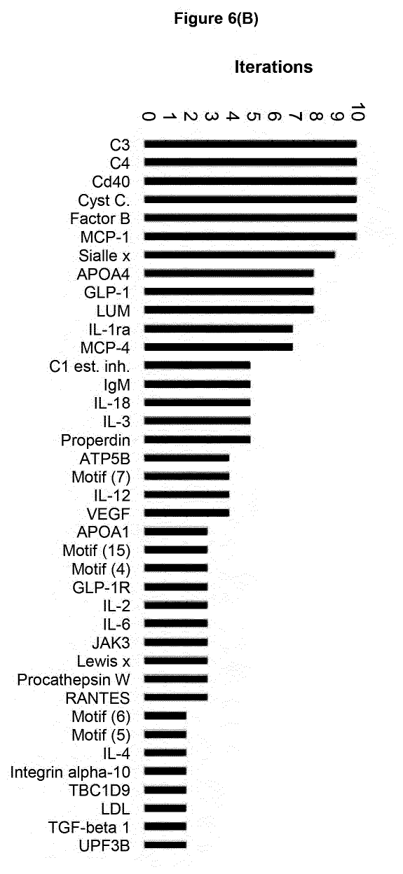

FIG. 6.

Robustness of the data set on the classification of SL3E vs. healthy controls. A. Boxplot of the ROC AUC values for the test, based on the frozen SVM model and 25-plex antibody signature, iterated ten times, i.e. using ten different pairs of training and test sets. B. Frequency at which each biomarker occurred in the ten 25-plex antibody signatures.

TABLE A
Biomarkers for determining a systemic lupus erythematosus-associated disease state

| (i): Core | Biomarker | Diagnostic | Exemplary sequence(s) |
|---|---|---|---|
| 1. | MYOM2 | X | P54296 |
| 2. | ORP-3 | X | Q9H4L5 |
| (ii): Preferred | | | |
| 3. | APOA1 | X | P02647 |
| 4. | APOA4 | X | P06727 |
| 5. | ATP5B | X | P06576 |
| 6. | CHX10 | X | P58304 |
| 7. | TBC1D9 | X | Q6ZT07 |
| 8. | UPF3B | X | Q9BZI7 |
| 9. | LUM | X | P51884 |
| 10. | Digoxin | X | NA-small molecule |
| 11. | Surface Ag X | X | NA-antigen not known |
| 12. | Motif (10) | X | SGSG-SEAHLR (-COOH) [SEQ |
| | | | ID NO: 3] |
| 13. | Motif (13) | X | SGSG-QEASFK (-COOH) [SEQ |
| | | | ID NO: 4] |
| 14. | Motif (14) | X | SGSG-EDFR (-COOH) [SEQ ID |
| | | | NO: 5] |
| 15. | Motif (15) | X | SGSG-GIVKYLYEDEG (-COOH) |
| | | | [SEQ ID NO: 6] |
| 16. | Motif (2) | X | SGSG-SSAYSR (-COOH) |
| | | | [SEQ ID NO: 7] |
| 17. | Motif (4) | X | SGSG-TEEQLK (-COOH) [SEQ |
| | | | ID NO: 8] |
| 18. | Motif (5) | X | SGSG-LSADHR (-COOH) [SEQ |
| | | | ID NO: 9] |
| 19. | Motif (6) | X | SGSG-LTEFAK (-COOH) [SEQ |
| | | | ID NO: 10] |
| 20. | Motif (7) | X | SGSG-TEEQLK (-COOH) [SEQ |
| | | | ID NO: 8] |
| 21. | Motif (8) | X | SGSG-TEEQLK (-COON) [SEQ |
| | | | ID NO: 8] |
| (iii): Optional | | | |
| 22. | Angiomotin | X | Q4VCS5 |
| 23. | C1-INH | X | P05155 |
| 24. | C1q | X | P02745, P02746, P02747 |
| 25. | C3 | X | P01024 |
| 26. | C4 | X | P0COL4, P0COL5 |
| 27. | CD40 | X | Q6P2H9 |
| 28. | CD40 ligand | X | P29965 |
| 29. | Cystatin C | X | P01034 |
| 30. | Factor B | X | P00751 |
| 31. | GLP-1 | X | P01275 |
| 32. | GLP-1R | X | P43220 |
| 33. | IgM | X | e.g., P01871 (not complete |
| | | | protein); isotype-specific for IgM |
| | | | on Ramos B cells) |
| 34. | IL-11 | X | P20809 |
| 35. | IL-12 | X | O60595 |
| 36. | IL-13 | X | P35225 |
| 37. | IL-16 | X | Q05BE6, Q8IUU6, B5TY35 |
| 38. | IL-18 | X | Q14116 |
| 39. | IL-1ra | X | P18510 |
| 40. | IL-2 | X | P60568 |
| 41. | IL-3 | X | P08700 |
| 42. | IL-4 | X | P05112 |
| 43. | IL-5 | X | BC066282, CH471062, P05113 |
| 44. | IL-6 | X | P05231 |
| 45. | IL-8 | X | CR623827, CR623683, |
| | | | DQ893727, DQ890564, P10145 |
| 46. | IL-9 | X | P15248 |
| 47. | Integrin α -10 | X | Hs158237 |
| 48. | JAK3 | X | P52333 |
| 49. | LDL | X | P04114 |
| 50. | Lewis X | X | Carbohydrate structure [NA] |
| 51. | Lewis Y | X | Carbohydrate structure [NA] |
| 52. | MCP-1 | X | P13500 |
| 53. | MCP-3 | X | P80098 |
| 54. | MCP-4 | X | Q99616 |
| 55. | Procathepsin W | X | P56202 |
| 56. | Properdin | X | P27918 |
| 57. | RANTES | X | P13501 |
| 58. | Sialle Lewis X | X | Carbohydrate structure [NA] |
| 59. | TGF-β1 | X | P01137 |
| 60. | TM peptide | X | NA |
| 61. | TNF-α | X | P01375 |
| 62. | TNF-β | X | P01374 |
| 63. | VEGF | X | P15692 |

TABLE B
Motif sequences and corresponding antibody CDR sequences

| | | CDR regions of the selected antibody | |
|---|---|---|---|
| MOTIF | SELECTION MOTIF | CDR H1/H2/H3 | CDR L2/L2/L3 |
| (1) | SGSG-SSAYSR (-COOH) | FSDYYMSWVRQAPG [SEQ | CTGSSSNIGAGYDVH [SEQ |
| | [SEQ ID NO: 7] | ID NO: 11]/ | ID NO: 14]/ |
| | | ADIKRDGSTRYYGDSVKGR | GNSNRPS [SEQ ID NO: 15]/ |
| | | [SEQ ID NO: 12]/ | CAAWDDSLSVL [SEQ ID |
| | | ARDRLVAGLFDY [SEQ ID | NO: 16] |
| | | NO: 13] | |
| (2) | SGSG-SSAYSR (-COOH) | FSSYAMSWVRQAPG [SEQ | CTGSSSNIGAGYDVH [SEQ |
| | [SEQ ID NO: 7] | ID NO: 17]/ | ID NO: 14]/ |
| | | SAISGSGGRTYYTDSVRDR | SNNQRPS [SEQ ID NO: 20]/ |
| | | [SEQ ID NO: 18]/ | CQSYDSSLNKDVV [SEQ ID |
| | | ARDLMPVCQYCYGMDV | NO: 21] |
| | | [SEQ ID NO: 19] | |
| (3) | SGSG-DFAEDK (-COOH) [SEQ | FSSYAMSWVRQAPG [SEQ | CSGGSSNIGSNTVN [SEQ ID |
| | ID NO: 22] | ID NO: 17]/ | NO: 25]/ |
| | | SSISSSSSYIYYADSVKGR | GNSNRPS [SEQ ID NO: 15]/ |
| | | [SEQ ID NO: 23]/ | CAAWDDSLNGRV [SEQ ID |
| | | ARLFFSGGATRAAFDI [SEQ | NO: 27] |
| | | ID NO: 24] | |
| (4) | SGSG-TEEQLK (-COOH) [SEQ | FTSYSIHWVRQAPG [SEQ ID | CSGSSSNIGSNTVN [SEQ ID |
| | ID NO: 8] | NO: 28]/ | NO: 31]/ |
| | | SAIGTGGGTYYADSVKGR | GNSNRPS [SEQ ID NO: 15]/ |
| | | [SEQ ID NO: 29]/ | CQSYDRSLSVNVV [SEQ ID |
| | | ARGGYFLD [SEQ ID NO: 30] | NO: 32] |
| (5) | SGSG-LSADHR (-COOH) [SEQ | FSNYAMSWVRQAPG [SEQ | CSGSSSNIGSNAVN [SEQ ID |
| | ID NO: 9] | ID NO: 33]/ | NO: 36]/ |
| | | AFIRYDGSNKYYADSVKGR | GNSNRPS [SEQ ID NO: 15]/ |
| | | [SEQ ID NO: 34]/ | CAAWDDSLNGWV [SEQ ID |
| | | ARDAVGGDSYVLDY [SEQ ID | NO: 37] |
| | | NO: 35] | |
| (6) | SGSG-LTEFAK (-COOH) [SEQ | FSDYYMSWIRQAPG [SEQ ID | CTGSSSNIGAGYDVH [SEQ |
| | ID NO: 10] | NO: 38]/ | ID NO: 14]/ |
| | | SSISSRSSYIYYADSVKGR | DNNKRPS [SEQ ID NO: 41]/ |
| | | [SEQ ID NO: 39]/ | CSAWDESLSGVV [SEQ ID |
| | | AKDREYYDILTGYPSMDV | NO: 42] |
| | | [SEQ ID NO: 40] | |
| (7) | SGSG-TEEQLK (-COOH) [SEQ | FSSYGMHWVRQAPG [SEQ | CSGSSSNVGVNYVY [SEQ ID |
| | ID NO: 8] | ID NO: 43]/ | NO: 46]/ |
| | | SAISGSGGSTYYADSVKGR | SHNQRPS [SEQ ID NO: 47]/ |
| | | [SEQ ID NO: 44]/ | CAAWDDSLNGVV [SEQ ID |
| | | ARSRYGSGMDV [SEQ | ID NO: 48] |
| | | NO: 45] | |
| (8) | SGSG-TEEQLK (-COOH) [SEQ | FSSYGMHWVRQAPG [SEQ | CSGSSSNIGNNYVS [SEQ ID |
| | ID NO: 8] | ID NO: 43]/ | NO: 50]/ |
| | | SAISGSGGSTYYADSVKGR | SNNQRPS [SEQ ID NO: 20]/ |
| | | [SEQ ID NO: 44]/ | CATWDDSLSGGV [SEQ ID |
| | | ARGGVGRYGMDV [SEQ ID | NO: 51] |
| | | NO: 49] | |
| (9) | SGSG-EDFR (-COOH) [SEQ ID | FNTAMSWVRQAPG [SEQ ID | CSGSSSNIGSNSVN [SEQ ID |
| | NO: 5] | NO: 52]/ | NO: 55]/ |
| | | SSISAGGTRTFYADSVRGR | DNNRRPS [SEQ ID NO: 56]/ |
| | | [SEQ ID NO: 53]/ | CAAWDDSLNWV [SEQ ID |
| | | ARHRAAGGGYYYGMDV | NO: 57] |
| | | [SEQ ID NO: 54] | |
| (10) | SGSG-SEAHLR (-COOH) [SEQ | FSSYAMSWVRQAPG [SEQ | CSGSSSNIGSNTVN [SEQ ID |
| | ID NO: 3] | ID NO: 17]/ | NO: 31]/ |
| | | AAIWSDGSNKYYADSVKGR | GNSNRPS [SEQ ID NO: 15]/ |
| | | [SEQ ID NO: 58]/ | CAAWDDSLNGPV [SEQ ID |
| | | AKVGATDDAFDI [SEQ ID | NO: 60] |
| | | NO: 59] | |
| (11) | SGSG-SEAHLR (-COOH) [SEQ | FSSYAMSWVRQAPG [SEQ | CSGGSSNIGSNTVN [SEQ ID |
| | ID NO: 3] | ID NO: 17]/ | NO: 25]/ |
| | | SSISSSSSYIYYADSVKGR | RNNQRPS [SEQ ID NO: 26]/ |
| | | [SEQ ID NO: 23]/ | CAAWDDSLSGVV [SEQ ID |
| | | ARHIQGSGGLDV [SEQ ID | NO: 62] |
| | | NO: 61] | |
| (12) | SGSG-SEAHLR (-COOH) [SEQ | FTSYSMSWVRQAPG [SEQ | CSGSSSNIGNNAVN [SEQ ID |
| | ID NO: 3] | ID NO: 63]/ | NO: 65]/ |
| | | SAIGTGGGTYYADSVKGR | RNDQRPS [SEQ ID NO: 66]/ |
| | | [SEQ ID NO: 29]/ | CSTWDDSLSGVF [SEQ ID |
| | | ARVNWNDAFDY [SEQ ID | NO: 67] |
| | | NO: 64] | |
| (13) | SGSG-QEASFK (-COOH) [SEQ | FSSYAMSWVRQAPG [SEQ | CSGSSSNIGTNYVY [SEQ ID |
| | ID NO: 4] | ID NO: 17]/ | NO: 70]/ |
| | | SAISGSGGRTYYADAVKGR | SNNQRPS [SEQ ID NO: 20]/ |
| | | [SEQ ID NO: 68]/ | CAAWDDSLSVWV [SEQ ID |
| | | ARHLKHDDGNSGAFDI [SEQ | NO: 71] |
| | | ID NO: 69] | |
| (14) | SGSG-EDFR (-COOH) [SEQ ID | FDDYGMSWVRQAPG [SEQ | CSGSSSNIGSNYVY [SEQ ID |
| | NO: 5] | ID NO: 72]/ | NO: 74]/ |
| | | SAISGSGGSTYYADPVKGR | KSNQRPS [SEQ ID NO: 75]/ |
| | | [SEQ ID NO: 73]/ | CAAWDDRLNAVV [SEQ ID |
| | | ARSRYGSGMDV [SEQ ID | NO: 76] |
| | | NO: 45] | |
| (15) | SGSG-GIVKYLYEDEG (-COOH) | FSNYAMHWVRQAPG [SEQ | CTGSSSNIGADYDVH [SEQ |
| | [SEQ ID NO: 6] | ID NO: 77]/ | ID NO: 80]/ |
| | | SSISNRGSRTFYADSVKGR | GNSNRPS [SEQ ID NO: 15]/ |
| | | [SEQ ID NO: 78]/ | CAAWDDGLSGVV [SEQ ID |
| | | ARDHRWDPGAFDI [SEQ ID | NO: 81] |
| | | NO: 79] | |

TABLE C
ROC-AUCs of biomarker signatures ranging from 2 to 18 of the Table A(i), (ii)
and (iii) biomarkers (core is composed of biomarkers 1 and 2 of Table A. 14
Table A(i), (ii) and (iii) biomarkers are added, one-by-one.

| | | Recommended | |
|---|---|---|---|
| Marker | Uniprot entry ID | protein name | Short name |
| 1 | na carbohydrate | Sialle Lewis x | Sialle x |
| 2 | P01871 | IgM | IgM |
| 3 | SGSG-TEEQLK (-COOH) | motif (4) | motif (4) |
| | [SEQ ID NO: 8] | | |
| 4 | P01583 | Interleukin-1 alpha | IL-1a |
| 5 | P01024 | Complement C3 | C3 |
| 6 | SGSG-LTEFAK (-COOH) | motif (6) | motif (6) |
| | [SEQ ID NO: 10] | | |
| 7 | SGSG-SEAHLR (-COOH) | motif (10) | motif (10) |
| | [SEQ ID NO: 3] | | |
| 8 | Q6P2H9 | CD40 protein | CD40 |
| 9 | O75578 | Integrin alpha-10 | Integrin a-10 |
| 10 | P01375 | Tumor necrosis factor | TNF-a |
| 11 | P13500 | C-C motif chemokine 2 | MCP-1 |
| 12 | P51884 | Lumican | Lumican |
| 13 | P01034 | Cystatin C | Cystatin C |
| 14 | Q9BZI7 | Regulator of nonsense | UPF3B |
| | | transcripts 3B | |
| 15 | P01375 | Tumor necrosis factor | TNF-a |
| 16 | P01137 | Transforming growth factor | TGF-b1 |
| | | beta-1 | |

ROC AUC LADDER

| AUC ROC | Marker |
|---|---|
| 0.89 | 1+2 |
| 0.91 | 1+2+3 |
| 0.9 | 1+2+3+4 |
| 0.91 | 1+2+3+4+5 |
| 0.92 | 1+2+3+4+5+6 |
| 0.91 | 1+2+3+4+5+6+7 |
| 0.95 | 1+2+3+4+5+6+7+8 |
| 0.97 | 1+2+3+4+5+6+7+8+9 |
| 0.96 | 1+2+3+4+5+6+7+8+9+10 |
| 0.96 | 1+2+3+4+5+6+7+8+9+10+11 |
| 0.98 | 1+2+3+4+5+6+7+8+9+10+11+12 |
| 0.97 | 1+2+3+4+5+6+7+8+9+10+11+12+13 |
| 0.97 | 1+2+3+4+5+6+7+8+9+10+11+12+13+14 |
| 0.97 | 1+2+3+4+5+6+7+8+9+10+11+12+13+14+15 |
| 0.97 | 1+2+3+4+5+6+7+8+9+10+11+12+13+14+15+16 |

TABLE D
ROC-AUCs of biomarker signatures ranging from 2 to 20 of the
Table A(i) and (ii) biomarkers (core is composed of biomarkers
1 and 2 of Table A. The next 18 Table A(i) and (ii) biomarkers
are added, in turn, in the order in which they appear in Table A.
ROC AUC LADDER

| AUC ROC | Markers |
|---|---|
| 0.73 | 1 + 2 |
| 0.72 | 1 + 2 + 3 |
| 0.75 | 1 + 2 + 3 + 4 |
| 0.75 | 1 + 2 + 3 + 4 + 5 |
| 0.74 | 1 + 2 + 3 + 4 + 5 + 6 |
| 0.75 | 1 + 2 + 3 + 4 + 5 + 6 + 7 |
| 0.8 | 1 + 2 + 3 + 4 + 5 + 6 + 7 + 8 |
| 0.85 | 1 + 2 + 3 + 4 + 5 + 6 + 7 + 8 + 9 |
| 0.85 | 1 + 2 + 3 + 4 + 5 + 6 + 7 + 8 + 9 + 10 |
| 0.85 | 1 + 2 + 3 + 4 + 5 + 6 + 7 + 8 + 9 + 10 + 11 |
| 0.85 | 1 + 2 + 3 + 4 + 5 + 6 + 7 + 8 + 9 + 10 + 11 + 12 |
| 0.85 | 1 + 2 + 3 + 4 + 5 + 6 + 7 + 8 + 9 + 10 + 11 + |
| | 12 + 13 |
| 0.89 | 1 + 2 + 3 + 4 + 5 + 6 + 7 + 8 + 9 + 10 + 11 + |
| | 12 + 13 + 14 |
| 0.88 | 1 + 2 + 3 + 4 + 5 + 6 + 7 + 8 + 9 + 10 + 11 + |
| | 12 + 13 − 14 + 15 |
| 0.89 | 1 + 2 + 3 + 4 + 5 + 6 + 7 + 8 + 9 + 10 + 11 + |
| | 12 + 13 − 14 + 15 + 16 |
| 0.88 | 1 + 2 + 3 + 4 + 5 + 6 + 7 + 8 + 9 + 10 + 11 + |
| | 12 + 13 − 14 + 15 + 16 + 17 |
| 0.91 | 1 + 2 + 3 + 4 + 5 + 6 + 7 + 8 + 9 + 10 + 11 + |
| | 12 + 13 − 14 + 15 + 16 + 17 + 18 |
| 0.9 | 1 + 2 + 3 + 4 + 5 + 6 + 7 + 8 + 9 + 10 + 11 + |
| | 12 + 13 − 14 + 15 + 16 + 17 + 18 + 19 |
| 0.89 | 1 + 2 + 3 + 4 + 5 + 6 + 7 + 8 + 9 + 10 + 11 + |
| | 12 + 13 − 14 + 15 + 16 + 17 + 18 + 19 + 20 |

TABLE E TABLE C
Amino acid sequences of the scFv antibodies used in the Examples

| Ab | Full protein Sequence (VH-linker-VL-tag) |
|---|---|
| IL-1α (1) | EVQLLESGGGLVQPGGSLRLSCAASGFTFSSYAMHWVRQAPGKGLEWVSGVSWNGSRTHYADSVKGRFTISRDNSKNTLYLQMNSLRAEDTAVYYCARSSGYYSWAFDIW |
| | GQGTLVTVSSGGGGSGGGGSGGGGSQSVLTQPPSASGTPGQRVTISCSGSSSNIGRNTVNWYQQLPGTAPKLLIYGNSNRPSXVPDRFSGSKSGTSASLAISGLRSEDEADYY |
| | CAAWDDSLNGWAFGGXTKLTVLGEQKLISXXXLSGSAA [SEQ ID NO: 82] |
| IL-1α (2) | EVQLLESGGGLVQPGGSLRLSCAASGFTFSSYSMNWVRQAPGKGLEWVALISYDGSQKYYADSMKGRFTISRDNSKNTLYLQMNSLRAEDTAVYYCAKGHTSGTKAYYFDS |
| | WGQGTLVTVSSGGGGSGGGGSGGGGSQSVLTQPPSASGTPGQRVTISCTGTSSNIGAGYSVHWYQQLPGTAPKLLIYGNSNRPSGVPDRFSGSKSGTSASLAISGLRSEDXA |
| | DYYCQSYDSSLSGWVFGGXTKLTVLGEQKLISEEDLSGSAA [SEQ ID NO: 83] |
| IL-2 (1) | EVQLLESGGGLVQPGGSLRLSCAASGFTFGDYAMSWVRQAPGKGLEWVSSISSRGSYIYFADSVKGRFTISRDNSKNTLYLQMNSLRAEDTAVYYCAKKKTGYYGLDAWGQG |
| | TLVTVSSGGGGSGGGGSGGGGSQSVLTQPPSASGTPGQRVTISCTGSSSNIGAGYDVHWYQQLPGTAPKLLIYGNSNRPSGVPDRFSGSKSGTSASLAISGLRSEDEADYYCSS |
| | YAGSNNLVFGGXTKLTXLGEQKLISXXDLSGSAA [SEQ ID NO: 84] |
| IL-2 (2) | EVXXLESGGGLVQPGGSLRLSCAASGFTFGDYAMSWVRQAPGKGLEWVSSISSRGSYIYFADSVKGRFTISRDNSKNTLYLQMNSLRAEDTAVYYCAKKKTGYYGLDAWGQG |
| | TLVTVSSGGGGSGGGGSGGGGSQSVLTQPPSASGTPGQRVTISCTGSSSNIGAGYDVHWYQQLPGTAPKLLIYGNSNRPSGVPDRFSGSKSGTSASLAISGLRSEDEADYYCSS |
| | YAGSNNLVFGGXXKLTVLGEQKLISEXXLSGSAA [SEQ ID NO: 85] |
| IL-2 (3) | EVQLLESGGGLVQPGGSLRLSCAASGFTFGDYAMSWVRQAPGKGLEWVSSISSRGSYIYFADSVKGRFTISRDNSKNTLYLQMNSLRAEDTAVYYCAKKKTGYYGLDAWGQG |
| | TLVTVSSGGGGSGGGGSGGGGSQSVLTQPPSASGTPGQRVTISCTGSSSXIGAGYDVHWYQQLPGTAPKLLIYGNSNRP [SEQ ID NO: 86] |
| IL-3 (1) | EVQLLESGGGLVQPGGSLRLSCAASGFTFGRYTMHWVRQAPGKGLEWVSSISSSSSYIYYADSVKGRFTISRDNSKNTLYLQMNSLRAEDTAVYYCARHFFESSGGYFDYWGQ |
| | GTLVTVSSGGGGSGGGGSGGGGSQSVLIQPPSASGTPGQRVTISCSGSSSNIGSNTVNWYQQLPGTAPKLLIYRNNQRPSGVPDRFSGSKSGTSASLAISGLRSEDXADYYCA |
| | AWDDSLNGWVFGGXXKLTVLGEQKLISXXXLSGXAA [SEQ ID NO: 87] |
| IL-3 (2) | EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCARGGARYDYWGQGTL |
| | VTVSSGGGGSGGGGSGGGGSQSVLTQPPSASGTPGQRVTISCSGSSSNIGSNTVNWYQQLPGTAPKLLIYDNNKRPSGVPDRFSGSKSGTSASLAISGLRSEDEADYYCQSYD |
| | NILRGVVFGGGTKLTVLXEQKLISEXDLSGSAA [SEQ ID NO: 88] |
| IL-3 (3) | EVXXXESGGGLVQPGGSLRLSCAASGFTFSSYAMSWVRQAPGKGLEWVSAISGRGEYTYYAGSVKGRFTISRDNSKNTLYLQMNSLRAEDTAVYYCATGATRFGYWGQGTL |
| | VTVSSGGGGSGGGGSGGGGSQSVLTQPPSASGTPGQRVTISCTGSSSNIGAGYGVQWYQQLPGTAPKWYRNNQRPSGVPDRFSGSKSGTSXSLAISGLRSEDEADYYCQSY |
| | DSSLSYSVFGGXTKLTVLGEQKLISXXDLSGSAA [SEQ ID NO: 89] |
| IL-4 (1) | EVQLLESGGGLVQPGGSLRLSCAASGFTFSNAWMSWVRQAPGKGLEWVSSLHGGGDTFYTDSVKGRFTISRDNSKNTLYLQMNSLRAEDTAVYYCASLYGSGSYYYYYYGM |
| | DVWGQGTLVTVSSGGGGSGGGGSGGGGSQSVLTQPPSASGTPGQRVTISCSGNNSNTGNNAVNWYQQLPGTAPKLLIYDNNKRPSGVPDRFSGSKSGTSASLAISGLRSE |
| | DEADYYCCSYAGSYIWVFGGXTKLTVLGEQKLISXEXLSGSAA [SEQ ID NO: 90] |
| IL-4 (2) | EVQLLESGGGLVQPGGSLRLSCAASGFTFSDYGMHWVRQAPGKGLEWVSGISWNGGKTHYVDSVKGQFTISRDNSKNTLYLQMNSLRAEDTAVYYCARDRGYCSNGVCYT |
| | ILDYWGQGTLVTVSSGGGGSGGGGSGGGGSQSVLTQPPSASGTPGQRVTISCSGSSSNIGSNTINWYQQLPGTAPKWYGNSNRPSGVPDRFSGSKSGTSASLAISGLRSEDE |
| | ADYYCQSYDSSLSGWVFGGXTKLXVLXEQKLISXXDLSGSAA [SEQ ID NO: 91] |
| IL-5 (1) | EVQLLESGGGLVQPGGSLRLSCAASGFTFSSYAMSWVRQAPGKGLEWVSSISSRSNYIYYSDSVKGRFTISRDNSKNTLYLQMNSLRAEDTAVYYCARNFRFFDKWGQGTLVT |
| | VSSGGGGSGGGGSGGGGSQSVLTQPPSASGTPGQRVTISCSGGSSXIGANPVSWYQQLPGTAPKLLIYGNSNRP [SEQ ID NO: 92] |
| IL-5 (2) | EVQLLESGGGLVQPGGSLRLSCAASGFTESSYAMSWVRQAPGKGLEWVSSISSRSNYIYYSDSVKGRFTISRDNSKNTLYLQMNSLRAEDTAVYYCARNFRFFDKWGQGTLVT |
| | VSSGGGGSGGGGSGGGGSQSVLTQPPSASGTPGQRVTISCSGGSSNIGANPVSWYQQLPGTAPKLLIYGNSNRPSGVPDRFSGSKSGTSASLAISGLRSEDEADYYCQSYDSS |
| | LSGSVFGGXTKLTVLXEQKLISEXXLSGSAA [SEQ ID NO: 93] |
| IL-5 (3) | EVQLLESGGGLVQPGGSLRLSCAASGFTFSSYAMSWVRQAPGKGLEWVSSISSRSNYIYYSDSVKGRFTISRDNSKNTLYLQMNSLRAEDTAVYYCARNFRFFDKWGQGTLVT |
| | VSSGGGGSGGGGSGGGGSQSVLTQPPSASGTPGQRVTISCSGGSSNIGANPVSWYQQLPGTAPKLLIYGNSNRPSGVPDRFSGSKSGTSASLAISGLRSEDEADYYCQSYDSS |
| | LSGSVEGGXTKLTVLGEQKLISXEDLSGSAA [SEQ ID NO: 94] |
| IL-6 (1) | EVQLLESGGGLVQPGGSLRLSCAASGFTFSSYGMHWVRQAPGKGLEWVSGINWNGGSTGYADSVKGRFTISRDNSKNTLYLQMNSLRAEDTAVYYCARNRGSSLYYGMDV |
| | WGQGTLVTVSSGGGGSGGGGSGGGGSQSVLTQPPSASGTPGQRVTISCAGSSSNIGSKSVHWYQQLPGTAPKLLIYRNNRRPSGVPDRFSGSXSGTSXSLAIXGLRSXDXAD |
| | YYCXXWDDRVNXXXFGGXTXLTVLXXQKLISXXXLSGSXXXPSSSXXLIXXGXXXXLX-XXLXFTGRXEXTX-LXXX [SEQ ID NO: 95] |
| IL-6 (2) | EVQLLESGGGLVQPGGSLRLSCAASGFTFSNYGMHWVRQAPGKGLEWVSSITSSGDGTYFADSVKGRFTISRDNSKNTLYLQMNSLRAEDTAVYYCARAGGIAAAYAFDIW |
| | GQGTLVTVSSGGGGSGGGGSGGGGSQSVLTQPPSASGTPGQRVTISCSGSSSNVGSNYVYWYQQLPGTAPKLLIYDNNKRPSGVPDRFSGSKSGTSASLAISGLRSEDEADYY |
| | CQSYDSSRWVFGGXTKLTVLGEQXLISEEXLSGSAA [SEQ ID NO: 96] |
| IL-7 (1) | EVQLLESGGGLVQPGGSLRLSCAASGFTESSYGMHWVRQAPGKGLEWVSGITWNSGSIGYVDSVKGRFTISRDNSKNTLYLQMNSLRAEDTAVYYCARGPSVAARRIGRHW |
| | YNWFDPWGQGTLVTVSSGGGGSGGGGSGGGGSQSVLTQPPSASGTPGQRVTISCSGSSSNIGSNSVYWYQQLPGTAPKLLIYDNNKRPSGVPDRFSGSKSGTSASLAISGLR |
| | SEXXADYYCQSYDSSLSGSVEGGXXKLXVLGEQKLISEXXLSGSAA [SEQ ID NO: 97] |
| IL-7 (2) | EVQLLESGGGLVQPGGSLRLSCAASGFTFSDYNIHWVRQPPGKGLEWVSGVSWNGSRTHYADSVKGQFTISRDNSKNTLYLQMNSLRAEDTAVYYCARDPAMVRGVVLPN |
| | YYGLDVWGQGTLVTVSSGGGGSGGGGSGGGGSQSVLTQPPSASGTPGQRVTISCTGSSSNIGAGYDVHWYQQLPGTAPKLLIYGHSNRPSGVPDRFSGSKSGTSASLAISGL |
| | RSEXXADYYCQSYDSSLSYPVFGGXTKLTVLGEQ [SEQ ID NO: 98] |
| IL-8 (1) | EVQLLESGGGLVQPGGSLRLSCAASGFTEDDYGMSWVRQAPGKGLEWVSLISWDGGSTYYADSVKGRFTISRDNSKNTLYLQMNSLRAEDTAVYYCARDDLYGMDVWGQ |
| | GTLVTVSSGGGGSGGGGSGGGGSQSVLIQPPSASGTPGQRVTISCTGSSSNIGAGYDVHWYQQLPGTAPKLLINIDNNKRPSXVPDRFSGSKSGTSASLAISGLRSEDEADYYC |
| | AAWDDSLSGWVFGGXTKLTVLXEQKLISEXXLSGSAA [SEQ ID NO: 99] |
| IL-8 (2) | EVQLLESGGGLVQPGGSLRLSCAASGFTESSYEMNWVRQAPGKGLEWVSSISSSSSYIFYADSMKGRFTISRDNSKNTLYLQMNSLRAEDTAVYYCARNESVDPLGGQYFQH |
| | WGQGTLVTVSSGGGGSGGGGSGGGGSQSVLTQPPSASGTPGQRVTISCTGSSSNIGAGYDVHWYQQLPGTAPKLLIYDNNKRPSGVPDRFSGSKSGTSASLAISGLRSEDEA |
| | DYYCSAWDDNLDGPVFGGXTKLTVLXEQKLISXXXLSGSAA [SEQ ID NO: 100] |
| IL-9 (1) | EVQLLESGGGLVQPGGSLRLSCAASGFTESSYAMSWVRQAPGKGLEWVSSISSSSSYIYYADSVKGRFTISRDNSKNTLYLQMNSLRAEDTAVYKTTFGHWGQGTLVTVSSG |
| | GGGSGGGGSGGGGSQSVLTQPPSASGTPGQRVTISCSGSGSNIGDNSVNWYQQLPGTAPKLLIYGNNNRPSGVPDRFSGSKSGTSASLAISGLRSEDEADYYCSSYTSSSVVF |
| | GGXTKLTVLXEQKLISEXDLSGSAA [SEQ ID NO: 101] |
| IL-9 (2) | EVQLLESGGGLVQPGGSLRLSCAASGFTFSSYGMHWVRQAPGKGLEWVAVISYDGSNKYYADSVKGRFTISRDNSKNTLYLQMNSLRAEDTAVYYCAKSPGGSPYYFDYWG |
| | QGTLVTVSSGGGGSGGGGSGGGGSQSVLTQPPSASGTPGQRVTISCSGSVSNIGSNVVSWYQQLPGTAPKLLIYDNNKRPS [SEQ ID NO: 102] |
| IL-9 (3) | EVQLLESGGGLVQPGGSLRLSCAASGFTFSSYGMHWVRQAPGKGLEWVAVISYDGSNKYYADSVKGRFTISRDNSKNTLYLQMNSLRAEDTAVYYCAKSPGGSPYYFDYWG |
| | QGTLVTVSSGGGGSGGGGSGGGGSQSVLTQPPSASGTPGQRVTISCSGSVSNIGSNVVSWYQQLPGTAPKLLIYDNNKRPSGVPDRFSGSKSGTSASLAISGLRSEDEADYK |
| | QSYDSSLGGWVFGGXTKLTVLGEQKLISEXDLSGSAA [SEQ ID NO: 103] |
| IL-10 (1) | EVQLLESGGGLVQPGGSLRLSCAASGFTFRSYVMSWVRQAPGKGLEWVSAISGSGGSTYYADSVKGRFTISRDNSKNTLYLQMNSLRAEDTAVYYCARGKGRWAFDIWGQ |
| | GTLVTVSSGGGGSGGGGSGGGGSQSVLTQPPSASGTPGQRVTISCTGSSSNVGAGYDVHWYQQLPGTAPKLLIYRNNQRPSGVPDRFSGSKSGTSASLAISGLRSXDXADYY |
| | CAAWDDSLSAHVVEGGXTKLWLGEQKLISXXDLSGSAA [SEQ ID NO: 104] |
| IL-10 (2) | EVQLLESGGGLVQPGGSLRLSCAASGFTFRSYVMSWVRQAPGKGLEWVSAISGSGGSTYYADSVKGRFTISRDNSKNTLYLQMNSLRAEDTAVYYCARGKGRWAFDIWGQ |
| | GTLVTVSSGGGGSGGGGSGGGGSQSVLTQPPSASGTPGQRVTISCTGSSSNVGAGYDVHWYQQLPGTAPKLLIYRNNQRPSGVPDRFSGSKSGTSASLAISGLRSEDEADYY |
| | CAAWDDSLSAHVVFGGXTKLTVLGEQKLISEXDLSGSAA [SEQ ID NO: 105] |
| IL-10 (3) | EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCARGKGRWAFDIWGQG |
| | TLVTVSSGGGGSGGGGSGGGGSQSVLTQPPSASGTPGORVTISCTGSSSNIGAGYGVHWYQQLPGTAPKLLIYGNSNRPSGVPDRFSGSKSGTSASLAISGLRSEDXADYYCA |
| | AWDDSLSGLVFGGXTKLTVLXEQKLISEXXLSGSAA [SEQ ID NO: 106] |
| IL-11 (1) | EVQLLESGGGLVQPGGSLRLSCAASGFTFSNFGMHWVRQAPGKGLEWVAFIRYDGSNKYYADSVKGRFTISRDNSKNTLYLQMNSLRAEDTAVYYCARHYYYSETSGHPGG |
| | FDPWGQGTLVTVSSGGGGSGGGGSGGGGSQSVLTQPPSASGTPGQRVTISCSGSSSNIGSYPVNWYQQLPGTAPKLLIYGNSNRPSGVPDRFSGSKSGTSASLAISGLRSEDE |
| | ADYYCQXWGTGVFGGXTKLTVLGEQKLISXEXLSGSAA [SEQ ID NO: 107] |
| IL-11 (2) | EVQLLESGGGLVQPGGSLRLSCAASGFTFSSYGMHWVRQAPGKGLEWVAVISYDGSNKYYADSVKGRFTISRDNSKNTLYLQMNSLRAEDTAVYYCARHYYDVSYRGQQDA |
| | FDIWGQGTLVTVSSGGGGSGGGGSGGGGSQSVLTQPPSASGTPGQRVTISCTGSSSNLGSPYDVHWYQQLPGTAPKLLIYHRNDQRASGVPDRFSGSXSGTSASLAISGLRSE |
| | DEADYYCAAWDDSLNAWVFGGXTKLTVLGEQKLISEXXLSGSAA [SEQ ID NO: 108] |
| IL-11 (3) | EVQLLESGGGLVQPGGSLRLSCAASGFTFSDYYMSWIRQAPGKGLEWVAYISGISGYTNYADSVRGRFTISRDNSKNTLYLQMNSLRAEDTAVYYCAKSKDWVNGGEMDVW |
| | GQGTLVTVSSGGGGSGGGGSGGGGSQSVLTQPPSASGTPGQRVTISCTGSSSNIGAGYVVHWYQQLPGTAPKLLIYSNNQRPSGVPDRFSGSKSGTSASLAISGLRSEDEADY |
| | YCAAWDDSLRGWVFGGXTKLTVLGEQKLISEEDLSGSAA [SEQ ID NO: 109] |
| IL-12 (1) | EVQLLESGGGLVQPGGSLRLSCAASGFTFSSYSMNWVRQAPGKGLEWVSAIGTGGGTYYADSVKGRFTISRDNSKNTLYLQMNSLRAEDTAVYYCARAFRAFDIWGQGTLV |
| | TVSSGGGGSGGGGSGGGGSQSVLTQPPSASGTPGQRVTISCSGSRSNIGNNFVSWYQQLPGTAPKLLIYGNSNRPSGVPDRFSGSKSGTSASLAISGLRSEDEADYYCAAWD |
| | DSLSGPVFGGXTKLTVLGEQKLISEXDLSGSAA [SEQ ID NO: 110] |
| IL-12 (2) | EVQLLESGGGLVQPGGSLRLSCAASGFTFSDYYMSWVRQAPGKGLEWVSGVSWNGSRTHYADSVKGQFTISRDNSKNTLYLQMNSLRAEDTAVYYCARGSRSSPDAFDIW |
| | GQGTLVTVSSGGGGSGGGGSGGGGSQSVLTQPPSASGTPGQRVTISCTGSSSNIGAGYDVHWYQQLPGTAPKLLIYGNSNRPSGVPDRFSGSKSGTSASLAISGLRSEDEADY |
| | YCAAWDDRVNGRVFGGGTKLTVLGEQKLISEXXLSGSAA [SEQ ID NO: 111] |
| IL-13 (1) | EVQLLESGGGLVQPGGSLRLSCAASGFTFSSYSMNWVRQAPGKGLEWVSSISSGSSYIYYADSVKGRFTISRDNSKNTLYLQMNSLRAEDTAVYYCARSQGWWTYYYGMDV |
| | WGQGTLVTVSSGGGGSGGGGSGGGGSQSVLTQPPSASGTPGQRVTISCSGSSSNIGSNTVNWYQQLPGTAPKLLIYSNNQRPSGVPDRFSGSKSGTSASLAISGLRSEDEAD |
| | YYCETWGQ [SEQ ID NO: 112] |
| IL-13 (2) | EVQLLESGGGLVQPGGSLRLSCAASGFTFSSYSMNWVRQAPGKGLEWVSSISSGSSYIYYADSVKGRFTISRDNSKNTLYLQMNSLRAEDTAVYYCARSQGWWTYYYGMDV |
| | WGQGTLVTVSSGGGGSGGGGSGGGGSQSVLTQPPSASGTPGQRVTISCSGSSSNIGSNTVNWYQQLPGTAPKLLIYSNNQRPSGVPDRFSGSKSGTSASLAISGLRSEDXAD |
| | YYCETXDSNTQIFGGXTKLTVLGEQKLISEEXLSGSAXAHHHHHH-SXRXPIXXIVSXITIHXXSFXNVVTGKXXALPXXXALQHIPXXXAXXXX [SEQ ID NO: 113] |
| IL-13 (3) | EVQLLESGGGLVQPGGSLRLSCAASGFTFSSYSMNWVRQAPGKGLEWVSSISSGSSYIYYADSVKGRFTISRDNSKNTLYLQMNSLRAEDTAVYYCARSQGWWTYYYGMDV |
| | WGQGTLVTVSSGGGGSGGGGSGGGGSQSVLTQPPSASGTPGQRVTISCSGSSSNIGSNTVNWYQQLPGTAPKLLIYSNNQRPSGVPDRFSGSKSGTSASLAISGLRSEDEAD |
| | YYCETWDSNTQIFGGXTKLTVLGEQKLISEXDLSGSAA [SEQ ID NO: 114] |
| VEGF (1) | EVQLLESGGGLVQPGGSLRLSCAASGFTFSSNEMSWIRQAPGKGLEWVSSISGSGGFTYYADSVKGRYTISRDNSKNTLYLQMNSLRAEDTAVYYCARETTVRGNAFDIWGQ |
| | GTLVTVSSGGGGSGGGGSGGGGSQSVLTQPPSASGTPGQRVTISCTGGSSNIGAGYDVHWYQQLPGTAPKLLIYRNNQRPSGVPDRFSGSXSGTSASLAISGLRSEDEADYYC |
| | AAWDDSLSVPMFGGXTKLTVLGEQKLISEXDLSGSAA [SEQ ID NO: 115] |
| VEGF (2) | EVQLLESGGGLVQPGGSLRLSCAASGFTFSSYSMNWVRQAPGKGLEWVSGINWNGGSTGYADSVKGRFTISRDNSKNTLYLQMNSLRAEDTAVYYCASSVGGWYEGDNW |
| | FDPWGQGTLVTVSSGGGGSGGGGSGGGGSQSVLTQPPSASGTPGQRVTISCTGSSSNIGAGYDVHWYQQLPGTAPKLLIYGNSNRPSXVPDRFSGSXSGTSASLAISGLRSE |
| | XEADYYCQSYDGSLSGSVFGGXTKLTVLGEXKLISEXXLSGSAA [SEQ ID NO: 116] |
| TGF-β1 (1) | EVQLLESGGGLVQPGGSLRLSCAASGFTFSTYAMSWVRQAPGKGLEWVAVVSIDGGTTYYGDPVKGRFTISRDNSKNTLYLQMNSLRAEDTAVYYCTRGPTLTYYFDYWGQ |
| | GTLVTVSSGGGGSGGGGSGGGGSQSVLTQPPSASGTPGQRVTISCSGSSSNIGSNTVNWYQQLPGTAPKLLIYSNNQRPSGVPDRFSGSKSGTSASLAISGLRSEDEADYYCQ |
| | SYDSSLSGWVFGGXTKMLGEQKLISEEDLSGSAA [SEQ ID NO: 117] |
| TGF-β1 (2) | EVQLLESGGGLVQPGGSLRLSCAASGFTFGDYAMSWFRQAPGKGLEWVSGVSWNGSRTHYADSVKGRFTISRDNSKNTLYLQMNSLRAEDTAVYYCARDGNRPLDYWGQ |
| | GTLVTVSSGGGGSGGGGSGGGGSQSVLTQPPSASGTPGQRVTISCIGSSSNIGAGYDVHWYQQLPGTAPKLLIYGNSNRPSGVPDRFSGSKSGTSASLAISGLRSEDEADYYC |
| | AAWDDRLNGWVFGGGTKLXVLGEQKLISEXDLSGSAA [SEQ ID NO: 118] |
| TGF-β1 (3) | EVQLLESGGGLVQPGGSLRLSCAASGFTFSDYYIGWIRQAPGKGLEWVSGINWNGGSTGYADSVKGRFTISRDNSKNTLYLQMNSLRAEDTAVYYCARRSTPSSSWALPDFF |
| | DYWGQGTLVTVSSGGGGSGGGGSGGGGSQSVLTQPPSASGTPGQRVTISCTGSSSNIGANYDVHWYQQLPGTAPKLLIYSNNQRPSGVPDRFSGSKSGTSASLAISGLRSED |
| | XADYYCQSYDSSLSGWVFGGXTKLTVLGEQKLISXXXLSGSAA [SEQ ID NO: 119] |
| TNF-α (1) | EVQLLESGGGLVQPGGSLRLSCAASGFTFDDYGMSWVRQAPGKGLEWVAVISYDGSNKYYADSVKGRFTISRDNSKNTLYLQMNSLRAEDTAVYYCTRHLGSAMGYWGQG |
| | TLVTVSSGGGGSGGGGSGGGGSQSVLTQPPSASGTPGQRVTISCTGSSSNIGAGYDVHWYQQLPGTAPKLLIYGNSNRPSXVPDRFSGSXSGTSASLAISGLRSEDEADYYCQ |
| | SYDSSLSGWVFGGXTKLTVLXEQKLISXXDLSGSAA [SEQ ID NO: 120] |
| TNF-α (2) | EVQLLESGGGLVQPGGSLRLSCAASGFTFSSYGMHWVRQAPGKGLEWVSAISGSGGSTYYADSVKGRFTISRDNSKNTLYLQMNSLRAEDTAVYYCARGGWGPRSAFDIWG |
| | QGTLVTVSSGGGGSGGGGSGGGGSQSVLTQPPSASGTPGQRVTISCSGSSSNIGSNTVTWYQQLPGTAPKLLIYGNTNRLSGVPDRFSGSKSGTSASLAISGLRSEDEADYYCE |
| | AWDDKLFGPVFGGXTXLTVLXEQKLISEXXLSGSAA [SEQ ID NO: 121] |
| TNF-α (3) | EVQLLESGGGLVQPGGSLRLSCAASGFTFSSYWMSWVRQAPGKGLEWVSGVNWNGSRTHYADSVKGRFTISRDNSKNTLYLQMNSLRAEDTAVYYCASIRANYYYGMDV |
| | WGQGTLVTVSSGGGGSGGGGSGGGGSQSVLTQPPSASGTPGQRVTISCSGGSSNIGSHPVNWYQQLPGTAPKLLIYGNSNRPSGVPDRFSGSKSGTSASLAISGLRSEDEAD |
| | YYCAAWDASLSGWVFGGGXKLTVLXEXKLISXXXLSGSAA [SEQ ID NO: 122] |
| GM-CSF (1) | EVQLLESGGGLVQPGGSLRLSCAASGFTFSSYGMHWVRQAPGKGLEWVAVISYDGSNKYYADSVKGRFTISRDNSKNTLYLQMNSLRAEDTAVYYCARVGGMSAPVDYWG |
| | QGTLVTVSSGGGGSGGGGSGGGGSQSVLTQPPSASGTPGQRVTISCSGSSSNIGSNTVNWYQQLPGTAPKLLIYDNNKRPSGVPDRXSGSKSGTSASLAISGLRSEDEADYYC |
| | AAWDDSLIGLVVFGGXTKLTVLGEQKLISEXXLSGSAA [SEQ ID NO: 123] |
| GM-CSF (2) | EVQLLESGGGLVQPGGSLRLSCAASGFTFSSYAMSWVRQAPGKGLEWVAVISYDGSNEDSADSVKGRFTISRDNSKNTLYLQMNSLRAEDTAVYYCARGPSLRGVSDYWGQ |
| | GTLVTVSSGGGGSGGGGSGGGGSQSVLTQPPSASGTPGQRVTISCTGSSSNIGAGYDVHWYQQLPGTAPKLLIYNDNQRPSXVPDRFSGSKSGTSASLAISGLRSEDEADYYC |
| | QTWGTGINVIFGGXTKLXVLGEQKLISXEDLSGSAA [SEQ ID NO: 124] |
| GM-CSF (3) | EVQLLESGGGLVQPGGSLRLSCAASGFTFSSYAMSWVRQAPGKGLEWVAVISYDGSNEDSADSVKGRFTISRDNSKNTLYLQMNSLRAEDTAVYYCARGPSLRGVSDYWGQ |
| | GTLVTVSSGGGGSGGGGSGGGGSQSVLTQPPSASGTPGQRVTISCIGSSSNIGAGYDVHWYQQLPGTAPKLLIYNDNQRPSGVPDRFSGSKSGTSASLAISGLRSEDXADYYC |
| | QTWGTGINVIFGGXTKLTVLGEXKLISEXXLSGSAA [SEQ ID NO: 125] |
| TNF-β (1) | EVQLLESGGGLVQPGGSLRLSCAASGFTFSSFAMHWVRQAPGKGLEWVSAISGSGGSTYYADSVKGRFTISRDNSKNTLYLQMNSLRAEDTAVYYCASRSTLYYYYGMDVW |
| | GQGTLVTVSSGGGGSGGGGSGGGGSQSVLTQPPSASGTPGQRVTISCSGSTSNIGNSHVYWYQQLPGTAPKLLIYGNSNRPSXVPDRFSGSXSGTSASLAISGLRSEDEADYY |
| | CSSXAGSNNLVFGGXTKLTVLGEQKLISXXDLSGSAA [SEQ ID NO: 126] |
| TNF-β (2) | EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCARSPYYGMDVWGQGT |
| | LVTVSSGGGGSGGGGSGGGGSQSVLTQPPSASGTPGQRVTISCTGSSSNIGAGYDVHWYQQLPGTAPKLLIYRNDQRPSGVPDRFSGSKSGTSASLAISGLRSEDEADYYCSS |
| | YGGRDNVVFGGXTKLTVLXEQKLISXXXLSGSAA [SEQ ID NO: 127] |
| IL-1ra (1) | EVQLLESGGGLVQPGGSLRLSCAASGFTFDTHWMSWVRQAPGKGLEWVSAISGSGGSTYYADSVKGRFTISRDNSKNTLYLQMNSLRAEDTAVYYCARHDYGDYRAFDIW |
| | GQGTLVTVSSGGGGSGGGGSGGGGSQSVLTQPPSASGTPGQRVTISCTGSSSNIGAGYWHWYQQLPGTAPKLLIYGNNNRPSGVPDRFSGSKSGTSASLAISGLRSEDEAD |
| | YYCQSYDSSLSGVVFGGXTKLXVLXEQKLISXEDLSGSAA [SEQ ID NO: 128] |
| IL-1ra (2) | EVQLLESGGGLVQPGGSLRLSCAASGFTFSKYAMTWVRQAPGKGLEWVSAISGSGGNTYYADSVKGRFTISRDNSKNTLYLQMNSLRAEDTAVYYCARLVRGLYYGMDVW |
| | GQGTLVTVSSGGGGSGGGGSGGGGSQSVLTQPPSASGTPGQRVTISCSGSSSNIGNNAVNWYQQLPGTAPKLLIYGNSNRPSGVPDRFSGSKSGTSASLAISGLRSXDEADYY |
| | CQTXGTGPVVFGGXTKLTVLGEQKLISXXXXSGSAA [SEQ ID NO: 129] |
| IL-1ra (3) | EVQLLESGGGLVQPGGSLRLSCAVSGFTFSSYSMNWVRQAPGKGLEWVAGIGGRGATTYYVDSVKGRFTISRDNSKNTLYLQMNSLRAEDTAVYYCARLRVVPAARFDYWG |
| | QGTLVTVSSGGGGSGGGGSGGGGSQSVLTQPPSASGTPGQRVTISCSGSSSNIGSNTVNWYQQLPGTAPKLLIYGNSNRPSGVPDRFSGSKSGTSASLAISGLRSEDEADYYC |
| | QSYDSSLSGPPWVFGGXXKLXVLXEQKLISEEDLSGSAA [SEQ ID NO: 130] |
| IL-16 (1) | EVQLLESGGGLVQPGGSLRLSCAASGFTFSNHAMSWVRQAPGKGLEWVSGVSWNGSRTHYADSVKGRFTISRDNSKNTLYLQMNSLRAEDTAVYYCARAALVQGVKHAFE |
| | IWGQGTLVTVSSGGGGSGGGGSGGGGSQSVLTQPPSASGTPGQRVTISCSGSSSNIGSNTVNWYQQLPGTALKLLIYRNNQRPSGVPDRFSGSKSGTSASLAISGLRSEDEAD |
| | YYCASWDDRLSGLVFGGXTKLTVLGEQKLISEXDLSGSAA [SEQ ID NO: 131] |
| IL-16 (2) | EVQLLESGGGLVQPGGSLRLSCAASGFTFSNHAMSWVRQAPGKGLEWVSGVSWNGSRTHYADSVKGRFTISRDNSKNTLYLQMNSLRAEDTAVYYCARAALVQGVKHAFE |
| | IWGQGTLVTVSSGGGGSGGGGSGGGGSQSVLTQPPSASGTPGQRVTISCSGSSSNIGSNTVNWYQQLPGTALKLLIYRNNCIRPSGVPDRFSGSXSGTSASLAISGLRSEXEAD |
| | YYCASWDDRLSGLVFGGXTKLTVLXEQKLISEEDLSGSAA [SEQ ID NO: 132] |
| IL-18 (1) | EVQLLESGGGLVQPGGSLRLSCAASGFTFSSYGMHWVRQAPGKGLEWVSGINWNGGSTGYADSVKGRFTISRDNSKNTLYLQMNSLRAEDTAVYYCARDLRGGRFDPWG |
| | QGTLVTVSSGGGGSGGGGSGGGGSQSVLTQPPSASGTPGQRVTISCTGSSSNIGAGYVVHWYQQLPGTAPKLLIYRNNQRPSGVPDRFSGSXSGTSASLAISGLRSEDEADYY |
| | CSSXAGSKNLIFGGXTKLTVLGEQKLISXXXLSGSAA [SEQ ID NO: 133] |
| IL-18 (2) | EVQLLESGRGLVQPGGSLRLSCAASGFTFSSYGMHWVRQAPGKGLEWVSAIGTGGDTYYADSVMGRFTISRDNSKNTLYLQMNSLRAEDTAVYYCARSPRRGATAGTFDY |
| | WGQGTLVTVSSGGGGSGGGGSGGGGSQSVLTQPPSASGTPGQRVTISCSGSSSNIGSNIVNWYQQLPGTAPKLLIYRNNQRPSGVPDRFSGSKSGTSASLAISGLRSEDEADY |
| | YCXSYDNSLSGWVFGGXXKLXVLGEXKLISEXDLSGSAA [SEQ ID NO: 134] |
| MCP-4 (1) | EVQLLESGGGLVQPGGSLRLSCAASGFTFSSYGMHWVRQAPGKGLEWVSGISWNGGKTHYVDSVKGQFTISRDNSKNTLYLQMNSLRAEDTAVYYCARGGYSSGWAFDY |
| | WGQGTLVTVSSGGGGSGGGGSGGGGSQSVLTQPPSASGTPGQRVTISCSGRSSNIESNIVNWYQQLPGTAPKLLIYGNSNRPSGVPDRFSGSKSGTSASLAISGLRSEDEAD |
| | YYCAAWDDRLNAVVFGGXTKLTVLGEQKLISEXDLSGSAA [SEQ ID NO: 135] |
| IFN-γ (1) | EVQLLESGGGLVQPGGSLRLSCAASGFTFSSYGMHWVRQAPGKGLEWVSGVSWNGSRTHYADSVKGRFTISRDNSKNTLYLQMNSLRAEDTAVYYCARGRTGHGWKYYF |
| | DLWGQGTLVTVSSGGGGSGGGGSGGGGSQSVLTQPPSASGTPGQRVTISCSGSSSNIGNNAVNWYQQLPGTAPKLLIYGNSNRPSXVPDRFSGSXSGTSASLAISGLRSEDE |
| | ADYYCQXWGTGLGVFGGXTKLTVLGEXKLISEEXLSGSAA [SEQ ID NO: 136] |
| IFN-γ (2) | EVQLLESGGGLVQPGGSLRLSCAASGFTFSRHGFHWVRQGPGKGLEWVSGVSWNGSRTHYADSVKGRFTISRDNSKNTLYLQMNSLRAEDTAVYYCARGNWYRAFDIWG |
| | QGTLVTVSSGGGGSGGGGSGGGGSQSVLTQPPSASGTPGQRVTISCSGGSSHIGRNFISWYQQLPGTAPKLLIYAGNSRP [SEQ ID NO: 137] |
| IL-1β (1) | EVQLLESGGGLVQPGGSLRLSCAASGFTFSSYAMSWVRQAPGKGLEWVSYISSSGSTIYYADSVKGRSTISRDNSKNTLYLQMNSLRAEDTAVYYCARVRQNSGSYAYWGQG |
| | TLVTVSSGGGGSGGGGSGGGGSQSVLTQPPSASGTPGQRVTISCIGTSSNIGAPYDVHWYQQLPGTAPKLLIYGNSNRPSGVPDRFSGSKSGTSASLAISGLRSEDXADYYCQ |
| | SYDSSLSAVVFGGXTKLTVLGEQKLISEXXLSGSAA [SEQ ID NO: 138] |
| IL-1β (2) | EVQLLESGGGLVQPGGSLRLSCAASGFTFSRYVMTWVRQAPGKGLEWVSLISGGGSAMADSMKGRFTISRDNSKNTLYLQMNSLRAEDTAVYYCAKRVPYDSSGYYPDAF |
| | DIWGQGTLVTVSSGGGGSGGGGSGGGGSQSVLTQPPSASGTPGQRVTISCTGSSSNIGAGYDVHWYQQLPGTAPKLLIYGNSNRPSGVPDQFSGSKSGTSASLAISGLRSED |
| | EADYYCAAWDDSLNGPVFGGXTXLTXLXEQKLISEEXLSGSAA [SEQ ID NO: 139] |
| IL-1β (3) | EVQLLESGGGLVQPGGSLRLSCAASGFTFSSYWMSWVRQAPGKGLEWVAVVSYDGNNKYYADSRKGRFTISRDNSKNTLYLQMNSLRAEDTAMYYCASYWYTSGWYPYG |
| | MDVWGQGTLGTVSSGGGGSGGGGSGGGGSQSVLTQPPSASGTPGQRVTISCTGSSSNIGAGYDLHWYQQLPGTAPKLLIYRNNQRPSGVPDRFSGSKSGTSASLAISGLRS |
| | EDEADYYCSSYVDNNNLVFGGXTKLTVLXEQKLISEXXLSGSAA [SEQ ID NO: 140] |
| Eotaxin (1) | EVCILLESGGGLVQPGGSLRLSCAASGFTFSSYWMSWVRQAPGKGLEWVSGVSWNGSRTHYADSVKGRFTISRDNSKNTLYLQMNSLRAEDTAVYYCVKGKGTIAMPGRAR |
| | VGWWGQGTLVTVSSGGGGSGGGGSGGGGSQSVLTQPPSASGTPGQRVTISCSGSSSNIGNNAVNWYQQLPGTAPKLLIYANSNRPSGVPDRFSGSXSGTSASLAISGLRSE |
| | DEADYYCAAWDDSLSGPVFGGXTKLTVLGEQKLISXXDLSXSAA [SEQ ID NO: 141] |
| Eotaxin (2) | EVQLLESGGGLVQPGGSLRLSCAASGFTFSAYWMTWVRQAPGKGLEWVSVIYSGGSMADSVKGRFTISRDNSKNTLYLQMNSLRAEDTAVYYCARQTQQEYFDYWGQG |
| | TLVTVSSGGGGSGGGGSGGGGSQSVLTQPPSASGTPGQRVTISCFGSNSNIGSSTVNWYQQLPGTAPKLLIYDNDKRPSGVPDRFSGSXSGTSASLAISGLRSEDEADYYCAA |
| | WDDSLNGPVEGGXTKLTVLGEQKLISXXXLSGSXAAHHHHHH-SPRXPIRPIVSXXTIHWPSFYNVXTGKXXXLPNXIXXXHIPLSPAXXIXXXPXXXXX [SEQ ID NO: 142] |
| Eotaxin (3) | EVQLLESGGGLVQPGGSLRLSCAASGFTFRGYAMSWVRQAPGKGLEWVSGVSWNGSRTHYADSVKGRFTISRDNSKNTLYLQMNSLRAEDTAVYYCARAPAVAGWFDPW |
| | GQGTLVTVSSGGGGSGGGGSGGGGSQSVLTQPPSASGTPGQRVTISCSGSSSNIGSHTVNWYQQLPGTAPKLLIYRNNQRPSGVPDRFSGSKSGTSASLAISGLRSEDXADYY |
| | CAAWDDSLSGRVXGGGXKLTVLGEQKLISEEDLSGSAA [SEQ ID NO: 143] |
| RANTES (1) | EVQLLESGGGLVQPGGSLRLSCAASGFTFSSYGMHWVRQAPGKGLEWVAVISNDGTKKDYADSVKGRFTISRDNSKNTLYLQMNSLRAEDTAVYYCARDASGYDDYYFDY |
| | WGQGTLVTVSSGGGGSGGGGSGGGGSQSVLICIPPSASGTPGQRVTISCTGSSSNIGAGSDVHWYQQLPGTAPKLLIYRDDQRSSGVPDRFSGSKSGTSAFLAISGLRSEDEA |
| | DYYCQSYDNSLSGWVFGGXTKLTVLGEQKLISEXXLSGSAA [SEQ ID NO: 144] |
| RANTES (2) | EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCARDNDYSSDTFDYWG |
| | QGTLVTVSSGGGGSGGGGSGGGGSQSVLTQPPSAFGTPGQRVTISCSGSSSNIGSDYVYWYQQLPGTAPKLLIYSDNQRP [SEQ ID NO: 145] |
| RANTES (3) | EVQLLESGGGLVQPGGSLRLSCAASGFTFSNYGMNWVRQAPGKGLEWVSGVSWNGSRTHYVDSVKRRFTISRDNSKNTLYLQMNSLRAEDTAVYYCARPRLRSHNYYGM |
| | DVWGQGTLVTVSSGGGGSGGGGSGGGGSQSVLTQPPSASGTPGQRVTISCSGSSFKSGKNYVSWYQQLPGTAPKLLIYRNNQRPSGVPDRFSGSKSGTSASLAISGLRSEDE |
| | ADYYCAAWDVRVKGVIFGGXTKLTVLGEQKLISEXDLSGSAA [SEQ ID NO: 146] |
| MCP-1 (1) | EVQLLESGGGLVQPGGSLRLSCAASGFTFSSYAMSWVRQAPGKGLEWVSGVSWNGSRTHYADSVKGRFTISRDNSKNTLYLQMNSLRAEDTAVYYCARGGHQQLGQWG |
| | QGTLVTVSSGGGGSGGGGSGGGGSQSVLTQPPSASGTPGQRVTISCSGSSSNIGNNYVSWYQQLPGTAPKLLIYRDSRRPSGVPDRFSGSKSGTSASLAISGLRSEXEADYYCA |
| | AWDDSLKGWLFGGXTKLTVLXEQKLISEXXLSGSAA [SEQ ID NO: 147] |
| MCP-1 (2) | EVQLLESGGGLVQPGGSLRLSCAASGFTFSSYAMSWVRQAPGKGLEWVSYISSSSSYTNYADSVKGRFTISRDNSKNTLYLQMNSLRAEDTAVYYCARFRYNSGKMFDYWGQ |
| | GTLVTVSSGGGGSGGGGSGGGGSQSVLTQPPSASGTPGQRVTISCSGSSSNIGRNIVNWYQQLPGTAPKLLIYGNSNRRSGVPDRFSGSKSGTSASLAISGLRSEDEADYYCA |
| | AWDDSLSGVVFGGXTKLTVLXEQKLISEXDLSGSAA [SEQ ID NO: 148] |
| MCP-1 (3) | EVQLLESGGGLVQPGGSLRLSCAASGFTFSNYGMHWVRQAPGKGLEWVAVISYDGSNKYYADSVKGRFTISRDNSKNTLYLQMNSLRAEDTAVYYCAKSHYYDTTSFDYWG |
| | QGTLVTVSSGGGGSGGGGSGGGGSQSVLTQPPSASGTPGQRVTISCSGSSSNIGTNPVNWYQQLPGTAPKLLIYDNNKRPSGVPDRFSGSKSGTSASLAISGLRSEDXADYYC |
| | AAWDDSLSGVVFGGXTKLTVLGEQKLISXEDLSGSAA [SEQ ID NO: 149] |
| MCP-3 (1) | EVQLLESGGGLVQPGGSLRLSCAASGFTFSTYGMHWVRQAPGKGLEWVSGVSWNGSRTHYVNSVKRRFTISRDNSKNTLYLQMNSLRAEDTAVYYCARVAPGSGKRLRAF |
| | DIWGQGTLVTVSSGGGGSGGGGSGGGGSQSVLTQPPSASGTPGQRVTISCSGSSSNIGNNAVNWYQQLPGTAPKLLIYEVSKRPPGVPDRFSGSKSGTSASLAISGLRSEDXA |
| | DYYCSSYAGSSKWVFGGXTKLTVLGEQKLISEEDLSGSAA [SEQ ID NO: 150] |
| MCP-3 (2) | EVQLLESGGGLVQPGGSLRLSCAASGFTLSSNYMSWVRQAPGKGLEWVSGISASGHSTHYADSGKARFTISRDNSKNTLYLQMNSLRAEDTAVYYCARGKSLAYWGQGTLV |
| | TVSSGGGGSGGGGSGGGGSQSVLTQPPSASGTPGQRVTISCSGSSSNIGNNAVNWYQQLPGTAPKLLIYRNNQRPSGVPDRFSGSKSGTSASLAISGLRSEDEADYYCAAWD |
| | DSLSVVVFGGXTKLTVLGEQKLISXXXLSGSAA [SEQ ID NO: 151] |
| MCP-3 (3) | EVQLLESGGGLVQPGGSLRLSCAASGFTFSIYWMSWVRQAPGKGLEWVAYIGGISNTVSYSDSVKGRFTISRDNSKNTLYLQMNSLRAEDTAVYYCAKAPGYSSGWGWFDP |
| | WGQGTLVTVSSGGGGSGGGGSGGGGSQSVLTQPPSASGTPGQRVTISCSGSSSNIGTNSVFWYQQLPGTAPKLLIYGNNNRPSGVPDRFSGSKSGTSASLAISGLRSEDXAD |
| | YYCMIWHSSASVFGXXTKLTVLGEQKLISEXXLSGSAA [SEQ ID NO: 152] |
| β-gal | EVQLLESGGGLVQPGGSLRLSCAASGFTFSSYAMHWVRQAPGKGLEWVAVIAYDGINEYYGDSVKGRFTISRDNSKNTLYLQMNSLRAEDTAVYYCARGGIYHGFDIWGQG |
| | TLVTVSSGGGGSGGGGSGGGGSQSVLTQPPSASGTPGQRVTISCSGSSSNIGSNYVYWYQQLPGTAPKLLIYDNHKRPSGVPDRFSGSXSGTSASLAISGLRSEDEADYYCAA |
| | WDDNSWVFGGXTKLTVLGXYKDDDDKAA [SEQ ID NO: 153] |
| Angiomotin (1) | EVQLLESGGGLVQPGGSLRLSCAASGFTFSDHYMDWVRQAPGKGLEWVSGVSWNGSRTHYADSVKGRFTISRDNSKNTLYLQMNSLRAEDTAVYYCARDTWAYGAFDIW |
| | GQGTLVTVSSGGGGSGGGGSGGGGSQSVLTQPPSASGTPGQRVTISCSGSNSNIGRNTVNWYQQLPGTAPKWYRDNQRPSGVPDRFSGSXSGTPASLAISGLRSEDXADY |
| | YCAAWDVSLNGWVFGGXTKLTVLGDYXDHDGDYKDHDIDXXDDDDXXAAHHHHHH-SPRWXIRPIVSRITIXWXXFYXVXXXKXX [SEQ ID NO: 154] |
| Angiomotin (2) | EVQLLESGGGLVQPGGSLRLSCAASGFTFNDYYMTWIRQAPGKGLEWVSYISSSGSTIYYADSVKGRFTISRDNSKNTLYLQMNSLRAEDTAVYYCARERLPDVFDVWGQGTL |
| | VTVSSGGGGSGGGGSGGGGSQSVLTQPPSASGTPGQRVTISCSGSGSNIGTNSVSWYQQLPGTAPKWYFDDLLPSGVPDRFSGSKSGTSASLAISGLRSEDEADYYCAAWD |
| | DSLSGVVFGGXTKLTVLGXYKDHDGDYKDHDIDYKDDDXKAXAHHHHHH-SPRXXXRXIVSXIXIHXXXFYNXXTGKTXXXXXXIXXAAXXXFXX [SEQ ID NO: 155] |
| Leptin | EVQLLESGGGLVQPGGSLRLSCAASGFTFGDFAMSWVRQAPGKGLEWVANIKQDGSVKYYVDSVKGRFTISRDNSKNTLYLQMNSLRAEDTAVYYCARFLAGFYYGMDVW |
| | GQGTLVTVSSGGGGSGGGGSGGGGSQSVLTQPPSASGTPGQRVTISCSGSDSNIGGNTVNWYQQLPGMAPKLLIYYDDLLPSGVPDRFSGSKSGTSASLAISGLRSEDEADY |
| | YCAAYDDTMNGWGFGGXTKLTVLGXYKDXDDKAA [SEQ ID NO: 156] |
| Integrin α-10 | EVQLLESGGGLVQPGGSLRLSCAASGFTFSTYNMNWVRQAPGKGLEWVSTISGSGGRTYYADSVKGRFTISRDNSKNTLYLQMNSLRAEDTAVYYCARDRVATLDAFDIWG |
| | QGTLVTVSSGGGGSGGGGSGGGGSQSVLTQPPSASGTPGQRVTISCSGSSSNIGSNSVSWYQQLPGTAPKLLIYSNNQRPSGVPDRFSGSKSGTSASLAISGLRSEDEADYYCA |
| | AWDDSLSGVVFGGXTKLTVLGEQKLISEXDLSGSAA [SEQ ID NO: 157] |
| Integrin α-11 | EVQLLESGGGLVQPGGSLRLSCAASGFTFRRDWMSWVRQVPGKGLEWVSVISGSDGSTYYADSVKGRFTISRDNSKNTLYLQMNSLRAEDTAVYYCASYSPLGNWFDSWG |
| | QGTLVTVSSGGGGSGGGGSGGGGSQSVLTQPPSASGTPGQRVTISCTGSSSNIGAGYDVHWYQQLPGTAPKLLIYSDTYRPSGVPDRFSGSXSGTSASLAISGLRSEDEADYYC |
| | QSYDSSLXGFVVFGGXTKLTVLXEQKLISEXXLSGSAA [SEQ ID NO: 158] |
| IgM (1) | EVQLLESGGGLVQPGGSLRLSCAASGFTFSDYYMSWIRQAPGKGLEWVSAIGSGPYYAHSVRDRFTISRDNSKNTLYLQMNSLRAEDTAVYYCARGGVEASFDYWGQGTLVT |
| | VSSGGGGSGGGGSGGGGSQSVLTQPPSASGTPGQRVTISCTGSSSNIGAGYDVHWYQQLPGTAPKLLIYGNTNRPSGVPNRFSGSKSGTSASLAISGLRSEDEADYYCQSYDN |
| | DLSGWVFGGXTKLXVLGEQKLISXXXLSGSAA [SEQ ID NO: 159] |
| LDL (1) | EVQLLESGGGLVQPGGSLRLSCAASGFTFSDYYMSWVRQAPGKGLEWVSGVSWNGSRTHYADSVKGRFTISRDNSKNTLYLQMNSLRAEDTAVYYCARAARYSYYYYGMD |
| | VWGQGTLVTVSSGGGGSGGGGSGGGGSQSVLTQPPSASGTPGQRVTISCSGSSSNIGNNAVNWYQQLPGTAPKLLIYGNDRRPSGVPDRFSGSKSGTSASLAISGLRSEDEA |
| | DYYCQTWGTGRGVFGGGTKLTVLGEQKLISEXXLSGSAA [SEQ ID NO: 160] |
| LDL (2) | EVQLLESGGGLVQPGGSLRLSCAASGFTFSNAWMSWVRQAPGKGLEWVSSISTSSNYIYYADSVKGRFTISRDNSKNTLYLQMNSLRAEDTAVYYCARVKKYSSGWYSNYAF |
| | DIWGQGTLVTVSSGGGGSGGGGSGGGGSQSVLTQPPSASGTPGQRVTISCSGSSSSIGNNFVSWYQQLPGTAPKLLIYDNNKRPSXVPDRFSGSXSGTSASLAISGLRSEDXA |
| | DYYCAAWDDSLNGWVFGGXTKLTVLXXYKDHDGDYXDHDIDYKDXXDKAA [SEQ ID NO: 161] |
| PSA | EVQLLESGGGLVQPGGSLRLSCAASGFTFRSYEMNWVRQAPGKGLEWVAVIGGNGVDTDYADSVKGRFTISRDNSKNTLYLQMNSLRAEDTAVYYCVREEVDFWSGYYSY |
| | GMDVWGQGTLVTVSSGGGGSGGGGSGGGGSQSVLTQPPSASGTPGQRVTISCSGSSSNIGDNFVSWYQQLPGTAPKLLIYRTNGRPSGVPDRFSGSXSGTSASLAISGLRSE |
| | DEADYYCATWDDNLNGRVVFGGXTKLTVLGDYKDXXDKAA [SEQ ID NO: 162] |
| Lewis^{x} (1) | EVQLLESGGGLVQPGGSLRLSCAASGFTFSNYWMHWVRQAPGKGLEWVANIKEDGSEKYYVDSVKGRFTISRDNSKNTLYLQMNSLRAEDTAVYYCAREGETSFGLDVWG |
| | QGTLVTVSSGGGGSGGGGSGGGGSQSVLTQPPSASGTPGQRVTISCSGSSSNIGSNTVNWYQQLPGTAPKLLIYSNNQRPSGVPDRFSGSKSGTSASLAISGLRSEDEADYYC |
| | ASWDDSLSGWVFGGXTKLTVLGDYKDDDDKAA [SEQ ID NO: 163] |
| Lewisx (2) | EVQLLESGGGLVQPGGSLRLSCAASGFTFSRYWMHWVRQAPGKGLEWVANIKPDGSEQYYADSVKGRFTISRDNSKNTLYLQMNSLRAEDTAVYYCAREGLSSGWSYGMD |
| | VWGQGTLVTVSSGGGGSGGGGSGGGGSQSVLTQPPSASGTPGQRVTISCSGSNSNIGSNTVNWYQQLPGTAPKLLIYTNINRPSGVPDRFSGSKSGTSASLAISGLRSXDEA |
| | DYYCATWDDSLSGWVFGGXTKLTVLGXYKDXXDKAA [SEQ ID NO: 164] |
| Lewis Y | EVQLLESGGGLVQSGGSLRLSCAASGFTFSSYTLHWVRQAPGKGLEYVSAISSNGGSTYYADSVKGRFTISRDNSKNTLYLQMNSLRAEDTAVYYCASDVYGDYPRGLDYWGQ |
| | GTLVTVSSGGGGSGGGGSGGGGSQSVLTQPPSASGTPGQRVTISCSGTTSNIGSNYVHWYQQLPGTAPKLLIYGNNNRPSGVPDRFSGSKSGTSASLAISGLRSEDXADYYCQ |
| | SYDRSLGGLRVFGGXTKLTVLXDYKXDDDKAA [SEQ ID NO: 165] |
| Sialle x | EVQLLESGGGLVQPGGSLRLSCAASGFTLSSYAMSWVRQAPGKGLEWVSSISSGNSYIYYADSVKGRFTISRDNSKNTLYLQMNSLRAEDTAVYYCARGRGRGGGFELWGQG |
| | TLVTVSSGGGGSGGGGSGGGGSQSVLTQPPSASGTPGQRVTISCSGSSSNIGTYTVNWYQQLPGTAPKLLIYSNNQRPSGVPDRFSGSKSGISASLAISGLRSEXEADYYCSSN |
| | AGIDNILFGGXTKLTVLGEQKLISEXDLSGSXAAHHHHXXXXXXXXIXXXXXXXXXXXXXXXXXXLXX [SEQ ID NO: 166] |
| TM peptide | EVQLLESGGGLVQPGGSLRLSCAASGFTFSSYGFHWVRQAPGKGLEWVSLISWDGGSTYYADSVKGRFTISRDNSKNTLYLQMNSLRAEDTAVYYCARGTWFDPWGQGTLV |
| | TVSSGGGGSGGGGSGGGGSQSVLTQPPSASGTPGQRVTISCSGSSSXIGNNAVNWYQQLPGTAPKLLIYRNNQRPSXVPDRFSGXXSGTSASLAIXGLRSEDEADYYCAAWD |
| | DSLSWVFGGXTKLTVLGDXXTMXVIIKIMTSXXXMTMXRRP [SEQ ID NO: 167] |
| Procathepsin | EVQLLESGGGLVQPGGSLRLSCAASGFTFSSYAMSWVRQAPGKGLEWVSSMSASGGSTYYADSVKGRFTISRDNSKNTLYLQMNSLRAEDTAVYYCARDRGSYGMDVWG |
| W | QGTLVTVSSGGGGSGGGGSGGGGSQSVLTQPPSASGTPGQRVTISCSGSTSNIGSYAVNWYQQLPGTAPKLLIYGNNNRPSGVPDRFSGSXSGTSASLAISGPRSEDEADYYC |
| | AAWDDSLNGGVFGGXTKLTVLGXYKXDDDKAA [SEQ ID NO: 168] |
| BTK (1) | EVQLLESGGGLVQPGGSLRLSCAASGFTFSNYAMSWVRQAPGKGLEWVSGINWNGGSTGYADSVKGRFTISRDNSKNTLYLQMNSLRAEDTAVYYCAKHLKRYSGSSYLFD |
| | YWGQGTLVTVSSGGGGSGGGGSGGGGSQSVLTQPPSASGTPGQRVTISCSGSSSXIGSNYVYWYQQLPGTAPKLLIY [SEQ ID NO: 169] |
| Digoxin | EVQLLESGGGLVQPGGSLRLSCAASGFTFSSYAMSWVRQAPGKGLEWVAVIWHDGSSKYYADSVKGRFTISRDNSKNTLYLQMNSLRAEDTAVYYCARATGDGFDYWGQG |
| | TLVTVSSGGGGSGGGGSGGGGSQSVLTQPPSASGTPGQRVTISCSGSSSNIGSNYVYWYQQLPGTAPKLLIYRNNQRPSGVPDRFSGSKSGTSASLAISGLRSEDEADYYCAA |
| | WDDSLNGVVFGGXTKLTVLGEQKLISXXXLSXSAA [SEQ ID NO: 170] |
| GLP-1 R | EVQLLESGGGLVQPGGSLRLSCAASGFTFRSYGMHWVRQAPGKGLEWVSGLSWNSAGTGYADSVKGRFTISRDNSKNTLYLQMNSLRAEDTAVYYCAKEMGNNWDHIDY |
| | WGQGTLVTVSSGGGGSGGGGSGGGGSQSVLTQPPSASGTPGQRVTISCTGSSSNIGAGYDVHWYQQLPGTAPKLLIYGNSNRPSGVPDRFSGSKSGTSASLAISGLRSEDEA |
| | DYYCAAWDDGLSGPVFGGGTKLTXLGEQKLISEEDLSGSAA [SEQ ID NO: 171] |
| GLP-1 | EVQLLESGGGLVQPGGSLRLSCAASGFTFNSYGMHWVRQAPGKGLEWVSAISGSGGSTYYAESVKGRSTISRDNSKNTLYLQMNSLRAEDTAVYYCVTRNAVFGFDVWGQ |
| | GTLVTVSSGGGGSGGGGSGGGGSQSVLTQPPSASGTPGQRVTISCTGSSSNIGAGFDVHWYQQLPGTAPKLLIYDNNKRPSGVPDRFSGSKSGTSASLAISGLRSEDEADYYC |
| | QSFDSSLSGVVFGGXTKLTVLXEQKLISXEXLSGSAA [SEQ ID NO: 172] |
| C1q | EVQLLESGGGLVQPGGSLRLSCAASGFTFDDYGMSWVRQVPGKGLEWVSAISGSGATTFYAHSVQGRFTISRDNSKNTLYLQMNSLRAEDTAVYYCARGGRGYDWPSGAF |
| | DIWGQGTLVTVSSGGGGSGGGGSGGGGSQSVLTQPPSASGTPGQRVTISCTGSSSNIGAGYDVHWYQQLPGTAPKWYENNKRPSXVPDRFSGSKSGTSASLAISGLRSEDX |
| | ADYYCAAWDDSVNGYVVFGGXTKLTVLGEQKLISEXXLSGSAAXXHHHHH-SPRWPIRPIXSRXTIXXPSFYXXXXXXTXXLPXXIXXXHXPXXXXXX [SEQ ID NO: 173] |
| C1s | EVQLLESGGGLVQPGGSLRLSCAASGFTFSSYAMSWVRQAPGKGLEWVSGVSWNGSRTHYADSVKGRFTISRDNSKNTLYLQMNSLRAEDTAVYYCARHMKAAAYVFEIW |
| | GQGTLVTVSSGGGGSGGGGSGGGGSQSVLTQPPSASGTPGQRVTISCSGSSSNIGSTAVNWYQQLPGTAPKLLIYSNNKRPSGVPDRFSGSXSGTSASLAISGLRSEDEADYY |
| | CAAWDDRLNGNVLFGGXXKLTVLXEQXLISXXXLSGSAA [SEQ ID NO: 174] |
| C3 (1) | EVQLLESGGGLVQPGGSLRLSCAASGFTFSSYAMSWVRQAPGKGLEWVSSVTGSGGGTYYADSVEGRFTISRDNSKNTLYLQMNSLRAEDTAVYYCARYRWFGNDAFDIW |
| | GQGTLVTVSSGGGGSGGGGSGGGGSQSVLTQPPSASGTPGQRVTISCSGSASNLGMHFVSWYQQLPGTAPKLLIYGNSNRPSGVPDRFSGSKSGTSASLAISGLRSEDEADY |
| | YCAAWDDTLNIWVFGGXTKLTVLGEQKLISXXXLSGSAA [SEQ ID NO: 175] |
| C3 (2) | EVQLLESGGGLVQPGGSLRLSCAASGFTFSTYRMIWVRQAPGKGLEWVSSISGSNTYIHYADSVRGRFTISRDNSKNTLYLQMNSLRAEDTAVYYCARDRHPLLPSGMDVWG |
| | QGTLVTVSSGGGGSGGGGSGGGGSQSVLTQPPSASGTPGQRVTISCSGSSSNIGKHPVNWYQQLPGTAPKLLIYRNDQRPSGVPDRFSGSKSGTSASLAISGLRSEDXADYYC |
| | QSYDSSLSGSWVFGGXTKLTVLGXQKLISEEDLSGSAA [SEQ ID NO: 176] |
| C4 (1) | EVQLLESGGGLVQPGGSLRLSCAASGFTFSSYPMSWVRQAPGKGLEWVSTLYAGGWTSYADSVWGRFTISRDNSKNTLYLQMNSLRAEDTAVYYCARPKVESLSRYGMDV |
| | WGQGTLVTVSSGGGGSGGGGSGGGGSQSVLTQPPSASGTPGQRVTISCTGSSSNIGAGYVVHWYQQLPGTAPKLLIYDNSKRPSGVPDRFSGSKSGTSASLAISGLRSEDEA |
| | DYYCQSYDSSLSGVVFGGXTKLTVLXEQKLISEXXLSGSAA [SEQ ID NO: 177] |
| C5 (1) | EVQLLESGGGLVQPGGSLRLSCAASGFTFSSYRMNWVRQAPGKGLEWVSAISGSGGSTYYADSVKGRFTISRDNSKNTLYLQMNSLRAEDTAVYYCARGGGWFSGHYYFDY |
| | WGQGTLVTVSSGGGGSGGGGSGGGGSQSVLTQPPSASGTPGQRVTISCTGATSNIGAGYDVHWYQQLPGTAPKLLIYRNNQRPSXVPDRFSGSXSGTSASLAIXGLRSEDXA |
| | DYYCQSYDSSLRHWVFXGXXKLTVLXEQKLISEXXLSGSXA [SEQ ID NO: 178] |
| C5 (2) | EVQLLESGGGLVQPGGSLRLSCAASGFTFSAYSMNWVRQAPGKGLEWVSGVSWNGSRTHYADSVKGRFTISRDNSKNTLYLQMNSLRAEDTAVYYCARENSGFFDYWGQ |
| | GTLVTVSSGGGGSGGGGSGGGGSQSVLTQPPSASGTPGQRVTISCTGSSSNIGSNTVNWYQQLPGTAPKLLIYGNSNRPSGVPDRFSGSKSGTSASLTISGLRSEDXADYYCA |
| | AWDDSLSGWVFGGXTKLTVLXEQKLISEEXLSGSAA [SEQ ID NO: 179] |
| C1 inh (1) | EVQLLESGGGLVQPGGSLXLSCAASGFTFSDYYMSWIRQAPGKGLEWVSGISRGGEYTFYVDSVKGRFTISRDNSKNTLYLQMNSLRAEDTAVYYCARDPGGLDAFDIWGQG |
| | TLVTVSSGGGGSGGGGSGGGGSQSVLTQPPSASGTPGQRVTISCTGSSSNIGARYDVQWYQQLPGTAPKLLIYGNSNRPSGVPDRFSGSKSGTSASLAISGLRSEXXADYYCAS |
| | WDDSLSGPVFGGXTKLTVLXEQKLISEXXLSXSAA [SEQ ID NO: 180] |
| Factor B (1) | EVQLLESGGGLVQPGGSLRLSCAASGFTFSSYSMNWVRQAPGKGLEWVAVISYDGRFIYYSDSVKGRFTISRDNSKNTLYLQMNSLRAEDTAVYYCARSYGGNLAMDVWGQ |
| | GTLVTVSSGGGGSGGGGSGGGGSQSVLTQPPSASGTPGQRVTISCTGSSSNIGAGYDVHWYQQLPGTAPKLLIYDNNKRPSGVPDRFSGSNSGTSASLAISGLRSEDXADYYC |
| | AAWDDRLNGRVVFGGXTKLTVLGEQKLISEXDLSGSAA [SEQ ID NO: 181] |
| IL-12 (3) | EVQLLESGGGLVQPGGSLRLSCAASGFTFSRYGMHWVRQAPGKGLEWVASIRGNARGSFYADSVKGRFTISRDNSKNTLYLQMNSLRAEDTAVYYCAKGDSSGWYFFDYW |
| | GQGTLVTVSSGGGGSGGGGSGGGGSQSVLTQPPSASGTPGQRVTISCTGSDSXIGAGFDVHWYQQLPGTAPKLLIYGNNNRPSGVPDRFSGSKSGTSASLAISGLRSEDXAD |
| | YYCQSYDTSLSGVLFGGXXKLTVLGEQKLISEXDLSGSAA [SEQ ID NO: 182] |
| IL-12 (4) | EVQLLESGGGLVQPGGSLRLSCAASGFTFSDYGMHWVRQAPGKGLEWVSTVSGSGDNTYYADSVKGRFTISRDNSKNTLYLQMNSLRAEDTAVYYCiIiWRYWGQGTLVT |
| | VSSGGGGSGGGGSGGGGSQSVLTQPPSASGTPGQRVTISCSGSSSNIGSNTVNWYQQLPGTAPKLLIYGNSNRPSGVPDRFSGSKSGTSASLAISGLRSEDEADYYCQSYDSSL |
| | SGWVFGGXTKLTVLXEQKLISXEDLSGSAA [SEQ ID NO: 183] |
| IL-16 (3) | EVXLLESGGGLVQPGGSLRLSCAASGFTFSSYGMHWVRQAPGKGLEWVSGINWNGGSTGYADSVKGRFTISRDNSKNTLYLQMNSLRAEDTAVYYCARERGDAFDIWGQG |
| | TLVTVSSGGGGSGGGGSGGGGSQSVLTQPPSASGTPGQRVTISCSGSSSNIGSNTVNWYQQLPGTAPKLLIYSDNQRPSGVPDRFSGSKSGTSASLAISGLRSXXEADYYCAA |
| | WXDSLNGPWVFGGXTKLXVLGEQKLISEEDLSGSAA [SEQ ID NO: 184] |
| IL-18 (3) | EVQLLESGGGLVQPGGSLRLSCAASGFTFSRYGMHWVRQAPGKGLEWVAVISYDGSNKYYADSVKGRFTISRDNSKNTLYLQMNSLRAEDTAVYYCARHGYGDSRSAFDIW |
| | GQGTLVTVSSGGGGSGGGGSGGGGSQSVLTQPPSASGTPGQRVTISCTGSSSNIGAGYDVHWYQQLPGTAPKLLIYRNNQRPSGVPDRFSGSXSGTSASLAISGLRSEXXADY |
| | YCQSYDSSLSRWVFGGXTKLXVLGEQKLISXXXLSXSAA [SEQ ID NO: 185] |
| IL-1a (3) | EVQLLESGGGLVQPGGSLRLSCAASGFTFSSYSMNWVRQAPGKGLEWVSYISSSSSYTNYADSVKGRFTISRDNSKNTLYLQMNSLRAEDTAVYYCARSVTRRAGYYYYYSGM |
| | DVWGQGTLVTVSSGGGGSGGGGSGGGGSQSVLTQPPSASGTPGQRVTISCSGSSSXIGSNTVNWYQQLPGTAPKLLIYRNNQRPSXVPDRFSGSXSGTSASLAISGLRSEDE |
| | AXYYCSSXAGSNSXVFGGXTKLTVLGEQKLISXXXLSGSAA [SEQ ID NO: 186] |
| IL-6 (3) | EVQLLESGGGLVQPGGSLRLSCAASGFTFSNYGMHWVRQAPGKGLEWVSSITSSGDGTYFADSVKGRFTISRDNSKNTLYLQMNSLRAEDTAVYYCARAGGIAAAYAFDIW |
| | GQGTLVTVSSGGGGSGGGGSGGGGSQSVLTQPPSASGTPGQRVTISCSGSSSNVGSNYVYWYQQLPGTAPMAYDNNKRPSGVPDRFSGSKSGTSASLAISGLRSEXEADYY |
| | CQSYDSSRWVFGGXTKLTVLGEQKLISEXXLSGSAA [SEQ ID NO: 187] |
| IL-6 (4) | EVQLLESGGGLVQPGGSLRLSCAASGFTFSSNYMSWVRQAPGKGLEWVSSISSSSTIYYADSVKGRFTISRDNSKNTLYLQMNSLRAEDTAVYYCARQPASGTYDAFDIWGQG |
| | TLVTVSSGGGGSGGGGSGGGGSQSVLTQPPSXSGTPGQRVTISCTGSSSNIGAGYDVHWYQQLPGTAPKLLIYYDDLLPSGVPDRFSGSKSGTSASLAISXLRSEDEADYYCAV |
| | WDDSLSGWVFGGXTKLTVLXEQKLISXXDLSGSAXAHHHHHHXSPRXXIRPIVSXITIHXXVVLXRRDWEXPXXTQLNXXXAHXPFXXXXNX [SEQ ID NO: 188] |
| IL-8 (3) | EVQXLESGGGLVQPGGSLRLSCAASGFTFDDYGMSWVRQAPGKGLEWVSLISWDGGSTYYADSVKGRFTISRDNSKNTLYLQMNSLRAEDTAVYYCARDDLYGMDVWGQ |
| | GTLVTVSSGGGGSGGGGSGGGGSQSVLTQPPSASGTPGQRVTISCIGSSSNIGAGYDVHWYQQLPGTAPKLLIYDNNKRPSGVPDRFSGSKSGTSASLAISGLRSXDEADYYC |
| | AAWDDSLSGWVFGGXTKLTVLGEQKLISEXXLSGSAA [SEQ ID NO: 189] |
| MCP-4 (3) | EVQLLESGGGLVQPGGSLRLSCAASGFTFSSYGMHWVRQAPGKGLEWVSGISWNGGKTHYVDSVKGQFTISRDNSKNTLYLQMNSLRAEDTAVYYCARGGYSSGWAFDY |
| | WGQGTLVTVSSGGGGSGGGGSGGGGSQSVLTQPPSASGTPGQRVTISCSGRSSNIESNTVNWYQQLPGTAPKLLIYGNSNRPSGVPDRFSGSKSGTSASLAISGLRSEXEADY |
| | YCAAWDDRLNAVVFGGXTKLXVLXEQKLISEXXLSGSAA [SEQ ID NO: 190] |
| Properdin | EVQLLESGGGLVQPGGSLRLSCAASGFTFSSNYMSWVRQAPGKGLEWVSAISGSGGSTYYADSVKGRFTISRDNSKNTLYLQMNSLRAEDTAVYYCAKGGSGWYDYFDYWG |
| | QGTLVTVSSGGGGSGGGGSGGGGSQSVLTQPPSASGTPGQRVTISCTGSSSNIGAGYDVHWYQQLPGTAPKLLIYRNNQRPSGVPDRFSGSKSGTSASLAISGLRSEDEAXYY |
| | CAAXDDGLNSPVFGGGTKLXVLXEQKLISEEDLSGSAXAHHHHHH-SPRXXIRPIVSRITIHWXXFXXXXXGKTXXXPXLXXXXXXPPFX [SEQ ID NO: 191] |
| TNF-β (3) | EVQLLESGGGLVQPGGSLRLSCAASGFTFSDYYMSWIRQAPGKGLEWVSGLSGSAGRTHYADSVRGRFTISRDNSKNTLYLQMNSLRAEDTAMYYCASSLFDYWGQGTLVT |
| | VSSGGGGSGGGGSGGGGSQSVLTOPPSASGTPGQRVTISCSGSSSNIGSNYVYWYQQLPGTAPKLLIYSNNQRPSGVPDRFSGSKSGTSASLAISGLRSEDXADYYCAAWDDS |
| | LNAVVFGGXTKLTVLGEQKLISEXDLSGSAA [SEQ ID NO: 192] |
| TNF-β (4) | EVQLLESGGGLVQPGGSLRLSCAASGFTFSDYYMNWVRQAPGKGLEWVSGINWNSDDIDYVDSVKGRFTISRDNSKNTLYLQMNSLRAEDTAMYYCAIDSRYSSGWSFEY |
| | WGQGTLVTVSSGGGGSGGGGSGGGGSQSVLTQPPSASGTPGQRVTISCSGSTSNIGNSHVYWYQQLPGTAPKLLIYSNNQRPSGVPDRFSGSKSGTSASLAISGLRSEDEAD |
| | YYCQSYDSSLSGVVFGGXTKLTVLGEQKLISXXXLSGSAA [SEQ ID NO: 193] |
| VEGF (3) | EVQLLESGGGLVQPGGSLRLSCAASGFTFSSYEMNWVRQAPGKGLEWVSGISGSGGFTYYADSVKGRFTISRDNSKNTLYLQMNSLRAEDTAMYYCAREGYQDAFDIWGQ |
| | GTLVTVSSGGGGSGGGGSGGGGSQSVLTQPPSASGTPGQRVTISCTGSSSNIGAGYDVHWYQQLPGTAPKWYSNNQRPSXVPDRFSGSXSGTSASLAISGLRSEDXADYYC |
| | AAWDDSLSGPPWVFGGGXKLXVLXEQKLISXXXLSGSXAAHHHHHH-SPRXPIRPIVSXIXIHWPXFYNVXXXXTXXXPXDX [SEQ ID NO: 194] |
| VEGF (4) | EVQLLESGGGLVQPGGSLRLSCAASGFTFXXXYXSWVRQAPGKGLEWVSXISWXXGSIGYADSVKGRFTISRDNSKNTLYLQMNSLRAEDTAVYYCXXXXXXXXNYFDYWGQ |
| | GTLVTVSSGGGGSGGGGSGGGGSQSVLTQPPSASGTPGQRVTISCSGSNSNIGGNFVYWYQQLPGTAPKLLIYENSKRPSXVPDRFSGSXSGTSASLAISGLRSEDXADYYCAA |
| | WDDSLXXVVFGGXTKLTVLGEQKLISXXXLSGSAA [SEQ ID NO: 195] |
| IL-4 (3) | EVQLLESGGGLVQPGGSLRLSCAASGFTFSNAWMSWVRQAPGKGLEWVSAISGSGGSTYYADSVKGRFTISRDNSKNTLYLQMNSLRAEDTAVYYCARAIAARPFDYWGQ |
| | GTLVTVSSGGGGSGGGGSGGGGSQSVLIQPPSASGTPGQRVTISCTGATSNIGAGYDIHWYQQLPGTAPKLLIYSTNNRPSGVPDRFSGSKSGTSASLAISGLRSEDXADYYCA |
| | AWDDSLNGPVFGGXXKLTVLGEQKLISEXDLSGSAA [SEQ ID NO: 196] |
| CD40 (2) | EVQLLESGGGLVQPGGSLRLSCAASGFTFSAYWMHWVRQAPGKGLEWVSGISGGGGSTYYADSVKGRFTISRDNSKNTLYLQMNSLRAEDTAVYYCARMTPWYYGMDV |
| | WGQGTLVTVSSGGGGSGGGGSGGGGSQSMLTQPPSASGTPGQRVTISCSGSTS [SEQ ID NO: 197] |
| CD40 (3) | EVQLLESGGGLVQPGGSLRLSCAASGFTFSTYGMHWVRQAPGKGLEWLSYISGGSSYIFYADSVRGRFTISRDNSENALYLQMNSLRAEDTAVYYCARILRGGSGMDLWGQG |
| | TLVTVSSGGGGSGGGGSGGGGSQSVLTQPPXXSGTPGQRVTISC [SEQ ID NO: 198] |
| CD40 (4) | EVQLLESGGGLVQPGGSLRLSCAASGFTFSTYGMHWVRQAPGKGLEWLSYISGGSSYIFYADSVRGRFTISRDNSENALYLQMNSLRAEDTAVYYCARILRGGSGMDLWGQG |
| | TLVTVSSGGGGSGGGGSGGGGSQSVLTQPPSASGTPGQRVTISCTGSSSNIGAGYDVYWYQQLPGTAPKLLIYGNINRPSGVPDRFSGSKSGTSASLAISGLRSEDXADYYCAA |
| | WDDSLXGLVFGGXXKLTVLXXYKDDDDKAA [SEQ ID NO: 199] |
| CT17 | EVQLLESGGGLVQPGGSLRLSCAASGFTFSSSAMHWVRQAPGKGLEWVSAISGSGGSTYYADSVKGRFTISRDNSKNTLYLQMNSLRAEDTAVYYCARVKGRVTIFGVVINSN |
| | YGMDVWGQGTLVTVSSGGGGSGGGGSGGGGSQSVLTQPPSASGTPGQRVTISCSGSISSIGSNAVSWYQQLPGTAPKLLIYGNSNRPSGVPDRFSGSKSGTSASLAISGLRSE |
| | DXADYYCAAWDDSLNGHDVVFGGXTKLTVLXDYKDXDXKAA [SEQ ID NO: 200] |
| IgM (3) | EVQLLESGGGLVQPGGSLRLSCAASGFTFSSYAMSWVRQAPGKGLEWVSGISWNSGSIGYADSVKGRFTISRDNSKNTLYLQMNSLRAEDTAVYYCARGDYSSSPGGYYYYM |
| | DVWGQGTLVTVSSGGGGSGGGGSGGGGSQSVLTQPPSASGTPGQRVTISCSGSSSXIGSNIVNWYQQLPGTAPKLLIYGNSNRPSXVPDRFSGXXSGTSASLAIXGLRSXDX |
| | ADYYCSSXXSTNTVIFGGXTKLTVLGEQKLISXXDLSGSAA [SEQ ID NO: 201] |
| IgM (4) | EVQLLESGGGLVQPGGSLRLSCAASGFTFSSNEMSWIRQAPGKGLEWVSAIYSGGGTYYADSVKGRFTISRDNSKNTLYLQMNSLRAEDTAVYYCARVNDYGDNVYFDHWG |
| | QGTLVTVSSGGGGSGGGGSGGGGSQSVLTQPPSASGTPGQRVTISCSGSSSNIGNNYVSWYQQLPGTAPKLLIYENNKRPSXVPDRFSGSXSGTSASLAISGLRSEDEADYYCA |
| | AWDDSLSVYVVFGGXTKLXVLGEQKLISXXDLSGSAA [SEQ ID NO: 202] |
| IgM (5) | EVQLLESGGGLVQPGGSLRLSCAASGFTFGSYEMNWVRQAPGKGLEWVSVIYSGGSTYYADSVEGRFTISRDNSKNTLYLQMNSLRAEDTAVYYCARDTNPYYYYGMDVW |
| | GQGTLVTVSSGGGGSGGGGSGGGGSQSVLTQPPSASGTPGQRVTISCSGSSSNIGNNAVNWYQQLPGTAPKLLIYRNNQRPSXVPDRFSGSXSGTSASLAISGLRSEDEADYY |
| | CQSYDSSLNGQVFGGXTKLTVLXEQKLISXEXLSGSAA [SEQ ID NO: 203] |
| HLA-DR/DP | EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCARDGLLPLDYWGQGTL |
| | VTVSSGGGGSGGGGSGGGGSQSVLTQPPSASGTPGQRVTISCSGGSSNIGGNAVNWYQQLPGTAPKLLIYENNKRPSXVPDRFSGSXSGTSASLAISGLRSEDXADYYCSSYA |
| | VSNNFEVLFGGXTKLTVLXEQKLISXXDLSGSAA [SEQ ID NO: 204] |
| ICAM-1 | EVQLLESGGGLVQPGGSLRLSCAASGFTFSNAWMSWVRQAPGKGLEWVAFIWYDGSNKYYADSVKGRFTISRDNSKNTLYLQMNSLRAEDTAVYYCARYSGWYFDYWGQ |
| | GTLVTVSSGGGGSGGGGSGGGGSQSVLTQPPSASGTPGQRVTISCTGSSSXIGAGYDVHWYQQLPGTAPKLLIYDNNNRPSXVPDRFSGSXSGTSASLAISGLRSEDEADYYC |
| | QSYDSSLSAWLFGGXTKLTVLGEQKLISXXDLSGSXAAHHHHHH-SPRWPIRXIVSXXTIXXPXFYXVXXXKPXXTXLXRXXAHPXX [SEQ ID NO: 205] |
| IgM (2) | EVQLLESGGGLVQPGGSLRLSCAASGFTFSDYYMSWIRQAPGKGLEWVSAIGSGPYYAHSVRDRFTISRDNSKNTLYLQMNSLRAEDTAVYYCARGGVEASFDYWGQGTLVT |
| | VSSGGGGSGGGGSGGGGSQSVLTQPPSASGTPGQRVTISCTGSSSNIGAGYDVHWYQQLPGTAPKLLIYGNTNRPSXVPNRFSGSXSGTSASLAISGLRSEDEADYYCQSYDN |
| | DLSGWVFGGXTKLTVLGEQKLISEEXLSGSAA [SEQ ID NO: 206] |
| MCP-1 (4) | QSVLTQPPSASGTPGQRVTISCTGSSSNIGSDYGVQWYQQLPGTAPKLLIYSNNQRPSGVPDRFSGSKSGTSASLAISGLRSEDEADYYCAAWDDSLSGPVFGGGTKLTVLG |
| | [SEQ ID NO: 207] |
| MCP-1 (5) | QSVLTQPASASGTPGQRVTISCTGNSSNIGAGYDVHWYQQLPGTAPKLLIYRNNQRPSGVPDRFSGSKSGTSASLAISGLLSEDEADYYCAAWDYSLNGWVFGGGTKLTVLG |
| | [SEQ ID NO: 208] |
| MCP-1 (6) | QSVLTQPSSASGTPGQRVTISCTGNSSNIGAGYDVHWYQQLPGTAPNLLIYRNNQRPSGVPDRFSGSKSGTSASLAISGLRSEDEADYYCAAWDDSLNGWVFGGGTKLTVLG |
| | Q [SEQ ID NO: 209] |
| MCP-1 (7) | EVQLLESGGGLVQPGGSLRLSCAASGFTFSSYAMSWVRQAPGKGLEWVAYINRGSTYTNYADSMKGRFTISRDNSKNTLYLQMNSLRAEDTAVYYCARRGYGSGSYYAFDI |
| | WGQGTLVTVSSGGGGSGGGGSGGGGSQSVLTQPPSASGTPGQRVTISCTGSSSNIGSDYGVQWYQQLPGTAPKWYSNNQRPSXVPDRFSGSKSGTSASLAISGLRSEDXA |
| | DYYCAAWDDSLSGPVFGGX [SEQ ID NO: 210] |
| MCP-1 (8) | EVQLLESGGGLVQPGGSLRLSCAASGFTFGDYAMSWVRQAPGKGLEWVAVISYDGSNKYYADSVKGRFTISRDNSKNTLYLQMNSLRAEDTAVYYCARDPDPSGTDAFDFW |
| | GQGTLVTVSSGGGGSGGGGSGGGGSQSVLTQPPSASGTPGQRVTISCSGSSSNIGAGYDVHWYQQLPGTAPKLLIYGNSNRPSXVPDRFSGSXSGTSASLAISGLRSEDXADY |
| | YCAAWDDSLSAWAFGG [SEQ ID NO: 211] |
| MCP-1 (9) | EVQLLESGGGLVQPGGSLRLSCAASGFTFSSYEMNWVRQAPGKGLEWVSAISGPGGSTYYADSVKGRFTISRDNSKNTLYLQMNSLRAEDTAVYYCARDLSDYGDFDYWGQ |
| | GTLVTVSSGGGGSGGGGSGGGGSQSVLTQPPSASGTPGQRVTISCTGSSSNIGAGYDAHWYQQLPGTAPKLLIYDNNKRPXXVPDRFSGSXSGTSASLAISGLRSEDEADYYC |
| | ATWDDSLRGWVFG [SEQ ID NO: 212] |
| Cystatin C (1) | EVQLLESGGGLVQPGGSLRLSCAASGFTFSSYAMNWVRQAPGKGLEWVGLISYDGRTTYYADSVKGRSTISRDNSKNTLYLQMNSLRAEDTAVYYCATTTGTTLDYWGQGTL |
| | VTVSSGGGGSGGGGSGGGGSQSVLTQPPSASGTPGQRVTISCTGSSSNIGAGYDVHWYQQLPGTAPKLLIYGNTNRPSXVPDRFSGSXSGTSASLAISGLRSEDEADYYCAA |
| | WDDSLYGWVFGGXTKLTVLGDYXDHDGDYXDHDIDXXDDDDKAA [SEQ ID NO: 213] |
| Cystatin C (2) | EVQLLESGGGLVQPGGSLRLSCAASGFTFSSYSMNWVRQAPGKGLEWVAFISYDGSNKYYVDSVKGRFTISRDNSKNTLYLQMNSLRAEDTAVYYCARDGVPAVPFDYWGQ |
| | GTLVTVSSGGGGSGGGGSGGGGSQSVLTQPPSASGTPGQRVTISCSGSSSNIGAGYDVHWYQQLPGTAPKLLIYSNNQRPS [SEQ ID NO: 214] |
| Cystatin C (3) | EVQLLESGGGLVQPGGSLRLSCAASGFTFSNYAMTWVRQAPGKGLEWVADISHDGFHKYYADSVRGRFTISRDNSKNTLYLQMNSLRAEDTAVYYCARYGRVLPYYYYYGM |
| | DVWGQGTLVTVSSGGGGSGGGGSGGGGSQSVLTQPPSASGTPRQRVTISCTGSSSXIGAGYDVHWYQQLPGTAPKLLIYGNSNRP [SEQ ID NO: 215] |
| Cystatin C (4) | EVQLLESGGGLVQPGGSLRLSCAASGFTFSSYAMNWVRQAPGKGLEWVGLISYDGRTTYYADSVKGRSTISRDNSKNTLYLQMNSLRAEDTAVYYCATTTGTTLDYWGQGTL |
| | VTVSSGGGGSGGGGSGGGGSQSVLTQPPSASGTPGQRVTISCTGSSSNIGAGYDVHWYQQLPGTAPKLLIYGNTNRPSXVPDRFSGSXSGTSXSLAISGLRSXDEADYYCAA |
| | WDDSLYGWVFGG [SEQ ID NO: 216] |
| Apo-A1 (1) | EVQLLESGGGLVQPGGSLRLSCAASGFTFSNNGMHWVRQAPGKGLEWVSAISASGGSTYYADSVKGRFTISRDNSKNTLYLQMNSLRAEDTAVYYCATHGGSSYDAFDIWG |
| | QGTLVTVSSGGGGSGGGGSGGGGSQSVLTQPPSASGTPGQRVTISCTGSSSNIGAGYVVHWYQQLPGTAPKLLIYG [SEQ ID NO: 217] |
| Apo-A1 (2) | EVQLLESGGGLVQPGGSLRLSCAASGFTFRDYYMSWIRQAPGKGLEWVAVTSYDGSKKYYADSVKGRFTISRDNSKNTLYLQMNSLRAEDTAVYYCAKDYADDSIAAPAFDI |
| | WGQGTLVTVSSGGGGSGGGGSGGGGSQSVLTQPPSASGTPGQRVTISCTGSSSXIGAGYDVHWYQQLPGTAPKLLIYGNSNRPS [SEQ ID NO: 218] |
| Apo-A1 (3) | EVXXLESGGGLVQPGGSLRLSCAASGFTFRDYYMSWIRQAPGKGLEWVAVTSYDGSKKYYADSVKGRFTISRDNSKNTLYLQMNSLRAEDTAVYYCAKDYADDSIAAPAFDI |
| | WGQGTLVTVSSGGGGSGGGGSGGGGSQSVLTQPPSASGTPGQRVTISCTGSSSNIGAGYDVHWYQQLPGTAPKLLIYGNSNRPSGVPDRFSGSKSGTSASLAISGLRSXDEA |
| | XYYCQSYDSSLSVVFGGGTKLTVLXXYXDHDGDYKDHDIDYXDDXXXAXAHHHHHH-SPXXXIRXXXSXXTIHXXXXXXXXDWXXXXXXXXX [SEQ ID NO: 219] |
| Factor B (2) | EVQLLESGGGLVQPGGSLRLSCAASGFTFSSYSMNWVRQAPGKGLEWVAVISYDGRFIYYSDSVKGRFTISRDNSKNTLYLQMNSLRAEDTAVYYCARSYGGNLAMDVWGQ |
| | GTLVTVSSGGGGSGGGGSGGGGSQSVLTQPPSASGTPGQRVTISCTGSSSNIGAGYDVHWYQQLPGTAPKLLIYDNNKRPSGVPDRFSGSNSGTSASLAISGLRSEDEADYYC |
| | AAWDDRLNGRVVFGGXTKLTVLGDYXDHDGDYKDHDIDXKDDDXKAA [SEQ ID NO: 220] |
| Factor B (3) | EVQLLESGGGLVQPGGSLRLSCAASGFTFSSYSMNWVRQAPGKGLEWVAVISYDGSNQYYADSVRGRFTISKDNSKNTLYLQMNSLRAEDTAVYYCAREWHYSLDVWGQG |
| | TLVTVSSGGGGSGGGGSGGGGSQSVLTQPPSASGTPGQRVTISCSGSSSXIGSNTVNWYQQLPGTAPKLLIYRNNQRPSXVPDRFSGSXSGTSASLAIXGLRSEDXADYYCAA |
| | WDD [SEQ ID NO: 221] |
| Factor B (4) | EVQLLESGGGLVQPGGSLRLSCAASGFTFSKHSMNWVRQAPGKGLEWVATVSYDGNYKYYADSVKGRFTISRDNSKNTLYLQMNSLRAEDTAVYYCAREGYYYYGMDVWG |
| | QGTLVTVSSGGGGSGGGGSGGGGSQSVLTQPPSASGTPGQRVTISCTGSSSNIGNNAVNWYQQLPGTAPKLLIYNNNQRPXXVPDRFSGSXSGTSXSLAISGLRSEDEADYY |
| | CQPYXDXLSSVVFGGXTXLTVLXDXXDHDXXYKDHDIXYXDXDXXXXAHHHHHH-SPRWPIRPIVSXIXIXWXXVLXRXXXXNXXXXXXXXXXXXHXXXXXX [SEQ ID |
| | NO: 222] |
| C1 inh (2) | EVQLLESGGGLVQPGGSLRLSCAASGFTFSNAWMSWVRQAPGKGLEWVSAISGSGGSTYYADSVKGRFTISRDNSKNTLYLQMNSLRAEDTAVYYCARNRGNWGTYYFDY |
| | WGQGTLVTVSSGGGGSGGGGSGGGGSQSVLTQPPSASGTPGQRVTISCSGSSSNIGSNYVSWYQQLPGTAPKLLIYGSSNRPSGVPDRFSGSXSGTSASLAISGLRSEDEADY |
| | YCQSYDSSLSDHVVFGGXTKLTVLXDYXDHDGDYKDHDIDXXDDDDXAA [SEQ ID NO: 223] |
| C1 inh (3) | EVQLLESGGGLVQPGGSLRLSCAASGFTFSNAWMSWVRQAPGKGLEWVSAISGSGGSTYYADSVKGRFTISRDNSKNTLYLQMNSLRAEDTAVYYCARNRGNWGTYYFDY |
| | WGQGTLVTVSSGGGGSGGGGSGGGGSQSVLTQPPSASGTPGQRVTISCSGSSSXIGSNYVSWYQQLPGTAPKLLIYGSSNRPSXVPDRFSGSXSGTSASLAISGLRSEDXADYY |
| | CQSYDSSLSDHVVFGGXTKLTVLGDYXDHDGDYKDHDXDXXDDXXXAA [SEQ ID NO: 224] |
| C1 inh (4) | EVQLLESGGGLVQPGGSLRLSCAASGFTFSNAWMSWVRQAPGKGLEWVSAISGSGGSTYYADSVKGRFTISRDNSKNTLYLQMNSLRAEDTAVYYCARNRGNWGTYYFDY |
| | WGQGTLVTVSSGGGGSGGGGSGGGGSQSVLTQPPSASGTPGQRVTISCSGSSSXIGSNWSWYQQLPGTAPKLLIYGSSNRPSXVPDRFSGXXSGTSASLAISGLRSEDEADY |
| | YCQSYDSSLSXHVVFGGXTKLTVL [SEQ ID NO: 225] |
| C5 (3) | EVQLLESGGGLVQPGGSLRLSCAASGFTFSSYGMHWVRQAPGKGLEWVSYISSSGSTIYYADSVKGRFTISRDNSKNTLYLQMNSLRAEDTAVYYCLTLGGYWGQGTLVTVSS |
| | GGGGSGGGGSGGGGSQSVLIQPPSASGTPGQRVTISCSGSSSNIGSNIVNWYQQLPGTAPKWYSNNQRPSGVPDRFSGSKSGTSASLAISGLRSXDEADYYCQSYDSSLSG |
| | WVFGGXTKLTVLXDYKXHDGDYKDHDIDXKDDDXXAA [SEQ ID NO: 226] |
| C4 (2) | EVQLLESGGGLVQPGGSLRLSCAASGFTFSDYYMSWVRQAPGKGLEWVSGVSWNGSRTHYADSVKGRFTISRDNSKNTLYLQMNSLRAEDTAVYYCAGYGSGSRATGYNW |
| | FAPWGQGTLVTVSSGGGGSGGGGSGGGGSQSVLTQPPSASGTPGQRVTISCTGSSSNIGAGYDVHWYQQLPGTAPKLLIYRNNQRPSGVPDRFSGSXSGTSXSLAISGLRSE |
| | DXADYYCQSYDSSLXGPYWVFXXXNQXDGPRXXXKTMTXXXXXXDIDYXXXXXQXRXAXXXHHH-SPXXP [SEQ ID NO: 227] |
| C4 (3) | EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCARGWSTSSFDYWGQG |
| | TLVTVSSGGGGSGGGGSGGGGSQSVLTQPPSASGTPGQRVTISCSGSSSXIGNHYVSWYQQLPGTATKLLIYXDDLLPSXVPDRFSGSXSGTSASLAIXGLRSEDEADYYCAAW |
| | DDRSGQVLFGGXTKLTVLGDYXDHDGDYXDHDIDXXDDDXKAXAHHHHHH-XXRWPIRPXVSXXTIHXXXFXXXXXXKT [SEQ ID NO: 228] |
| C4 (4) | EVQLLESGGGLVQPGGSLRLSCAASGFTFSSYSMNWVRQAPGKGLEWVSGISGSGGSTYYADSVKGRFTISRDNSKNTLYLQMNSLRAEDTAVYYCAKHSGYGFDIWGQGTL |
| | VTVSSGGGGSGGGGSGGGGSQSVLTQPPSASGTPGQRVTISCSGGASXLGMHFVSWYQQLPGTAPKLLIYYDDLLPSGVPDRFSGXXSGTSASLAISGLRSEDEADYYCAAW |
| | DDSLNGWVFGGXTKLXVLGDYXDXXGDYKDHDIDXKDXXXXAXAHXHHHH-SPXWXXRPIVXXITXXXXVXLQRXDWXXPXVXXXXXXXXXXPX [SEQ ID NO: 229] |
| C3 (3) | EVQLLESGGGLVQPGGSLRLSCAASGFTFSSYAMNWVRQAPGKGLEWVANINQDGSTKFYVDSVKGRFTISRDNSKNTLYLQMNSLRAEDTAVYYCARDTGGNYLGGYYYY |
| | GMDVWGQGTLVTVSSGGGGSGGGGSGGGGSQSVLTQPPSASGTPGQRVTISCSGSSSNIGSNYVYWYQQLPGTAPKLLIYHRNDQRPSXVPDRFSGSXSGTSASLAISGLRSX |
| | DXADYYCSSYAGNNNLVFGGXTKLTVLGDYXDHDGDYKDHDIDYXDXDXXAA [SEQ ID NO: 230] |
| C3 (4) | EVQLLESGGGLVQPGGSLRLSCAASGFTFSDHYMDWVRQAPGKGLEWVSGISGNGATIDYADSVKGRFTISRDNSKNTLYLQMNSLRAEDTAVYYCARPSITAAGSEDAFDL |
| | WGQGTLVTVSSGGGGSGGGGSGGGGSQSVLTQPPSASGTPGQRVTISCSGSSSNIGSNYVYWYQQLPGTAPKWYGNSNRPSGVPDRFSGSKSGTSASLAISGLRSXDGAD |
| | YYCQSYDSSLSGWVFGGXTKLTVLGXYXDHDGDYKDXDIDYKDDXXKAA [SEQ ID NO: 231] |
| C3 (5) | EVQLLESGGGLVQPGGSLRLSCAASGFTFSNYWMSWVRQAPGKGLEWVSGISGSGGTTYYADFVKGRFTISRDNSKNTLYLQMNSLRAEDTAVYYCARKYYYGSSGAFDIW |
| | GQGTLVTVSSGGGGSGGGGSGGGGSQSVLTQPPSASGTPGQRVTISCTGSSSNIGAGYDVHWYQQLPGTAPKLLIYRNNQRPSXVPDRFSGSXSGTSASLAIXGLRSEDXAD |
| | YYCAAWDDSLXGPVFXGXTKLTVL [SEQ ID NO: 232] |
| C3 (6) | EVQLLESGGGLVQPGGSLRLSCAASGFTFSSYAMNWVRQAPGKGLEWVANINQDGSTKFYVDSVKGRFTISRDNSKNTLYLQMNSLRAEDTAVYYCARDTGGNYLGGYYYY |
| | GMDVWGQGTLVTVSSGGGGSGGGGSGGGGSQSVLTQPPSASGTPGQRVTISCSGSSSNIGSNYVYWYQQLPGTAPKWYRNDQRPSGVPDRFSGSXSGTSASLAISGLRSE |
| | DXADYYCSSYAGNNNLVFGGXXKLTVLGXXXDHDGDYKDHDIDXXDXDXXAA [SEQ ID NO: 233] |
| MYOM 2 (1) | EVQLLESGGGLVQPGGSLRLSCAASGFTFSSYAMSWVRQAPGKGLEWVSGISGSGGSTYYADSVKGRFTISRDNSKNTLYLQMNSLRAEDTAVYYCARGVVAGSWGQGTLV |
| | TVSSGGGGSGGGGSGGGGSQSVLTQPPSASGTPGQRVTISCSGSSSXIGNNAVNWYQQLPGTAPKLLIYDNNKRPSXVPDRFSGXXSGTSXSLAIXGLRSEDEADYYCA |
| | [SEQ ID NO: 234] |
| MYOM2 (2) | EVQLLESGGGLVQPGGSLRLSCAASGFTFSNEWMAWVRQAPGKGLEWVSSISSSSSYIYYADSVKGRFTISRDNSKNTLYLQMNSLRAEDTAVYYCAGTYHDFWSATYWGQ |
| | GTLVTVSSGGGGSGGGGSGGGGSQSVLTQPPSASGTPGQRVTISCSGSSSNIGSNTVNWYQQLPGTAPKLLIYGNSNRPSXVPDRFSGSXSGTSASLAISGLRSEDXADYYCAA |
| | WDDSLNGWVFGGXTKLTVLGD [SEQ ID NO: 235] |
| LUM | EVQLLESGGGLVQPGGSLRLSCAASGFTFSSNYMSWVRQAPGKGLEWVSAISASGTYTYYTDSVNGRFTISRDNSKNTLYLQMNSLRAEDTAVYYCARVNTVGLGTPFDNW |
| | GQGTLVTVSSGGGGSGGGGSGGGGSQSVLTQPPSASGTPGQRVTISCSGSSSNIGSNTVNWYQQLPGTAPKLLIYGNRNRPSGVPDRFSGSXSGTSASLAISGLRSEDEADYY |
| | CAAWDDSLSGWVFGGXTKLTVLXDYXDHDGDYKDHDIDXXXDDXXAA [SEQ ID NO: 236] |
| DUSP9 | EVQLLESGGGLVQPGGSLRLSCAASGFTFSSYGFHWVRQAPGKGLEWVAVISYDGSNKYYADSVKGRFTISRDNSKNTLYLQMNSLRAEDTAVYYCARGEFGVYWGQGTLV |
| | TVSSGGGGSGGGGSGGGGSQSVITQPPSASGTPGQRVTISCSGSSSNIGSNYVYWYQQLPGTAPKLLIYGNRNRPSGVPDRFSGSXSGTSASLAISGLRSEDEADYYCSSYAGS |
| | NNFEVVFGGXTKLTVLGDYXDHDGDYKDHDIDYKDDDXKAA [SEQ ID NO: 237] |
| CHX10 (1) | EVQLLESGGGLVQPGGSLRLSCAASGFTFSSYGMHWVRQAPGKGLEWVAVISYDGSNKYYADSVKGRFTISRDNSKNTLYLQMNSLRAEDTAVYYCARNSDYYGMDVWGQ |
| | GTLVTVSSGGGGSGGGGSGGGGSQSVLTQPPSASGTPGQRVTISCSGSSSNIGYSDVYWYQQLPGTAPKLLIYENNKRPSXVPDRFSGSXSGTSASLAISGLRSEDEADYYCST |
| | WDDSLNGHVIFGG [SEQ ID NO: 238] |
| CHX10 (3) | EVQLLESGGGLVQPGGSLRLSCAASGFTFSSYAMSWVRQAPGKGLEWVAVISYDGSNKYYADSVKGRFTISRDNSKNTLYLQMNSLRAEDTAVYYCARNYGDSINWFDPWG |
| | QGTLVTVSSGGGGSGGGGSGGGGSQSVLTQPPSASGTPGQRVTISCSGSSSXIRSNTVNWYQQLPGTAPKLLIYGNSNRPSXVPDRFSGXXSGTSXSLAISGLRSEDXADYYCA |
| | XWDDSLN [SEQ ID NO: 239] |
| ATP-5B (1) | EVQLLESGGGLVQPGGSLRLSCAASGFTFSSYAMHWVRQAPGKGLEWVAVISYDGSKTYHADSVEGRFTISRDNSKNTLYLQMNSLRAEDTAVYYCARHLRPYYFDYWGQG |
| | TLVTVSSGGGGSGGGGSGGGGSQSVLTQPPSASGTPGQRVTISCSGSSSXIGSNTVNWYQQLPGTAPKLLIYGNSNRPSXVPDRFSGXXSGTSASLAISGLRSEDXADYYCSA |
| | WDDRLRGRVFGG [SEQ ID NO: 240] |
| ATP-5B (2) | EVQLLESGGGLVQPGGSLRLSCAASGFTFSSYGMHWVRQAPGKGLEWVSLISSASSYIYHADSVKGRFTISRDNSKNTLYLQMNSLRAEDTAVYYCARAGRVCTNGVCHTTFD |
| | YWGQGTLVTVSSGGGGSGGGGSGGGGSQSVLTQPPSASGTPGQRVTISCSGDRSNIGSNTVNWYQQLPGTAPKLLIYGNSNRPSGVPXRFSGSXSGTSXSLAISGLRSEDEA |
| | DYYCQSYDSSLSAVVFGGXTKLTVLGDYXXHDXXYKDHDIDYXXDXDXAXAHXHHHH-SPRXXXXPIVSXXXXXXXXXXXXXXLXKXXXXPTXXXXXXXX [SEQ ID NO: 241] |
| ATP-5B (3) | EVQLLESGGGLVQPGGSLRLSCAASGFTFSTYAMSWVRQAPGKGLEWVSSISSTSTYIHYADSVKGRFTISRDNSKNTLYLQMNSLRAEDTAVYYCARVSSWYSAFDIWGQGT |
| | LVTVSSGGGGSGGGGSGGGGSQSVLTQPPSASGTPGQRVTISCTGSSSNIGNNAVNWYQQLPGTAPKLLLIYSNNQRPSXVPDRFSGSXSGTSASLAISGLRSEDXADYYCQSY |
| | DSSLSGVIFGGXTKLXVLXDYXDHDGDYXDHDIDXXXDDDKAA [SEQ ID NO: 242] |
| Sox11a | EVQLLESGGGLVQPGGSLRLSCAASGFTFSDFWMSWVRQAPGKGLEWVSSISGGGGTAFYVDSVKGRFTISRDNSKNTLYLQMNSLRAEDTALYYCARMTDLESGDAFDIW |
| | GQGTLVTVSSGGGGSGGGGSGGGGSQSVLTQPPSASGTPGQRVTISCSGSSSNIGSNYVNWYQQLPGTAPKLLIYNDNVRPSGVPDRFSGSXSGTSASLAISGLRSEDXADYY |
| | CQXWGTGVFGGXTKLTVLXDYXDHDGDXXDHDIDXKDXDXKAA [SEQ ID NO: 243] |
| TBC1D9 (1) | EVQLLESGGGLVQPGGSLRLSCAASGFTFSSYSMSWVRQAPGKGLEWVAVISYDGSNKYYADSVKGRFTISRDNSKNTLYLQMNSLRAEDTAVYYCARDRTRGSTALDIWGQ |
| | GTLVTVSSGGGGSGGGGSGGGGSQSVLTQPPSASGTPGQRVTISCSGSSSYIGSNYVYWYQQLPGTAPKLLIYRNNQRPXXVPDRFSGXXSGTSASLAISGLRSEDEADYYCAA |
| | WDDSLSGWVFGGXTKLTVLGD [SEQ ID NO: 244] |
| UPF3B (1) | EVQLLESGGGLVQPGGSLRLSCAASGFTFSDYYMTWIRQAPGKGLEWVSDISWNGSRTHYADSVKGRFTISRDNSKNTLYLQMNSLRAEDTAVYYCSSHLVYWGQGTLVTV |
| | SSGGGGSGGGGSGGGGSQSVLTQPPSASGTPGQRVTISCTGSSSNIGAGYDVHWYQQLPGTAPKLLIYDNNKRPSXVPDRFSGSXSGTSASLAIXGLRSEXXADYYCQTYDSS |
| | LSGSVVFGGXTKLTVLGDYXDHDXDY [SEQ ID NO: 245] |
| UPF3B (2) | EVQLLESGGGLVQPGGSLRLSCAASGFTESSYAMSWVRQAPGKGLEWVSYISSSSSYANYADSVKGRFTISRDNSKNTLYLQMNSLRAEDTAVYYCARLGVYSGTYLFAFDIW |
| | GQGTLVTVSSGGGGSGGGGSGGGGSQSVLTQPPSASGTPGQRVTISCTGSSSXIGAGYDVHWYQQLPGTAPKLLIYGNSNRPSXVPDRFSGSXSGTSASLAISGLRSXDEADY |
| | YCQSRDSSLSGWVFGGXTKLTVLGD [SEQ ID NO: 246] |
| Apo-A4 (1) | EVQLLESGGGLVQPGGSLRLSCAASGFTFSDYYMSWVRQAPGKGLEWVSGVSWNGSRTHYADSVKGRFTISRDNSKNTLYLQMNSLRAEDTAVYYCARVAYDIDAFDMW |
| | GQGTLVTVSSGGGG [SEQ ID NO: 247] |
| Apo-A4 (2) | EVQLLESGGGLVQPGGSLRLSCAASGFTFSDYYMSWVRQAPGKGLEWVSGVSWNGSRTHYADSVKGRFTISRDNSKNTLYLQMNSLRAEDTAVYYCARVAYDIDAFDMW |
| | GQGTLVTVSSGGGGSGGGGSGGGGSQSVLTQPPSASGTPGQRVTISCSGSFSNIGSNYVYWYQQLPGTAPKLLIYENNKRPSGVPDRFSGSXSGTSASLAISGLRSEDEADYYC |
| | AAWDDSLNGPMFGGXTKLTVLXDYKDHDGDYKDHDIDYKDDXXXXAAHHHHHH-SPRWXIRPXXSXXTIHXXXXLXXXD [SEQ ID NO: 248] |
| Apo-A4 (3) | EVQLLESGGGLVQPGGSLRLSCAASGFTFSSYSMNWVRQAPGKGLEWVSAITGSGNATFYADSVKGRFTISRDNSKNTLYLQMNSLRAEDTAVYYCTTGATTRWGQGTLVT |
| | VSSGGGGSGGGGSGGGGSQSVLTQPPSASGTPGQRVTISCSGSRSNIGSNHVFWYQQLPGTAPKLLIYENNKRPSGVPDRFSGSXSGTSASLAISGLRSEDXADYYCAAWDD |
| | SLSGWVFGG [SEQ ID NO: 249] |
| TBC1D9 (2) | EVQLLESGGGLVQPGGSLRLSCAASGFTFSNAWMSWVRQAPGKGLEWVSFISSSSSYIYYADSVKGRFTISRDNSKNTLYLQMNSLRAEDTAVYYCARVNLVGCTNGVCNGH |
| | DYWGQGTLVTVSSGGGGSGGGGSGGGGSQSVLTQPPSASGTPGQRVTISCSGSSSXIGSNTVNWYQQLPGTAPKLLIYDNNKRP [SEQ ID NO: 250] |
| TBC1D9 (3) | EVQLLESGGGLVQPGGSLRLSCAASGFTFGDYAMSWVRQAPGKGLEWVSAISGSGGSTYYADSVKGRFTISRDNSKNTLYLQMNSLRAEDTAVYYCAKGRTMASHWGQGT |
| | LVTVSSGGGGSGGGGSGGGGSQSVLTQPPSASGTPGQRVTISCSGSSSXIGNNHVSWYQQLPGTAPKLLIYGNSNRPSXVPDRFSGSXSGTSASLAISGLRSEDXADYYCAAW |
| | DNSLKVWMEGG [SEQ ID NO: 251] |
| ORP-3 (1) | EVQLLESGGGLVQPGGSLRLSCAASGFTFSSNYMSWVRQAPGKGLEWVSYISGNSGYTNYADSVKGRFTISRDNSKNTLYLQMNSLRAEDTAVYYCARHAGSYDMYGMDV |
| | WGQGTLVTVSSGGGGSGGGGSGGGGSQSVLTQPPSASGTPGQRVTISCSGSTSXIGSHYVYWYQQLPGTAPKWYGNSNRPXXVPDRFSGXXSGTSXSLAISGLRSEDXADY |
| | YCQSYDSRLSGWVFGG [SEQ ID NO: 252] |
| ORP-3 (2) | EVQLLESGGGLVQPGGSLRLSCAASGFTFSSYAMHWVRQAPGKGLEWVAVISYDGSNKYYADSVKGRFTISRDNSKNTLYLQMNSLRAEDTAVYYCARKSSLDVWGQGTLV |
| | TVSSGGGGSGGGGSGGGGSQSVLTQPPSASGTPGQRVTISCSGSSSXIGNNYVSWYQQLPGTAPKLLIYDDNKRPSGVPDRFSGSXSDTSASLAISGLRSEDEADYYCAAWDD |
| | SLXGRVEGGXTKLTVLG [SEQ ID NO: 253] |
| CIMS (5) | EVXLLESGGGLVQPGGSLRLSCAASGFTFSDHYMDWVRQAPGKGLEWVSGISGSGGSTYYGDSVKGRFTISRDNSKNTLYLQMNSLRAEDTAVYYCASRLYWGQGTLVTVSS |
| | GGGGSGGGGSGGGGSQSVLTQPPSASGTPGQRVTISCTGSSSNIGAGYVVHWYQQLPGTAPKLLIYDNDKRPSGVPDRFSGSKSGTSASLAISGLRSEDEADYYCAAWDDSL |
| | DAVLFGGXXKLTVLGEQKLISEXDLSGSAA [SEQ ID NO: 254] |
| CIMS (13) | EVQLLESGGGLVQPGGSLRLSCAASGFTFSSYAMSWVRQAPGKGLEWVSAISGSGGRTYYTDSVRDRFTISRDNSKNTLYLQMNSLRAEDTAVYYCARDLMPVCQYCYGMD |
| | VWGQGTLVTVSSGGGGSGGGGSGGGGSQSVLTQPPSASGTPGQRVTISCTGSSSNIGAGYDVHWYQQLPGTAPKLLIYSNNQRPSGVPDRFSGSXSGTSASLAISGLRSEDE |
| | ADYXCQSYDSSLNKDVVFGGXTKLTVLGEQKLISXXDLSGSAXAHHHHHH-SPRXPIRPIVSRXTIHWXXXLXXXDWENXXXTXLXXXAXXPPFXXXXX [SEQ ID NO: 255] |

*The structure of the scFv antibodies is described in Söderlind et al., 2000, ′Recombining germline-derived CDR sequences for creating diverse single-framework antibody libraries′ Nature Biotechnol., 18(8):852-6, which is incorporated herein by reference in its entirety.

### Example A—Diagnosis

Abstract

Objective.

To define a multiplex serum biomarker signature associated with systemic lupus erythematosus (SLE).

Methods.

Affinity proteomics, represented by 195-plex recombinant antibody microarrays, targeting mainly immunoregulatory proteins, was used to perform protein expression profiling of crude, biotinylated serum samples. State of the art bioinformatics was used to define condensed multiplex signatures associated with SLE, and the classification power was evaluated in terms of receiver operating characteristic curves.

Results.

The results showed that a condensed (25-plex), pre-validated serum biomarker signature classifying SLE vs. healthy controls with high specificity and sensitivity could be pin-pointed. The panel was composed of novel as well as already known candidate markers. Further, the data indicated that SLE vs. healthy controls could be classified irrespective of the phenotype, reflecting the severity of the disease.

The biological relevance of the biomarkers was supported by data mining and pathway analysis.

Conclusion.

Our study showed that the immune system could be exploited as a specific and sensitive sensor for SLE. SLE-associated serum biomarker panels have been identified, enhancing our fundamental knowledge of SLE, and in the long-term run allowing serum-based diagnosis of SLE.

Introduction

Systemic lupus erythematosus (SLE) is a chronic and multisystem autoimmune connective-tissue disease (1, 2), with disease spectra ranging from subtle symptoms to life-threatening multi-organ failure (3, 4). Some hallmarks characteristics of SLE include production of autoantibodies, deposition of immune complexes in tissues, and excessive complement activation (4, 5). Despite major efforts, the complex etiology and pathogenesis, heterogeneous presentation and unpredictable course still pose major challenges in the monitoring and diagnosis of the disease (5-7).

In more detail, the clinical manifestations vary widely among patients, and the signs and symptoms evolve overtime, and overlap with those of other autoimmune diseases, why SLE is often misdiagnosed and/or overlooked (6, 8, 9). In fact, patients may spend up to four years and see three or more physicians before the disease is correctly diagnosed (8). On the other hand, SLE is also often over-diagnosed (10). The diagnosis of SLE in clinical practice is usually made according to the principles outlined by Fries and Holman (11); presence of typical manifestations from at least two organ systems in combination with immunological abnormality consistent with SLE in the absence of a better diagnostic alternative. However, it has during last years been concluded that a biopsy verified lupus glomerulonephritis in combination with immunological abnormality should be accepted for SLE diagnosis. Hence, novel means for improved diagnosis of SLE are needed.

Further, SLE classification criteria have been defined by the American College for Rheumatology (ACR) (12, 13) and more recently from systemic lupus International Collaborating Clinics (SLICC) (14). According to ACR, SLE is classified when at least 4 of 11 clinical and/or immunological criteria, shared by many diseases, are fulfilled. In the case of SLICC, SLE is classified if i) at least 4 of 17 clinical and immunological criteria, or ii) biopsy verified lupus nephritis in the presence of antinuclear antibodies (ANA) or anti-dsDNA antibodies are met. In practice, this means that patients can display a very diverse set of symptoms, but all still be classified as similar.

Although major efforts have been made to decipher SLE-associated biomarkers, the output of validated and clinically useful biomarkers is still limited (6, 15-19). In fact, there is no single laboratory blood- or urine-based test yet at hand that specifically and accurately can confirm or rule out the diagnosis of SLE (6, 15, 18, 19). This lack of adequate biomarkers for SLE has hampered proper clinical management of patients with SLE (15). Considering the complexity and heterogeneity of SLE, a multiplex biomarker panel, rather than a single biomarker may be required to resolve this clinical need (20), placing high demands on the technologies used for biomarker discovery.

In this regards, omic-based technologies holds great promise as one route for biomarker discovery in SLE (17). We have recently used affinity proteomics, represented by recombinant antibody microarrays (21, 22), for serum biomarker discovery in SLE (23) (Carlsson et al, unpublished observations). Targeting mainly immunoregulatory proteins in crude, non-fractionated serum samples, the results showed that candidate serum biomarker panels associated with SLE could be deciphered.

In this study, we have extended our previous efforts, and performed differential serum protein expression profiling of a large cohort of SLE patients vs. healthy controls. To this end, a re-optimized recombinant antibody microarray platform, displaying superior performances (23) (Delfani et al, unpublished data), and targeting a larger set of immuneregulatory analytes, was applied. In addition, an optimized procedure for handling and analysing the microarray data was also adopted (Delfani et al, unpublished data). The results showed that a condensed (25-plex), pre-validated serum biomarker signature classifying SLE vs. healthy controls with high specificity and sensitivity could be identified. Further, the data also outlined that SLE could be classified irrespective of the phenotype, reflecting the severity of the disease.

Materials and Methods

Clinical Samples

In total, 197 serum samples were collected at the Department of Rheumatology, Skane University Hospital (Lund, Sweden), including SLE patients (n=86) and normal controls (n=50) (Table I). The SLE patients had clinical SLE diagnosis and displayed four or more American College of Rheumatology classification criteria (13, 24). The SLE samples were collected over time during follow-up and the patients were presented with either flare or remission, i.e. for some patients up to four samples were collected at different time-points. The SLE patients (samples) were grouped according to disease severity as previously described (25): 1) skin and musculoskeletal involvement (SLE1, n=30); 2) serositis, systemic vasculitis but not kidney involvement (SLE2, n=30); 3) presence of SLE glomerulonephritis (SLE3, n=87). The clinical disease activity was defined as SLE disease activity index 2000 (SLEDAI-2K) score (26). All samples were aliquoted and stored at −80° C. until analysis. This retrospective study was approved by the regional ethics review board in Lund, Sweden.

Labeling of Serum Samples

The serum samples were labelled with EZ-link Sulfo-NHS-LC-Biotin (Pierce, Rockford, Ill., USA) using a previously optimized labelling protocol for serum proteomes (21, 22, 27). Briefly, the samples were diluted 1:45 in PBS (about 2 mg protein/ml), and biotinylated at a molar ratio of biotin:protein of 15:1. Unreacted biotin was removed by extensive dialysis against PBS (pH 7.4) for 72 h at 4° C. The samples were aliquoted and stored at −20° C. until further use.

Production and Purification of Antibodies

In total, 195 human recombinant single-chain fragment variable (scFv) antibodies, including 180 antibodies targeting 73 mainly immunoregulatory analytes, anticipated to reflect the events taking place in SLE, and 15 scFv antibodies targeting 15 short amino acid motifs (4 to 6 amino acids long) (28) were selected from a large phage display library (Table II) (29) (Persson et al, unpublished data). The specificity, affinity, and on-chip functionality of the scFv antibodies have been previously validated (see Supplementary Appendix 1 for details).

All scFv antibodies were produced in 100 ml E. coli and purified from expression supernatants using affinity chromatography on Ni²⁺-NTA agarose (Qiagen, Hilden, Germany) validated (see Supplementary Appendix 1 for details).

Production and Analysis of Antibody Microarrays

The scFv microarrays were produced an handled using a previously optimized and validated set-up (23) (Delfani et al, unpublished data) (see Supplementary Appendix 1 for details). Briefly, 14 identical 25×28 subarrays were printed on each black polymer MaxiSorp microarray slide (NUNC A/S, Roskilde, Denmark) using a non-contact printer (SciFlexarrayer S11, Scienion, Berlin, Germany). Biotinylated samples were added and any bound analytes were visualized using Alexa 647-labelled streptavidin (SA647) (Invitrogen). Finally, the slides were scanned with a confocal microarray scanner (ScanArray Express, PerkinElmer Life & Analytical Sciences).

Data Pre-Processing

The ScanArray Express software v4.0 (PerkinElmer Life & Analytical Sciences) was used to quantify spot signal intensities, using the fixed circle method. Signal intensities with local background subtraction were used for data analysis. Each data point represents the mean value of all three replicate spots unless any replicate CV exceeded 15%, in which case the worst performing replicate was eliminated and the average value of the two remaining replicates was used instead. Log¹⁰ values of signal intensities were used for subsequent analysis. The microarray data was normalized in a two-step procedure using a semi-global normalization method (23, 30, 31) and the “subtract by group mean” approach (see Supplementary Appendix 1 for details).

Data Analysis

Where applicable, the sample cohort was randomly divided into a training set (⅔ of the samples) and a test set (⅓ of the samples), making sure that the distribution of SLE vs. controls and/or samples with active vs. inactive disease was similar between the two sets. It should be noted that for those SLE patients where more than one sample was at hand, the sample was randomly selected for each comparison, and only one sample per patient was included in each subset comparison in order to avoid bias (i.e. over-representation of certain patients).

The support vector machine (SVM) is a supervised learning method in R (32-34) that we used to classify the samples (see Supplementary Appendix 1 for details). For classification of SLE1 vs. N and SLE2 vs. N, the SVM was trained using a leave-one-out cross-validation procedure (30), and the prediction performance of the classifier was evaluated by constructing a receiver operating characteristics (ROC) curve and calculating the area under the curve (AUC).

In the case of SLE vs. N and SLE3 vs. N, the samples were divided into a training set and a test set, and a backward elimination algorithm (35) combined with a leave-one-out cross-validation procedure was applied on the training set to determine a condensed panel of antibodies displaying the highest combined discriminatory power. A single SVM model was then calibrated on the training set using the condensed antibody panel, whereafter the model (classifier) was frozen and evaluated on the test set.

Significantly differentially expressed analytes (p<0.05) were identified based on Wilcoxon rank sum tests. Heat maps and visualization of the samples by principal component analysis (PCA) were carried using Qlucore Omics Explorer 2.2. (Qlucore AB, Lund, Sweden). Data-mining and pathway analysis was conducted using Metacore (Thomson Reuters, New York, N.Y., USA).

Results

In this study, we have applied recombinant scFv antibody microarrays for deciphering serum biomarker signatures reflecting SLE. In total, 197 crude, biotinylated serum samples (SLE n=147, healthy controls n=50) representing 136 patients (86 SLE and 50 controls) were profiled using 195-antibody microarrays, targeting mainly immunoregulatory analytes (Table 1). The scanned microarray images were converted into protein expression profiles, or protein maps, and disease-associated serum biomarker panels were delineated.

Serum Biomarker Panel Discriminating SLE Vs. Healthy Controls

First, we determined whether a multiplex serum biomarker signature discriminating SLE vs. healthy controls could be deciphered. To this end, the data set was randomly divided into a training set (⅔ of all samples) and a test set (⅓ of all samples). A stepwise backward elimination procedure was then applied to the training set in order to identify the smallest set of antibodies, i.e. biomarkers, required for differentiating SLE vs. healthy controls. The results showed that a combination of 16 antibodies, evaluated in terms of the smallest error, provided the best classification (FIG. 1A). In order to allow some flexibility in the signature, the top 25 antibodies were selected to represent the condensed biomarker panel (FIG. 1B). The panel was found to be composed of both up- (e.g. IL-8, Cystatin C, MCP-1, and TGF-3) and down-regulated (e.g. C3, CD40, and LUM) analytes, although the former dominated (FIG. 1B). It should be noted that we did not differentiate whether the observed up- and down-regulated levels of a protein was due to an in-/decreased production or in-/decreased consumption.

In order to evaluate the classification power of this 25-biomarker signature, the panel was first used to train a single SVM model, denoted frozen SVM, on the training set. Next, the frozen SVM model was applied to the independent test set. The results showed that a ROC AUC value of 0.94 was obtained (FIG. 1C), demonstrating that SLE vs. healthy controls could be differentiated with a discriminatory power. Visualizing the data using a principle component analysis (PCA) based approach, a similar distinct discrimination was observed (FIGS. 1D and 1F).

To test the robustness of the data set with respect to the classification, we randomly divided the entire data set in 9 additional pairs of training and test sets, and re-ran the above process. The results showed that the 10 comparisons resulted in a median AUC value of 0.86 (range 0.79 to 0.95) (FIG. 2A), illustrating the robustness of the data set (and the classification approach). Furthermore, the frequency at which each biomarker occurred in these ten 25-plex signatures is shown in FIG. 2B for all markers present two or more times. The data showed, as could be expected, that the identity of the top 25 biomarkers varied, but a core of 6 biomarkers was constant (C3, CD40, Cystatin C, MCP-1, Sialyl lewis x, and TGF-β) and an additional 7 biomarkers were present at a high frequency (50-70%), outlining their diagnostic potential.

Data-Mining and Pathway Analysis

To explore the biological relevance of the observed serum biomarkers, we attempted to perform a focused data-mining and pathway analysis using Metacore™. The analyze single experiment workflow tool was used for conducting enrichment analysis of the data set by mapping it onto selected MetaCore's ontologies, including disease by biomarkers (FIG. 3A), gene ontology processes (FIG. 3B), pathway maps (FIG. 3C), and process networks (FIG. 3D). The data was also used to build the most relevant networks (FIG. 3E). We used those biomarkers (n=28) among the ten 25-plex signatures that displayed i) a P<0.05 and ii) occurrence frequency ≥3 as input data.

When searching for disease by the identified biomarkers, the data analysis showed that SLE was the top hit, followed by 2 other autoimmune conditions, rheumatoid arthritis and connective tissue disease, (FIG. 3A). Hence, the results outlined the biological (disease) relevance of the observed biomarkers. Further, the immune response (and regulation thereof) was suggested as the top process(es) by gene ontology processes (FIG. 3B), which also reflected the fact that mainly immunoregulatory analytes were targeted by our microarray set-up. Furthermore, immune responses—classical and alternative complement activation—were identified as the top pathway maps (FIG. 3C), and inflammation—complement system—was pin-pointed as the top statistically significant process network (FIG. 3D). Finally, using the analyse network algorithm, the top network indicated was found to involve top processes, such as apoptotic process and programmed cell death (FIG. 3E), both known to be associated with SLE.

Serum Biomarker Panels Discriminating Phenotypic Subsets of SLE Vs. Healthy Controls

To investigate whether disease severity was a confounding factor for discriminating SLE vs. healthy controls, the SLE samples were grouped according to phenotype (SLE1, SLE2, and SLE3), and the data analysis were re-run. The disease severity is reflected by the phenotype, with SLE1 displaying the least symptoms and SLE3 the most and severe symptoms. The classification was performed adopting a leave-one-out cross-validation, the most stringent approach that can be employed when the sample cohorts are too small to justify the samples to be split into training and test sets.

The results showed that the all three phenotypes could be discriminated from healthy controls, with ROC AUC values of 0.92 (SLE1) (FIG. 4A), 0.94 (SLE2) (FIG. 4B), and 0.89 (SLE3) (FIG. 4C). When viewing the top 30 significantly (p<0.05) differentially expressed serum biomarkers, the signatures displayed on overall similar expression pattern, although the levels of the individual markers differed (FIG. 4D). In more detail, a majority of the biomarkers were found to be up-regulated in all three SLE phenotypes, while a core of mainly 5 biomarkers, including complement proteins (C1q, C3, C4, and factor B) and ApoA1, were found to be down-regulated (FIG. 4). Taken together, the results showed that SLE vs. healthy controls could be discriminated irrespective of the disease severity (phenotype).

In FIG. 5, the expression profiles of five key proteins, including three complement proteins (C1q, C3, and C4) and two cytokines (IL-6 and IL-12) are shown for all three phenotypes as well as healthy controls. While the complement proteins were down-regulated in the phenotypes compared to the healthy controls, the two cytokines were found to be up-regulated.

Refined Serum Biomarker Panel Discriminating SLE3 vs. Healthy Controls

Finally, we refined the serum biomarker panel discriminating SLE3 vs. healthy control (the only phenotype with a sufficient number of samples allowing the data set to be split into training and tests). Hence, the smallest set of antibodies, i.e. biomarkers, required for differentiating SLE3 vs. healthy controls was determined as described above (backward elimination algorithm), and the procedure was iterated 10 times. The smallest number of biomarkers required for the best classification was found to be 9, and to allow some flexibility in the signature, the top 25 antibodies were selected to represent the condensed biomarker panel (data not shown). Applying the frozen SVMs on the test set resulted in a median ROC AUC value of 0.94 (range 0.84 to 0.97) (FIG. 6A), demonstrating the robustness of the data set and the high discriminatory power of the 25-plex panels.

The frequency at which each biomarker occurred in these ten 25-plex signatures is shown in FIG. 6B for all markers present two or more times. The data showed, as could be expected, that the identity of the top 25 biomarkers varied, but a core of 6 biomarkers was constant (C3, C4, CD40, Cystatin C, factor B, and MCP-1) and an additional 11 biomarkers were present at a high frequency (50-90%), demonstrating their discriminatory potential.

### DISCUSSION

Once clinical symptoms have developed, prompt diagnosis and adequate management of SLE remains great challenges (8). In fact, laboratory tests and biomarker panels that enables early and accurate diagnosis of SLE are still not at hand, for review see (8, 9, 15, 16, 18, 19, 36). In this context, autoantibodies, such as ANA and anti-dsDNA, have frequently been exploited, but the use of these immunological markers for diagnosis is associated with considerable drawbacks (4). Additional biomarkers that have been suggested in the quest of improving the specificity and sensitivity of the diagnosis, include e.g. abnormal levels of erythrocyte-bound complement activation product C4d and complement receptor 1 (37), platelet bound C4d (38), and lymphocyte bound C4d (39). Hence, additional panels of high-performing serum, plasma, and/or urine biomarker panels would thus be essential.

Spurred by two recent discovery studies (23) (Carlsson et al, unpublished observations), we have in this study extended our efforts in harnessing the diagnostic power of the immune system. More specifically, we further explored the fact that immunoregulation is a central phenomenon of SLE. To this end, we designed our 195 antibody microarrays to target predominantly key regulatory serum proteins, including 73 unique proteins and 15 peptide motifs. Despite only targeting a focused window of the entire serum proteome, the results showed that we could extract condensed (<25-plex) serum biomarker panels differentiating SLE vs. healthy controls irrespective of the disease phenotype (reflecting the disease severity). The classification was accomplished displaying a high discriminatory power, illustrated by a (median) ROC AUC of 0.86 to 0.94. In this context, it should be noted that the bioinformatic analyses were performed using two of the most stringent procedures at hand (training and test sets, combined with backward elimination and frozen SVM versus leave-one-out cross-validation).

In the case of SLE vs. healthy controls, the SLE-associated biomarker panel was identified through backward elimination (35), defining the condensed signature displaying the best classification. Such panels are designed to contain biomarkers providing as orthogonal information as possible, while when viewed alone, an individual marker might not be significantly (p<0.05) differentially expressed. Noteworthy, the core signature, composed of six proteins (C3, CD40, Cystatin C, MCP-1, Sialyl lewis x, and TGF-β), identified in all ten iterative comparisons irrespectively of how the training and test sets were defined, were also found to be differentially expressed. In addition, five of these proteins were also targeted in our recent discovery studies, and four of these were then found to be differentially expressed (C3, CD40, Sialyl lewis x, and TGF-3) (23) (Carlsson et al, unpublished observations). While Sialyl lewis x appeared to be a novel marker, the other five proteins have previously been found to be associated with SLE. C3 and interferon-regulated cytokines, such as MCP-1, have been indicated as potential markers for disease activity (16, 40). TGF-β plays a large role in the control of autoimmunity, and it has been suggested that it might be involved in pathogenesis of renal damage (41). CD40 has been identified as susceptibility locus, and altered levels might have implications for the regulation of aberrant immune response in the disease (42). In addition, Cystatin C serum levels have been found to be dependent on renal function (43).

In addition to the 6 core markers, the overall list of variables was composed of novel markers as well additional markers already reported to be associated with SLE (8, 9, 15, 16, 18, 19, 36). As for example, several complement proteins (C4, C1 esterase inhibitor, factor b, C1q, and properdin) were found to be deregulated, and complement proteins (e.g. C4 and C1q) have also been frequently implicated in the pathogenesis of SLE (5, 44, 45). Several cytokines (e.g. IL-2, IL-4, IL-6, IL-12, IL-16, and TNF-α) were also found to be de-regulated as previously indicated, and could play a key role in the immune dysregulation in SLE (46). It should, however, be noted that these a priori known candidate biomarkers have mainly been reported as individual markers, and not in the context of a high-performing multiplex serum biomarker signature for SLE.

The biological relevance of the SLE-associated condensed serum biomarker panel was also highlighted by the data mining and pathway analysis, further supporting our approach of using the immune system as a sensor for SLE. As for example, when searching for disease by biomarkers, the software tool proposed SLE as the top indication. Further, the pathway analysis also indicated apoptosis, or programmed cell-death as a top process. Abnormal immunoregulation, as reflected by defective clearance of immune complexes and apoptopic cells (materials), have also been identified as a feature in SLE (5). The reason(s) for this defect is not clear, but might be due to quantitative or qualitative defects of early complement proteins, such as C2, C4, or C1q.

Finally, we also investigated whether disease severity, as reflected by the three phenotypes of SLE (44), was a confounding factor for the classification. In our recent discovery studies, the data indicated the classification was challenging for the phenotype displaying the least symptoms (SLE1), but improved with increasing symptoms, i.e. SLE1<SLE2<SL3 (23). In this study, the biomarker signatures were improved and refined, which could be explained by three key factors, namely, i) we analysed a significantly larger sample cohort, ii) we targeted a larger set of immunoregulatory analytes, and iii) we used a re-optimized microarray platform with significantly improved performances (23) (Carlsson et al, unpublished observations) (Delfani et al, unpublished observations). In this study, the data thus showed that the classification of SLE vs. healthy controls was high (ROC AUC of 0.90 to 0.94) irrespective of disease severity (phenotype). In other words, the disease severity was not a confounding factor for classification. Again, the biological relevance of several of the observed biomarkers, such as C3, C4, CD40, MCP-1, IL-6, IL12, and cystatine C was supported by the literature. As above, these markers have been reported mainly as individual markers and not in the context of a multiplex high-performing serum biomarker signature (8, 9, 15, 16, 18, 19, 36).

Taken together, among other things, we have defined a condensed 25-plex serum biomarker signature reflecting SLE using affinity proteomics, thereby enabling serum-based diagnosis of SLE.

TABLE 1
Demographic data of SLE patients and healthy
controls included in the study.

| Parameter | SLE | Healthy controls |
|---|---|---|
| No. of patients | 86 | 50 |
| No. of serum samples | 147* | 50 |
| SLE1:SLE2:SLE3 (ratio) | (30:30:87) | |
| Gender (female:male | (76:10) | (48:2) |
| ratio) | | |
| Mean age (range age) | 39 (18-72) | 50 (19-68) |

*The samples were collected over time during follow-up and the patients were presented with either flare or remission, i.e. for some patients up to four samples were collected at different time-points.

### REFERENCES

1. D'Cruz D P K M, Hughes G R. systemic lupus erythematosus. Lancet. 2007 369(9561):587-96.
2. Tsokos G C. systemic lupus erythematosus. The New England journal of medicine. 2011 Dec. 1; 365(22):2110-21. PubMed PMID: 22129255.
3. V C. K. systemic lupus erythematosus: from genes to organ damage. Methods Mol Biol 2010; 662:265-83.
4. Rahman A I D. systemic lupus erythematosus. N Engl J Med. 2008 358(9):929-39.
5. Mok C C L C. Pathogenesis of systemic lupus erythematosus. J Clin Pathol. 2003 56(7): 481-490.
6. Liu C C M S, Kao A H, Navratil J S, Ahearn J M. Cell-bound complement biomarkers for systemic lupus erythematosus: from benchtop to bedside. Rheum Dis Clin North Am 2010 36(1):161-72, x.
7. Varga J. systemic sclerosis: an update. Bulletin of the NYU hospital for joint diseases. 2008; 66(3):198-202. PubMed PMID: 18937632. eng.
8. S. M. Lupus update: perspective and clinical pearls. Cleve Clin J Med. 2009 76(2):137-42.
9. Merrill J T B J. The role of biomarkers in the assessment of lupus. Best Pract Res Clin Rheumatol. 2005 19(5):709-26.
10. Narain S R H, Satoh M, Sarmiento M, Davidson R, Shuster J, Sobel E, Hahn P, Reeves W H. Diagnostic accuracy for lupus and other systemic autoimmune diseases in the community setting. Arch Intern Med. 2004 164(22):2435-41.
11. Fries J F, Holman H R. systemic lupus erythematosus: a clinical analysis. Major problems in internal medicine. 1975; 6:v-199. PubMed PMID: 1177502.
12. Smith E L S R. The American College of Rheumatology criteria for the classification of systemic lupus erythematosus: strengths, weaknesses, and opportunities for improvement. Lupus 1999; 8(8):586-95.
13. Tan E M C A, Fries J F, Masi A T, McShane D J, Rothfield N F, Schaller J G, Talal N, Winchester R J. The 1982 revised criteria for the classification of systemic lupus erythematosus. Arthritis Rheum 1982 25(11):1271-7.
14. Petri M, Orbai A M, Alarcon G S, Gordon C, Merrill J T, Fortin P R, et al. Derivation and validation of the systemic lupus International Collaborating Clinics classification criteria for systemic lupus erythematosus. Arthritis and rheumatism. 2012 August; 64(8):2677-86. PubMed PMID: 22553077. Pubmed Central PMCID: 3409311.
15. Ahearn J M L C, Kao A H, Manzi S. Biomarkers for systemic lupus erythematosus. Transl Res 2012 159(4):326-42.
16. Rovin B H, Birmingham D J, Nagaraja H N, Yu C Y, Hebert L A. Biomarker discovery in human SLE nephritis. Bulletin of the NYU hospital for joint diseases. 2007; 65(3):187-93. PubMed PMID: 17922668. eng.
17. Sui W H X, Che W, Yang M, Dai Y. The applied basic research of systemic lupus erythematosus based on the biological omics. Genes Immun. 2013 14(3):133-46. doi: 10.1038/gene.2013.3.
18. Liu C C, Ahearn J M. The search for lupus biomarkers. Best practice & research. 2009 August; 23(4):507-23. PubMed PMID: 19591781. eng.
19. Liu C C, Manzi S, Ahearn J M. Biomarkers for systemic lupus erythematosus: a review and perspective. Current opinion in rheumatology. 2005 September; 17(5):543-9. PubMed PMID: 16093831. eng.
20. Gibson D S, Banha J, Penque D, Costa L, Conrads T P, Cahill D J, et al. Diagnostic and prognostic biomarker discovery strategies for autoimmune disorders. Journal of proteomics. 2010 Apr. 18; 73(6):1045-60. PubMed PMID: 19995622.
21. Ingvarsson J L A, Sjöholm A G, Truedsson L, Jansson B, Borrebaeck C A, Wingren C. Design of recombinant antibody microarrays for serum protein profiling: targeting of complement proteins. J Proteome Res. 2007 6(9):3527-36.
22. Wingren C I J, Dexlin L, Szul D, Borrebaeck C A. Design of recombinant antibody microarrays for complex proteome analysis: choice of sample labeling-tag and solid support. Proteomics 2007 7(17):3055-65.
23. Carlsson A W D, Ingvarsson J, Bengtsson A A, Sturfelt G, Borrebaeck C A, Wingren C. Serum protein profiling of systemic lupus erythematosus and systemic sclerosis using recombinant antibody microarrays. Mol Cell Proteomics. 2011 M110.005033.
24. M C. H. Updating the American College of Rheumatology revised criteria for the classification of systemic lupus erythematosus. Arthritis Rheum 1997 40(9):1725.
25. Sturfelt G, Sjoholm A G. Complement components, complement activation, and acute phase response in systemic lupus erythematosus. International archives of allergy and applied immunology. 1984; 75(1):75-83. PubMed PMID: 6086533. Epub 1984/01/01. eng.
26. Gladman D D, Ibafiez D, Urowitz M B. systemic lupus erythematosus disease activity index 2000. The Journal of Rheumatology. 2002 Feb. 1, 2002; 29(2):288-91.
27. Wingren C B C. Antibody microarray analysis of directly labelled complex proteomes. Curr Opin Biotechnol. 2008 19(1):55-61.
28. Olsson N, Wallin S, James P, Borrebaeck C A K, Wingren C. Epitope-specificity of recombinant antibodies reveals promiscuous peptide-binding properties. Protein Science. 2012; 21(12):1897-910.
29. Soderlind E S L, Jirhol P, Kobayashi N, Alexeiva V, Aberg A M, Nilsson A, Jansson B, Ohlin M, Wingren C, Danielsson L, Carlsson R, Borrebaeck C A. Recombining germline-derived CDR sequences for creating diverse single-framework antibody libraries. Nat Biotechnol 2000 18(8):852-6.
30. Carlsson A W C, Ingvarsson J, Ellmark P, Baldertorp B, Fernb M, Olsson H, Borrebaeck C A. Serum proteome profiling of metastatic breast cancer using recombinant antibody microarrays. Eur J Cancer. 2008 44(3):472-80.
31. Carlsson A P O, Ingvarsson J, Widegren B, Salford L, Borrebaeck C A, Wingren C. Plasma proteome profiling reveals biomarker patterns associated with prognosis and therapy selection in glioblastoma multiforme patients. Proteomics Clin Appl. 2010 4(6-7):591-602.
32. Chih-chung C, Chih-Jen L. LIBSVM: a library for support vector machines. http/::wwwcsientuedutw/cjlin/libsvm.2007.
33. Cristianini N, Shawe-Taylor J. An introduction to support vector machines and other kernel-based learning methods. Cambridge Univeristy Press. 2000.
34. Ihaka R, Gentleman R. A language for data analysis and graphics. J Comp Graph Stat. 1996; 5:299-314.
35. Carlsson A W C, Kristensson M, Rose C, Ferno M, Olsson H, Jernstrim H, Ek S, Gustavsson E, Ingvar C, Ohlsson M, Peterson C, Borrebaeck C A. Molecular serum portraits in patients with primary breast cancer predict the development of distant metastases. Proc Natl Acad Sci USA. 2011 108(34):14252-7.
36. Mok C C. Biomarkers for lupus nephritis: a critical appraisal. Journal of biomedicine & biotechnology. 2010; 2010:638413. PubMed PMID: 20414362. Pubmed Central PMCID: 2857808.
37. Manzi S, Navratil J S, Ruffing M J, Liu C C, Danchenko N, Nilson S E, et al. Measurement of erythrocyte C4d and complement receptor 1 in systemic lupus erythematosus. Arthritis and rheumatism. 2004 November; 50(11):3596-604. PubMed PMID: 15529364.
38. Navratil J S, Manzi S, Kao A H, Krishnaswami S, Liu C C, Ruffing M J, et al. Platelet C4d is highly specific for systemic lupus erythematosus. Arthritis and rheumatism. 2006 February; 54(2):670-4. PubMed PMID: 16447243.
39. Liu C C, Kao A H, Hawkins D M, Manzi S, Sattar A, Wilson N, et al. Lymphocyte-bound complement activation products as biomarkers for diagnosis of systemic lupus erythematosus. Clinical and translational science. 2009 August; 2(4):300-8. PubMed PMID: 20161444. Pubmed Central PMCID: 2790176.
40. Bauer J W P M, Batliwalla F M, Koeuth T, Wilson J, Slattery C, Panoskaltsis-Mortari A, Gregersen P K, Behrens T W, Baechler E C. Interferon-regulated chemokines as biomarkers of systemic lupus erythematosus disease activity: a validation study. Arthritis Rheum 2009 60(10):3098-107.
41. Jin T, Almehed K, Carlsten H, Forsblad-d'Elia H. Decreased serum levels of TGF-beta1 are associated with renal damage in female patients with systemic lupus erythematosus. Lupus. 2012 March; 21(3):310-8. PubMed PMID: 22072025.
42. Vazgiourakis V M, Zervou M I, Choulaki C, Bertsias G, Melissourgaki M, Yilmaz N, et al. A common SNP in the CD40 region is associated with systemic lupus erythematosus and correlates with altered CD40 expression: implications for the pathogenesis. Annals of the rheumatic diseases. 2011 December; 70(12):2184-90. PubMed PMID: 21914625.
43. Chew C, Pemberton P W, Husain A A, Haque S, Bruce I N. Serum cystatin C is independently associated with renal impairment and high sensitivity C-reactive protein in systemic lupus erythematosus. Clinical and experimental rheumatology. 2013 March-April; 31(2):251-5. PubMed PMID: 23306163.
44. Sturfelt G, and Sjöholm, A. G. Complement components, complement activation, and acute phase response in systemic lupus erythematosus. Int Arch Allergy Appl Immunol. 1984; 75:75-83.
45. Walport M J. Complement and systemic lupus erythematosus. Arthritis research. 2002; 4 Suppl 3:S279-93. PubMed PMID: 12110148. Pubmed Central PMCID: 3240161.
46. Suh C H, Kim H A. Cytokines and their receptors as biomarkers of systemic lupus erythematosus. Expert review of molecular diagnostics. 2008 March; 8(2):189-98. PubMed PMID: 18366305.

Supplementary Materials and Methods

Production and Purification of Antibodies

In total, 195 human recombinant scFv antibodies, including 180 antibodies targeting 73 mainly immunoregulatory analytes, anticipated to reflect the events taking place in SLE, and 15 scFv antibodies targeting 15 short amino acid motifs (4 to 6 amino acids long) (8) were selected from a large phage display library (Table II) (9) (Persson et al, unpublished data). The specificity, affinity (normally in the nM range), and on-chip functionality of these phage display derived scFv antibodies was ensured by using i) stringent phage-display selection and screening protocols (9), ii) multiple clones (1-9) per target, and iii) a molecular design, adapted for microarray applications (10). In addition, the specificity of several of the antibodies have previously also been validated using well-characterized, standardized serum samples (with known analytes of the targeted analytes), and orthogonal methods, such as mass spectrometry (affinity pull-down experiments), ELISA, MesoScaleDiscovery (MSD) assay, cytometric bead assay, and MS, as well as using spiking and blocking (Table II) (5, 6, 11-17). Notably, the reactivity of some antibodies might be lost since the label (biotin) used to label the sample to enable detection could block the affinity binding to the antibodies (epitope masking). However, we addressed this potential problem by frequently including more than one antibody clone against the same protein, but directed against different epitopes (10).

All scFv antibodies were produced in 100 ml E. coli and purified from expression supernatants using affinity chromatography on Ni²⁺-NTA agarose (Qiagen, Hilden, Germany). ScFvs were eluted using 250 mM imidazole, extensively dialyzed against PBS (pH 7.4), and stored at 4° C. until use. The protein concentration was determined by measuring the absorbance at 280 nm (average 340 μg/ml, range 30-1500 μg/ml). The degree of purity and integrity of the scFv antibodies was evaluated by 10% SDS-PAGE (Invitrogen, Carlsbad, Calif., USA).

Production and Analysis of Antibody Microarrays

The scFv microarrays were produced using a previously optimized and validated set-up (14) (Delfani et al, unpublished data). Briefly, the antibodies were printed on black polymer MaxiSorp microarray slides (NUNC A/S, Roskilde, Denmark), by spotting one drop (˜330 pL) at each position, using a non-contact printer (SciFlexarrayer S11, Scienion, Berlin, Germany). Each microarray, composed of 195 scFvs antibodies, one negative control (PBS) and one positive control (biotinylated BSA, b-BSA), was split into 14 sub-arrays of 25×28 spots. Furthermore, each sub-array was divided in three segments where a row of b-BSA consisting of 25 replicate spots was printed at the beginning and the end of each segment. Each scFv antibody was dispensed in three replicates, one in each segment, to assure adequate reproducibility.

For handling the arrays, we used a recently optimized protocol (Delfani et al, unpublished data). Briefly, the printed microarrays were allowed to dry for 2 h at RT and were then mounted in a multi-well incubation chambers (NEXTERION® IC-16) (Schott, Jena, Germany). Next, the slides were blocked with 1% (v/v) Tween-20 (Merck Millipore) and 1% (w/v) fat-free milk powder (Semper, Sundbyberg, Sweden) in PBS (MT-PBS solution) for 2 h at RT. Subsequently, the slides were washed for four times with 150 μl 0.05% (v/v) Tween-20 in PBS (T-PBS solution), and then incubated with 100 μl biotinylated serum sample, diluted 1:10 in MT-PBS solution (corresponding to a total serum dilution of 1:450), for 2 h at RT under gentle agitation using an orbital shaker. After another washing, the slides were incubated with 100 μl 1 μg/ml Alexa 647-labelled streptavidin (SA647) (Invitrogen) in MT-PBS for 1 h at RT under agitation. Finally, the slides were washed in T-PBS, and dried under a stream of nitrogen gas, and immediately scanned with a confocal microarray scanner (ScanArray Express, PerkinElmer Life & Analytical Sciences) at 10 μm resolution, using fixed scanner settings of 60% PMT gain and 90% laser power.

Data Pre-Processing

The ScanArray Express software v4.0 (PerkinElmer Life & Analytical Sciences) was used to quantify spot signal intensities, using the fixed circle method. Signal intensities with local background subtraction were used for data analysis. Each data point represents the mean value of all three replicate spots unless any replicate CV exceeded 15%, in which case the worst performing replicate was eliminated and the average value of the two remaining replicates was used instead. Log¹⁰ values of signal intensities were used for subsequent analysis.

For evaluation of normalization strategies and initial analysis on variance, the data was visualized using principal component analysis (PCA) and hierarchical clustering In Qluecore Omics Explorer (Qlucore AB, Lund, Sweden). Subsequently, the data normalization procedure was carried out in two steps. First, the microarray data was normalized for array-to-array variations using a semi-global normalization method, where 20% of the analytes displaying the lowest CV-values over all samples were identified and used to calculate a scaling factor, as previously described (14, 18, 19). Second, the data was normalized for day-to-day variation using the “subtract by group mean” approach. In this approach, the mean value (x̅) of each analyte (i) within each day of analysis was calculated (=x̅ᵢ), and subtracted from the respective individual values (xᵢ), thus zero centering the data (=xᵢ−x̅ᵢ). Finally, the global mean signal for each antibody was calculated and added to each respective data point in order to avoid negative values in the data set.

Data Analysis

The support vector machine (SVM) is a supervised learning method in R (20-22) that we was used to classify the samples. The supervised classification was conducted using a linear kernel, and the cost of constraints was set to 1, which is the default value in the R function SVM, and no attempt was performed to tune it. This absence of parameter tuning was chosen to avoid over fitting. No filtration on the data was done before training the SVM, i.e. all antibodies used on the microarray were included in the analysis. Further, a receiver operating characteristics (ROC) curve, as constructed using the SVM decision values and the area under the curve (AUC), was calculated.

Depending on the size of the sample cohorts, two different strategies were applied. For classification of SLE vs. N and SLE3 vs. N, the samples were first randomly divided into a training set (⅔ of the data) and a test set (⅓ of the data) while maintaining the same ratios of samples from each group. It should be noted that for those SLE patients where more than one sample was at hand, the sample was randomly selected for each comparison, and only one sample per patient was included in each subset comparison in order to avoid bias. A backward elimination algorithm (23) combined with a leave-one-out cross-validation procedure was then applied to the training set to create a condensed panel of antibodies displaying the highest combined discriminatory power. The condensed panel of antibodies was then employed to train a single SVM model on the training set. The trained SVM model was then frozen and applied to the test set, and a ROC AUC was calculated and used to evaluate the performance of the SVM classifier. In order to demonstrate the robustness of the data set, 9 additional training and test sets were generated and the above data analysis process was repeated. Finally, the frequency at which each antibody was included in all 10 different defined antibody panels was assessed.

When classifying SLE1 vs. N and SLE2 vs. N the number of samples was not large enough to divide the sample set into a training set and a test set. Therefore, the SVM was trained using the leave-one-out cross-validation procedure as previously described (18). By iterating all samples, a ROC curve was constructed using the decision values and the corresponding AUC value was determined, and used for evaluating the prediction performance of the classifier.

Significantly differentially expressed analytes (p<0.05) were identified based on Wilcoxon rank sum tests. Heat maps and visualization of the samples by principal component analysis (PCA) were carried using Qlucore Omics Explorer. Data-mining and pathway analysis was conducted using Metacore (Thomson Reuters, New York, N.Y., USA).

### REFERENCES

1. M C. H. Updating the American College of Rheumatology revised criteria for the classification of systemic lupus erythematosus. Arthritis Rheum 1997 40(9):1725.
2. Tan E M C A, Fries J F, Masi A T, McShane D J, Rothfield N F, Schaller J G, Talal N, Winchester R J. The 1982 revised criteria for the classification of systemic lupus erythematosus. Arthritis Rheum 1982 25(11):1271-7.
3. Sturfelt G, Sjoholm A G. Complement components, complement activation, and acute phase response in systemic lupus erythematosus. International archives of allergy and applied immunology. 1984; 75(1):75-83.
4. Gladman D D, Ibaiez D, Urowitz M B. systemic lupus erythematosus disease activity index 2000. The Journal of Rheumatology. 2002; 29(2):288-91.
5. Ingvarsson J L A, Sjöholm A G, Truedsson L, Jansson B, Borrebaeck C A, Wingren C. Design of recombinant antibody microarrays for serum protein profiling: targeting of complement proteins. J Proteome Res. 2007 6(9):3527-36.
6. Wingren C I J, Dexlin L, Szul D, Borrebaeck C A. Design of recombinant antibody microarrays for complex proteome analysis: choice of sample labeling-tag and solid support. Proteomics 2007 7(17):3055-65.
7. Wingren C B C. Antibody microarray analysis of directly labelled complex proteomes. Curr Opin Biotechnol. 2008 19(1):55-61.
8. Olsson N, Wallin S, James P, Borrebaeck C A K, Wingren C. Epitope-specificity of recombinant antibodies reveals promiscuous peptide-binding properties. Protein Science. 2012; 21(12):1897-910.
9. Söderlind E S L, Jirholt P, Kobayashi N, Alexeiva V, Aberg A M, Nilsson A, Jansson B, Ohlin M, Wingren C, Danielsson L, Carlsson R, Borrebaeck C A. Recombining germline-derived CDR sequences for creating diverse single-framework antibody libraries. Nat Biotechnol 2000 18(8):852-6.
10. Borrebaeck C K, Wingren C. Recombinant Antibodies for the Generation of Antibody Arrays. In: Korf U, editor. Protein Microarrays. Methods in Molecular Biology. 785: Humana Press; 2011. p. 247-62.
11. Kristensson M O K, Carlson J, Wullt B, Sturfelt G, Borrebaeck C A, Wingren C. Design of recombinant antibody microarrays for urinary proteomics. Proteomics Clin Appi 2012 6(5-6):291-6.
12. Persson J, Backstrm M, Johansson H, Jirstrom K, Hansson G C, Ohlin M. Molecular Evolution of Specific Human Antibody against MUC1 Mucin Results in Improved Recognition of the Antigen on Tumor Cells. Tumor Biology. 2009; 30(4):221-31.
13. Gustavsson E, Ek S, Steen J, Kristensson M, Algenas C, Uhlen M, et al. Surrogate antigens as targets for proteome-wide binder selection. New Biotechnology. 2011; 28(4):302-11.
14. Carlsson A W D, Ingvarsson J, Bengtsson A A, Sturfelt G, Borrebaeck C A, Wingren C. Serum protein profiling of systemic lupus erythematosus and systemic sclerosis using recombinant antibody microarrays. Mol Cell Proteomics. 2011 M110.005033.
15. Dexlin-Mellby L S A, Centlow M, Nygren S, Hansson S R, Borrebaeck C A, Wingren C. Tissue proteome profiling of preeclamptic placenta using recombinant antibody microarrays. Proteomics Clin Appl. 2010 4(10-11):794-807.
16. Ingvarsson J W C, Carlsson A, Ellmark P, Wahren B, Engstrom G, Harmenberg U, Krogh M, Peterson C, Borrebaeck C A. Detection of pancreatic cancer using antibody microarray-based serum protein profiling. Proteomics. 2008 8(11):2211-9.
17. Pauly F, Dexlin-Mellby L, Ek S, Ohlin M, Olsson N, Jirstrim K, et al. Protein Expression Profiling of Formalin-Fixed Paraffin-Embedded Tissue Using Recombinant Antibody Microarrays. Journal of Proteome Research. 2013; 12(12):5943-53.
18. Carlsson A W C, Ingvarsson J, Ellmark P, Baldertorp B, Ferno M, Olsson H, Borrebaeck C A. Serum proteome profiling of metastatic breast cancer using recombinant antibody microarrays. Eur J Cancer. 2008 44(3):472-80.
19. Carlsson A P O, Ingvarsson J, Widegren B, Salford L, Borrebaeck C A, Wingren C. Plasma proteome profiling reveals biomarker patterns associated with prognosis and therapy selection in glioblastoma multiforme patients. Proteomics Clin Appl. 2010 4(6-7):591-602.
20. Chih-chung C, Chih-Jen L. LIBSVM: a library for support vector machines. http/::wwwcsientuedutw/cjlin/libsvm.2007.
21. Cristianini N, Shawe-Taylor J. An introduction to support vector machines and other kernel-based learning methods. Cambridge Univeristy Press. 2000.
22. Ihaka R, Gentleman R. A language for data analysis and graphics. J Comp Graph Stat. 1996; 5:299-314.
23. Carlsson A W C, Kristensson M, Rose C, Fernö M, Olsson H, Jernstrom H, Ek S, Gustavsson E, Ingvar C, Ohlsson M, Peterson C, Borrebaeck C A. Molecular serum portraits in patients with primary breast cancer predict the development of distant metastases. Proc Natl Acad Sci USA. 2011 108(34):14252-7.

SUPPLEMENTARY TABLE 1
Antigens targeted on the antibody microarray

| | | No of antibody |
|---|---|---|
| Protein | Full name | clones |
| Angiomotin | Angiomotin | 2 |
| APOA1 | Apolipoprotein A1 | 3 |
| APOA4 | Apolipoprotein A4 | 3 |
| ATP5B | ATP synthase subunit beta, | 3 |
| | mitochondrial | |
| Beta- | Beta-galactosidase | 1 |
| galactosidase | | |
| BTK | Tyrosine-protein kinase BTK | 1 |
| C1 est. inh. | Plasma protease C1 inhibitor | 4 |
| C1q* | Complement C1q | 1 |
| C1s | Complement C1s | 1 |
| C3* | Complement C3 | 6 |
| C4* | Complement C4 | 4 |
| C5* | Complement C5 | 3 |
| CD40 | CD40 protein | 4 |
| CD40 ligand | CD40 ligand | 1 |
| CHX10 | Visual system homeobox 2 | 3 |
| CT | Cholera toxin subunit B (Control) | 1 |
| Cyst. C | Cystatin-C | 4 |
| Digoxin | Digoxin | 1 |
| DUSP9 | Dual specificity protein | 1 |
| | phosphatase 9 | |
| Eotaxin | Eotaxin | 3 |
| Factor B* | Complement factor B | 4 |
| GLP-1 | Glucagon-like peptide-1 | 1 |
| GLP-1 R | Glucagon-like peptide 1 receptor | 1 |
| GM-CSF | Granulocyte-macrophage colony- | 3 |
| | stimulating factor | |
| HLA-DR/DP | HLA-DR/DP | 1 |
| ICAM-1 | Intercellular adhesion molecule 1 | 1 |
| IFN-gamma | Interferon gamma | 3 |
| IgM | Immunoglobulin M | 5 |
| IL-1 alpha* | Interleukin-1 alpha | 3 |
| IL-1 beta | Interleukin-1 beta | 3 |
| IL-10* | Interleukin-10 | 3 |
| IL-11 | Interleukin-11 | 3 |
| IL-12* | Interleukin-12 | 4 |
| IL-13* | Interleukin-13 | 3 |
| IL-16 | Interleukin-16 | 3 |
| IL-18 | Interleukin-18 | 3 |
| IL-1ra | Interleukin-1 receptor antagonist | 3 |
| | protein | |
| IL-2 | Interleukin-2 | 3 |
| IL-3 | Interleukin-3 | 3 |
| IL-4* | Interleukin-4 | 4 |
| IL-5* | Interleukin-5 | 3 |
| IL-6* | Interleukin-6 | 4 |
| IL-7 | Interleukin-7 | 2 |
| IL-8* | Interleukin-8 | 3 |
| IL-9 | Interleukin-9 | 3 |
| Integrin alpha-10 | Integrin alpha-10 | 1 |
| Integrin alpha-11 | Integrin alpha-11 | 1 |
| JAK3 | Tyrosine-protein kinase JAK3 | 1 |
| LDL | Apolipoprotein B-100 | 2 |
| Leptin | Leptin | 1 |
| Lewisx | Lewis x | 2 |
| Lewisy | Lewis y | 1 |
| LUM | Lumican | 1 |
| MCP-1* | C-C motif chemokine 2 | 9 |
| MCP-3 | C-C motif chemokine 7 | 3 |
| MCP-4 | C-C motif chemokine 13 | 3 |
| Motif | Peptide motifs | 15 |
| MYOM2 | Myomesin-2 | 2 |
| ORP-3 | Oxysterol-binding protein-related | 2 |
| | protein 3 | |
| Procathepsin W | Procathepsin W | 1 |
| Properdin* | Properdin | 1 |
| PSA | Prostate-specific antigen | 1 |
| RANTES | C-C motif chemokine 5 | 3 |
| Sialle x | Sialyl Lewis x | 1 |
| Sox11a | Transcription factor SOX-11 | 1 |
| Surface Ag X | Surface Ag X | 1 |
| TBC1D9 | TBC1 domain family member 9 | 3 |
| TGF-beta1 | Transforming growth factor beta-1 | 3 |
| TM peptide | Transmembrane peptide | 1 |
| TNF-alpha | Tumor necrosis factor | 3 |
| TNF-beta* | Lymphotoxin-alpha | 4 |
| UPF3B | Regulator of nonsense transcripts 3B | 2 |
| VEGF* | Vascular endothelial growth factor | 4 |

*Antibody specificity determined by protein arrays, MSD, ELISA, blocking/spiking experiments, and/or mass spectrometry.

1. A method for determining a systemic lupus erythematosus-associated disease state in a subject comprising or consisting of the steps of:
a) providing one or more sample to be tested and an array comprising a plurality of binding agents that bind to a biomarker selected from the group defined in Table A; and
b) using said array, measuring the presence and/or amount in the test sample of one or more biomarker selected from the group defined in Table A;
wherein the presence and/or amount in the one or more test sample of the one or more biomarker(s) selected from the group defined in Table A is indicative of a systemic lupus erythematosus-associated disease state.

2. The method according to claim 1 further comprising or consisting of the steps of:
c) providing one or more control sample from an individual with a different systemic lupus erythematosus-associated disease state to the test subject; and
d) using said array, measuring the presence and/or amount in the control sample of the one or more biomarkers measured in step (b);
wherein the systemic lupus erythematosus-associated disease state is identified in the event that the presence and/or amount in the one or more test sample of the one or more biomarkers measured in step (b) is different from the presence and/or amount in the control sample.

3. The method according to claim 2 wherein the control sample of step (c) is provided from a healthy individual or an individual with systemic lupus erythematosus.

4-7. (canceled)

8. The method according to claim 2 further comprising or consisting of the steps of:
e) providing one or more control sample from an individual afflicted with the same systemic lupus erythematosus-associated disease state to the test subject (i.e., a positive control); and
f) using said array, measuring the presence and/or amount in the control sample of the one or more biomarkers measured in step (b);
wherein the systemic lupus erythematosus-associated disease state is identified in the event that the presence and/or amount in the test sample of the one or more biomarkers measured in step (b) corresponds to the presence and/or amount in the control sample of the one or more biomarkers measured in step (f).

9. The method according to claim 1, wherein step (b) comprises or consists of measuring the presence and/or amount in the test sample of two or more of the biomarkers defined in Table A.

10-12. (canceled)

13. The method according to claim 1, wherein the method is for diagnosing systemic lupus erythematosus in an individual.

14. The method according to claim 13 wherein step (b) comprises or consists of measuring the presence and/or amount in the test sample of biomarkers defined in Table A(i), Table A(ii) and/or Table A(iii).

15. The method according to claim 1, wherein the method is for characterising systemic lupus erythematosus in an individual (determining whether the individual has systemic lupus erythematosus, subtype 1, subtype 2 or subtype 3).

16. The method according to claim 1, wherein step (b) comprises or consists of measuring the presence and/or amount in the test sample of one or more of the biomarkers defined in FIG. 1B, FIG. 4A, FIG. 4C, and/or FIG. 4D.

17-22. (canceled)

23. The method according to claim 1 wherein step (b) comprises measuring the expression of the protein, polypeptide or nucleic acid of the one or more biomarker(s).

24. (canceled)

25. The method according to claim 1 wherein each of the plurality of binding agents is an antibody or a fragment thereof.

26-27. (canceled)

28. The method according to claim 1 wherein the one or more biomarker(s) in the test sample and/or the one or more biomarker(s) in the control sample is labelled with a detectable moiety.

29. (canceled)

30. The method according to claim 28 wherein the detectable moiety is selected from the group consisting of: a fluorescent moiety, a luminescent moiety, a chemiluminescent moiety, a radioactive moiety, and an enzymatic moiety.

31. The method according to claim 1 wherein step (b) comprises measuring the expression of the nucleic acid of the one or more biomarker(s).

32-36. (canceled)

37. The method according to claim 1 wherein the array is selected from the group consisting of a bead-based array and a surface-based array.

38. (canceled)

39. The method according to claim 1 wherein the array is selected from the group consisting of macroarray, microarray and nanoarray.

40-45. (canceled)

46. An array for determining a systemic lupus erythematosus-associated disease state in an individual comprising a plurality of one or more binding agents in the form of an antibody or fragment thereof, or a nucleic acid molecule, that binds to one or more biomarkers selected from the group defined in Table A.

47. (canceled)

48. An array according to claim 46 wherein the array is a bead-based array, a surface-based array, or a macroarray, microarray, or nanoarray.

49. An array according to claim 46 wherein collectively the plurality of binding agents are capable of binding to all of the proteins defined in Table A.

50-51. (canceled)

52. A kit for determining a systemic lupus erythematosus-associated disease state in an individual comprising:
a plurality of one or more first binding agents each in the form of an antibody or fragment thereof, or a nucleic acid molecule, that binds to one or more biomarkers selected from the group defined in Table A, or an array according to claim 46.

53. The kit of claim 52 further comprising:
one or more second binding agent capable of binding to the one or more proteins defined in Table A, the second binding agent having a detectable moiety.

54-55. (canceled)


SEQUENCE LISTING (255 sequences)

SEQ ID NO: 1 - protein, 242 aa, Artificial Sequence
  anti-TM peptide scFv antibody
MAEVQLLESGGGLVQPGGSLRLSCAASGFTFSSYGFHWVRQAPGKGLEWVSLISWDGGSTYYADSVKGRFTISRDNSKNTLYLQMNSLRAEDTAVYYCARGTWFDPWGQGTLVTVSSGGGGSGGGGSGGGGSQSVLTQPPSASGTPGQRVTISCSGSSSNIGNNAVNWYQQLPGTAPKLLIYRNNQRPSGVPDRFSGSKSGTSASLAISGLRSEDEADYYCAAWDDSLSWVFGGGTKLTVLG

SEQ ID NO: 2 - protein, 31 aa, Artificial Sequence
  Affinity tag
DYKDHDGDYKDHDIDYKDDDDKAAAHHHHHH

SEQ ID NO: 3 - protein, 10 aa, Artificial Sequence
  Motif (10) or Motif (11) or Motif (12)
SGSGSEAHLR

SEQ ID NO: 4 - protein, 10 aa, Artificial Sequence
  Motif (13)
SGSGQEASFK

SEQ ID NO: 5 - protein, 8 aa, Artificial Sequence
  Motif (9) or Motif (14)
SGSGEDFR

SEQ ID NO: 6 - protein, 15 aa, Artificial Sequence
  Motif (15)
SGSGGIVKYLYEDEG

SEQ ID NO: 7 - protein, 10 aa, Artificial Sequence
  Motif (1) or Motif (2)
SGSGSSAYSR

SEQ ID NO: 8 - protein, 10 aa, Artificial Sequence
  Motif (4) or Motif (7) or Motif (8)
SGSGTEEQLK

SEQ ID NO: 9 - protein, 10 aa, Artificial Sequence
  Motif (5)
SGSGLSADHR

SEQ ID NO: 10 - protein, 10 aa, Artificial Sequence
  Motif (6)
SGSGLTEFAK

SEQ ID NO: 11 - protein, 14 aa, Artificial Sequence
  Motif (1) CDR H1
FSDYYMSWVRQAPG

SEQ ID NO: 12 - protein, 19 aa, Artificial Sequence
  Motif (1) CDR H2
ADIKRDGSTRYYGDSVKGR

SEQ ID NO: 13 - protein, 12 aa, Artificial Sequence
  Motif (1) CDR H3
ARDRLVAGLFDY

SEQ ID NO: 14 - protein, 15 aa, Artificial Sequence
  Motif (1) CDR L1 or Motif (2) CDR L1 or Motif (6) CDR L1
CTGSSSNIGAGYDVH

SEQ ID NO: 15 - protein, 7 aa, Artificial Sequence
  Motif (1) CDR L2 or Motif (3) CDR L2 or Motif (4) CDR L2 or Motif (5) CDR L2 or Motif (10) CDR L2 or Motif (15) CDR L2
GNSNRPS

SEQ ID NO: 16 - protein, 11 aa, Artificial Sequence
  Motif (1) CDR L3
CAAWDDSLSVL

SEQ ID NO: 17 - protein, 14 aa, Artificial Sequence
  Motif (2) CDR H1 or Motif (3) CDR H1 or Motif (10) CDR H1 or Motif (11) CDR H1 or Motif (13) CDR H1
FSSYAMSWVRQAPG

SEQ ID NO: 18 - protein, 19 aa, Artificial Sequence
  Motif (2) CDR H2
SAISGSGGRTYYTDSVRDR

SEQ ID NO: 19 - protein, 16 aa, Artificial Sequence
  Motif (2) CDR H3
ARDLMPVCQYCYGMDV

SEQ ID NO: 20 - protein, 7 aa, Artificial Sequence
  Motif (2) CDR L2 or Motif (8) CDR L2 or Motif (13) CDR L2
SNNQRPS

SEQ ID NO: 21 - protein, 13 aa, Artificial Sequence
  Motif (2) CDR L3
CQSYDSSLNKDVV

SEQ ID NO: 22 - protein, 10 aa, Artificial Sequence
  Motif (3)
SGSGDFAEDK

SEQ ID NO: 23 - protein, 19 aa, Artificial Sequence
  Motif (3) CDR H2 or Motif (11) CDR H2
SSISSSSSYIYYADSVKGR

SEQ ID NO: 24 - protein, 16 aa, Artificial Sequence
  Motif (3) CDR H3
ARLFFSGGATRAAFDI

SEQ ID NO: 25 - protein, 14 aa, Artificial Sequence
  Motif (3) CDR L1 or Motif (11) CDR L1
CSGGSSNIGSNTVN

SEQ ID NO: 26 - protein, 7 aa, Artificial Sequence
  Motif (11) CDR L2
RNNQRPS

SEQ ID NO: 27 - protein, 12 aa, Artificial Sequence
  Motif (3) CDR L3
CAAWDDSLNGRV

SEQ ID NO: 28 - protein, 14 aa, Artificial Sequence
  Motif (4) CDR H1
FTSYSIHWVRQAPG

SEQ ID NO: 29 - protein, 18 aa, Artificial Sequence
  Motif (4) CDR H2 or Motif (12) CDR H2
SAIGTGGGTYYADSVKGR

SEQ ID NO: 30 - protein, 9 aa, Artificial Sequence
  Motif (4) CDR H3
ARGGYFLDY

SEQ ID NO: 31 - protein, 14 aa, Artificial Sequence
  Motif (4) CDR L1 or Motif (10) CDR L1
CSGSSSNIGSNTVN

SEQ ID NO: 32 - protein, 13 aa, Artificial Sequence
  Motif (4) CDR L3
CQSYDRSLSVNVV

SEQ ID NO: 33 - protein, 14 aa, Artificial Sequence
  Motif (5) CDR H1
FSNYAMSWVRQAPG

SEQ ID NO: 34 - protein, 19 aa, Artificial Sequence
  Motif (5) CDR H2
AFIRYDGSNKYYADSVKGR

SEQ ID NO: 35 - protein, 14 aa, Artificial Sequence
  Motif (5) CDR H3
ARDAVGGDSYVLDY

SEQ ID NO: 36 - protein, 14 aa, Artificial Sequence
  Motif (5) CDR L1
CSGSSSNIGSNAVN

SEQ ID NO: 37 - protein, 12 aa, Artificial Sequence
  Motif (5) CDR L3
CAAWDDSLNGWV

SEQ ID NO: 38 - protein, 14 aa, Artificial Sequence
  Motif (6) CDR H1
FSDYYMSWIRQAPG

SEQ ID NO: 39 - protein, 19 aa, Artificial Sequence
  Motif (6) CDR H2
SSISSRSSYIYYADSVKGR

SEQ ID NO: 40 - protein, 18 aa, Artificial Sequence
  Motif (6) CDR H3
AKDREYYDILTGYPSMDV

SEQ ID NO: 41 - protein, 7 aa, Artificial Sequence
  Motif (6) CDR L2
DNNKRPS

SEQ ID NO: 42 - protein, 12 aa, Artificial Sequence
  Motif (6) CDR L3
CSAWDESLSGVV

SEQ ID NO: 43 - protein, 14 aa, Artificial Sequence
  Motif (7) CDR H1 or Motif (8) CDR H1
FSSYGMHWVRQAPG

SEQ ID NO: 44 - protein, 19 aa, Artificial Sequence
  Motif (7) CDR H2 or Motif (8) CDR H2
SAISGSGGSTYYADSVKGR

SEQ ID NO: 45 - protein, 11 aa, Artificial Sequence
  Motif (7) CDR H3 or Motif (14) CDR H3
ARSRYGSGMDV

SEQ ID NO: 46 - protein, 14 aa, Artificial Sequence
  Motif (7) CDR L1
CSGSSSNVGVNYVY

SEQ ID NO: 47 - protein, 7 aa, Artificial Sequence
  Motif (7) CDR L2
SHNQRPS

SEQ ID NO: 48 - protein, 12 aa, Artificial Sequence
  Motif (7) CDR L3
CAAWDDSLNGVV

SEQ ID NO: 49 - protein, 12 aa, Artificial Sequence
  Motif (8) CDR H3
ARGGVGRYGMDV

SEQ ID NO: 50 - protein, 14 aa, Artificial Sequence
  Motif (8) CDR L1
CSGSSSNIGNNYVS

SEQ ID NO: 51 - protein, 12 aa, Artificial Sequence
  Motif (8) CDR L3
CATWDDSLSGGV

SEQ ID NO: 52 - protein, 13 aa, Artificial Sequence
  Motif (9) CDR H1
FNTAMSWVRQAPG

SEQ ID NO: 53 - protein, 19 aa, Artificial Sequence
  Motif (9) CDR H2
SSISAGGTRTFYADSVRGR

SEQ ID NO: 54 - protein, 16 aa, Artificial Sequence
  Motif (9) CDR H3
ARHRAAGGGYYYGMDV

SEQ ID NO: 55 - protein, 14 aa, Artificial Sequence
  Motif (9) CDR L1
CSGSSSNIGSNSVN

SEQ ID NO: 56 - protein, 7 aa, Artificial Sequence
  Motif (9) CDR L2
DNNRRPS

SEQ ID NO: 57 - protein, 11 aa, Artificial Sequence
  Motif (9) CDR L3
CAAWDDSLNWV

SEQ ID NO: 58 - protein, 19 aa, Artificial Sequence
  Motif (10) CDR H2
AAIWSDGSNKYYADSVKGR

SEQ ID NO: 59 - protein, 12 aa, Artificial Sequence
  Motif (10) CDR H3
AKVGATDDAFDI

SEQ ID NO: 60 - protein, 12 aa, Artificial Sequence
  Motif (10) CDR L3
CAAWDDSLNGPV

SEQ ID NO: 61 - protein, 12 aa, Artificial Sequence
  Motif (11) CDR H3
ARHIQGSGGLDV

SEQ ID NO: 62 - protein, 12 aa, Artificial Sequence
  Motif (11) CDR L3
CAAWDDSLSGVV

SEQ ID NO: 63 - protein, 14 aa, Artificial Sequence
  Motif (12) CDR H1
FTSYSMSWVRQAPG

SEQ ID NO: 64 - protein, 11 aa, Artificial Sequence
  Motif (12) CDR H3
ARVNWNDAFDY

SEQ ID NO: 65 - protein, 14 aa, Artificial Sequence
  Motif (12) CDR L1
CSGSSSNIGNNAVN

SEQ ID NO: 66 - protein, 7 aa, Artificial Sequence
  Motif (12) CDR L2
RNDQRPS

SEQ ID NO: 67 - protein, 12 aa, Artificial Sequence
  Motif (12) CDR L3
CSTWDDSLSGVF

SEQ ID NO: 68 - protein, 19 aa, Artificial Sequence
  Motif (13) CDR H2
SAISGSGGRTYYADAVKGR

SEQ ID NO: 69 - protein, 16 aa, Artificial Sequence
  Motif (13) CDR H3
ARHLKHDDGNSGAFDI

SEQ ID NO: 70 - protein, 14 aa, Artificial Sequence
  Motif (13) CDR L1
CSGSSSNIGTNYVY

SEQ ID NO: 71 - protein, 12 aa, Artificial Sequence
  Motif (13) CDR L3
CAAWDDSLSVWV

SEQ ID NO: 72 - protein, 14 aa, Artificial Sequence
  Motif (14) CDR H1
FDDYGMSWVRQAPG

SEQ ID NO: 73 - protein, 19 aa, Artificial Sequence
  Motif (14) CDR H2
SAISGSGGSTYYADPVKGR

SEQ ID NO: 74 - protein, 14 aa, Artificial Sequence
  Motif (14) CDR L1
CSGSSSNIGSNYVY

SEQ ID NO: 75 - protein, 7 aa, Artificial Sequence
  Motif (14) CDR L2
KSNQRPS

SEQ ID NO: 76 - protein, 12 aa, Artificial Sequence
  Motif (14) CDR L3
CAAWDDRLNAVV

SEQ ID NO: 77 - protein, 14 aa, Artificial Sequence
  Motif (15) CDR H1
FSNYAMHWVRQAPG

SEQ ID NO: 78 - protein, 19 aa, Artificial Sequence
  Motif (15) CDR H2
SSISNRGSRTFYADSVKGR

SEQ ID NO: 79 - protein, 13 aa, Artificial Sequence
  Motif (15) CDR H3
ARDHRWDPGAFDI

SEQ ID NO: 80 - protein, 15 aa, Artificial Sequence
  Motif (15) CDR L1
CTGSSSNIGADYDVH

SEQ ID NO: 81 - protein, 12 aa, Artificial Sequence
  Motif (15) CDR L3
CAAWDDGLSGVV

SEQ ID NO: 82 - protein, 261 aa, Artificial Sequence
  IL-1alpha (1) scFv antibody
  VARIANT (193)..(193): Any amino acid
  VARIANT (239)..(239): Any amino acid
  VARIANT (253)..(255): Any amino acids
EVQLLESGGGLVQPGGSLRLSCAASGFTFSSYAMHWVRQAPGKGLEWVSGVSWNGSRTHYADSVKGRFTISRDNSKNTLYLQMNSLRAEDTAVYYCARSSGYYSWAFDIWGQGTLVTVSSGGGGSGGGGSGGGGSQSVLTQPPSASGTPGQRVTISCSGSSSNIGRNTVNWYQQLPGTAPKLLIYGNSNRPSXVPDRFSGSKSGTSASLAISGLRSEDEADYYCAAWDDSLNGWAFGGXTKLTVLGEQKLISXXXLSGSAA

SEQ ID NO: 83 - protein, 264 aa, Artificial Sequence
  IL-1alpha (2) scFv antibody
  VARIANT (222)..(222): Any amino acid
  VARIANT (242)..(242): Any amino acid
EVQLLESGGGLVQPGGSLRLSCAASGFTFSSYSMNWVRQAPGKGLEWVALISYDGSQKYYADSMKGRFTISRDNSKNTLYLQMNSLRAEDTAVYYCAKGHTSGTKAYYFDSWGQGTLVTVSSGGGGSGGGGSGGGGSQSVLTQPPSASGTPGQRVTISCTGTSSNIGAGYSVHWYQQLPGTAPKLLIYGNSNRPSGVPDRFSGSKSGTSASLAISGLRSEDXADYYCQSYDSSLSGWVFGGXTKLTVLGEQKLISEEDLSGSAA

SEQ ID NO: 84 - protein, 260 aa, Artificial Sequence
  IL-2 (1) scFv antibody
  VARIANT (238)..(238): Any amino acid
  VARIANT (243)..(243): Any amino acid
  VARIANT (252)..(253): Any amino acids
EVQLLESGGGLVQPGGSLRLSCAASGFTFGDYAMSWVRQAPGKGLEWVSSISSRGSYIYFADSVKGRFTISRDNSKNTLYLQMNSLRAEDTAVYYCAKKKTGYYGLDAWGQGTLVTVSSGGGGSGGGGSGGGGSQSVLTQPPSASGTPGQRVTISCTGSSSNIGAGYDVHWYQQLPGTAPKLLIYGNSNRPSGVPDRFSGSKSGTSASLAISGLRSEDEADYYCSSYAGSNNLVFGGXTKLTXLGEQKLISXXDLSGSAA

SEQ ID NO: 85 - protein, 260 aa, Artificial Sequence
  IL-2 (2) scFv antibody
  VARIANT (3)..(4): Any amino acids
  VARIANT (238)..(239): Any amino acids
  VARIANT (253)..(254): Any amino acids
EVXXLESGGGLVQPGGSLRLSCAASGFTFGDYAMSWVRQAPGKGLEWVSSISSRGSYIYFADSVKGRFTISRDNSKNTLYLQMNSLRAEDTAVYYCAKKKTGYYGLDAWGQGTLVTVSSGGGGSGGGGSGGGGSQSVLTQPPSASGTPGQRVTISCTGSSSNIGAGYDVHWYQQLPGTAPKLLIYGNSNRPSGVPDRFSGSKSGTSASLAISGLRSEDEADYYCSSYAGSNNLVFGGXXKLTVLGEQKLISEXXLSGSAA

SEQ ID NO: 86 - protein, 191 aa, Artificial Sequence
  IL-2 (3) scFv antibody
  VARIANT (162)..(162): Any amino acid
EVQLLESGGGLVQPGGSLRLSCAASGFTFGDYAMSWVRQAPGKGLEWVSSISSRGSYIYFADSVKGRFTISRDNSKNTLYLQMNSLRAEDTAVYYCAKKKTGYYGLDAWGQGTLVTVSSGGGGSGGGGSGGGGSQSVLTQPPSASGTPGQRVTISCTGSSSXIGAGYDVHWYQQLPGTAPKLLIYGNSNRP

SEQ ID NO: 87 - protein, 262 aa, Artificial Sequence
  IL-3 (1) scFv antibody
  VARIANT (220)..(220): Any amino acid
  VARIANT (240)..(241): Any amino acids
  VARIANT (254)..(256): Any amino acids
  VARIANT (260)..(260): Any amino acid
EVQLLESGGGLVQPGGSLRLSCAASGFTFGRYTMHWVRQAPGKGLEWVSSISSSSSYIYYADSVKGRFTISRDNSKNTLYLQMNSLRAEDTAVYYCARHFFESSGGYFDYWGQGTLVTVSSGGGGSGGGGSGGGGSQSVLTQPPSASGTPGQRVTISCSGSSSNIGSNTVNWYQQLPGTAPKLLIYRNNQRPSGVPDRFSGSKSGTSASLAISGLRSEDXADYYCAAWDDSLNGWVFGGXXKLTVLGEQKLISXXXLSGXAA

SEQ ID NO: 88 - protein, 257 aa, Artificial Sequence
  IL-3 (2) scFv antibody
  VARIANT (242)..(242): Any amino acid
  VARIANT (250)..(250): Any amino acid
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCARGGARYDYWGQGTLVTVSSGGGGSGGGGSGGGGSQSVLTQPPSASGTPGQRVTISCSGSSSNIGSNTVNWYQQLPGTAPKLLIYDNNKRPSGVPDRFSGSKSGTSASLAISGLRSEDEADYYCQSYDNILRGVVFGGGTKLTVLXEQKLISEXDLSGSAA

SEQ ID NO: 89 - protein, 258 aa, Artificial Sequence
  IL-3 (3) scFv antibody
  VARIANT (3)..(5): Any amino acids
  VARIANT (204)..(204): Any amino acid
  VARIANT (236)..(236): Any amino acid
  VARIANT (250)..(251): Any amino acids
EVXXXESGGGLVQPGGSLRLSCAASGFTFSSYAMSWVRQAPGKGLEWVSAISGRGEYTYYAGSVKGRFTISRDNSKNTLYLQMNSLRAEDTAVYYCATGATRFGYWGQGTLVTVSSGGGGSGGGGSGGGGSQSVLTQPPSASGTPGQRVTISCTGSSSNIGAGYGVQWYQQLPGTAPKLLIYRNNQRPSGVPDRFSGSKSGTSXSLAISGLRSEDEADYYCQSYDSSLSYSVFGGXTKLTVLGEQKLISXXDLSGSAA

SEQ ID NO: 90 - protein, 264 aa, Artificial Sequence
  IL-4 (1) scFv antibody
  VARIANT (242)..(242): Any amino acid
  VARIANT (256)..(256): Any amino acid
  VARIANT (258)..(258): Any amino acid
EVQLLESGGGLVQPGGSLRLSCAASGFTFSNAWMSWVRQAPGKGLEWVSSLHGGGDTFYTDSVKGRFTISRDNSKNTLYLQMNSLRAEDTAVYYCASLYGSGSYYYYYYGMDVWGQGTLVTVSSGGGGSGGGGSGGGGSQSVLTQPPSASGTPGQRVTISCSGNNSNTGNNAVNWYQQLPGTAPKLLIYDNNKRPSGVPDRFSGSKSGTSASLAISGLRSEDEADYYCCSYAGSYIWVFGGXTKLTVLGEQKLISXEXLSGSAA

SEQ ID NO: 91 - protein, 266 aa, Artificial Sequence
  IL-4 (2) scFv antibody
  VARIANT (244)..(244): Any amino acid
  VARIANT (248)..(248): Any amino acid
  VARIANT (251)..(251): Any amino acid
  VARIANT (258)..(259): Any amino acids
EVQLLESGGGLVQPGGSLRLSCAASGFTFSDYGMHWVRQAPGKGLEWVSGISWNGGKTHYVDSVKGQFTISRDNSKNTLYLQMNSLRAEDTAVYYCARDRGYCSNGVCYTILDYWGQGTLVTVSSGGGGSGGGGSGGGGSQSVLTQPPSASGTPGQRVTISCSGSSSNIGSNTINWYQQLPGTAPKLLIYGNSNRPSGVPDRFSGSKSGTSASLAISGLRSEDEADYYCQSYDSSLSGWVFGGXTKLXVLXEQKLISXXDLSGSAA

SEQ ID NO: 92 - protein, 187 aa, Artificial Sequence
  IL-5 (1) scFv antibody
  VARIANT (159)..(159): Any amino acid
EVQLLESGGGLVQPGGSLRLSCAASGFTFSSYAMSWVRQAPGKGLEWVSSISSRSNYIYYSDSVKGRFTISRDNSKNTLYLQMNSLRAEDTAVYYCARNFRFFDKWGQGTLVTVSSGGGGSGGGGSGGGGSQSVLTQPPSASGTPGQRVTISCSGGSSXIGANPVSWYQQLPGTAPKLLIYGNSNRP

SEQ ID NO: 93 - protein, 257 aa, Artificial Sequence
  IL-5 (2) scFv antibody
  VARIANT (235)..(235): Any amino acid
  VARIANT (242)..(242): Any amino acid
  VARIANT (250)..(251): Any amino acids
EVQLLESGGGLVQPGGSLRLSCAASGFTFSSYAMSWVRQAPGKGLEWVSSISSRSNYIYYSDSVKGRFTISRDNSKNTLYLQMNSLRAEDTAVYYCARNFRFFDKWGQGTLVTVSSGGGGSGGGGSGGGGSQSVLTQPPSASGTPGQRVTISCSGGSSNIGANPVSWYQQLPGTAPKLLIYGNSNRPSGVPDRFSGSKSGTSASLAISGLRSEDEADYYCQSYDSSLSGSVFGGXTKLTVLXEQKLISEXXLSGSAA

SEQ ID NO: 94 - protein, 257 aa, Artificial Sequence
  IL-5 (3) scFv antibody
  VARIANT (235)..(235): Any amino acid
  VARIANT (249)..(249): Any amino acid
EVQLLESGGGLVQPGGSLRLSCAASGFTFSSYAMSWVRQAPGKGLEWVSSISSRSNYIYYSDSVKGRFTISRDNSKNTLYLQMNSLRAEDTAVYYCARNFRFFDKWGQGTLVTVSSGGGGSGGGGSGGGGSQSVLTQPPSASGTPGQRVTISCSGGSSNIGANPVSWYQQLPGTAPKLLIYGNSNRPSGVPDRFSGSKSGTSASLAISGLRSEDEADYYCQSYDSSLSGSVFGGXTKLTVLGEQKLISXEDLSGSAA

SEQ ID NO: 95 - protein, 297 aa, Artificial Sequence
  IL-6 (1) scFv antibody
  VARIANT (203)..(203): Any amino acid
  VARIANT (208)..(208): Any amino acid
  VARIANT (213)..(213): Any amino acid
  VARIANT (218)..(218): Any amino acid
  VARIANT (220)..(220): Any amino acid
  VARIANT (226)..(227): Any amino acids
  VARIANT (234)..(236): Any amino acids
  VARIANT (240)..(240): Any amino acid
  VARIANT (242)..(242): Any amino acid
  VARIANT (247)..(248): Any amino acids
  VARIANT (254)..(256): Any amino acids
  VARIANT (261)..(263): Any amino acids
  VARIANT (268)..(269): Any amino acids
  VARIANT (272)..(273): Any amino acids
  VARIANT (275)..(278): Any amino acids
  VARIANT (280)..(282): Any amino acids
  VARIANT (284)..(284): Any amino acid
  VARIANT (289)..(289): Any amino acid
  VARIANT (291)..(291): Any amino acid
  VARIANT (293)..(293): Any amino acid
  VARIANT (295)..(297): Any amino acids
EVQLLESGGGLVQPGGSLRLSCAASGFTFSSYGMHWVRQAPGKGLEWVSGINWNGGSTGYADSVKGRFTISRDNSKNTLYLQMNSLRAEDTAVYYCARNRGSSLYYGMDVWGQGTLVTVSSGGGGSGGGGSGGGGSQSVLTQPPSASGTPGQRVTISCAGSSSNIGSKSVHWYQQLPGTAPKLLIYRNNRRPSGVPDRFSGSXSGTSXSLAIXGLRSXDXADYYCXXWDDRVNXXXFGGXTXLTVLXXQKLISXXXLSGSXXXPSSSXXLIXXGXXXXLXXXLXFTGRXFXTXLXXX

SEQ ID NO: 96 - protein, 260 aa, Artificial Sequence
  IL-6 (2) scFv antibody
  VARIANT (238)..(238): Any amino acid
  VARIANT (248)..(248): Any amino acid
  VARIANT (254)..(254): Any amino acid
EVQLLESGGGLVQPGGSLRLSCAASGFTFSNYGMHWVRQAPGKGLEWVSSITSSGDGTYFADSVKGRFTISRDNSKNTLYLQMNSLRAEDTAVYYCARAGGIAAAYAFDIWGQGTLVTVSSGGGGSGGGGSGGGGSQSVLTQPPSASGTPGQRVTISCSGSSSNVGSNYVYWYQQLPGTAPKLLIYDNNKRPSGVPDRFSGSKSGTSASLAISGLRSEDEADYYCQSYDSSRWVFGGXTKLTVLGEQXLISEEXLSGSAA

SEQ ID NO: 97 - protein, 269 aa, Artificial Sequence
  IL-7 (1) scFv antibody
  VARIANT (226)..(227): Any amino acids
  VARIANT (247)..(248): Any amino acids
  VARIANT (251)..(251): Any amino acid
  VARIANT (262)..(263): Any amino acids
EVQLLESGGGLVQPGGSLRLSCAASGFTFSSYGMHWVRQAPGKGLEWVSGITWNSGSIGYVDSVKGRFTISRDNSKNTLYLQMNSLRAEDTAVYYCARGPSVAARRIGRHWYNWFDPWGQGTLVTVSSGGGGSGGGGSGGGGSQSVLTQPPSASGTPGQRVTISCSGSSSNIGSNSVYWYQQLPGTAPKLLIYDNNKRPSGVPDRFSGSKSGTSASLAISGLRSEXXADYYCQSYDSSLSGSVFGGXXKLXVLGEQKLISEXXLSGSAA

SEQ ID NO: 98 - protein, 256 aa, Artificial Sequence
  IL-7 (2) scFv antibody
  VARIANT (226)..(227): Any amino acid
  VARIANT (247)..(247): Any amino acid
EVQLLESGGGLVQPGGSLRLSCAASGFTFSDYNIHWVRQPPGKGLEWVSGVSWNGSRTHYADSVKGQFTISRDNSKNTLYLQMNSLRAEDTAVYYCARDPAMVRGVVLPNYYGLDVWGQGTLVTVSSGGGGSGGGGSGGGGSQSVLTQPPSASGTPGQRVTISCTGSSSNIGAGYDVHWYQQLPGTAPKLLIYGHSNRPSGVPDRFSGSKSGTSASLAISGLRSEXXADYYCQSYDSSLSYPVFGGXTKLTVLGEQ

SEQ ID NO: 99 - protein, 259 aa, Artificial Sequence
  IL-8 (1) scFv antibody
  VARIANT (191)..(191): Any amino acid
  VARIANT (237)..(237): Any amino acid
  VARIANT (244)..(244): Any amino acid
  VARIANT (252)..(253): Any amino acids
EVQLLESGGGLVQPGGSLRLSCAASGFTFDDYGMSWVRQAPGKGLEWVSLISWDGGSTYYADSVKGRFTISRDNSKNTLYLQMNSLRAEDTAVYYCARDDLYGMDVWGQGTLVTVSSGGGGSGGGGSGGGGSQSVLTQPPSASGTPGQRVTISCTGSSSNIGAGYDVHWYQQLPGTAPKLLIYDNNKRPSXVPDRFSGSKSGTSASLAISGLRSEDEADYYCAAWDDSLSGWVFGGXTKLTVLXEQKLISEXXLSGSAA

SEQ ID NO: 100 - protein, 265 aa, Artificial Sequence
  IL-8 (2) scFv antibody
  VARIANT (243)..(243): Any amino acid
  VARIANT (250)..(250): Any amino acid
  VARIANT (257)..(259): Any amino acids
EVQLLESGGGLVQPGGSLRLSCAASGFTFSSYEMNWVRQAPGKGLEWVSSISSSSSYIFYADSMKGRFTISRDNSKNTLYLQMNSLRAEDTAVYYCARNESVDPLGGQYFQHWGQGTLVTVSSGGGGSGGGGSGGGGSQSVLTQPPSASGTPGQRVTISCTGSSSNIGAGYDVHWYQQLPGTAPKLLIYDNNKRPSGVPDRFSGSKSGTSASLAISGLRSEDEADYYCSAWDDNLDGPVFGGXTKLTVLXEQKLISXXXLSGSAA

SEQ ID NO: 101 - protein, 251 aa, Artificial Sequence
  IL-9 (1) scFv antibody
  VARIANT (229)..(229): Any amino acid
  VARIANT (236)..(236): Any amino acid
  VARIANT (244)..(244): Any amino acid
EVQLLESGGGLVQPGGSLRLSCAASGFTFSSYAMSWVRQAPGKGLEWVSSISSSSSYIYYADSVKGRFTISRDNSKNTLYLQMNSLRAEDTAVYYCTTFGHWGQGTLVTVSSGGGGSGGGGSGGGGSQSVLTQPPSASGTPGQRVTISCSGSGSNIGDNSVNWYQQLPGTAPKLLIYGNNNRPSGVPDRFSGSKSGTSASLAISGLRSEDEADYYCSSYTSSSVVFGGXTKLTVLXEQKLISEXDLSGSAA

SEQ ID NO: 102 - protein, 192 aa, Artificial Sequence
  IL-9 (2) scFv antibody
EVQLLESGGGLVQPGGSLRLSCAASGFTFSSYGMHWVRQAPGKGLEWVAVISYDGSNKYYADSVKGRFTISRDNSKNTLYLQMNSLRAEDTAVYYCAKSPGGSPYYFDYWGQGTLVTVSSGGGGSGGGGSGGGGSQSVLTQPPSASGTPGQRVTISCSGSVSNIGSNVVSWYQQLPGTAPKLLIYDNNKRPS

SEQ ID NO: 103 - protein, 261 aa, Artificial Sequence
  IL-9 (3) scFv antibody
  VARIANT (239)..(239): Any amino acid
  VARIANT (254)..(254): Any amino acid
EVQLLESGGGLVQPGGSLRLSCAASGFTFSSYGMHWVRQAPGKGLEWVAVISYDGSNKYYADSVKGRFTISRDNSKNTLYLQMNSLRAEDTAVYYCAKSPGGSPYYFDYWGQGTLVTVSSGGGGSGGGGSGGGGSQSVLTQPPSASGTPGQRVTISCSGSVSNIGSNVVSWYQQLPGTAPKLLIYDNNKRPSGVPDRFSGSKSGTSASLAISGLRSEDEADYYCQSYDSSLGGWVFGGXTKLTVLGEQKLISEXDLSGSAA

SEQ ID NO: 104 - protein, 261 aa, Artificial Sequence
  IL-10 (1) scFv antibody
  VARIANT (216)..(216): Any amino acid
  VARIANT (218)..(218): Any amino acid
  VARIANT (239)..(239): Any amino acid
  VARIANT (253)..(254): Any amino acids
EVQLLESGGGLVQPGGSLRLSCAASGFTFRSYVMSWVRQAPGKGLEWVSAISGSGGSTYYADSVKGRFTISRDNSKNTLYLQMNSLRAEDTAVYYCARGKGRWAFDIWGQGTLVTVSSGGGGSGGGGSGGGGSQSVLTQPPSASGTPGQRVTISCTGSSSNVGAGYDVHWYQQLPGTAPKLLIYRNNQRPSGVPDRFSGSKSGTSASLAISGLRSXDXADYYCAAWDDSLSAHVVFGGXTKLTVLGEQKLISXXDLSGSAA

SEQ ID NO: 105 - protein, 261 aa, Artificial Sequence
  IL-10 (2) scFv antibody
  VARIANT (239)..(239): Any amino acid
  VARIANT (254)..(254): Any amino acid
EVQLLESGGGLVQPGGSLRLSCAASGFTFRSYVMSWVRQAPGKGLEWVSAISGSGGSTYYADSVKGRFTISRDNSKNTLYLQMNSLRAEDTAVYYCARGKGRWAFDIWGQGTLVTVSSGGGGSGGGGSGGGGSQSVLTQPPSASGTPGQRVTISCTGSSSNVGAGYDVHWYQQLPGTAPKLLIYRNNQRPSGVPDRFSGSKSGTSASLAISGLRSEDEADYYCAAWDDSLSAHVVFGGXTKLTVLGEQKLISEXDLSGSAA

SEQ ID NO: 106 - protein, 260 aa, Artificial Sequence
  IL-10 (3) scFv antibody
  VARIANT (218)..(218): Any amino acid
  VARIANT (238)..(238): Any amino acid
  VARIANT (245)..(245): Any amino acid
  VARIANT (253)..(254): Any amino acids
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCARGKGRWAFDIWGQGTLVTVSSGGGGSGGGGSGGGGSQSVLTQPPSASGTPGQRVTISCTGSSSNIGAGYGVHWYQQLPGTAPKLLIYGNSNRPSGVPDRFSGSKSGTSASLAISGLRSEDXADYYCAAWDDSLSGLVFGGXTKLTVLXEQKLISEXXLSGSAA

SEQ ID NO: 107 - protein, 262 aa, Artificial Sequence
  IL-11 (1) scFv antibody
  VARIANT (231)..(231): Any amino acid
  VARIANT (240)..(240): Any amino acid
  VARIANT (254)..(254): Any amino acid
  VARIANT (256)..(256): Any amino acid
EVQLLESGGGLVQPGGSLRLSCAASGFTFSNFGMHWVRQAPGKGLEWVAFIRYDGSNKYYADSVKGRFTISRDNSKNTLYLQMNSLRAEDTAVYYCARHYYYSETSGHPGGFDPWGQGTLVTVSSGGGGSGGGGSGGGGSQSVLTQPPSASGTPGQRVTISCSGSSSNIGSYPVNWYQQLPGTAPKLLIYGNSNRPSGVPDRFSGSKSGTSASLAISGLRSEDEADYYCQXWGTGVFGGXTKLTVLGEQKLISXEXLSGSAA

SEQ ID NO: 108 - protein, 267 aa, Artificial Sequence
  IL-11 (2) scFv antibody
  VARIANT (208)..(208): Any amino acid
  VARIANT (245)..(245): Any amino acid
  VARIANT (260)..(261): Any amino acids
EVQLLESGGGLVQPGGSLRLSCAASGFTFSSYGMHWVRQAPGKGLEWVAVISYDGSNKYYADSVKGRFTISRDNSKNTLYLQMNSLRAEDTAVYYCARHYYDVSYRGQQDAFDIWGQGTLVTVSSGGGGSGGGGSGGGGSQSVLTQPPSASGTPGQRVTISCTGSSSNLGSPYDVHWYQQLPGTAPKLLIYRNDQRASGVPDRFSGSXSGTSASLAISGLRSEDEADYYCAAWDDSLNAWVFGGXTKLTVLGEQKLISEXXLSGSAA

SEQ ID NO: 109 - protein, 263 aa, Artificial Sequence
  IL-11 (3) scFv antibody
  VARIANT (241)..(241): Any amino acid
EVQLLESGGGLVQPGGSLRLSCAASGFTFSDYYMSWIRQAPGKGLEWVAYISGISGYTNYADSVRGRFTISRDNSKNTLYLQMNSLRAEDTAVYYCAKSKDWVNGGEMDVWGQGTLVTVSSGGGGSGGGGSGGGGSQSVLTQPPSASGTPGQRVTISCTGSSSNIGAGYVVHWYQQLPGTAPKLLIYSNNQRPSGVPDRFSGSKSGTSASLAISGLRSEDEADYYCAAWDDSLRGWVFGGXTKLTVLGEQKLISEEDLSGSAA

SEQ ID NO: 110 - protein, 256 aa, Artificial Sequence
  IL-12 (1) scFv antibody
  VARIANT (234)..(234): Any amino acid
  VARIANT (249)..(249): Any amino acid
EVQLLESGGGLVQPGGSLRLSCAASGFTFSSYSMNWVRQAPGKGLEWVSAIGTGGGTYYADSVKGRFTISRDNSKNTLYLQMNSLRAEDTAVYYCARAFRAFDIWGQGTLVTVSSGGGGSGGGGSGGGGSQSVLTQPPSASGTPGQRVTISCSGSRSNIGNNFVSWYQQLPGTAPKLLIYGNSNRPSGVPDRFSGSKSGTSASLAISGLRSEDEADYYCAAWDDSLSGPVFGGXTKLTVLGEQKLISEXDLSGSAA

SEQ ID NO: 111 - protein, 262 aa, Artificial Sequence
  IL-12 (2) scFv antibody
  VARIANT (255)..(256): Any amino acids
EVQLLESGGGLVQPGGSLRLSCAASGFTFSDYYMSWVRQAPGKGLEWVSGVSWNGSRTHYADSVKGQFTISRDNSKNTLYLQMNSLRAEDTAVYYCARGSRSSPDAFDIWGQGTLVTVSSGGGGSGGGGSGGGGSQSVLTQPPSASGTPGQRVTISCTGSSSNIGAGYDVHWYQQLPGTAPKLLIYGNSNRPSGVPDRFSGSKSGTSASLAISGLRSEDEADYYCAAWDDRVNGRVFGGGTKLTVLGEQKLISEXXLSGSAA

SEQ ID NO: 112 - protein, 231 aa, Artificial Sequence
  IL-13 (1) scFv antibody
EVQLLESGGGLVQPGGSLRLSCAASGFTFSSYSMNWVRQAPGKGLEWVSSISSGSSYIYYADSVKGRFTISRDNSKNTLYLQMNSLRAEDTAVYYCARSQGWWTYYYGMDVWGQGTLVTVSSGGGGSGGGGSGGGGSQSVLTQPPSASGTPGQRVTISCSGSSSNIGSNTVNWYQQLPGTAPKLLIYSNNQRPSGVPDRFSGSKSGTSASLAISGLRSEDEADYYCETWGQ

SEQ ID NO: 113 - protein, 317 aa, Artificial Sequence
  IL-13 (2) scFv antibody
  VARIANT (221)..(221): Any amino acid
  VARIANT (229)..(229): Any amino acid
  VARIANT (239)..(239): Any amino acid
  VARIANT (255)..(255): Any amino acid
  VARIANT (261)..(261): Any amino acid
  VARIANT (270)..(270): Any amino acid
  VARIANT (272)..(272): Any amino acid
  VARIANT (275)..(276): Any amino acids
  VARIANT (280)..(280): Any amino acid
  VARIANT (285)..(286): Any amino acids
  VARIANT (289)..(289): Any amino acid
  VARIANT (296)..(297): Any amino acids
  VARIANT (301)..(303): Any amino acids
  VARIANT (310)..(312): Any amino acids
  VARIANT (314)..(317): Any amino acids
EVQLLESGGGLVQPGGSLRLSCAASGFTFSSYSMNWVRQAPGKGLEWVSSISSGSSYIYYADSVKGRFTISRDNSKNTLYLQMNSLRAEDTAVYYCARSQGWWTYYYGMDVWGQGTLVTVSSGGGGSGGGGSGGGGSQSVLTQPPSASGTPGQRVTISCSGSSSNIGSNTVNWYQQLPGTAPKLLIYSNNQRPSGVPDRFSGSKSGTSASLAISGLRSEDXADYYCETXDSNTQIFGGXTKLTVLGEQKLISEEXLSGSAXAHHHHHHSXRXPIXXIVSXITIHXXSFXNVVTGKXXALPXXXALQHIPXXXAXXXX

SEQ ID NO: 114 - protein, 261 aa, Artificial Sequence
  IL-13 (3) scFv antibody
  VARIANT (239)..(239): Any amino acid
  VARIANT (254)..(254): Any amino acid
EVQLLESGGGLVQPGGSLRLSCAASGFTFSSYSMNWVRQAPGKGLEWVSSISSGSSYIYYADSVKGRFTISRDNSKNTLYLQMNSLRAEDTAVYYCARSQGWWTYYYGMDVWGQGTLVTVSSGGGGSGGGGSGGGGSQSVLTQPPSASGTPGQRVTISCSGSSSNIGSNTVNWYQQLPGTAPKLLIYSNNQRPSGVPDRFSGSKSGTSASLAISGLRSEDEADYYCETWDSNTQIFGGXTKLTVLGEQKLISEXDLSGSAA

SEQ ID NO: 115 - protein, 262 aa, Artificial Sequence
  VEGF (1) scFv antibody
  VARIANT (203)..(203): Any amino acid
  VARIANT (240)..(240): Any amino acid
  VARIANT (255)..(255): Any amino acid
EVQLLESGGGLVQPGGSLRLSCAASGFTFSSNEMSWIRQAPGKGLEWVSSISGSGGFTYYADSVKGRYTISRDNSKNTLYLQMNSLRAEDTAVYYCARETTVRGNAFDIWGQGTLVTVSSGGGGSGGGGSGGGGSQSVLTQPPSASGTPGQRVTISCTGGSSNIGAGYDVHWYQQLPGTAPKLLIYRNNQRPSGVPDRFSGSXSGTSASLAISGLRSEDEADYYCAAWDDSLSVPMFGGXTKLTVLGEQKLISEXDLSGSAA

SEQ ID NO: 116 - protein, 265 aa, Artificial Sequence
  VEGF (2) scFv antibody
  VARIANT (197)..(197): Any amino acid
  VARIANT (206)..(206): Any amino acid
  VARIANT (222)..(222): Any amino acid
  VARIANT (243)..(243): Any amino acid
  VARIANT (252)..(252): Any amino acid
  VARIANT (258)..(259): Any amino acids
EVQLLESGGGLVQPGGSLRLSCAASGFTFSSYSMNWVRQAPGKGLEWVSGINWNGGSTGYADSVKGRFTISRDNSKNTLYLQMNSLRAEDTAVYYCASSVGGWYEGDNWFDPWGQGTLVTVSSGGGGSGGGGSGGGGSQSVLTQPPSASGTPGQRVTISCTGSSSNIGAGYDVHWYQQLPGTAPKLLIYGNSNRPSXVPDRFSGSXSGTSASLAISGLRSEXEADYYCQSYDGSLSGSVFGGXTKLTVLGEXKLISEXXLSGSAA

SEQ ID NO: 117 - protein, 260 aa, Artificial Sequence
  TGF-beta1 (1) scFv antibody
  VARIANT (238)..(238): Any amino acid
  VARIANT (242)..(242): Any amino acid
EVQLLESGGGLVQPGGSLRLSCAASGFTFSTYAMSWVRQAPGKGLEWVAVVSIDGGTTYYGDPVKGRFTISRDNSKNTLYLQMNSLRAEDTAVYYCTRGPTLTYYFDYWGQGTLVTVSSGGGGSGGGGSGGGGSQSVLTQPPSASGTPGQRVTISCSGSSSNIGSNTVNWYQQLPGTAPKLLIYSNNQRPSGVPDRFSGSKSGTSASLAISGLRSEDEADYYCQSYDSSLSGWVFGGXTKLXVLGEQKLISEEDLSGSAA

SEQ ID NO: 118 - protein, 259 aa, Artificial Sequence
  TGF-beta1 (2) scFv antibody
  VARIANT (241)..(241): Any amino acid
  VARIANT (252)..(252): Any amino acid
EVQLLESGGGLVQPGGSLRLSCAASGFTFGDYAMSWFRQAPGKGLEWVSGVSWNGSRTHYADSVKGRFTISRDNSKNTLYLQMNSLRAEDTAVYYCARDGNRPLDYWGQGTLVTVSSGGGGSGGGGSGGGGSQSVLTQPPSASGTPGQRVTISCTGSSSNIGAGYDVHWYQQLPGTAPKLLIYGNSNRPSGVPDRFSGSKSGTSASLAISGLRSEDEADYYCAAWDDRLNGWVFGGGTKLXVLGEQKLISEXDLSGSAA

SEQ ID NO: 119 - protein, 267 aa, Artificial Sequence
  TGF-beta1 (3) scFv antibody
  VARIANT (225)..(225): Any amino acid
  VARIANT (245)..(245): Any amino acid
  VARIANT (259)..(261): Any amino acids
EVQLLESGGGLVQPGGSLRLSCAASGFTFSDYYIGWIRQAPGKGLEWVSGINWNGGSTGYADSVKGRFTISRDNSKNTLYLQMNSLRAEDTAVYYCARRSTPSSSWALPDFFDYWGQGTLVTVSSGGGGSGGGGSGGGGSQSVLTQPPSASGTPGQRVTISCTGSSSNIGANYDVHWYQQLPGTAPKLLIYSNNQRPSGVPDRFSGSKSGTSASLAISGLRSEDXADYYCQSYDSSLSGWVFGGXTKLTVLGEQKLISXXXLSGSAA

SEQ ID NO: 120 - protein, 259 aa, Artificial Sequence
  TNF-alpha (1) scFv antibody
  VARIANT (191)..(191): Any amino acid
  VARIANT (200)..(200): Any amino acid
  VARIANT (237)..(237): Any amino acid
  VARIANT (244)..(244): Any amino acid
  VARIANT (251)..(252): Any amino acids
EVQLLESGGGLVQPGGSLRLSCAASGFTFDDYGMSWVRQAPGKGLEWVAVISYDGSNKYYADSVKGRFTISRDNSKNTLYLQMNSLRAEDTAVYYCTRHLGSAMGYWGQGTLVTVSSGGGGSGGGGSGGGGSQSVLTQPPSASGTPGQRVTISCTGSSSNIGAGYDVHWYQQLPGTAPKLLIYGNSNRPSXVPDRFSGSXSGTSASLAISGLRSEDEADYYCQSYDSSLSGWVFGGXTKLTVLXEQKLISXXDLSGSAA

SEQ ID NO: 121 - protein, 261 aa, Artificial Sequence
  TNF-alpha (2) scFv antibody
  VARIANT (239)..(239): Any amino acid
  VARIANT (241)..(241): Any amino acid
  VARIANT (246)..(246): Any amino acid
  VARIANT (254)..(255): Any amino acids
EVQLLESGGGLVQPGGSLRLSCAASGFTFSSYGMHWVRQAPGKGLEWVSAISGSGGSTYYADSVKGRFTISRDNSKNTLYLQMNSLRAEDTAVYYCARGGWGPRSAFDIWGQGTLVTVSSGGGGSGGGGSGGGGSQSVLTQPPSASGTPGQRVTISCSGSSSNIGSNTVTWYQQLPGTAPKLLIYGNTNRLSGVPDRFSGSKSGTSASLAISGLRSEDEADYYCEAWDDKLFGPVFGGXTXLTVLXEQKLISEXXLSGSAA

SEQ ID NO: 122 - protein, 261 aa, Artificial Sequence
  TNF-alpha (3) scFv antibody
  VARIANT (240)..(240): Any amino acid
  VARIANT (246)..(246): Any amino acid
  VARIANT (248)..(248): Any amino acid
  VARIANT (253)..(255): Any amino acids
EVQLLESGGGLVQPGGSLRLSCAASGFTFSSYWMSWVRQAPGKGLEWVSGVNWNGSRTHYADSVKGRFTISRDNSKNTLYLQMNSLRAEDTAVYYCASIRANYYYGMDVWGQGTLVTVSSGGGGSGGGGSGGGGSQSVLTQPPSASGTPGQRVTISCSGGSSNIGSHPVNWYQQLPGTAPKLLIYGNSNRPSGVPDRFSGSKSGTSASLAISGLRSEDEADYYCAAWDASLSGWVFGGGXKLTVLXEXKLISXXXLSGSAA

SEQ ID NO: 123 - protein, 261 aa, Artificial Sequence
  GM-CSF (1) scFv antibody
  VARIANT (197)..(197): Any amino acid
  VARIANT (239)..(239): Any amino acid
  VARIANT (254)..(255): Any amino acids
EVQLLESGGGLVQPGGSLRLSCAASGFTFSSYGMHWVRQAPGKGLEWVAVISYDGSNKYYADSVKGRFTISRDNSKNTLYLQMNSLRAEDTAVYYCARVGGMSAPVDYWGQGTLVTVSSGGGGSGGGGSGGGGSQSVLTQPPSASGTPGQRVTISCSGSSSNIGSNTVNWYQQLPGTAPKLLIYDNNKRPSGVPDRXSGSKSGTSASLAISGLRSEDEADYYCAAWDDSLIGLVVFGGXTKLTVLGEQKLISEXXLSGSAA

SEQ ID NO: 124 - protein, 260 aa, Artificial Sequence
  GM-CSF (2) scFv antibody
  VARIANT (193)..(193): Any amino acid
  VARIANT (238)..(238): Any amino acid
  VARIANT (242)..(242): Any amino acid
  VARIANT (252)..(252): Any amino acid
EVQLLESGGGLVQPGGSLRLSCAASGFTFSSYAMSWVRQAPGKGLEWVAVISYDGSNEDSADSVKGRFTISRDNSKNTLYLQMNSLRAEDTAVYYCARGPSLRGVSDYWGQGTLVTVSSGGGGSGGGGSGGGGSQSVLTQPPSASGTPGQRVTISCTGSSSNIGAGYDVHWYQQLPGTAPKLLIYNDNQRPSXVPDRFSGSKSGTSASLAISGLRSEDEADYYCQTWGTGINVIFGGXTKLXVLGEQKLISXEDLSGSAA

SEQ ID NO: 125 - protein, 260 aa, Artificial Sequence
  GM-CSF (3) scFv antibody
  VARIANT (219)..(219): Any amino acid
  VARIANT (238)..(238): Any amino acid
  VARIANT (247)..(247): Any amino acid
  VARIANT (253)..(254): Any amino acids
EVQLLESGGGLVQPGGSLRLSCAASGFTFSSYAMSWVRQAPGKGLEWVAVISYDGSNEDSADSVKGRFTISRDNSKNTLYLQMNSLRAEDTAVYYCARGPSLRGVSDYWGQGTLVTVSSGGGGSGGGGSGGGGSQSVLTQPPSASGTPGQRVTISCTGSSSNIGAGYDVHWYQQLPGTAPKLLIYNDNQRPSGVPDRFSGSKSGTSASLAISGLRSEDXADYYCQTWGTGINVIFGGXTKLTVLGEXKLISEXXLSGSAA

SEQ ID NO: 126 - protein, 261 aa, Artificial Sequence
  TNF-beta (1) scFv antibody
  VARIANT (194)..(194): Any amino acid
  VARIANT (203)..(203): Any amino acid
  VARIANT (228)..(228): Any amino acid
  VARIANT (239)..(239): Any amino acid
  VARIANT (253)..(254): Any amino acids
EVQLLESGGGLVQPGGSLRLSCAASGFTFSSFAMHWVRQAPGKGLEWVSAISGSGGSTYYADSVKGRFTISRDNSKNTLYLQMNSLRAEDTAVYYCASRSTLYYYYGMDVWGQGTLVTVSSGGGGSGGGGSGGGGSQSVLTQPPSASGTPGQRVTISCSGSTSNIGNSHVYWYQQLPGTAPKLLIYGNSNRPSXVPDRFSGSXSGTSASLAISGLRSEDEADYYCSSXAGSNNLVFGGXTKLTVLGEQKLISXXDLSGSAA

SEQ ID NO: 127 - protein, 258 aa, Artificial Sequence
  TNF-beta (2) scFv antibody
  VARIANT (236)..(236): Any amino acid
  VARIANT (243)..(243): Any amino acid
  VARIANT (250)..(252): Any amino acids
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCARSPYYGMDVWGQGTLVTVSSGGGGSGGGGSGGGGSQSVLTQPPSASGTPGQRVTISCTGSSSNIGAGYDVHWYQQLPGTAPKLLIYRNDQRPSGVPDRFSGSKSGTSASLAISGLRSEDEADYYCSSYGGRDNVVFGGXTKLTVLXEQKLISXXXLSGSAA

SEQ ID NO: 128 - protein, 262 aa, Artificial Sequence
  IL-1ra (1) scFv antibody
  VARIANT (240)..(240): Any amino acid
  VARIANT (244)..(244): Any amino acid
  VARIANT (247)..(247): Any amino acid
  VARIANT (254)..(254): Any amino acid
EVQLLESGGGLVQPGGSLRLSCAASGFTFDTHWMSWVRQAPGKGLEWVSAISGSGGSTYYADSVKGRFTISRDNSKNTLYLQMNSLRAEDTAVYYCARHDYGDYRAFDIWGQGTLVTVSSGGGGSGGGGSGGGGSQSVLTQPPSASGTPGQRVTISCTGSSSNIGAGYVVHWYQQLPGTAPKLLIYGNNNRPSGVPDRFSGSKSGTSASLAISGLRSEDEADYYCQSYDSSLSGVVFGGXTKLXVLXEQKLISXEDLSGSAA

SEQ ID NO: 129 - protein, 259 aa, Artificial Sequence
  IL-1ra (2) scFv antibody
  VARIANT (217)..(217): Any amino acid
  VARIANT (227)..(227): Any amino acid
  VARIANT (237)..(237): Any amino acid
  VARIANT (251)..(254): Any amino acids
EVQLLESGGGLVQPGGSLRLSCAASGFTFSKYAMTWVRQAPGKGLEWVSAISGSGGNTYYADSVKGRFTISRDNSKNTLYLQMNSLRAEDTAVYYCARLVRGLYYGMDVWGQGTLVTVSSGGGGSGGGGSGGGGSQSVLTQPPSASGTPGQRVTISCSGSSSNIGNNAVNWYQQLPGTAPKLLIYGNSNRPSGVPDRFSGSKSGTSASLAISGLRSXDEADYYCQTXGTGPVVFGGXTKLTVLGEQKLISXXXXSGSAA

SEQ ID NO: 130 - protein, 263 aa, Artificial Sequence
  IL-1ra (3) scFv antibody
  VARIANT (241)..(242): Any amino acids
  VARIANT (245)..(245): Any amino acid
  VARIANT (248)..(248): Any amino acid
EVQLLESGGGLVQPGGSLRLSCAVSGFTFSSYSMNWVRQAPGKGLEWVAGIGGRGATTYYVDSVKGRFTISRDNSKNTLYLQMNSLRAEDTAVYYCARLRVVPAARFDYWGQGTLVTVSSGGGGSGGGGSGGGGSQSVLTQPPSASGTPGQRVTISCSGSSSNIGSNTVNWYQQLPGTAPKLLIYGNSNRPSGVPDRFSGSKSGTSASLAISGLRSEDEADYYCQSYDSSLSGPPWVFGGXXKLXVLXEQKLISEEDLSGSAA

SEQ ID NO: 131 - protein, 263 aa, Artificial Sequence
  IL-16 (1) scFv antibody
  VARIANT (241)..(241): Any amino acid
  VARIANT (256)..(256): Any amino acid
EVQLLESGGGLVQPGGSLRLSCAASGFTFSNHAMSWVRQAPGKGLEWVSGVSWNGSRTHYADSVKGRFTISRDNSKNTLYLQMNSLRAEDTAVYYCARAALVQGVKHAFEIWGQGTLVTVSSGGGGSGGGGSGGGGSQSVLTQPPSASGTPGQRVTISCSGSSSNIGSNTVNWYQQLPGTALKLLIYRNNQRPSGVPDRFSGSKSGTSASLAISGLRSEDEADYYCASWDDRLSGLVFGGXTKLTVLGEQKLISEXDLSGSAA

SEQ ID NO: 132 - protein, 263 aa, Artificial Sequence
  IL-16 (2) scFv antibody
  VARIANT (204)..(204): Any amino acid
  VARIANT (220)..(220): Any amino acid
  VARIANT (241)..(241): Any amino acid
  VARIANT (248)..(248): Any amino acid
EVQLLESGGGLVQPGGSLRLSCAASGFTFSNHAMSWVRQAPGKGLEWVSGVSWNGSRTHYADSVKGRFTISRDNSKNTLYLQMNSLRAEDTAVYYCARAALVQGVKHAFEIWGQGTLVTVSSGGGGSGGGGSGGGGSQSVLTQPPSASGTPGQRVTISCSGSSSNIGSNTVNWYQQLPGTALKLLIYRNNQRPSGVPDRFSGSXSGTSASLAISGLRSEXEADYYCASWDDRLSGLVFGGXTKLTVLXEQKLISEEDLSGSAA

SEQ ID NO: 133 - protein, 259 aa, Artificial Sequence
  IL-18 (1) scFv antibody
  VARIANT (201)..(201): Any amino acid
  VARIANT (226)..(226): Any amino acid
  VARIANT (237)..(237): Any amino acid
  VARIANT (251)..(253): Any amino acids
EVQLLESGGGLVQPGGSLRLSCAASGFTFSSYGMHWVRQAPGKGLEWVSGINWNGGSTGYADSVKGRFTISRDNSKNTLYLQMNSLRAEDTAVYYCARDLRGGRFDPWGQGTLVTVSSGGGGSGGGGSGGGGSQSVLTQPPSASGTPGQRVTISCTGSSSNIGAGYVVHWYQQLPGTAPKLLIYRNNQRPSGVPDRFSGSXSGTSASLAISGLRSEDEADYYCSSXAGSKNLIFGGXTKLTVLGEQKLISXXXLSGSAA

SEQ ID NO: 134 - protein, 262 aa, Artificial Sequence
  IL-18 (2) scFv antibody
  VARIANT (226)..(226): Any amino acid
  VARIANT (240)..(241): Any amino acids
  VARIANT (244)..(244): Any amino acid
  VARIANT (249)..(249): Any amino acid
  VARIANT (255)..(255): Any amino acid
EVQLLESGRGLVQPGGSLRLSCAASGFTFSSYGMHWVRQAPGKGLEWVSAIGTGGDTYYADSVMGRFTISRDNSKNTLYLQMNSLRAEDTAVYYCARSPRRGATAGTFDYWGQGTLVTVSSGGGGSGGGGSGGGGSQSVLTQPPSASGTPGQRVTISCSGSSSNIGSNIVNWYQQLPGTAPKLLIYRNNQRPSGVPDRFSGSKSGTSASLAISGLRSEDEADYYCXSYDNSLSGWVFGGXXKLXVLGEXKLISEXDLSGSAA

SEQ ID NO: 135 - protein, 261 aa, Artificial Sequence
  MCP-4 (1) scFv antibody
  VARIANT (239)..(239): Any amino acid
  VARIANT (254)..(254): Any amino acid
EVQLLESGGGLVQPGGSLRLSCAASGFTFSSYGMHWVRQAPGKGLEWVSGISWNGGKTHYVDSVKGQFTISRDNSKNTLYLQMNSLRAEDTAVYYCARGGYSSGWAFDYWGQGTLVTVSSGGGGSGGGGSGGGGSQSVLTQPPSASGTPGQRVTISCSGRSSNIESNTVNWYQQLPGTAPKLLIYGNSNRPSGVPDRFSGSKSGTSASLAISGLRSEDEADYYCAAWDDRLNAVVFGGXTKLTVLGEQKLISEXDLSGSAA

SEQ ID NO: 136 - protein, 261 aa, Artificial Sequence
  IFN-gamma (1) scFv antibody
  VARIANT (195)..(195): Any amino acid
  VARIANT (204)..(204): Any amino acid
  VARIANT (228)..(228): Any amino acid
  VARIANT (239)..(239): Any amino acid
  VARIANT (248)..(248): Any amino acid
  VARIANT (255)..(255): Any amino acid
EVQLLESGGGLVQPGGSLRLSCAASGFTFSSYGMHWVRQAPGKGLEWVSGVSWNGSRTHYADSVKGRFTISRDNSKNTLYLQMNSLRAEDTAVYYCARGRTGHGWKYYFDLWGQGTLVTVSSGGGGSGGGGSGGGGSQSVLTQPPSASGTPGQRVTISCSGSSSNIGNNAVNWYQQLPGTAPKLLIYGNSNRPSXVPDRFSGSXSGTSASLAISGLRSEDEADYYCQXWGTGLGVFGGXTKLTVLGEXKLISEEXLSGSAA

SEQ ID NO: 137 - protein, 189 aa, Artificial Sequence
  IFN-gamma (2) scFv antibody
EVQLLESGGGLVQPGGSLRLSCAASGFTFSRHGFHWVRQGPGKGLEWVSGVSWNGSRTHYADSVKGRFTISRDNSKNTLYLQMNSLRAEDTAVYYCARGNWYRAFDIWGQGTLVTVSSGGGGSGGGGSGGGGSQSVLTQPPSASGTPGQRVTISCSGGSSHIGRNFISWYQQLPGTAPKLLIYAGNSRP

SEQ ID NO: 138 - protein, 261 aa, Artificial Sequence
  IL-1beta (1) scFv antibody
  VARIANT (219)..(219): Any amino acid
  VARIANT (239)..(239): Any amino acid
  VARIANT (254)..(255): Any amino acids
EVQLLESGGGLVQPGGSLRLSCAASGFTFSSYAMSWVRQAPGKGLEWVSYISSSGSTIYYADSVKGRSTISRDNSKNTLYLQMNSLRAEDTAVYYCARVRQNSGSYAYWGQGTLVTVSSGGGGSGGGGSGGGGSQSVLTQPPSASGTPGQRVTISCTGTSSNIGAPYDVHWYQQLPGTAPKLLIYGNSNRPSGVPDRFSGSKSGTSASLAISGLRSEDXADYYCQSYDSSLSAVVFGGXTKLTVLGEQKLISEXXLSGSAA

SEQ ID NO: 139 - protein, 267 aa, Artificial Sequence
  IL-1beta (2) scFv antibody
  VARIANT (245)..(245): Any amino acid
  VARIANT (247)..(247): Any amino acid
  VARIANT (250)..(250): Any amino acid
  VARIANT (252)..(252): Any amino acid
  VARIANT (261)..(261): Any amino acid
EVQLLESGGGLVQPGGSLRLSCAASGFTFSRYVMTWVRQAPGKGLEWVSLISGGGSATYYADSMKGRFTISRDNSKNTLYLQMNSLRAEDTAVYYCAKRVPYDSSGYYPDAFDIWGQGTLVTVSSGGGGSGGGGSGGGGSQSVLTQPPSASGTPGQRVTISCTGSSSNIGAGYDVHWYQQLPGTAPKLLIYGNSNRPSGVPDQFSGSKSGTSASLAISGLRSEDEADYYCAAWDDSLNGPVFGGXTXLTXLXEQKLISEEXLSGSAA

SEQ ID NO: 140 - protein, 264 aa, Artificial Sequence
  IL-1beta (3) scFv antibody
  VARIANT (242)..(242): Any amino acid
  VARIANT (249)..(249): Any amino acid
  VARIANT (257)..(258): Any amino acids
EVQLLESGGGLVQPGGSLRLSCAASGFTFSSYWMSWVRQAPGKGLEWVAVVSYDGNNKYYADSRKGRFTISRDNSKNTLYLQMNSLRAEDTAMYYCASYWYTSGWYPYGMDVWGQGTLGTVSSGGGGSGGGGSGGGGSQSVLTQPPSASGTPGQRVTISCTGSSSNIGAGYDLHWYQQLPGTAPKLLIYRNNQRPSGVPDRFSGSKSGTSASLAISGLRSEDEADYYCSSYVDNNNLVFGGXTKLTVLXEQKLISEXXLSGSAA

SEQ ID NO: 141 - protein, 265 aa, Artificial Sequence
  Eotaxin (1) scFv antibody
  VARIANT (206)..(206): Any amino acid
  VARIANT (243)..(243): Any amino acid
  VARIANT (257)..(258): Any amino acids
  VARIANT (262)..(262): Any amino acid
EVQLLESGGGLVQPGGSLRLSCAASGFTFSSYWMSWVRQAPGKGLEWVSGVSWNGSRTHYADSVKGRFTISRDNSKNTLYLQMNSLRAEDTAVYYCVKGKGTIAMPGRARVGWWGQGTLVTVSSGGGGSGGGGSGGGGSQSVLTQPPSASGTPGQRVTISCSGSSSNIGNNAVNWYQQLPGTAPKLLIYANSNRPSGVPDRFSGSXSGTSASLAISGLRSEDEADYYCAAWDDSLSGPVFGGXTKLTVLGEQKLISXXDLSXSAA

SEQ ID NO: 142 - protein, 322 aa, Artificial Sequence
  Eotaxin (2) scFv antibody
  VARIANT (199)..(199): Any amino acid
  VARIANT (236)..(236): Any amino acid
  VARIANT (250)..(252): Any amino acids
  VARIANT (257)..(257): Any amino acid
  VARIANT (269)..(269): Any amino acid
  VARIANT (277)..(278): Any amino acids
  VARIANT (289)..(289): Any amino acid
  VARIANT (293)..(295): Any amino acids
  VARIANT (299)..(299): Any amino acid
  VARIANT (301)..(303): Any amino acids
  VARIANT (311)..(312): Any amino acids
  VARIANT (314)..(316): Any amino acids
  VARIANT (318)..(322): Any amino acids
EVQLLESGGGLVQPGGSLRLSCAASGFTFSAYWMTWVRQAPGKGLEWVSVIYSGGSTYYADSVKGRFTISRDNSKNTLYLQMNSLRAEDTAVYYCARQTQQEYFDYWGQGTLVTVSSGGGGSGGGGSGGGGSQSVLTQPPSASGTPGQRVTISCFGSNSNIGSSTVNWYQQLPGTAPKLLIYDNDKRPSGVPDRFSGSXSGTSASLAISGLRSEDEADYYCAAWDDSLNGPVFGGXTKLTVLGEQKLISXXXLSGSXAAHHHHHHSPRXPIRPIVSXXTIHWPSFYNVXTGKXXXLPNXIXXXHIPLSPAXXIXXXPXXXXX

SEQ ID NO: 143 - protein, 260 aa, Artificial Sequence
  Eotaxin (3) scFv antibody
  VARIANT (218)..(218): Any amino acid
  VARIANT (235)..(235): Any amino acid
  VARIANT (239)..(239): Any amino acid
EVQLLESGGGLVQPGGSLRLSCAASGFTFRGYAMSWVRQAPGKGLEWVSGVSWNGSRTHYADSVKGRFTISRDNSKNTLYLQMNSLRAEDTAVYYCARAPAVAGWFDPWGQGTLVTVSSGGGGSGGGGSGGGGSQSVLTQPPSASGTPGQRVTISCSGSSSNIGSHTVNWYQQLPGTAPKLLIYRNNQRPSGVPDRFSGSKSGTSASLAISGLRSEDXADYYCAAWDDSLSGRVXGGGXKLTVLGEQKLISEEDLSGSAA

SEQ ID NO: 144 - protein, 263 aa, Artificial Sequence
  RANTES (1) scFv antibody
  VARIANT (241)..(241): Any amino acid
  VARIANT (256)..(257): Any amino acids
EVQLLESGGGLVQPGGSLRLSCAASGFTFSSYGMHWVRQAPGKGLEWVAVISNDGTKKDYADSVKGRFTISRDNSKNTLYLQMNSLRAEDTAVYYCARDASGYDDYYFDYWGQGTLVTVSSGGGGSGGGGSGGGGSQSVLTQPPSASGTPGQRVTISCTGSSSNIGAGSDVHWYQQLPGTAPKLLIYRDDQRSSGVPDRFSGSKSGTSAFLAISGLRSEDEADYYCQSYDNSLSGWVFGGXTKLTVLGEQKLISEXXLSGSAA

SEQ ID NO: 145 - protein, 191 aa, Artificial Sequence
  RANTES (2) scFv antibody
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCARDNDYSSDTFDYWGQGTLVTVSSGGGGSGGGGSGGGGSQSVLTQPPSAFGTPGQRVTISCSGSSSNIGSDYVYWYQQLPGTAPKLLIYSDNQRP

SEQ ID NO: 146 - protein, 263 aa, Artificial Sequence
  RANTES (3) scFv antibody
  VARIANT (241)..(241): Any amino acid
  VARIANT (256)..(256): Any amino acid
EVQLLESGGGLVQPGGSLRLSCAASGFTFSNYGMNWVRQAPGKGLEWVSGVSWNGSRTHYVDSVKRRFTISRDNSKNTLYLQMNSLRAEDTAVYYCARPRLRSHNYYGMDVWGQGTLVTVSSGGGGSGGGGSGGGGSQSVLTQPPSASGTPGQRVTISCSGSSFKSGKNYVSWYQQLPGTAPKLLIYRNNQRPSGVPDRFSGSKSGTSASLAISGLRSEDEADYYCAAWDVRVKGVIFGGXTKLTVLGEQKLISEXDLSGSAA

SEQ ID NO: 147 - protein, 258 aa, Artificial Sequence
  MCP-1 (1) scFv antibody
  VARIANT (215)..(215): Any amino acid
  VARIANT (236)..(236): Any amino acid
  VARIANT (243)..(243): Any amino acid
  VARIANT (251)..(252): Any amino acids
EVQLLESGGGLVQPGGSLRLSCAASGFTFSSYAMSWVRQAPGKGLEWVSGVSWNGSRTHYADSVKGRFTISRDNSKNTLYLQMNSLRAEDTAVYYCARGGHQQLGQWGQGTLVTVSSGGGGSGGGGSGGGGSQSVLTQPPSASGTPGQRVTISCSGSSSNIGNNYVSWYQQLPGTAPKLLIYRDSRRPSGVPDRFSGSKSGTSASLAISGLRSEXEADYYCAAWDDSLKGWLFGGXTKLTVLXEQKLISEXXLSGSAA

SEQ ID NO: 148 - protein, 261 aa, Artificial Sequence
  MCP-1 (2) scFv antibody
  VARIANT (239)..(239): Any amino acid
  VARIANT (246)..(246): Any amino acid
  VARIANT (254)..(254): Any amino acid
EVQLLESGGGLVQPGGSLRLSCAASGFTFSSYAMSWVRQAPGKGLEWVSYISSSSSYTNYADSVKGRFTISRDNSKNTLYLQMNSLRAEDTAVYYCARFRYNSGKMFDYWGQGTLVTVSSGGGGSGGGGSGGGGSQSVLTQPPSASGTPGQRVTISCSGSSSNIGRNTVNWYQQLPGTAPKLLIYGNSNRRSGVPDRFSGSKSGTSASLAISGLRSEDEADYYCAAWDDSLSGVVFGGXTKLTVLXEQKLISEXDLSGSAA

SEQ ID NO: 149 - protein, 261 aa, Artificial Sequence
  MCP-1 (3) scFv antibody
  VARIANT (219)..(219): Any amino acid
  VARIANT (239)..(239): Any amino acid
  VARIANT (253)..(253): Any amino acid
EVQLLESGGGLVQPGGSLRLSCAASGFTFSNYGMHWVRQAPGKGLEWVAVISYDGSNKYYADSVKGRFTISRDNSKNTLYLQMNSLRAEDTAVYYCAKSHYYDTTSFDYWGQGTLVTVSSGGGGSGGGGSGGGGSQSVLTQPPSASGTPGQRVTISCSGSSSNIGTNPVNWYQQLPGTAPKLLIYDNNKRPSGVPDRFSGSKSGTSASLAISGLRSEDXADYYCAAWDDSLSGVVFGGXTKLTVLGEQKLISXEDLSGSAA

SEQ ID NO: 150 - protein, 263 aa, Artificial Sequence
  MCP-3 (1) scFv antibody
  VARIANT (222)..(222): Any amino acid
  VARIANT (241)..(241): Any amino acid
EVQLLESGGGLVQPGGSLRLSCAASGFTFSTYGMHWVRQAPGKGLEWVSGVSWNGSRTHYVNSVKRRFTISRDNSKNTLYLQMNSLRAEDTAVYYCARVAPGSGKRLRAFDIWGQGTLVTVSSGGGGSGGGGSGGGGSQSVLTQPPSASGTPGQRVTISCSGSSSNIGNNAVNWYQQLPGTAPKLLIYEVSKRPPGVPDRFSGSKSGTSASLAISGLRSEDXADYYCSSYAGSSKWVFGGXTKLTVLGEQKLISEEDLSGSAA

SEQ ID NO: 151 - protein, 256 aa, Artificial Sequence
  MCP-3 (2) scFv antibody
  VARIANT (234)..(234): Any amino acid
  VARIANT (248)..(250): Any amino acids
EVQLLESGGGLVQPGGSLRLSCAASGFTLSSNYMSWVRQAPGKGLEWVSGISASGHSTHYADSGKARFTISRDNSKNTLYLQMNSLRAEDTAVYYCARGKSLAYWGQGTLVTVSSGGGGSGGGGSGGGGSQSVLTQPPSASGTPGQRVTISCSGSSSNIGNNAVNWYQQLPGTAPKLLIYRNNQRPSGVPDRFSGSKSGTSASLAISGLRSEDEADYYCAAWDDSLSVVVFGGXTKLTVLGEQKLISXXXLSGSAA

SEQ ID NO: 152 - protein, 261 aa, Artificial Sequence
  MCP-3 (3) scFv antibody
  VARIANT (221)..(221): Any amino acid
  VARIANT (238)..(239): Any amino acids
  VARIANT (254)..(255): Any amino acids
EVQLLESGGGLVQPGGSLRLSCAASGFTFSIYWMSWVRQAPGKGLEWVAYIGGISNTVSYSDSVKGRFTISRDNSKNTLYLQMNSLRAEDTAVYYCAKAPGYSSGWGWFDPWGQGTLVTVSSGGGGSGGGGSGGGGSQSVLTQPPSASGTPGQRVTISCSGSSSNIGTNSVFWYQQLPGTAPKLLIYGNNNRPSGVPDRFSGSKSGTSASLAISGLRSEDXADYYCMIWHSSASVFGXXTKLTVLGEQKLISEXXLSGSAA

SEQ ID NO: 153 - protein, 252 aa, Artificial Sequence
  beta-gal scFv antibody
  VARIANT (200)..(200): Any amino acid
  VARIANT (235)..(235): Any amino acid
  VARIANT (243)..(243): Any amino acid
EVQLLESGGGLVQPGGSLRLSCAASGFTFSSYAMHWVRQAPGKGLEWVAVIAYDGINEYYGDSVKGRFTISRDNSKNTLYLQMNSLRAEDTAVYYCARGGIYHGFDIWGQGTLVTVSSGGGGSGGGGSGGGGSQSVLTQPPSASGTPGQRVTISCSGSSSNIGSNYVYWYQQLPGTAPKLLIYDNHKRPSGVPDRFSGSXSGTSASLAISGLRSEDEADYYCAAWDDNSWVFGGXTKLTVLGXYKDDDDKAA

SEQ ID NO: 154 - protein, 305 aa, Artificial Sequence
  Angiomotin (1) scFv antibody
  VARIANT (201)..(201): Any amino acid
  VARIANT (218)..(218): Any amino acid
  VARIANT (238)..(238): Any amino acid
  VARIANT (248)..(248): Any amino acid
  VARIANT (261)..(262): Any amino acids
  VARIANT (267)..(268): Any amino acids
  VARIANT (281)..(281): Any amino acid
  VARIANT (292)..(292): Any amino acid
  VARIANT (294)..(295): Any amino acids
  VARIANT (298)..(298): Any amino acid
  VARIANT (300)..(302): Any amino acids
  VARIANT (304)..(305): Any amino acids
EVQLLESGGGLVQPGGSLRLSCAASGFTFSDHYMDWVRQAPGKGLEWVSGVSWNGSRTHYADSVKGRFTISRDNSKNTLYLQMNSLRAEDTAVYYCARDTWAYGAFDIWGQGTLVTVSSGGGGSGGGGSGGGGSQSVLTQPPSASGTPGQRVTISCSGSNSNIGRNTVNWYQQLPGTAPKLLIYRDNQRPSGVPDRFSGSXSGTPASLAISGLRSEDXADYYCAAWDVSLNGWVFGGXTKLTVLGDYXDHDGDYKDHDIDXXDDDDXXAAHHHHHHSPRWXIRPIVSRITIXWXXFYXVXXXKXX

SEQ ID NO: 155 - protein, 320 aa, Artificial Sequence
  Angiomotin (2) scFv antibody
  VARIANT (237)..(237): Any amino acid
  VARIANT (245)..(245): Any amino acid
  VARIANT (265)..(265): Any amino acid
  VARIANT (268)..(268): Any amino acid
  VARIANT (279)..(281): Any amino acids
  VARIANT (283)..(283): Any amino acid
  VARIANT (287)..(287): Any amino acid
  VARIANT (289)..(289): Any amino acid
  VARIANT (292)..(294): Any amino acids
  VARIANT (298)..(299): Any amino acids
  VARIANT (304)..(309): Any amino acids
  VARIANT (311)..(312): Any amino acids
  VARIANT (315)..(317): Any amino acids
  VARIANT (319)..(320): Any amino acids
EVQLLESGGGLVQPGGSLRLSCAASGFTFNDYYMTWIRQAPGKGLEWVSYISSSGSTIYYADSVKGRFTISRDNSKNTLYLQMNSLRAEDTAVYYCARERLPDVFDVWGQGTLVTVSSGGGGSGGGGSGGGGSQSVLTQPPSASGTPGQRVTISCSGSGSNIGTNSVSWYQQLPGTAPKLLIYFDDLLPSGVPDRFSGSKSGTSASLAISGLRSEDEADYYCAAWDDSLSGVVFGGXTKLTVLGXYKDHDGDYKDHDIDYKDDDXKAXAHHHHHHSPRXXXRXIVSXIXIHXXXFYNXXTGKTXXXXXXIXXAAXXXFXX

SEQ ID NO: 156 - protein, 256 aa, Artificial Sequence
  Leptin scFv antibody
  VARIANT (239)..(239): Any amino acid
  VARIANT (247)..(247): Any amino acid
  VARIANT (251)..(251): Any amino acid
EVQLLESGGGLVQPGGSLRLSCAASGFTFGDFAMSWVRQAPGKGLEWVANIKQDGSVKYYVDSVKGRFTISRDNSKNTLYLQMNSLRAEDTAVYYCARFLAGFYYGMDVWGQGTLVTVSSGGGGSGGGGSGGGGSQSVLTQPPSASGTPGQRVTISCSGSDSNIGGNTVNWYQQLPGMAPKLLIYYDDLLPSGVPDRFSGSKSGTSASLAISGLRSEDEADYYCAAYDDTMNGWGFGGXTKLTVLGXYKDXDDKAA

SEQ ID NO: 157 - protein, 261 aa, Artificial Sequence
  Integrin alpha-10 scFv antibody
  VARIANT (239)..(239): Any amino acid
  VARIANT (254)..(254): Any amino acid
EVQLLESGGGLVQPGGSLRLSCAASGFTFSTYNMNWVRQAPGKGLEWVSTISGSGGRTYYADSVKGRFTISRDNSKNTLYLQMNSLRAEDTAVYYCARDRVATLDAFDIWGQGTLVTVSSGGGGSGGGGSGGGGSQSVLTQPPSASGTPGQRVTISCSGSSSNIGSNSVSWYQQLPGTAPKLLIYSNNQRPSGVPDRFSGSKSGTSASLAISGLRSEDEADYYCAAWDDSLSGVVFGGXTKLTVLGEQKLISEXDLSGSAA

SEQ ID NO: 158 - protein, 262 aa, Artificial Sequence
  Integrin alpha-11 scFv antibody
  VARIANT (202)..(202): Any amino acid
  VARIANT (232)..(232): Any amino acid
  VARIANT (240)..(240): Any amino acid
  VARIANT (247)..(247): Any amino acid
  VARIANT (255)..(256): Any amino acids
EVQLLESGGGLVQPGGSLRLSCAASGFTFRRDWMSWVRQVPGKGLEWVSVISGSDGSTYYADSVKGRFTISRDNSKNTLYLQMNSLRAEDTAVYYCASYSPLGNWFDSWGQGTLVTVSSGGGGSGGGGSGGGGSQSVLTQPPSASGTPGQRVTISCTGSSSNIGAGYDVHWYQQLPGTAPKLLIYSDTYRPSGVPDRFSGSXSGTSASLAISGLRSEDEADYYCQSYDSSLXGFVVFGGXTKLTVLXEQKLISEXXLSGSAA

SEQ ID NO: 159 - protein, 257 aa, Artificial Sequence
  IgM (1) scFv antibody
  VARIANT (235)..(235): Any amino acid
  VARIANT (239)..(239): Any amino acid
  VARIANT (249)..(251): Any amino acids
EVQLLESGGGLVQPGGSLRLSCAASGFTFSDYYMSWIRQAPGKGLEWVSAIGSGPYYAHSVRDRFTISRDNSKNTLYLQMNSLRAEDTAVYYCARGGVEASFDYWGQGTLVTVSSGGGGSGGGGSGGGGSQSVLTQPPSASGTPGQRVTISCTGSSSNIGAGYDVHWYQQLPGTAPKLLIYGNTNRPSGVPNRFSGSKSGTSASLAISGLRSEDEADYYCQSYDNDLSGWVFGGXTKLXVLGEQKLISXXXLSGSAA

SEQ ID NO: 160 - protein, 261 aa, Artificial Sequence
  LDL (1) scFv antibody
  VARIANT (254)..(255): Any amino acids
EVQLLESGGGLVQPGGSLRLSCAASGFTFSDYYMSWVRQAPGKGLEWVSGVSWNGSRTHYADSVKGRFTISRDNSKNTLYLQMNSLRAEDTAVYYCARAARYSYYYYGMDVWGQGTLVTVSSGGGGSGGGGSGGGGSQSVLTQPPSASGTPGQRVTISCSGSSSNIGNNAVNWYQQLPGTAPKLLIYGNDRRPSGVPDRFSGSKSGTSASLAISGLRSEDEADYYCQTWGTGRGVFGGGTKLTVLGEQKLISEXXLSGSAA

SEQ ID NO: 161 - protein, 275 aa, Artificial Sequence
  LDL (2) scFv antibody
  VARIANT (198)..(198): Any amino acid
  VARIANT (207)..(207): Any amino acid
  VARIANT (224)..(224): Any amino acid
  VARIANT (244)..(244): Any amino acid
  VARIANT (251)..(252): Any amino acids
  VARIANT (261)..(261): Any amino acid
  VARIANT (270)..(271): Any amino acids
EVQLLESGGGLVQPGGSLRLSCAASGFTFSNAWMSWVRQAPGKGLEWVSSISTSSNYIYYADSVKGRFTISRDNSKNTLYLQMNSLRAEDTAVYYCARVKKYSSGWYSNYAFDIWGQGTLVTVSSGGGGSGGGGSGGGGSQSVLTQPPSASGTPGQRVTISCSGSSSSIGNNFVSWYQQLPGTAPKLLIYDNNKRPSXVPDRFSGSXSGTSASLAISGLRSEDXADYYCAAWDDSLNGWVFGGXTKLTVLXXYKDHDGDYXDHDIDYKDXXDKAA

SEQ ID NO: 162 - protein, 262 aa, Artificial Sequence
  PSA scFv antibody
  VARIANT (207)..(207): Any amino acid
  VARIANT (245)..(245): Any amino acid
  VARIANT (257)..(258): Any amino acids
EVQLLESGGGLVQPGGSLRLSCAASGFTFRSYEMNWVRQAPGKGLEWVAVIGGNGVDTDYADSVKGRFTISRDNSKNTLYLQMNSLRAEDTAVYYCVREEVDFWSGYYSYGMDVWGQGTLVTVSSGGGGSGGGGSGGGGSQSVLTQPPSASGTPGQRVTISCSGSSSNIGDNFVSWYQQLPGTAPKLLIYRTNGRPSGVPDRFSGSXSGTSASLAISGLRSEDEADYYCATWDDNLNGRVVFGGXTKLTVLGDYKDXXDKAA

SEQ ID NO: 163 - protein, 255 aa, Artificial Sequence
  Lewis x (1) scFv antibody
  VARIANT (238)..(238): Any amino acid
EVQLLESGGGLVQPGGSLRLSCAASGFTFSNYWMHWVRQAPGKGLEWVANIKEDGSEKYYVDSVKGRFTISRDNSKNTLYLQMNSLRAEDTAVYYCAREGETSFGLDVWGQGTLVTVSSGGGGSGGGGSGGGGSQSVLTQPPSASGTPGQRVTISCSGSSSNIGSNTVNWYQQLPGTAPKLLIYSNNQRPSGVPDRFSGSKSGTSASLAISGLRSEDEADYYCASWDDSLSGWVFGGXTKLTVLGDYKDDDDKAA

SEQ ID NO: 164 - protein, 258 aa, Artificial Sequence
  Lewis x (2) scFv antibody
  VARIANT (219)..(219): Any amino acid
  VARIANT (241)..(241): Any amino acid
  VARIANT (249)..(249): Any amino acid
  VARIANT (253)..(254): Any amino acids
EVQLLESGGGLVQPGGSLRLSCAASGFTFSRYWMHWVRQAPGKGLEWVANIKPDGSEQYYADSVKGRFTISRDNSKNTLYLQMNSLRAEDTAVYYCAREGLSSGWSYGMDVWGQGTLVTVSSGGGGSGGGGSGGGGSQSVLTQPPSASGTPGQRVTISCSGSNSNIGSNTVNWYQQLPGTAPKLLIYTNINRPSGVPDRFSGSKSGTSASLAISGLRSXDEADYYCATWDDSLSGWVFGGXTKLTVLGXYKDXXDKAA

SEQ ID NO: 165 - protein, 258 aa, Artificial Sequence
  Lewis y scFv antibody
  VARIANT (220)..(220): Any amino acid
  VARIANT (241)..(241): Any amino acid
  VARIANT (248)..(248): Any amino acid
  VARIANT (252)..(252): Any amino acid
EVQLLESGGGLVQSGGSLRLSCAASGFTFSSYTLHWVRQAPGKGLEYVSAISSNGGSTYYADSVKGRFTISRDNSKNTLYLQMNSLRAEDTAVYYCASDVYGDYPRGLDYWGQGTLVTVSSGGGGSGGGGSGGGGSQSVLTQPPSASGTPGQRVTISCSGTTSNIGSNYVHWYQQLPGTAPKLLIYGNNNRPSGVPDRFSGSKSGTSASLAISGLRSEDXADYYCQSYDRSLGGLRVFGGXTKLTVLXDYKXDDDKAA

SEQ ID NO: 166 - protein, 294 aa, Artificial Sequence
  Sialle x scFv antibody
  VARIANT (217)..(217): Any amino acid
  VARIANT (237)..(237): Any amino acid
  VARIANT (252)..(252): Any amino acid
  VARIANT (258)..(258): Any amino acid
  VARIANT (265)..(272): Any amino acids
  VARIANT (274)..(291): Any amino acids
  VARIANT (293)..(294): Any amino acids
EVQLLESGGGLVQPGGSLRLSCAASGFTLSSYAMSWVRQAPGKGLEWVSSISSGNSYIYYADSVKGRFTISRDNSKNTLYLQMNSLRAEDTAVYYCARGRGRGGGFELWGQGTLVTVSSGGGGSGGGGSGGGGSQSVLTQPPSASGTPGQRVTISCSGSSSNIGTYTVNWYQQLPGTAPKLLIYSNNQRPSGVPDRFSGSKSGTSASLAISGLRSEXEADYYCSSNAGIDNILFGGXTKLTVLGEQKLISEXDLSGSXAAHHHHXXXXXXXXIXXXXXXXXXXXXXXXXXXLXX

SEQ ID NO: 167 - protein, 264 aa, Artificial Sequence
  TM peptide scFv antibody
  VARIANT (158)..(158): Any amino acid
  VARIANT (188)..(188): Any amino acid
  VARIANT (196)..(197): Any amino acids
  VARIANT (207)..(207): Any amino acid
  VARIANT (233)..(233): Any amino acid
  VARIANT (242)..(243): Any amino acids
  VARIANT (246)..(246): Any amino acid
  VARIANT (255)..(257): Any amino acids
  VARIANT (261)..(261): Any amino acid
EVQLLESGGGLVQPGGSLRLSCAASGFTFSSYGFHWVRQAPGKGLEWVSLISWDGGSTYYADSVKGRFTISRDNSKNTLYLQMNSLRAEDTAVYYCARGTWFDPWGQGTLVTVSSGGGGSGGGGSGGGGSQSVLTQPPSASGTPGQRVTISCSGSSSXIGNNAVNWYQQLPGTAPKLLIYRNNQRPSXVPDRFSGXXSGTSASLAIXGLRSEDEADYYCAAWDDSLSWVFGGXTKLTVLGDXXTMXVIIKIMTSXXXMTMXRRP

SEQ ID NO: 168 - protein, 254 aa, Artificial Sequence
  Procathepsin W scFv antibody
  VARIANT (200)..(200): Any amino acid
  VARIANT (237)..(237): Any amino acid
  VARIANT (245)..(245): Any amino acid
  VARIANT (248)..(248): Any amino acid
EVQLLESGGGLVQPGGSLRLSCAASGFTFSSYAMSWVRQAPGKGLEWVSSMSASGGSTYYADSVKGRFTISRDNSKNTLYLQMNSLRAEDTAVYYCARDRGSYGMDVWGQGTLVTVSSGGGGSGGGGSGGGGSQSVLTQPPSASGTPGQRVTISCSGSTSNIGSYAVNWYQQLPGTAPKLLIYGNNNRPSGVPDRFSGSXSGTSASLAISGPRSEDEADYYCAAWDDSLNGGVFGGXTKLTVLGXYKXDDDKAA

SEQ ID NO: 169 - protein, 188 aa, Artificial Sequence
  BTK (1) scFv antibody
  VARIANT (166)..(166): Any amino acid
EVQLLESGGGLVQPGGSLRLSCAASGFTFSNYAMSWVRQAPGKGLEWVSGINWNGGSTGYADSVKGRFTISRDNSKNTLYLQMNSLRAEDTAVYYCAKHLKRYSGSSYLFDYWGQGTLVTVSSGGGGSGGGGSGGGGSQSVLTQPPSASGTPGQRVTISCSGSSSXIGSNYVYWYQQLPGTAPKLLIY

SEQ ID NO: 170 - protein, 258 aa, Artificial Sequence
  Digoxin scFv antibody
  VARIANT (236)..(236): Any amino acid
  VARIANT (250)..(252): Any amino acids
  VARIANT (255)..(255): Any amino acid
EVQLLESGGGLVQPGGSLRLSCAASGFTFSSYAMSWVRQAPGKGLEWVAVIWHDGSSKYYADSVKGRFTISRDNSKNTLYLQMNSLRAEDTAVYYCARATGDGFDYWGQGTLVTVSSGGGGSGGGGSGGGGSQSVLTQPPSASGTPGQRVTISCSGSSSNIGSNYVYWYQQLPGTAPKLLIYRNNQRPSGVPDRFSGSKSGTSASLAISGLRSEDEADYYCAAWDDSLNGVVFGGXTKLTVLGEQKLISXXXLSXSAA

SEQ ID NO: 171 - protein, 262 aa, Artificial Sequence
  GLP-1 R scFv antibody
  VARIANT (245)..(245): Any amino acid
EVQLLESGGGLVQPGGSLRLSCAASGFTFRSYGMHWVRQAPGKGLEWVSGLSWNSAGTGYADSVKGRFTISRDNSKNTLYLQMNSLRAEDTAVYYCAKEMGNNWDHIDYWGQGTLVTVSSGGGGSGGGGSGGGGSQSVLTQPPSASGTPGQRVTISCTGSSSNIGAGYDVHWYQQLPGTAPKLLIYGNSNRPSGVPDRFSGSKSGTSASLAISGLRSEDEADYYCAAWDDGLSGPVFGGGTKLTXLGEQKLISEEDLSGSAA

SEQ ID NO: 172 - protein, 260 aa, Artificial Sequence
  GLP-1 scFv antibody
  VARIANT (238)..(238): Any amino acid
  VARIANT (245)..(245): Any amino acid
  VARIANT (252)..(252): Any amino acid
  VARIANT (254)..(254): Any amino acid
EVQLLESGGGLVQPGGSLRLSCAASGFTFNSYGMHWVRQAPGKGLEWVSAISGSGGSTYYAESVKGRSTISRDNSKNTLYLQMNSLRAEDTAVYYCVTRNAVFGFDVWGQGTLVTVSSGGGGSGGGGSGGGGSQSVLTQPPSASGTPGQRVTISCTGSSSNIGAGFDVHWYQQLPGTAPKLLIYDNNKRPSGVPDRFSGSKSGTSASLAISGLRSEDEADYYCQSFDSSLSGVVFGGXTKLTVLXEQKLISXEXLSGSAA

SEQ ID NO: 173 - protein, 320 aa, Artificial Sequence
  C1q scFv antibody
  VARIANT (197)..(197): Any amino acid
  VARIANT (223)..(223): Any amino acid
  VARIANT (244)..(244): Any amino acid
  VARIANT (259)..(260): Any amino acids
  VARIANT (267)..(268): Any amino acids
  VARIANT (283)..(283): Any amino acid
  VARIANT (286)..(286): Any amino acid
  VARIANT (289)..(290): Any amino acids
  VARIANT (295)..(300): Any amino acids
  VARIANT (302)..(303): Any amino acids
  VARIANT (306)..(307): Any amino acids
  VARIANT (309)..(311): Any amino acids
  VARIANT (313)..(313): Any amino acid
  VARIANT (315)..(320): Any amino acids
EVQLLESGGGLVQPGGSLRLSCAASGFTFDDYGMSWVRQVPGKGLEWVSAISGSGATTFYAHSVQGRFTISRDNSKNTLYLQMNSLRAEDTAVYYCARGGRGYDWPSGAFDIWGQGTLVTVSSGGGGSGGGGSGGGGSQSVLTQPPSASGTPGQRVTISCTGSSSNIGAGYDVHWYQQLPGTAPKLLIYENNKRPSXVPDRFSGSKSGTSASLAISGLRSEDXADYYCAAWDDSVNGYVVFGGXTKLTVLGEQKLISEXXLSGSAAXXHHHHHSPRWPIRPIXSRXTIXXPSFYXXXXXXTXXLPXXIXXXHXPXXXXXX

SEQ ID NO: 174 - protein, 262 aa, Artificial Sequence
  C1s scFv antibody
  VARIANT (202)..(202): Any amino acid
  VARIANT (240)..(241): Any amino acids
  VARIANT (247)..(247): Any amino acid
  VARIANT (250)..(250): Any amino acid
  VARIANT (254)..(256): Any amino acids
EVQLLESGGGLVQPGGSLRLSCAASGFTFSSYAMSWVRQAPGKGLEWVSGVSWNGSRTHYADSVKGRFTISRDNSKNTLYLQMNSLRAEDTAVYYCARHMKAAAYVFEIWGQGTLVTVSSGGGGSGGGGSGGGGSQSVLTQPPSASGTPGQRVTISCSGSSSNIGSTAVNWYQQLPGTAPKLLIYSNNKRPSGVPDRFSGSXSGTSASLAISGLRSEDEADYYCAAWDDRLNGNVLFGGXXKLTVLXEQXLISXXXLSGSAA

SEQ ID NO: 175 - protein, 261 aa, Artificial Sequence
  C3 (1) scFv antibody
  VARIANT (239)..(239): Any amino acid
  VARIANT (253)..(255): Any amino acids
EVQLLESGGGLVQPGGSLRLSCAASGFTFSSYAMSWVRQAPGKGLEWVSSVTGSGGGTYYADSVEGRFTISRDNSKNTLYLQMNSLRAEDTAVYYCARYRWFGNDAFDIWGQGTLVTVSSGGGGSGGGGSGGGGSQSVLTQPPSASGTPGQRVTISCSGSASNLGMHFVSWYQQLPGTAPKLLIYGNSNRPSGVPDRFSGSKSGTSASLAISGLRSEDEADYYCAAWDDTLNIWVFGGXTKLTVLGEQKLISXXXLSGSAA

SEQ ID NO: 176 - protein, 263 aa, Artificial Sequence
  C3 (2) scFv antibody
  VARIANT (220)..(220): Any amino acid
  VARIANT (241)..(241): Any amino acid
  VARIANT (249)..(249): Any amino acid
EVQLLESGGGLVQPGGSLRLSCAASGFTFSTYRMIWVRQAPGKGLEWVSSISGSNTYIHYADSVRGRFTISRDNSKNTLYLQMNSLRAEDTAVYYCARDRHPLLPSGMDVWGQGTLVTVSSGGGGSGGGGSGGGGSQSVLTQPPSASGTPGQRVTISCSGSSSNIGKHPVNWYQQLPGTAPKLLIYRNDQRPSGVPDRFSGSKSGTSASLAISGLRSEDXADYYCQSYDSSLSGSWVFGGXTKLTVLGXQKLISEEDLSGSAA

SEQ ID NO: 177 - protein, 263 aa, Artificial Sequence
  C4 (1) scFv antibody
  VARIANT (241)..(241): Any amino acid
  VARIANT (248)..(248): Any amino acid
  VARIANT (256)..(257): Any amino acids
EVQLLESGGGLVQPGGSLRLSCAASGFTFSSYPMSWVRQAPGKGLEWVSTLYAGGWTSYADSVWGRFTISRDNSKNTLYLQMNSLRAEDTAVYYCARPKVESLSRYGMDVWGQGTLVTVSSGGGGSGGGGSGGGGSQSVLTQPPSASGTPGQRVTISCTGSSSNIGAGYVVHWYQQLPGTAPKLLIYDNSKRPSGVPDRFSGSKSGTSASLAISGLRSEDEADYYCQSYDSSLSGVVFGGXTKLTVLXEQKLISEXXLSGSAA

SEQ ID NO: 178 - protein, 264 aa, Artificial Sequence
  C5 (1) scFv antibody
  VARIANT (196)..(196): Any amino acid
  VARIANT (205)..(205): Any amino acid
  VARIANT (215)..(215): Any amino acid
  VARIANT (222)..(222): Any amino acid
  VARIANT (240)..(240): Any amino acid
  VARIANT (242)..(243): Any amino acids
  VARIANT (249)..(249): Any amino acid
  VARIANT (257)..(258): Any amino acids
  VARIANT (263)..(263): Any amino acid
EVQLLESGGGLVQPGGSLRLSCAASGFTFSSYRMNWVRQAPGKGLEWVSAISGSGGSTYYADSVKGRFTISRDNSKNTLYLQMNSLRAEDTAVYYCARGGGWFSGHYYFDYWGQGTLVTVSSGGGGSGGGGSGGGGSQSVLTQPPSASGTPGQRVTISCTGATSNIGAGYDVHWYQQLPGTAPKLLIYRNNQRPSXVPDRFSGSXSGTSASLAIXGLRSEDXADYYCQSYDSSLRHWVFXGXXKLTVLXEQKLISEXXLSGSXA

SEQ ID NO: 179 - protein, 258 aa, Artificial Sequence
  C5 (2) scFv antibody
  VARIANT (216)..(216): Any amino acid
  VARIANT (236)..(236): Any amino acid
  VARIANT (243)..(243): Any amino acid
  VARIANT (252)..(252): Any amino acid
EVQLLESGGGLVQPGGSLRLSCAASGFTFSAYSMNWVRQAPGKGLEWVSGVSWNGSRTHYADSVKGRFTISRDNSKNTLYLQMNSLRAEDTAVYYCARENSGFFDYWGQGTLVTVSSGGGGSGGGGSGGGGSQSVLTQPPSASGTPGQRVTISCTGSSSNIGSNTVNWYQQLPGTAPKLLIYGNSNRPSGVPDRFSGSKSGTSASLTISGLRSEDXADYYCAAWDDSLSGWVFGGXTKLTVLXEQKLISEEXLSGSAA

SEQ ID NO: 180 - protein, 261 aa, Artificial Sequence
  C1 inh. (1) scFv antibody
  VARIANT (19)..(19): Any amino acid
  VARIANT (218)..(219): Any amino acids
  VARIANT (239)..(239): Any amino acid
  VARIANT (246)..(246): Any amino acid
  VARIANT (254)..(255): Any amino acids
  VARIANT (258)..(258): Any amino acid
EVQLLESGGGLVQPGGSLXLSCAASGFTFSDYYMSWIRQAPGKGLEWVSGISRGGEYTFYVDSVKGRFTISRDNSKNTLYLQMNSLRAEDTAVYYCARDPGGLDAFDIWGQGTLVTVSSGGGGSGGGGSGGGGSQSVLTQPPSASGTPGQRVTISCTGSSSNIGARYDVQWYQQLPGTAPKLLIYGNSNRPSGVPDRFSGSKSGTSASLAISGLRSEXXADYYCASWDDSLSGPVFGGXTKLTVLXEQKLISEXXLSXSAA

SEQ ID NO: 181 - protein, 262 aa, Artificial Sequence
  Factor B (1) scFv antibody
  VARIANT (219)..(219): Any amino acid
  VARIANT (240)..(240): Any amino acid
  VARIANT (255)..(255): Any amino acid
EVQLLESGGGLVQPGGSLRLSCAASGFTFSSYSMNWVRQAPGKGLEWVAVISYDGRFIYYSDSVKGRFTISRDNSKNTLYLQMNSLRAEDTAVYYCARSYGGNLAMDVWGQGTLVTVSSGGGGSGGGGSGGGGSQSVLTQPPSASGTPGQRVTISCTGSSSNIGAGYDVHWYQQLPGTAPKLLIYDNNKRPSGVPDRFSGSNSGTSASLAISGLRSEDXADYYCAAWDDRLNGRVVFGGXTKLTVLGEQKLISEXDLSGSAA

SEQ ID NO: 182 - protein, 262 aa, Artificial Sequence
  IL-12 (3) scFv antibody
  VARIANT (163)..(163): Any amino acid
  VARIANT (220)..(220): Any amino acid
  VARIANT (240)..(241): Any amino acids
  VARIANT (255)..(255): Any amino acid
EVQLLESGGGLVQPGGSLRLSCAASGFTFSRYGMHWVRQAPGKGLEWVASIRGNARGSFYADSVKGRFTISRDNSKNTLYLQMNSLRAEDTAVYYCAKGDSSGWYFFDYWGQGTLVTVSSGGGGSGGGGSGGGGSQSVLTQPPSASGTPGQRVTISCTGSDSXIGAGFDVHWYQQLPGTAPKLLIYGNNNRPSGVPDRFSGSKSGTSASLAISGLRSEDXADYYCQSYDTSLSGVLFGGXXKLTVLGEQKLISEXDLSGSAA

SEQ ID NO: 183 - protein, 254 aa, Artificial Sequence
  IL-12 (4) scFv antibody
  VARIANT (232)..(232): Any amino acid
  VARIANT (239)..(239): Any amino acid
  VARIANT (246)..(246): Any amino acid
EVQLLESGGGLVQPGGSLRLSCAASGFTFSDYGMHWVRQAPGKGLEWVSTVSGSGDNTYYADSVKGRFTISRDNSKNTLYLQMNSLRAEDTAVYYCTTTWRYWGQGTLVTVSSGGGGSGGGGSGGGGSQSVLTQPPSASGTPGQRVTISCSGSSSNIGSNTVNWYQQLPGTAPKLLIYGNSNRPSGVPDRFSGSKSGTSASLAISGLRSEDEADYYCQSYDSSLSGWVFGGXTKLTVLXEQKLISXEDLSGSAA

SEQ ID NO: 184 - protein, 259 aa, Artificial Sequence
  IL-16 (3) scFv antibody
  VARIANT (3)..(3): Any amino acid
  VARIANT (214)..(215): Any amino acids
  VARIANT (225)..(225): Any amino acid
  VARIANT (237)..(237): Any amino acid
  VARIANT (241)..(241): Any amino acid
EVXLLESGGGLVQPGGSLRLSCAASGFTFSSYGMHWVRQAPGKGLEWVSGINWNGGSTGYADSVKGRFTISRDNSKNTLYLQMNSLRAEDTAVYYCARERGDAFDIWGQGTLVTVSSGGGGSGGGGSGGGGSQSVLTQPPSASGTPGQRVTISCSGSSSNIGSNTVNWYQQLPGTAPKLLIYSDNQRPSGVPDRFSGSKSGTSASLAISGLRSXXEADYYCAAWXDSLNGPWVFGGXTKLXVLGEQKLISEEDLSGSAA

SEQ ID NO: 185 - protein, 263 aa, Artificial Sequence
  IL-18 (3) scFv antibody
  VARIANT (204)..(204): Any amino acid
  VARIANT (220)..(221): Any amino acids
  VARIANT (241)..(241): Any amino acid
  VARIANT (245)..(245): Any amino acid
  VARIANT (255)..(257): Any amino acids
  VARIANT (260)..(260): Any amino acid
EVQLLESGGGLVQPGGSLRLSCAASGFTFSRYGMHWVRQAPGKGLEWVAVISYDGSNKYYADSVKGRFTISRDNSKNTLYLQMNSLRAEDTAVYYCARHGYGDSRSAFDIWGQGTLVTVSSGGGGSGGGGSGGGGSQSVLTQPPSASGTPGQRVTISCTGSSSNIGAGYDVHWYQQLPGTAPKLLIYRNNQRPSGVPDRFSGSXSGTSASLAISGLRSEXXADYYCQSYDSSLSRWVFGGXTKLXVLGEQKLISXXXLSXSAA

SEQ ID NO: 186 - protein, 266 aa, Artificial Sequence
  IL-1a (3) scFv antibody
  VARIANT (169)..(169): Any amino acid
  VARIANT (199)..(199): Any amino acid
  VARIANT (208)..(208): Any amino acid
  VARIANT (227)..(227): Any amino acid
  VARIANT (233)..(233): Any amino acid
  VARIANT (239)..(239): Any amino acid
  VARIANT (244)..(244): Any amino acid
  VARIANT (258)..(260): Any amino acids
EVQLLESGGGLVQPGGSLRLSCAASGFTFSSYSMNWVRQAPGKGLEWVSYISSSSSYTNYADSVKGRFTISRDNSKNTLYLQMNSLRAEDTAVYYCARSVTRRAGYYYYYSGMDVWGQGTLVTVSSGGGGSGGGGSGGGGSQSVLTQPPSASGTPGQRVTISCSGSSSXIGSNTVNWYQQLPGTAPKLLIYRNNQRPSXVPDRFSGSXSGTSASLAISGLRSEDEAXYYCSSXAGSNSXVFGGXTKLTVLGEQKLISXXXLSGSAA

SEQ ID NO: 187 - protein, 260 aa, Artificial Sequence
  IL-6 (3) scFv antibody
  VARIANT (219)..(219): Any amino acid
  VARIANT (238)..(238): Any amino acid
  VARIANT (253)..(254): Any amino acids
EVQLLESGGGLVQPGGSLRLSCAASGFTFSNYGMHWVRQAPGKGLEWVSSITSSGDGTYFADSVKGRFTISRDNSKNTLYLQMNSLRAEDTAVYYCARAGGIAAAYAFDIWGQGTLVTVSSGGGGSGGGGSGGGGSQSVLTQPPSASGTPGQRVTISCSGSSSNVGSNYVYWYQQLPGTAPKLLIYDNNKRPSGVPDRFSGSKSGTSASLAISGLRSEXEADYYCQSYDSSRWVFGGXTKLTVLGEQKLISEXXLSGSAA

SEQ ID NO: 188 - protein, 319 aa, Artificial Sequence
  IL-6 (4) scFv antibody
  VARIANT (145)..(145): Any amino acid
  VARIANT (214)..(214): Any amino acid
  VARIANT (240)..(240): Any amino acid
  VARIANT (247)..(247): Any amino acid
  VARIANT (254)..(255): Any amino acids
  VARIANT (262)..(262): Any amino acid
  VARIANT (270)..(270): Any amino acid
  VARIANT (274)..(275): Any amino acids
  VARIANT (282)..(282): Any amino acid
  VARIANT (287)..(288): Any amino acids
  VARIANT (292)..(292): Any amino acid
  VARIANT (298)..(298): Any amino acid
  VARIANT (300)..(301): Any amino acids
  VARIANT (306)..(308): Any amino acids
  VARIANT (311)..(311): Any amino acid
  VARIANT (314)..(317): Any amino acids
  VARIANT (319)..(319): Any amino acid
EVQLLESGGGLVQPGGSLRLSCAASGFTFSSNYMSWVRQAPGKGLEWVSSISSSSTIYYADSVKGRFTISRDNSKNTLYLQMNSLRAEDTAVYYCARQPASGTYDAFDIWGQGTLVTVSSGGGGSGGGGSGGGGSQSVLTQPPSXSGTPGQRVTISCTGSSSNIGAGYDVHWYQQLPGTAPKLLIYYDDLLPSGVPDRFSGSKSGTSASLAISXLRSEDEADYYCAVWDDSLSGWVFGGXTKLTVLXEQKLISXXDLSGSAXAHHHHHHXSPRXXIRPIVSXITIHXXVVLXRRDWEXPXXTQLNXXXAHXPFXXXXNX

SEQ ID NO: 189 - protein, 259 aa, Artificial Sequence
  IL-8 (3) scFv antibody
  VARIANT (4)..(4): Any amino acid
  VARIANT (215)..(215): Any amino acid
  VARIANT (237)..(237): Any amino acid
  VARIANT (252)..(253): Any amino acids
EVQXLESGGGLVQPGGSLRLSCAASGFTFDDYGMSWVRQAPGKGLEWVSLISWDGGSTYYADSVKGRFTISRDNSKNTLYLQMNSLRAEDTAVYYCARDDLYGMDVWGQGTLVTVSSGGGGSGGGGSGGGGSQSVLTQPPSASGTPGQRVTISCTGSSSNIGAGYDVHWYQQLPGTAPKLLIYDNNKRPSGVPDRFSGSKSGTSASLAISGLRSXDEADYYCAAWDDSLSGWVFGGXTKLTVLGEQKLISEXXLSGSAA

SEQ ID NO: 190 - protein, 261 aa, Artificial Sequence
  MCP-4 (3) scFv antibody
  VARIANT (218)..(218): Any amino acid
  VARIANT (239)..(239): Any amino acid
  VARIANT (243)..(243): Any amino acid
  VARIANT (246)..(246): Any amino acid
  VARIANT (254)..(255): Any amino acids
EVQLLESGGGLVQPGGSLRLSCAASGFTFSSYGMHWVRQAPGKGLEWVSGISWNGGKTHYVDSVKGQFTISRDNSKNTLYLQMNSLRAEDTAVYYCARGGYSSGWAFDYWGQGTLVTVSSGGGGSGGGGSGGGGSQSVLTQPPSASGTPGQRVTISCSGRSSNIESNTVNWYQQLPGTAPKLLIYGNSNRPSGVPDRFSGSKSGTSASLAISGLRSEXEADYYCAAWDDRLNAVVFGGXTKLXVLXEQKLISEXXLSGSAA

SEQ ID NO: 191 - protein, 313 aa, Artificial Sequence
  Properdin scFv antibody
  VARIANT (222)..(222): Any amino acid
  VARIANT (228)..(228): Any amino acid
  VARIANT (244)..(244): Any amino acid
  VARIANT (247)..(247): Any amino acid
  VARIANT (262)..(262): Any amino acid
  VARIANT (273)..(274): Any amino acids
  VARIANT (287)..(288): Any amino acids
  VARIANT (290)..(294): Any amino acids
  VARIANT (298)..(300): Any amino acids
  VARIANT (302)..(302): Any amino acid
  VARIANT (304)..(309): Any amino acids
  VARIANT (313)..(313): Any amino acid
EVQLLESGGGLVQPGGSLRLSCAASGFTFSSNYMSWVRQAPGKGLEWVSAISGSGGSTYYADSVKGRFTISRDNSKNTLYLQMNSLRAEDTAVYYCAKGGSGWYDYFDYWGQGTLVTVSSGGGGSGGGGSGGGGSQSVLTQPPSASGTPGQRVTISCTGSSSNIGAGYDVHWYQQLPGTAPKLLIYRNNQRPSGVPDRFSGSKSGTSASLAISGLRSEDEAXYYCAAXDDGLNSPVFGGGTKLXVLXEQKLISEEDLSGSAXAHHHHHHSPRXXIRPIVSRITIHWXXFXXXXXGKTXXXPXLXXXXXXPPFX

SEQ ID NO: 192 - protein, 255 aa, Artificial Sequence
  TNF-beta (3) scFv antibody
  VARIANT (213)..(213): Any amino acid
  VARIANT (233)..(233): Any amino acid
  VARIANT (248)..(248): Any amino acid
EVQLLESGGGLVQPGGSLRLSCAASGFTFSDYYMSWIRQAPGKGLEWVSGLSGSAGRTHYADSVRGRFTISRDNSKNTLYLQMNSLRAEDTAMYYCASSLFDYWGQGTLVTVSSGGGGSGGGGSGGGGSQSVLTQPPSASGTPGQRVTISCSGSSSNIGSNYVYWYQQLPGTAPKLLIYSNNQRPSGVPDRFSGSKSGTSASLAISGLRSEDXADYYCAAWDDSLNAVVFGGXTKLTVLGEQKLISEXDLSGSAA

SEQ ID NO: 193 - protein, 262 aa, Artificial Sequence
  TNF-beta (4) scFv antibody
  VARIANT (240)..(240): Any amino acid
  VARIANT (254)..(256): Any amino acids
EVQLLESGGGLVQPGGSLRLSCAASGFTFSDYYMNWVRQAPGKGLEWVSGINWNSDDIDYVDSVKGRFTISRDNSKNTLYLQMNSLRAEDTAMYYCAIDSRYSSGWSFEYWGQGTLVTVSSGGGGSGGGGSGGGGSQSVLTQPPSASGTPGQRVTISCSGSTSNIGNSHVYWYQQLPGTAPKLLIYSNNQRPSGVPDRFSGSKSGTSASLAISGLRSEDEADYYCQSYDSSLSGVVFGGXTKLTVLGEQKLISXXXLSGSAA

SEQ ID NO: 194 - protein, 304 aa, Artificial Sequence
  VEGF (3) scFv antibody
  VARIANT (192)..(192): Any amino acid
  VARIANT (201)..(201): Any amino acid
  VARIANT (218)..(218): Any amino acid
  VARIANT (241)..(241): Any amino acid
  VARIANT (244)..(244): Any amino acid
  VARIANT (247)..(247): Any amino acid
  VARIANT (254)..(256): Any amino acids
  VARIANT (261)..(261): Any amino acid
  VARIANT (273)..(273): Any amino acid
  VARIANT (281)..(281): Any amino acid
  VARIANT (283)..(283): Any amino acid
  VARIANT (288)..(288): Any amino acid
  VARIANT (293)..(296): Any amino acids
  VARIANT (298)..(300): Any amino acids
  VARIANT (302)..(302): Any amino acid
  VARIANT (304)..(304): Any amino acid
EVQLLESGGGLVQPGGSLRLSCAASGFTFSSYEMNWVRQAPGKGLEWVSGISGSGGFTYYADSVKGRFTISRDNSKNTLYLQMNSLRAEDTAMYYCAREGYQDAFDIWGQGTLVTVSSGGGGSGGGGSGGGGSQSVLTQPPSASGTPGQRVTISCTGSSSNIGAGYDVHWYQQLPGTAPKLLIYSNNQRPSXVPDRFSGSXSGTSASLAISGLRSEDXADYYCAAWDDSLSGPPWVFGGGXKLXVLXEQKLISXXXLSGSXAAHHHHHHSPRXPIRPIVSXIXIHWPXFYNVXXXXTXXXPXLX

SEQ ID NO: 195 - protein, 261 aa, Artificial Sequence
  VEGF (4) scFv antibody
  VARIANT (30)..(32): Any amino acids
  VARIANT (34)..(34): Any amino acid
  VARIANT (50)..(50): Any amino acid
  VARIANT (54)..(55): Any amino acids
  VARIANT (97)..(104): Any amino acids
  VARIANT (193)..(193): Any amino acid
  VARIANT (202)..(202): Any amino acid
  VARIANT (219)..(219): Any amino acid
  VARIANT (232)..(233): Any amino acids
  VARIANT (239)..(239): Any amino acid
  VARIANT (253)..(255): Any amino acids
EVQLLESGGGLVQPGGSLRLSCAASGFTFXXXYXSWVRQAPGKGLEWVSXISWXXGSIGYADSVKGRFTISRDNSKNTLYLQMNSLRAEDTAVYYCXXXXXXXXNYFDYWGQGTLVTVSSGGGGSGGGGSGGGGSQSVLTQPPSASGTPGQRVTISCSGSNSNIGGNFVYWYQQLPGTAPKLLIYENSKRPSXVPDRFSGSXSGTSASLAISGLRSEDXADYYCAAWDDSLXXVVFGGXTKLTVLGEQKLISXXXLSGSAA

SEQ ID NO: 196 - protein, 260 aa, Artificial Sequence
  IL-4 (3) scFv antibody
  VARIANT (218)..(218): Any amino acid
  VARIANT (238)..(239): Any amino acids
  VARIANT (253)..(253): Any amino acid
EVQLLESGGGLVQPGGSLRLSCAASGFTFSNAWMSWVRQAPGKGLEWVSAISGSGGSTYYADSVKGRFTISRDNSKNTLYLQMNSLRAEDTAVYYCARAIAARPFDYWGQGTLVTVSSGGGGSGGGGSGGGGSQSVLTQPPSASGTPGQRVTISCTGATSNIGAGYDIHWYQQLPGTAPKLLIYSTNNRPSGVPDRFSGSKSGTSASLAISGLRSEDXADYYCAAWDDSLNGPVFGGXXKLTVLGEQKLISEXDLSGSAA

SEQ ID NO: 197 - protein, 161 aa, Artificial Sequence
  CD40 (2) scFv antibody
EVQLLESGGGLVQPGGSLRLSCAASGFTFSAYWMHWVRQAPGKGLEWVSGISGGGGSTYYADSVKGRFTISRDNSKNTLYLQMNSLRAEDTAVYYCARMTPWYYGMDVWGQGTLVTVSSGGGGSGGGGSGGGGSQSMLTQPPSASGTPGQRVTISCSGSTS

SEQ ID NO: 198 - protein, 156 aa, Artificial Sequence
  CD40 (3) scFv antibody
  VARIANT (143)..(144): Any amino acids
EVQLLESGGGLVQPGGSLRLSCAASGFTFSTYGMHWVRQAPGKGLEWLSYISGGSSYIFYADSVRGRFTISRDNSENALYLQMNSLRAEDTAVYYCARILRGGSGMDLWGQGTLVTVSSGGGGSGGGGSGGGGSQSVLTQPPXXSGTPGQRVTISC

SEQ ID NO: 199 - protein, 256 aa, Artificial Sequence
  CD40 (4) scFv antibody
  VARIANT (219)..(219): Any amino acid
  VARIANT (232)..(232): Any amino acid
  VARIANT (239)..(240): Any amino acids
  VARIANT (246)..(247): Any amino acids
EVQLLESGGGLVQPGGSLRLSCAASGFTFSTYGMHWVRQAPGKGLEWLSYISGGSSYIFYADSVRGRFTISRDNSENALYLQMNSLRAEDTAVYYCARILRGGSGMDLWGQGTLVTVSSGGGGSGGGGSGGGGSQSVLTQPPSASGTPGQRVTISCTGSSSNIGAGYDVYWYQQLPGTAPKLLIYGNINRPSGVPDRFSGSKSGTSASLAISGLRSEDXADYYCAAWDDSLXGLVFGGXXKLTVLXXYKDDDDKAA

SEQ ID NO: 200 - protein, 267 aa, Artificial Sequence
  CT17 scFv antibody
  VARIANT (228)..(228): Any amino acid
  VARIANT (250)..(250): Any amino acid
  VARIANT (257)..(257): Any amino acid
  VARIANT (262)..(262): Any amino acid
  VARIANT (264)..(264): Any amino acid
EVQLLESGGGLVQPGGSLRLSCAASGFTFSSSAMHWVRQAPGKGLEWVSAISGSGGSTYYADSVKGRFTISRDNSKNTLYLQMNSLRAEDTAVYYCARVKGRVTIFGVVINSNYGMDVWGQGTLVTVSSGGGGSGGGGSGGGGSQSVLTQPPSASGTPGQRVTISCSGSISSIGSNAVSWYQQLPGTAPKLLIYGNSNRPSGVPDRFSGSKSGTSASLAISGLRSEDXADYYCAAWDDSLNGHDVVFGGXTKLTVLXDYKDXDXKAA

SEQ ID NO: 201 - protein, 265 aa, Artificial Sequence
  IgM (3) scFv antibody
  VARIANT (168)..(168): Any amino acid
  VARIANT (198)..(198): Any amino acid
  VARIANT (206)..(207): Any amino acids
  VARIANT (217)..(217): Any amino acid
  VARIANT (222)..(222): Any amino acid
  VARIANT (224)..(224): Any amino acid
  VARIANT (232)..(233): Any amino acids
  VARIANT (243)..(243): Any amino acid
  VARIANT (257)..(258): Any amino acids
EVQLLESGGGLVQPGGSLRLSCAASGFTFSSYAMSWVRQAPGKGLEWVSGISWNSGSIGYADSVKGRFTISRDNSKNTLYLQMNSLRAEDTAVYYCARGDYSSSPGGYYYYMDVWGQGTLVTVSSGGGGSGGGGSGGGGSQSVLTQPPSASGTPGQRVTISCSGSSSXIGSNTVNWYQQLPGTAPKLLIYGNSNRPSXVPDRFSGXXSGTSASLAIXGLRSXDXADYYCSSXXSTNTVIFGGXTKLTVLGEQKLISXXDLSGSAA

SEQ ID NO: 202 - protein, 262 aa, Artificial Sequence
  IgM (4) scFv antibody
  VARIANT (193)..(193): Any amino acid
  VARIANT (202)..(202): Any amino acid
  VARIANT (240)..(240): Any amino acid
  VARIANT (244)..(244): Any amino acid
  VARIANT (254)..(255): Any amino acids
EVQLLESGGGLVQPGGSLRLSCAASGFTFSSNEMSWIRQAPGKGLEWVSAIYSGGGTYYADSVKGRFTISRDNSKNTLYLQMNSLRAEDTAVYYCARVNDYGDNVYFDHWGQGTLVTVSSGGGGSGGGGSGGGGSQSVLTQPPSASGTPGQRVTISCSGSSSNIGNNYVSWYQQLPGTAPKLLIYENNKRPSXVPDRFSGSXSGTSASLAISGLRSEDEADYYCAAWDDSLSVYVVFGGXTKLXVLGEQKLISXXDLSGSAA

SEQ ID NO: 203 - protein, 261 aa, Artificial Sequence
  IgM (5) scFv antibody
  VARIANT (193)..(193): Any amino acid
  VARIANT (202)..(202): Any amino acid
  VARIANT (239)..(239): Any amino acid
  VARIANT (246)..(246): Any amino acid
  VARIANT (253)..(253): Any amino acid
  VARIANT (255)..(255): Any amino acid
EVQLLESGGGLVQPGGSLRLSCAASGFTFGSYEMNWVRQAPGKGLEWVSVIYSGGSTYYADSVEGRFTISRDNSKNTLYLQMNSLRAEDTAVYYCARDTNPYYYYGMDVWGQGTLVTVSSGGGGSGGGGSGGGGSQSVLTQPPSASGTPGQRVTISCSGSSSNIGNNAVNWYQQLPGTAPKLLIYRNNQRPSXVPDRFSGSXSGTSASLAISGLRSEDEADYYCQSYDSSLNGQVFGGXTKLTVLXEQKLISXEXLSGSAA

SEQ ID NO: 204 - protein, 259 aa, Artificial Sequence
  HLA-DR/DP scFv antibody
  VARIANT (190)..(190): Any amino acid
  VARIANT (199)..(199): Any amino acid
  VARIANT (216)..(216): Any amino acid
  VARIANT (237)..(237): Any amino acid
  VARIANT (244)..(244): Any amino acid
  VARIANT (251)..(252): Any amino acids
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCARDGLLPLDYWGQGTLVTVSSGGGGSGGGGSGGGGSQSVLTQPPSASGTPGQRVTISCSGGSSNIGGNAVNWYQQLPGTAPKLLIYENNKRPSXVPDRFSGSXSGTSASLAISGLRSEDXADYYCSSYAVSNNFEVLFGGXTKLTVLXEQKLISXXDLSGSAA

SEQ ID NO: 205 - protein, 308 aa, Artificial Sequence
  ICAM-1 scFv antibody
  VARIANT (160)..(160): Any amino acid
  VARIANT (191)..(191): Any amino acid
  VARIANT (200)..(200): Any amino acid
  VARIANT (237)..(237): Any amino acid
  VARIANT (251)..(252): Any amino acids
  VARIANT (258)..(258): Any amino acid
  VARIANT (274)..(274): Any amino acid
  VARIANT (278)..(279): Any amino acids
  VARIANT (282)..(283): Any amino acids
  VARIANT (285)..(285): Any amino acid
  VARIANT (288)..(288): Any amino acid
  VARIANT (290)..(292): Any amino acids
  VARIANT (295)..(296): Any amino acids
  VARIANT (298)..(298): Any amino acid
  VARIANT (300)..(300): Any amino acid
  VARIANT (302)..(303): Any amino acids
  VARIANT (307)..(308): Any amino acids
EVQLLESGGGLVQPGGSLRLSCAASGFTFSNAWMSWVRQAPGKGLEWVAFIWYDGSNKYYADSVKGRFTISRDNSKNTLYLQMNSLRAEDTAVYYCARYSGWYFDYWGQGTLVTVSSGGGGSGGGGSGGGGSQSVLTQPPSASGTPGQRVTISCTGSSSXIGAGYDVHWYQQLPGTAPKLLIYDNNNRPSXVPDRFSGSXSGTSASLAISGLRSEDEADYYCQSYDSSLSAWLFGGXTKLTVLGEQKLISXXDLSGSXAAHHHHHHSPRWPIRXIVSXXTIXXPXFYXVXXXKPXXTXLXRXXAHPXX

SEQ ID NO: 206 - protein, 257 aa, Artificial Sequence
  IgM (2) scFv antibody
  VARIANT (189)..(189): Any amino acid
  VARIANT (198)..(198): Any amino acid
  VARIANT (235)..(235): Any amino acid
  VARIANT (251)..(251): Any amino acid
EVQLLESGGGLVQPGGSLRLSCAASGFTFSDYYMSWIRQAPGKGLEWVSAIGSGPYYAHSVRDRFTISRDNSKNTLYLQMNSLRAEDTAVYYCARGGVEASFDYWGQGTLVTVSSGGGGSGGGGSGGGGSQSVLTQPPSASGTPGQRVTISCTGSSSNIGAGYDVHWYQQLPGTAPKLLIYGNTNRPSXVPNRFSGSXSGTSASLAISGLRSEDEADYYCQSYDNDLSGWVFGGXTKLTVLGEQKLISEEXLSGSAA

SEQ ID NO: 207 - protein, 112 aa, Artificial Sequence
  MCP-1 (4) scFv antibody
QSVLTQPPSASGTPGQRVTISCTGSSSNIGSDYGVQWYQQLPGTAPKLLIYSNNQRPSGVPDRFSGSKSGTSASLAISGLRSEDEADYYCAAWDDSLSGPVFGGGTKLTVLG

SEQ ID NO: 208 - protein, 112 aa, Artificial Sequence
  MCP-1 (5) scFv antibody
QSVLTQPASASGTPGQRVTISCTGNSSNIGAGYDVHWYQQLPGTAPKLLIYRNNQRPSGVPDRFSGSKSGTSASLAISGLLSEDEADYYCAAWDYSLNGWVFGGGTKLTVLG

SEQ ID NO: 209 - protein, 113 aa, Artificial Sequence
  MCP-1 (6) scFv antibody
QSVLTQPSSASGTPGQRVTISCTGNSSNIGAGYDVHWYQQLPGTAPNLLIYRNNQRPSGVPDRFSGSKSGTSASLAISGLRSEDEADYYCAAWDDSLNGWVFGGGTKLTVLGQ

SEQ ID NO: 210 - protein, 242 aa, Artificial Sequence
  MCP-1 (7) scFv antibody
  VARIANT (196)..(196): Any amino acid
  VARIANT (222)..(222): Any amino acid
  VARIANT (242)..(242): Any amino acid
EVQLLESGGGLVQPGGSLRLSCAASGFTFSSYAMSWVRQAPGKGLEWVAYINRGSTYTNYADSMKGRFTISRDNSKNTLYLQMNSLRAEDTAVYYCARRGYGSGSYYAFDIWGQGTLVTVSSGGGGSGGGGSGGGGSQSVLTQPPSASGTPGQRVTISCTGSSSNIGSDYGVQWYQQLPGTAPKLLIYSNNQRPSXVPDRFSGSKSGTSASLAISGLRSEDXADYYCAAWDDSLSGPVFGGX

SEQ ID NO: 211 - protein, 240 aa, Artificial Sequence
  MCP-1 (8) scFv antibody
  VARIANT (195)..(195): Any amino acid
  VARIANT (204)..(204): Any amino acid
  VARIANT (221)..(221): Any amino acid
EVQLLESGGGLVQPGGSLRLSCAASGFTFGDYAMSWVRQAPGKGLEWVAVISYDGSNKYYADSVKGRFTISRDNSKNTLYLQMNSLRAEDTAVYYCARDPDPSGTDAFDFWGQGTLVTVSSGGGGSGGGGSGGGGSQSVLTQPPSASGTPGQRVTISCSGSSSNIGAGYDVHWYQQLPGTAPKLLIYGNSNRPSXVPDRFSGSXSGTSASLAISGLRSEDXADYYCAAWDDSLSAWAFGG

SEQ ID NO: 212 - protein, 237 aa, Artificial Sequence
  MCP-1 (9) scFv antibody
  VARIANT (192)..(193): Any amino acids
  VARIANT (202)..(202): Any amino acid
EVQLLESGGGLVQPGGSLRLSCAASGFTFSSYEMNWVRQAPGKGLEWVSAISGPGGSTYYADSVKGRFTISRDNSKNTLYLQMNSLRAEDTAVYYCARDLSDYGDFDYWGQGTLVTVSSGGGGSGGGGSGGGGSQSVLTQPPSASGTPGQRVTISCTGSSSNIGAGYDAHWYQQLPGTAPKLLIYDNNKRPXXVPDRFSGSXSGTSASLAISGLRSEDEADYYCATWDDSLRGWVFG

SEQ ID NO: 213 - protein, 268 aa, Artificial Sequence
  Cystatin C (1) scFv antibody
  VARIANT (191)..(191): Any amino acid
  VARIANT (200)..(200): Any amino acid
  VARIANT (237)..(237): Any amino acid
  VARIANT (247)..(247): Any amino acid
  VARIANT (254)..(254): Any amino acid
  VARIANT (260)..(261): Any amino acids
EVQLLESGGGLVQPGGSLRLSCAASGFTFSSYAMNWVRQAPGKGLEWVGLISYDGRTTYYADSVKGRSTISRDNSKNTLYLQMNSLRAEDTAVYYCATTTGTTLDYWGQGTLVTVSSGGGGSGGGGSGGGGSQSVLTQPPSASGTPGQRVTISCTGSSSNIGAGYDVHWYQQLPGTAPKLLIYGNTNRPSXVPDRFSGSXSGTSASLAISGLRSEDEADYYCAAWDDSLYGWVFGGXTKLTVLGDYXDHDGDYXDHDIDXXDDDDKAA

SEQ ID NO: 214 - protein, 192 aa, Artificial Sequence
  Cystatin C (2) scFv antibody
EVQLLESGGGLVQPGGSLRLSCAASGFTFSSYSMNWVRQAPGKGLEWVAFISYDGSNKYYVDSVKGRFTISRDNSKNTLYLQMNSLRAEDTAVYYCARDGVPAVPFDYWGQGTLVTVSSGGGGSGGGGSGGGGSQSVLTQPPSASGTPGQRVTISCSGSSSNIGAGYDVHWYQQLPGTAPKLLIYSNNQRPS

SEQ ID NO: 215 - protein, 196 aa, Artificial Sequence
  Cystatin C (3) scFv antibody
  VARIANT (167)..(167): Any amino acid
EVQLLESGGGLVQPGGSLRLSCAASGFTFSNYAMTWVRQAPGKGLEWVADISHDGFHKYYADSVRGRFTISRDNSKNTLYLQMNSLRAEDTAVYYCARYGRVLPYYYYYGMDVWGQGTLVTVSSGGGGSGGGGSGGGGSQSVLTQPPSASGTPRQRVTISCTGSSSXIGAGYDVHWYQQLPGTAPKLLIYGNSNRP

SEQ ID NO: 216 - protein, 236 aa, Artificial Sequence
  Cystatin C (4) scFv antibody
  VARIANT (191)..(191): Any amino acid
  VARIANT (200)..(200): Any amino acid
  VARIANT (205)..(205): Any amino acid
  VARIANT (215)..(215): Any amino acid
EVQLLESGGGLVQPGGSLRLSCAASGFTFSSYAMNWVRQAPGKGLEWVGLISYDGRTTYYADSVKGRSTISRDNSKNTLYLQMNSLRAEDTAVYYCATTTGTTLDYWGQGTLVTVSSGGGGSGGGGSGGGGSQSVLTQPPSASGTPGQRVTISCTGSSSNIGAGYDVHWYQQLPGTAPKLLIYGNTNRPSXVPDRFSGSXSGTSXSLAISGLRSXDEADYYCAAWDDSLYGWVFGG

SEQ ID NO: 217 - protein, 187 aa, Artificial Sequence
  Apo-A1 (1) scFv antibody
EVQLLESGGGLVQPGGSLRLSCAASGFTFSNNGMHWVRQAPGKGLEWVSAISASGGSTYYADSVKGRFTISRDNSKNTLYLQMNSLRAEDTAVYYCATHGGSSYDAFDIWGQGTLVTVSSGGGGSGGGGSGGGGSQSVLTQPPSASGTPGQRVTISCTGSSSNIGAGYVVHWYQQLPGTAPKLLIYG

SEQ ID NO: 218 - protein, 196 aa, Artificial Sequence
  Apo-A1 (2) scFv antibody
  VARIANT (166)..(166): Any amino acid
EVQLLESGGGLVQPGGSLRLSCAASGFTFRDYYMSWIRQAPGKGLEWVAVTSYDGSKKYYADSVKGRFTISRDNSKNTLYLQMNSLRAEDTAVYYCAKDYADDSIAAPAFDIWGQGTLVTVSSGGGGSGGGGSGGGGSQSVLTQPPSASGTPGQRVTISCTGSSSXIGAGYDVHWYQQLPGTAPKLLIYGNSNRPS

SEQ ID NO: 219 - protein, 315 aa, Artificial Sequence
  Apo-A1 (3) scFv antibody
  VARIANT (3)..(4): Any amino acids
  VARIANT (221)..(221): Any amino acid
  VARIANT (225)..(225): Any amino acid
  VARIANT (249)..(250): Any amino acids
  VARIANT (252)..(252): Any amino acid
  VARIANT (266)..(266): Any amino acid
  VARIANT (269)..(271): Any amino acids
  VARIANT (273)..(273): Any amino acid
  VARIANT (283)..(285): Any amino acids
  VARIANT (288)..(290): Any amino acids
  VARIANT (292)..(293): Any amino acids
  VARIANT (297)..(304): Any amino acids
  VARIANT (307)..(315): Any amino acids
EVXXLESGGGLVQPGGSLRLSCAASGFTFRDYYMSWIRQAPGKGLEWVAVTSYDGSKKYYADSVKGRFTISRDNSKNTLYLQMNSLRAEDTAVYYCAKDYADDSIAAPAFDIWGQGTLVTVSSGGGGSGGGGSGGGGSQSVLTQPPSASGTPGQRVTISCTGSSSNIGAGYDVHWYQQLPGTAPKLLIYGNSNRPSGVPDRFSGSKSGTSASLAISGLRSXDEAXYYCQSYDSSLSVVFGGGTKLTVLXXYXDHDGDYKDHDIDYXDDXXXAXAHHHHHHSPXXXIRXXXSXXTIHXXXXXXXXDWXXXXXXXXX

SEQ ID NO: 220 - protein, 271 aa, Artificial Sequence
  Factor B (2) scFv antibody
  VARIANT (240)..(240): Any amino acid
  VARIANT (250)..(250): Any amino acid
  VARIANT (263)..(263): Any amino acid
  VARIANT (268)..(268): Any amino acid
EVQLLESGGGLVQPGGSLRLSCAASGFTFSSYSMNWVRQAPGKGLEWVAVISYDGRFIYYSDSVKGRFTISRDNSKNTLYLQMNSLRAEDTAVYYCARSYGGNLAMDVWGQGTLVTVSSGGGGSGGGGSGGGGSQSVLTQPPSASGTPGQRVTISCTGSSSNIGAGYDVHWYQQLPGTAPKLLIYDNNKRPSGVPDRFSGSNSGTSASLAISGLRSEDEADYYCAAWDDRLNGRVVFGGXTKLTVLGDYXDHDGDYKDHDIDXKDDDXKAA

SEQ ID NO: 221 - protein, 226 aa, Artificial Sequence
  Factor B (3) scFv antibody
  VARIANT (160)..(160): Any amino acid
  VARIANT (190)..(190): Any amino acid
  VARIANT (199)..(199): Any amino acid
  VARIANT (209)..(209): Any amino acid
  VARIANT (216)..(216): Any amino acid
EVQLLESGGGLVQPGGSLRLSCAASGFTFSSYSMNWVRQAPGKGLEWVAVISYDGSNQYYADSVRGRFTISKDNSKNTLYLQMNSLRAEDTAVYYCAREWHYSLDVWGQGTLVTVSSGGGGSGGGGSGGGGSQSVLTQPPSASGTPGQRVTISCSGSSSXIGSNTVNWYQQLPGTAPKLLIYRNNQRPSXVPDRFSGSXSGTSASLAIXGLRSEDXADYYCAAWDD

SEQ ID NO: 222 - protein, 323 aa, Artificial Sequence
  Factor B (4) scFv antibody
  VARIANT (191)..(192): Any amino acids
  VARIANT (201)..(201): Any amino acid
  VARIANT (206)..(206): Any amino acid
  VARIANT (227)..(227): Any amino acid
  VARIANT (229)..(229): Any amino acid
  VARIANT (238)..(238): Any amino acid
  VARIANT (240)..(240): Any amino acid
  VARIANT (245)..(245): Any amino acid
  VARIANT (247)..(248): Any amino acids
  VARIANT (252)..(253): Any amino acids
  VARIANT (260)..(260): Any amino acid
  VARIANT (262)..(262): Any amino acid
  VARIANT (264)..(264): Any amino acid
  VARIANT (266)..(269): Any amino acids
  VARIANT (288)..(288): Any amino acid
  VARIANT (290)..(290): Any amino acid
  VARIANT (292)..(292): Any amino acid
  VARIANT (294)..(295): Any amino acids
  VARIANT (298)..(298): Any amino acid
  VARIANT (300)..(303): Any amino acids
  VARIANT (305)..(316): Any amino acids
  VARIANT (318)..(323): Any amino acids
EVQLLESGGGLVQPGGSLRLSCAASGFTFSKHSMNWVRQAPGKGLEWVATVSYDGNYKYYADSVKGRFTISRDNSKNTLYLQMNSLRAEDTAVYYCAREGYYYYGMDVWGQGTLVTVSSGGGGSGGGGSGGGGSQSVLTQPPSASGTPGQRVTISCTGSSSNIGNNAVNWYQQLPGTAPKLLIYNNNQRPXXVPDRFSGSXSGTSXSLAISGLRSEDEADYYCQPYXDXLSSVVFGGXTXLTVLXDXXDHDXXYKDHDIXYXDXDXXXXAHHHHHHSPRWPIRPIVSXIXIXWXXVLXRXXXXNXXXXXXXXXXXXHXXXXXX

SEQ ID NO: 223 - protein, 272 aa, Artificial Sequence
  C1 inh. (2) scFv antibody
  VARIANT (203)..(203): Any amino acid
  VARIANT (241)..(241): Any amino acid
  VARIANT (248)..(248): Any amino acid
  VARIANT (251)..(251): Any amino acid
  VARIANT (264)..(265): Any amino acids
  VARIANT (270)..(270): Any amino acid
EVQLLESGGGLVQPGGSLRLSCAASGFTFSNAWMSWVRQAPGKGLEWVSAISGSGGSTYYADSVKGRFTISRDNSKNTLYLQMNSLRAEDTAVYYCARNRGNWGTYYFDYWGQGTLVTVSSGGGGSGGGGSGGGGSQSVLTQPPSASGTPGQRVTISCSGSSSNIGSNYVSWYQQLPGTAPKLLIYGSSNRPSGVPDRFSGSXSGTSASLAISGLRSEDEADYYCQSYDSSLSDHVVFGGXTKLTVLXDYXDHDGDYKDHDIDXXDDDDXAA

SEQ ID NO: 224 - protein, 272 aa, Artificial Sequence
  C1 inh. (3) scFv antibody
  VARIANT (164)..(164): Any amino acid
  VARIANT (194)..(194): Any amino acid
  VARIANT (203)..(203): Any amino acid
  VARIANT (220)..(220): Any amino acid
  VARIANT (241)..(241): Any amino acid
  VARIANT (251)..(251): Any amino acid
  VARIANT (262)..(262): Any amino acid
  VARIANT (264)..(265): Any amino acids
  VARIANT (268)..(270): Any amino acids
EVQLLESGGGLVQPGGSLRLSCAASGFTFSNAWMSWVRQAPGKGLEWVSAISGSGGSTYYADSVKGRFTISRDNSKNTLYLQMNSLRAEDTAVYYCARNRGNWGTYYFDYWGQGTLVTVSSGGGGSGGGGSGGGGSQSVLTQPPSASGTPGQRVTISCSGSSSXIGSNYVSWYQQLPGTAPKLLIYGSSNRPSXVPDRFSGSXSGTSASLAISGLRSEDXADYYCQSYDSSLSDHVVFGGXTKLTVLGDYXDHDGDYKDHDXDXXDDXXXAA

SEQ ID NO: 225 - protein, 247 aa, Artificial Sequence
  C1 inh. (4) scFv antibody
  VARIANT (164)..(164): Any amino acid
  VARIANT (194)..(194): Any amino acid
  VARIANT (202)..(203): Any amino acids
  VARIANT (234)..(234): Any amino acid
  VARIANT (241)..(241): Any amino acid
EVQLLESGGGLVQPGGSLRLSCAASGFTFSNAWMSWVRQAPGKGLEWVSAISGSGGSTYYADSVKGRFTISRDNSKNTLYLQMNSLRAEDTAVYYCARNRGNWGTYYFDYWGQGTLVTVSSGGGGSGGGGSGGGGSQSVLTQPPSASGTPGQRVTISCSGSSSXIGSNYVSWYQQLPGTAPKLLIYGSSNRPSXVPDRFSGXXSGTSASLAISGLRSEDEADYYCQSYDSSLSXHVVFGGXTKLTVL

SEQ ID NO: 226 - protein, 263 aa, Artificial Sequence
  C5 (3) scFv antibody
  VARIANT (210)..(210): Any amino acid
  VARIANT (232)..(232): Any amino acid
  VARIANT (239)..(239): Any amino acid
  VARIANT (243)..(243): Any amino acid
  VARIANT (255)..(255): Any amino acid
  VARIANT (260)..(261): Any amino acids
EVQLLESGGGLVQPGGSLRLSCAASGFTFSSYGMHWVRQAPGKGLEWVSYISSSGSTIYYADSVKGRFTISRDNSKNTLYLQMNSLRAEDTAVYYCLTLGGYWGQGTLVTVSSGGGGSGGGGSGGGGSQSVLTQPPSASGTPGQRVTISCSGSSSNIGSNTVNWYQQLPGTAPKLLIYSNNQRPSGVPDRFSGSKSGTSASLAISGLRSXDEADYYCQSYDSSLSGWVFGGXTKLTVLXDYKXHDGDYKDHDIDXKDDDXXAA

SEQ ID NO: 227 - protein, 291 aa, Artificial Sequence
  C4 (2) scFv antibody
  VARIANT (207)..(207): Any amino acid
  VARIANT (212)..(212): Any amino acid
  VARIANT (224)..(224): Any amino acid
  VARIANT (237)..(237): Any amino acid
  VARIANT (244)..(246): Any amino acids
  VARIANT (249)..(249): Any amino acid
  VARIANT (254)..(256): Any amino acids
  VARIANT (261)..(266): Any amino acids
  VARIANT (271)..(275): Any amino acids
  VARIANT (277)..(277): Any amino acid
  VARIANT (279)..(279): Any amino acid
  VARIANT (281)..(283): Any amino acids
  VARIANT (289)..(290): Any amino acids
EVQLLESGGGLVQPGGSLRLSCAASGFTFSDYYMSWVRQAPGKGLEWVSGVSWNGSRTHYADSVKGRFTISRDNSKNTLYLQMNSLRAEDTAVYYCAGYGSGSRATGYNWFAPWGQGTLVTVSSGGGGSGGGGSGGGGSQSVLTQPPSASGTPGQRVTISCTGSSSNIGAGYDVHWYQQLPGTAPKLLIYRNNQRPSGVPDRFSGSXSGTSXSLAISGLRSEDXADYYCQSYDSSLXGPYWVFXXXNQXDGPRXXXKTMTXXXXXXDIDYXXXXXQXRXAXXXHHHSPXXP

SEQ ID NO: 228 - protein, 303 aa, Artificial Sequence
  C4 (3) scFv antibody
  VARIANT (161)..(161): Any amino acid
  VARIANT (184)..(184): Any amino acid
  VARIANT (191)..(191): Any amino acid
  VARIANT (200)..(200): Any amino acid
  VARIANT (210)..(210): Any amino acid
  VARIANT (237)..(237): Any amino acid
  VARIANT (247)..(247): Any amino acid
  VARIANT (254)..(254): Any amino acid
  VARIANT (260)..(261): Any amino acids
  VARIANT (265)..(265): Any amino acid
  VARIANT (268)..(268): Any amino acid
  VARIANT (276)..(277): Any amino acids
  VARIANT (284)..(284): Any amino acid
  VARIANT (287)..(288): Any amino acids
  VARIANT (292)..(294): Any amino acids
  VARIANT (296)..(301): Any amino acids
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCARGWSTSSFDYWGQGTLVTVSSGGGGSGGGGSGGGGSQSVLTQPPSASGTPGQRVTISCSGSSSXIGNHYVSWYQQLPGTATKLLIYXDDLLPSXVPDRFSGSXSGTSASLAIXGLRSEDEADYYCAAWDDRSGQVLFGGXTKLTVLGDYXDHDGDYXDHDIDXXDDDXKAXAHHHHHHXXRWPIRPXVSXXTIHXXXFXXXXXXKT

SEQ ID NO: 229 - protein, 317 aa, Artificial Sequence
  C4 (4) scFv antibody
  VARIANT (160)..(160): Any amino acid
  VARIANT (198)..(199): Any amino acids
  VARIANT (236)..(236): Any amino acid
  VARIANT (240)..(240): Any amino acid
  VARIANT (246)..(246): Any amino acid
  VARIANT (248)..(249): Any amino acids
  VARIANT (259)..(259): Any amino acid
  VARIANT (262)..(265): Any amino acids
  VARIANT (267)..(267): Any amino acid
  VARIANT (270)..(270): Any amino acid
  VARIANT (277)..(277): Any amino acid
  VARIANT (279)..(280): Any amino acids
  VARIANT (285)..(286): Any amino acids
  VARIANT (289)..(292): Any amino acids
  VARIANT (294)..(294): Any amino acid
  VARIANT (298)..(298): Any amino acid
  VARIANT (301)..(302): Any amino acids
  VARIANT (304)..(304): Any amino acid
  VARIANT (306)..(315): Any amino acids
  VARIANT (317)..(317): Any amino acid
EVQLLESGGGLVQPGGSLRLSCAASGFTFSSYSMNWVRQAPGKGLEWVSGISGSGGSTYYADSVKGRFTISRDNSKNTLYLQMNSLRAEDTAVYYCAKHSGYGFDIWGQGTLVTVSSGGGGSGGGGSGGGGSQSVLTQPPSASGTPGQRVTISCSGGASXLGMHFVSWYQQLPGTAPKLLIYYDDLLPSGVPDRFSGXXSGTSASLAISGLRSEDEADYYCAAWDDSLNGWVFGGXTKLXVLGDYXDXXGDYKDHDIDXKDXXXXAXAHXHHHHSPXWXXRPIVXXITXXXXVXLQRXDWXXPXVXXXXXXXXXXPX

SEQ ID NO: 230 - protein, 275 aa, Artificial Sequence
  C3 (3) scFv antibody
  VARIANT (199)..(199): Any amino acid
  VARIANT (208)..(208): Any amino acid
  VARIANT (223)..(223): Any amino acid
  VARIANT (225)..(225): Any amino acid
  VARIANT (244)..(244): Any amino acid
  VARIANT (254)..(254): Any amino acid
  VARIANT (268)..(268): Any amino acid
  VARIANT (270)..(270): Any amino acid
  VARIANT (272)..(273): Any amino acids
EVQLLESGGGLVQPGGSLRLSCAASGFTFSSYAMNWVRQAPGKGLEWVANINQDGSTKFYVDSVKGRFTISRDNSKNTLYLQMNSLRAEDTAVYYCARDTGGNYLGGYYYYGMDVWGQGTLVTVSSGGGGSGGGGSGGGGSQSVLTQPPSASGTPGQRVTISCSGSSSNIGSNYVYWYQQLPGTAPKLLIYRNDQRPSXVPDRFSGSXSGTSASLAISGLRSXDXADYYCSSYAGNNNLVFGGXTKLTVLGDYXDHDGDYKDHDIDYXDXDXXAA

SEQ ID NO: 231 - protein, 273 aa, Artificial Sequence
  C3 (4) scFv antibody
  VARIANT (220)..(220): Any amino acid
  VARIANT (242)..(242): Any amino acid
  VARIANT (250)..(250): Any amino acid
  VARIANT (252)..(252): Any amino acid
  VARIANT (261)..(261): Any amino acid
  VARIANT (269)..(270): Any amino acids
EVQLLESGGGLVQPGGSLRLSCAASGFTFSDHYMDWVRQAPGKGLEWVSGISGNGATIDYADSVKGRFTISRDNSKNTLYLQMNSLRAEDTAVYYCARPSITAAGSEDAFDLWGQGTLVTVSSGGGGSGGGGSGGGGSQSVLTQPPSASGTPGQRVTISCSGSSSNIGSNYVYWYQQLPGTAPKLLIYGNSNRPSGVPDRFSGSKSGTSASLAISGLRSXDGADYYCQSYDSSLSGWVFGGXTKLTVLGXYXDHDGDYKDXDIDYKDDXXKAA

SEQ ID NO: 232 - protein, 247 aa, Artificial Sequence
  C3 (5) scFv antibody
  VARIANT (195)..(195): Any amino acid
  VARIANT (204)..(204): Any amino acid
  VARIANT (214)..(214): Any amino acid
  VARIANT (221)..(221): Any amino acid
  VARIANT (234)..(234): Any amino acid
  VARIANT (239)..(239): Any amino acid
  VARIANT (241)..(241): Any amino acid
EVQLLESGGGLVQPGGSLRLSCAASGFTFSNYWMSWVRQAPGKGLEWVSGISGSGGTTYYADFVKGRFTISRDNSKNTLYLQMNSLRAEDTAVYYCARKYYYGSSGAFDIWGQGTLVTVSSGGGGSGGGGSGGGGSQSVLTQPPSASGTPGQRVTISCTGSSSNIGAGYDVHWYQQLPGTAPKLLIYRNNQRPSXVPDRFSGSXSGTSASLAIXGLRSEDXADYYCAAWDDSLXGPVFXGXTKLTVL

SEQ ID NO: 233 - protein, 275 aa, Artificial Sequence
  C3 (6) scFv antibody
  VARIANT (208)..(208): Any amino acid
  VARIANT (225)..(225): Any amino acid
  VARIANT (244)..(245): Any amino acids
  VARIANT (252)..(254): Any amino acids
  VARIANT (267)..(268): Any amino acids
  VARIANT (270)..(270): Any amino acid
  VARIANT (272)..(273): Any amino acids
EVQLLESGGGLVQPGGSLRLSCAASGFTFSSYAMNWVRQAPGKGLEWVANINQDGSTKFYVDSVKGRFTISRDNSKNTLYLQMNSLRAEDTAVYYCARDTGGNYLGGYYYYGMDVWGQGTLVTVSSGGGGSGGGGSGGGGSQSVLTQPPSASGTPGQRVTISCSGSSSNIGSNYVYWYQQLPGTAPKLLIYRNDQRPSGVPDRFSGSXSGTSASLAISGLRSEDXADYYCSSYAGNNNLVFGGXXKLTVLGXXXDHDGDYKDHDIDXXDXDXXAA

SEQ ID NO: 234 - protein, 220 aa, Artificial Sequence
  MYOM2 (1) scFv antibody
  VARIANT (158)..(158): Any amino acid
  VARIANT (188)..(188): Any amino acid
  VARIANT (196)..(197): Any amino acids
  VARIANT (202)..(202): Any amino acid
  VARIANT (207)..(207): Any amino acid
EVQLLESGGGLVQPGGSLRLSCAASGFTFSSYAMSWVRQAPGKGLEWVSGISGSGGSTYYADSVKGRFTISRDNSKNTLYLQMNSLRAEDTAVYYCARGVVAGSWGQGTLVTVSSGGGGSGGGGSGGGGSQSVLTQPPSASGTPGQRVTISCSGSSSXIGNNAVNWYQQLPGTAPKLLIYDNNKRPSXVPDRFSGXXSGTSXSLAIXGLRSEDEADYYCA

SEQ ID NO: 235 - protein, 246 aa, Artificial Sequence
  MYOM2 (2) scFv antibody
  VARIANT (192)..(192): Any amino acid
  VARIANT (201)..(201): Any amino acid
  VARIANT (218)..(218): Any amino acid
  VARIANT (238)..(238): Any amino acid
EVQLLESGGGLVQPGGSLRLSCAASGFTFSNEWMAWVRQAPGKGLEWVSSISSSSSYIYYADSVKGRFTISRDNSKNTLYLQMNSLRAEDTAVYYCAGTYHDFWSATYWGQGTLVTVSSGGGGSGGGGSGGGGSQSVLTQPPSASGTPGQRVTISCSGSSSNIGSNTVNWYQQLPGTAPKLLIYGNSNRPSXVPDRFSGSXSGTSASLAISGLRSEDXADYYCAAWDDSLNGWVFGGXTKLTVLGD

SEQ ID NO: 236 - protein, 271 aa, Artificial Sequence
  LUM scFv antibody
  VARIANT (203)..(203): Any amino acid
  VARIANT (240)..(240): Any amino acid
  VARIANT (247)..(247): Any amino acid
  VARIANT (250)..(250): Any amino acid
  VARIANT (263)..(265): Any amino acids
  VARIANT (268)..(269): Any amino acids
EVQLLESGGGLVQPGGSLRLSCAASGFTFSSNYMSWVRQAPGKGLEWVSAISASGTYTYYTDSVNGRFTISRDNSKNTLYLQMNSLRAEDTAVYYCARVNTVGLGTPFDNWGQGTLVTVSSGGGGSGGGGSGGGGSQSVLTQPPSASGTPGQRVTISCSGSSSNIGSNTVNWYQQLPGTAPKLLIYGNRNRPSGVPDRFSGSXSGTSASLAISGLRSEDEADYYCAAWDDSLSGWVFGGXTKLTVLXDYXDHDGDYKDHDIDXXXDDXXAA

SEQ ID NO: 237 - protein, 266 aa, Artificial Sequence
  DUSP9 scFv antibody
  VARIANT (197)..(197): Any amino acid
  VARIANT (235)..(235): Any amino acid
  VARIANT (245)..(245): Any amino acid
  VARIANT (263)..(263): Any amino acid
EVQLLESGGGLVQPGGSLRLSCAASGFTFSSYGFHWVRQAPGKGLEWVAVISYDGSNKYYADSVKGRFTISRDNSKNTLYLQMNSLRAEDTAVYYCARGEFGVYWGQGTLVTVSSGGGGSGGGGSGGGGSQSVLTQPPSASGTPGQRVTISCSGSSSNIGSNYVYWYQQLPGTAPKLLIYGNRNRPSGVPDRFSGSXSGTSASLAISGLRSEDEADYYCSSYAGSNNFEVVFGGXTKLTVLGDYXDHDGDYKDHDIDYKDDDXKAA

SEQ ID NO: 238 - protein, 237 aa, Artificial Sequence
  CHX10 (1) scFv antibody
  VARIANT (191)..(191): Any amino acid
  VARIANT (200)..(200): Any amino acid
EVQLLESGGGLVQPGGSLRLSCAASGFTFSSYGMHWVRQAPGKGLEWVAVISYDGSNKYYADSVKGRFTISRDNSKNTLYLQMNSLRAEDTAVYYCARNSDYYGMDVWGQGTLVTVSSGGGGSGGGGSGGGGSQSVLTQPPSASGTPGQRVTISCSGSSSNIGYSDVYWYQQLPGTAPKLLIYENNKRPSXVPDRFSGSXSGTSASLAISGLRSEDEADYYCSTWDDSLNGHVIFGG

SEQ ID NO: 239 - protein, 232 aa, Artificial Sequence
  CHX10 (3) scFv antibody
  VARIANT (163)..(163): Any amino acid
  VARIANT (193)..(193): Any amino acid
  VARIANT (201)..(202): Any amino acids
  VARIANT (207)..(207): Any amino acid
  VARIANT (219)..(219): Any amino acid
  VARIANT (226)..(226): Any amino acid
EVQLLESGGGLVQPGGSLRLSCAASGFTFSSYAMSWVRQAPGKGLEWVAVISYDGSNKYYADSVKGRFTISRDNSKNTLYLQMNSLRAEDTAVYYCARNYGDSINWFDPWGQGTLVTVSSGGGGSGGGGSGGGGSQSVLTQPPSASGTPGQRVTISCSGSSSXIRSNTVNWYQQLPGTAPKLLIYGNSNRPSXVPDRFSGXXSGTSXSLAISGLRSEDXADYYCAXWDDSLN

SEQ ID NO: 240 - protein, 236 aa, Artificial Sequence
  ATP-5B (1) scFv antibody
  VARIANT (161)..(161): Any amino acid
  VARIANT (191)..(191): Any amino acid
  VARIANT (199)..(200): Any amino acids
  VARIANT (217)..(217): Any amino acid
EVQLLESGGGLVQPGGSLRLSCAASGFTFSSYAMHWVRQAPGKGLEWVAVISYDGSKTYHADSVEGRFTISRDNSKNTLYLQMNSLRAEDTAVYYCARHLRPYYFDYWGQGTLVTVSSGGGGSGGGGSGGGGSQSVLTQPPSASGTPGQRVTISCSGSSSXIGSNTVNWYQQLPGTAPKLLIYGNSNRPSXVPDRFSGXXSGTSASLAISGLRSEDXADYYCSAWDDRLRGRVFGG

SEQ ID NO: 241 - protein, 324 aa, Artificial Sequence
  ATP-5B (2) scFv antibody
  VARIANT (201)..(201): Any amino acid
  VARIANT (207)..(207): Any amino acid
  VARIANT (212)..(212): Any amino acid
  VARIANT (244)..(244): Any amino acid
  VARIANT (254)..(255): Any amino acids
  VARIANT (258)..(259): Any amino acids
  VARIANT (268)..(269): Any amino acids
  VARIANT (271)..(271): Any amino acid
  VARIANT (273)..(273): Any amino acid
  VARIANT (275)..(275): Any amino acid
  VARIANT (278)..(278): Any amino acid
  VARIANT (286)..(289): Any amino acids
  VARIANT (294)..(307): Any amino acids
  VARIANT (309)..(309): Any amino acid
  VARIANT (311)..(314): Any amino acids
  VARIANT (317)..(324): Any amino acids
EVQLLESGGGLVQPGGSLRLSCAASGFTFSSYGMHWVRQAPGKGLEWVSLISSASSYIYHADSVKGRFTISRDNSKNTLYLQMNSLRAEDTAVYYCARAGRVCTNGVCHTTFDYWGQGTLVTVSSGGGGSGGGGSGGGGSQSVLTQPPSASGTPGQRVTISCSGDRSNIGSNTVNWYQQLPGTAPKLLIYGNSNRPSGVPXRFSGSXSGTSXSLAISGLRSEDEADYYCQSYDSSLSAVVFGGXTKLTVLGDYXXHDXXYKDHDIDYXXDXDXAXAHXHHHHSPRXXXXPIVSXXXXXXXXXXXXXXLXKXXXXPTXXXXXXXX

SEQ ID NO: 242 - protein, 269 aa, Artificial Sequence
  ATP-5B (3) scFv antibody
  VARIANT (192)..(192): Any amino acid
  VARIANT (201)..(201): Any amino acid
  VARIANT (218)..(218): Any amino acid
  VARIANT (238)..(238): Any amino acid
  VARIANT (242)..(242): Any amino acid
  VARIANT (245)..(245): Any amino acid
  VARIANT (248)..(248): Any amino acid
  VARIANT (255)..(255): Any amino acid
  VARIANT (261)..(263): Any amino acids
EVQLLESGGGLVQPGGSLRLSCAASGFTFSTYAMSWVRQAPGKGLEWVSSISSTSTYIHYADSVKGRFTISRDNSKNTLYLQMNSLRAEDTAVYYCARVSSWYSAFDIWGQGTLVTVSSGGGGSGGGGSGGGGSQSVLTQPPSASGTPGQRVTISCTGSSSNIGNNAVNWYQQLPGTAPKLLIYSNNQRPSXVPDRFSGSXSGTSASLAISGLRSEDXADYYCQSYDSSLSGVIFGGXTKLXVLXDYXDHDGDYXDHDIDXXXDDDKAA

SEQ ID NO: 243 - protein, 267 aa, Artificial Sequence
  Sox11a scFv antibody
  VARIANT (203)..(203): Any amino acid
  VARIANT (220)..(220): Any amino acid
  VARIANT (227)..(227): Any amino acid
  VARIANT (236)..(236): Any amino acid
  VARIANT (243)..(243): Any amino acid
  VARIANT (246)..(246): Any amino acid
  VARIANT (252)..(253): Any amino acids
  VARIANT (259)..(259): Any amino acid
  VARIANT (262)..(262): Any amino acid
  VARIANT (264)..(264): Any amino acid
EVQLLESGGGLVQPGGSLRLSCAASGFTFSDFWMSWVRQAPGKGLEWVSSISGGGGTAFYVDSVKGRFTISRDNSKNTLYLQMNSLRAEDTALYYCARMTDLESGDAFDIWGQGTLVTVSSGGGGSGGGGSGGGGSQSVLTQPPSASGTPGQRVTISCSGSSSNIGSNYVNWYQQLPGTAPKLLIYNDNVRPSGVPDRFSGSXSGTSASLAISGLRSEDXADYYCQXWGTGVFGGXTKLTVLXDYXDHDGDXXDHDIDXKDXDXKAA

SEQ ID NO: 244 - protein, 247 aa, Artificial Sequence
  TBC1D9 (1) scFv antibody
  VARIANT (192)..(193): Any amino acids
  VARIANT (201)..(202): Any amino acids
  VARIANT (239)..(239): Any amino acid
EVQLLESGGGLVQPGGSLRLSCAASGFTFSSYSMSWVRQAPGKGLEWVAVISYDGSNKYYADSVKGRFTISRDNSKNTLYLQMNSLRAEDTAVYYCARDRTRGSTALDIWGQGTLVTVSSGGGGSGGGGSGGGGSQSVLTQPPSASGTPGQRVTISCSGSSSYIGSNYVYWYQQLPGTAPKLLIYRNNQRPXXVPDRFSGXXSGTSASLAISGLRSEDEADYYCAAWDDSLSGWVFGGXTKLTVLGD

SEQ ID NO: 245 - protein, 250 aa, Artificial Sequence
  UPF3B (1) scFv antibody
  VARIANT (187)..(187): Any amino acid
  VARIANT (196)..(196): Any amino acid
  VARIANT (206)..(206): Any amino acid
  VARIANT (212)..(213): Any amino acids
  VARIANT (234)..(234): Any amino acid
  VARIANT (244)..(244): Any amino acid
  VARIANT (248)..(248): Any amino acid
EVQLLESGGGLVQPGGSLRLSCAASGFTFSDYYMTWIRQAPGKGLEWVSDISWNGSRTHYADSVKGRFTISRDNSKNTLYLQMNSLRAEDTAVYYCSSHLVYWGQGTLVTVSSGGGGSGGGGSGGGGSQSVLTQPPSASGTPGQRVTISCTGSSSNIGAGYDVHWYQQLPGTAPKLLIYDNNKRPSXVPDRFSGSXSGTSASLAIXGLRSEXXADYYCQTYDSSLSGSVVFGGXTKLTVLGDYXDHDXDY

SEQ ID NO: 246 - protein, 251 aa, Artificial Sequence
  UPF3B (2) scFv antibody
  VARIANT (166)..(166): Any amino acid
  VARIANT (197)..(197): Any amino acid
  VARIANT (206)..(206): Any amino acid
  VARIANT (221)..(221): Any amino acid
  VARIANT (243)..(243): Any amino acid
EVQLLESGGGLVQPGGSLRLSCAASGFTFSSYAMSWVRQAPGKGLEWVSYISSSSSYANYADSVKGRFTISRDNSKNTLYLQMNSLRAEDTAVYYCARLGVYSGTYLFAFDIWGQGTLVTVSSGGGGSGGGGSGGGGSQSVLTQPPSASGTPGQRVTISCTGSSSXIGAGYDVHWYQQLPGTAPKLLIYGNSNRPSXVPDRFSGSXSGTSASLAISGLRSXDEADYYCQSRDSSLSGWVFGGXTKLTVLGD

SEQ ID NO: 247 - protein, 123 aa, Artificial Sequence
  Apo-A4 (1) scFv antibody
EVQLLESGGGLVQPGGSLRLSCAASGFTFSDYYMSWVRQAPGKGLEWVSGVSWNGSRTHYADSVKGRFTISRDNSKNTLYLQMNSLRAEDTAVYYCARVAYDIDAFDMWGQGTLVTVSSGGGG

SEQ ID NO: 248 - protein, 301 aa, Artificial Sequence
  Apo-A4 (2) scFv antibody
  VARIANT (201)..(201): Any amino acid
  VARIANT (238)..(238): Any amino acid
  VARIANT (245)..(245): Any amino acid
  VARIANT (265)..(268): Any amino acids
  VARIANT (281)..(281): Any amino acid
  VARIANT (285)..(286): Any amino acids
  VARIANT (288)..(289): Any amino acids
  VARIANT (293)..(296): Any amino acids
  VARIANT (298)..(300): Any amino acids
EVQLLESGGGLVQPGGSLRLSCAASGFTFSDYYMSWVRQAPGKGLEWVSGVSWNGSRTHYADSVKGRFTISRDNSKNTLYLQMNSLRAEDTAVYYCARVAYDIDAFDMWGQGTLVTVSSGGGGSGGGGSGGGGSQSVLTQPPSASGTPGQRVTISCSGSFSNIGSNYVYWYQQLPGTAPKLLIYENNKRPSGVPDRFSGSXSGTSASLAISGLRSEDEADYYCAAWDDSLNGPMFGGXTKLTVLXDYKDHDGDYKDHDIDYKDDXXXXAAHHHHHHSPRWXIRPXXSXXTIHXXXXLXXXD

SEQ ID NO: 249 - protein, 232 aa, Artificial Sequence
  Apo-A4 (3) scFv antibody
  VARIANT (196)..(196): Any amino acid
  VARIANT (213)..(213): Any amino acid
EVQLLESGGGLVQPGGSLRLSCAASGFTFSSYSMNWVRQAPGKGLEWVSAITGSGNATFYADSVKGRFTISRDNSKNTLYLQMNSLRAEDTAVYYCTTGATTRWGQGTLVTVSSGGGGSGGGGSGGGGSQSVLTQPPSASGTPGQRVTISCSGSRSNIGSNHVFWYQQLPGTAPKLLIYENNKRPSGVPDRFSGSXSGTSASLAISGLRSEDXADYYCAAWDDSLSGWVFGG

SEQ ID NO: 250 - protein, 196 aa, Artificial Sequence
  TBC1D9 (2) scFv antibody
  VARIANT (168)..(168): Any amino acid
EVQLLESGGGLVQPGGSLRLSCAASGFTFSNAWMSWVRQAPGKGLEWVSFISSSSSYIYYADSVKGRFTISRDNSKNTLYLQMNSLRAEDTAVYYCARVNLVGCTNGVCNGHDYWGQGTLVTVSSGGGGSGGGGSGGGGSQSVLTQPPSASGTPGQRVTISCSGSSSXIGSNTVNWYQQLPGTAPKLLIYDNNKRP

SEQ ID NO: 251 - protein, 234 aa, Artificial Sequence
  TBC1D9 (3) scFv antibody
  VARIANT (159)..(159): Any amino acid
  VARIANT (189)..(189): Any amino acid
  VARIANT (198)..(198): Any amino acid
  VARIANT (215)..(215): Any amino acid
EVQLLESGGGLVQPGGSLRLSCAASGFTFGDYAMSWVRQAPGKGLEWVSAISGSGGSTYYADSVKGRFTISRDNSKNTLYLQMNSLRAEDTAVYYCAKGRTMASHWGQGTLVTVSSGGGGSGGGGSGGGGSQSVLTQPPSASGTPGQRVTISCSGSSSXIGNNHVSWYQQLPGTAPKLLIYGNSNRPSXVPDRFSGSXSGTSASLAISGLRSEDXADYYCAAWDNSLKVWMFGG

SEQ ID NO: 252 - protein, 239 aa, Artificial Sequence
  ORP-3 (1) scFv antibody
  VARIANT (164)..(164): Any amino acid
  VARIANT (193)..(194): Any amino acid
  VARIANT (202)..(203): Any amino acids
  VARIANT (208)..(208): Any amino acid
  VARIANT (220)..(220): Any amino acid
EVQLLESGGGLVQPGGSLRLSCAASGFTFSSNYMSWVRQAPGKGLEWVSYISGNSGYTNYADSVKGRFTISRDNSKNTLYLQMNSLRAEDTAVYYCARHAGSYDMYGMDVWGQGTLVTVSSGGGGSGGGGSGGGGSQSVLTQPPSASGTPGQRVTISCSGSTSXIGSHYVYWYQQLPGTAPKLLIYGNSNRPXXVPDRFSGXXSGTSXSLAISGLRSEDXADYYCQSYDSRLSGWVFGG

SEQ ID NO: 253 - protein, 241 aa, Artificial Sequence
  ORP-3 (2) scFv antibody
  VARIANT (158)..(158): Any amino acid
  VARIANT (197)..(197): Any amino acid
  VARIANT (227)..(227): Any amino acid
  VARIANT (234)..(234): Any amino acid
EVQLLESGGGLVQPGGSLRLSCAASGFTFSSYAMHWVRQAPGKGLEWVAVISYDGSNKYYADSVKGRFTISRDNSKNTLYLQMNSLRAEDTAVYYCARKSSLDVWGQGTLVTVSSGGGGSGGGGSGGGGSQSVLTQPPSASGTPGQRVTISCSGSSSXIGNNYVSWYQQLPGTAPKLLIYDDNKRPSGVPDRFSGSXSDTSASLAISGLRSEDEADYYCAAWDDSLXGRVFGGXTKLTVLG

SEQ ID NO: 254 - protein, 254 aa, Artificial Sequence
  CIMS (5) scFv antibody
  VARIANT (3)..(3): Any amino acid
  VARIANT (232)..(233): Any amino acids
  VARIANT (247)..(247): Any amino acid
EVXLLESGGGLVQPGGSLRLSCAASGFTFSDHYMDWVRQAPGKGLEWVSGISGSGGSTYYGDSVKGRFTISRDNSKNTLYLQMNSLRAEDTAVYYCASRLYWGQGTLVTVSSGGGGSGGGGSGGGGSQSVLTQPPSASGTPGQRVTISCTGSSSNIGAGYVVHWYQQLPGTAPKLLIYDNDKRPSGVPDRFSGSKSGTSASLAISGLRSEDEADYYCAAWDDSLDAVLFGGXXKLTVLGEQKLISEXDLSGSAA

SEQ ID NO: 255 - protein, 321 aa, Artificial Sequence
  CIMS (13) scFv antibody
  VARIANT (206)..(206): Any amino acid
  VARIANT (227)..(227): Any amino acid
  VARIANT (244)..(244): Any amino acid
  VARIANT (258)..(259): Any amino acids
  VARIANT (266)..(266): Any amino acid
  VARIANT (277)..(277): Any amino acid
  VARIANT (286)..(286): Any amino acid
  VARIANT (291)..(293): Any amino acids
  VARIANT (295)..(297): Any amino acids
  VARIANT (302)..(304): Any amino acids
  VARIANT (306)..(306): Any amino acid
  VARIANT (308)..(310): Any amino acids
  VARIANT (312)..(313): Any amino acids
  VARIANT (317)..(321): Any amino acids
EVQLLESGGGLVQPGGSLRLSCAASGFTFSSYAMSWVRQAPGKGLEWVSAISGSGGRTYYTDSVRDRFTISRDNSKNTLYLQMNSLRAEDTAVYYCARDLMPVCQYCYGMDVWGQGTLVTVSSGGGGSGGGGSGGGGSQSVLTQPPSASGTPGQRVTISCTGSSSNIGAGYDVHWYQQLPGTAPKLLIYSNNQRPSGVPDRFSGSXSGTSASLAISGLRSEDEADYXCQSYDSSLNKDVVFGGXTKLTVLGEQKLISXXDLSGSAXAHHHHHHSPRXPIRPIVSRXTIHWXXXLXXXDWENXXXTXLXXXAXXPPFXXXXX